(19) United States
(12) Patent Application Publication
(10) Pub. No.: US 2025/0133957 A1
(43) Pub. Date: Apr. 24, 2025

(54) COMPOUND, LIGHT-EMITTING DEVICE INCLUDING THE SAME, AND ELECTRONIC APPARATUS INCLUDING THE LIGHT-EMITTING DEVICE

(71) Applicant: Samsung Display Co., Ltd., Yongin-si, Gyeonggi-do (KR)

(72) Inventors: Eunjae Jeong, Yongin-si (KR); Byeongwook Yoo, Yongin-si (KR); Jeongmin Lee, Yongin-si (KR); Sohee Jo, Yongin-si (KR)

(21) Appl. No.: 18/674,638

(22) Filed: May 24, 2024

(30) Foreign Application Priority Data
Oct. 23, 2023 (KR) 10-2023-0142339

(51) Int. Cl. H10K 85/60; C07C 211/61; C09K 11/06; H10K 50/12; H10K 50/15

(52) U.S. Cl. CPC H10K 85/633; C07C 211/61; C09K 11/06; C07C 2601/14; C07C 2603/18; C09K 2211/1003; H10K 50/12; H10K 50/15; H10K 85/615; H10K 85/626

(57) ABSTRACT

A compound represented by Formula 1, a light-emitting device including the compound represented by Formula 1, and an electronic apparatus including the light-emitting device are provided.

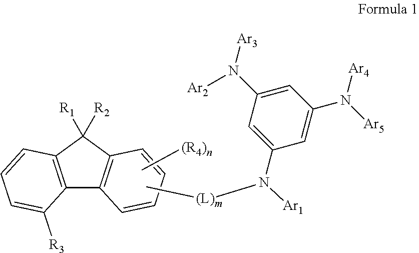

### CROSS-REFERENCE TO RELATED APPLICATION

The present application claims priority to and the benefit of Korean Patent Application No. 10-2023-0142339, filed on Oct. 23, 2023, in the Korean Intellectual Property Office, the entire content of which is incorporated herein by reference.

### BACKGROUND

### 1. Field

One or more embodiments of the present disclosure relate to a compound, a light-emitting device including the compound, and an electronic apparatus including the light-emitting device.

### 2. Description of the Related Art

Light-emitting devices are self-emissive devices that, as compared with comparable devices of the related art, have relatively wide viewing angles, relatively high contrast ratios, relatively short response times, and excellent or suitable characteristics in terms of luminance, driving voltage, and/or response speed.

In a light-emitting device, a first electrode is disposed on a substrate, and a hole transport region, an emission layer, an electron transport region, and a second electrode are sequentially formed on the first electrode. Holes provided from the first electrode move toward the emission layer through the hole transport region, and electrons provided from the second electrode move toward the emission layer through the electron transport region. Carriers, such as the holes and the electrons, recombine in the emission layer to produce light.

### SUMMARY

One or more aspects of embodiments of the present disclosure are directed toward a compound, a light-emitting device including the compound, and an electronic apparatus including the light-emitting device.

Additional aspects will be set forth in part in the description which follows and, in part, will be apparent from the description, or may be learned by practice of the presented embodiments of the disclosure.

According to one or more embodiments of the present disclosure,
there is provided a compound represented by Formula 1.

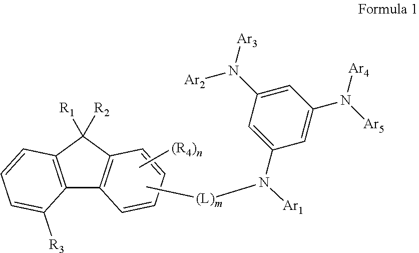

In Formula 1,
R₁, R₂, R₄, and Ar₁ to Ar₅ may each independently be hydrogen, deuterium, a C₁-C₆₀ alkyl group that is unsubstituted or substituted with at least one R₁₀ₐ, a C₃-C₁₀ cycloalkyl group that is unsubstituted or substituted with at least one R₁₀ₐ, a C₁-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ,
R₃ may be a C₁-C₆₀ alkyl group that is unsubstituted or substituted with at least one R₁₀ₐ, a C₃-C₁₀ cycloalkyl group that is unsubstituted or substituted with at least one R₁₀ₐ, or a C₆-C₆₀ aryl group that is unsubstituted or substituted with at least one R₁₀ₐ,
optionally, R₁ and R₂ may be linked together to form a ring,
L may be a single bond, a C₆-C₆₀ arylene group that is unsubstituted or substituted with at least one R₁₀ₐ, a C₁-C₆₀ heteroarylene group that is unsubstituted or substituted with at least one R₁₀ₐ, a divalent non-aromatic condensed polycyclic group that is unsubstituted or substituted with at least one R₁₀ₐ, or a divalent non-aromatic condensed heteropolycyclic group that is unsubstituted or substituted with at least one R₁₀ₐ,
m may be an integer from 0 to 3, wherein, when m is 2 or more, each L may be identical to or different from each other,
n may be an integer from 0 to 3, wherein, when n is 2 or more, each R₄ may be identical to or different from each other,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments of the present disclosure,
a light-emitting device includes the compound.

According to one or more embodiments of the present disclosure,
an electronic apparatus includes the light-emitting device.

According to one or more embodiments of the present disclosure,
electronic equipment includes the electronic apparatus.

### BRIEF DESCRIPTION OF DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a structure of a light-emitting device according to one or more embodiments of the present disclosure;

FIG. 2 is a cross-sectional view of an electronic apparatus according to one or more embodiments of the present disclosure;

FIG. 3 is a cross-sectional view of an electronic apparatus according to one or more embodiments of the present disclosure;

FIG. 4 is a schematic perspective view of electronic equipment including a light-emitting device according to one or more embodiments of the present disclosure;

FIG. 5 is a schematic view of an exterior of a vehicle as electronic equipment including a light-emitting device, according to one or more embodiments of the present disclosure; and

FIGS. 6A, 6B, and 6C are each a schematic view of an interior of a vehicle according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the related art, a phenylenediamine structure is utilized as a hole injection layer material utilized in organic light-emitting devices, and aromatic diamine derivatives, aromatic condensed ring diamine derivatives, etc. are utilized as hole transport layer materials. Carbazole-containing arylamine-based compound as improved compounds of these are utilized as hole transport layer materials. In addition, arylamine-based compounds in the art having carbazole at 3-position substituted are utilized as hole injection layer materials. Light-emitting devices utilizing these compounds in a hole injection layer or a hole transport layer still do not have sufficient luminescence efficiency, and thus, higher efficiency characteristics are still required and/or desired.

In addition, light-emitting devices utilizing two or more hole transport layers with ionization potential values set in a stepped manner in order to efficiently inject holes from an anode into an emission layer are investigated, but have insufficient luminescence efficiency and insufficient lifespan.

According to one or more aspects of the present disclosure a compound represented by Formula 1 is provided:

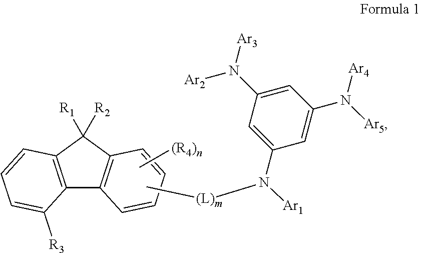

wherein, in Formula 1,
R₁, R₂, R₄, and Ar₁ to Ar₅ may each independently be hydrogen, deuterium, a C₁-C₆₀ alkyl group that is unsubstituted or substituted with at least one R₁₀ₐ, a C₃-C₁₀ cycloalkyl group that is unsubstituted or substituted with at least one R₁₀ₐ, a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ,
R₃ may be a C₁-C₆₀ alkyl group that is unsubstituted or substituted with at least one R₁₀ₐ, a C₃-C₁ cycloalkyl group that is unsubstituted or substituted with at least one R₁₀ₐ, or a C₆-C₆₀ aryl group that is unsubstituted or substituted with at least one R₁₀ₐ,
optionally, R₁ and R₂ may be linked together to form a ring,
L may be a single bond, a C₆-C₆₀ arylene group that is unsubstituted or substituted with at least one R₁₀ₐ, a C₁-C₆₀ heteroarylene group that is unsubstituted or substituted with at least one R₁₀ₐ, a divalent non-aromatic condensed polycyclic group that is unsubstituted or substituted with at least one R₁₀ₐ, or a divalent non-aromatic condensed heteropolycyclic group that is unsubstituted or substituted with at least one R₁₀ₐ,
m may be an integer from 0 to 3,
wherein,
when m is 0, -(L)ₘ- may be a single bond,
when m is 2 or more, each L may be identical to or different from each other,
n may be an integer from 0 to 3, wherein, when n is 2 or more, each R₄ may be identical to or different from each other,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In the compound represented by Formula 1 according to one or more embodiments, three amine groups are substituted on phenyl, and a fluorene group is substituted at one of the three amine groups. Here, R₃ is substituted at the 4-position of the fluorene group of Formula 1 according to one or more embodiments of the present disclosure (see herein).

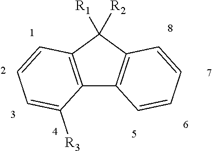

A substituent may be present at another position (for example, 1-, 2-, or 3-position or R₄ position) of the fluorene group of Formula 1 according to some embodiments, but in embodiments in which R₃ is present at the 4-position of the fluorene group, efficiency and lifespan of the light-emitting device including the compound represented by Formula 1 may be improved. Here, R₃ may be a C₁-C₆₀ alkyl group that is unsubstituted or substituted with at least one R₁₀ₐ, a C₃-C₁₀ cycloalkyl group that is unsubstituted or substituted with at least one R₁₀ₐ, or a C₆-C₆₀ aryl group that is unsubstituted or substituted with at least one R₁₀ₐ.

In one or more embodiments, Ar₁ to Ar₅ may each independently be a C₆-C₆₀ aryl group that is unsubstituted or substituted with at least one R₁₀ₐ.

In one or more embodiments, Ar₁ to Ar₅ may each independently be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group, each unsubstituted or substituted with at least one R₁₀ₐ.

For example, in some embodiments, Ar₁ to Ar₅ may each independently be a phenyl group or a naphthyl group, each unsubstituted or substituted with at least one R₁₀ₐ.

In one or more embodiments, each R₄ may independently be hydrogen or deuterium.

In one or more embodiments, R₁ to R₃ may each independently be a methyl group, an ethyl group, an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group, each unsubstituted or substituted with at least one R₁₀ₐ.

In one or more embodiments, the compound represented by Formula 1 may be a compound represented by Formula 2:

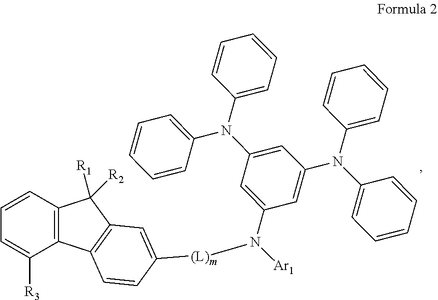

wherein, in Formula 2,
R₁ to R₃, Ar₁, L, and m may each be the same as defined in Formula 1.

In one or more embodiments, R₁ to R₃ may each independently be a methyl group, a phenyl group, or a naphthyl group, each unsubstituted or substituted with at least one R₁₀ₐ.

For example, in some embodiments, R₁ to R₃ may each independently be a methyl group or a phenyl group.

In one or more embodiments, Ar₁ may be a C₆-C₆₀ aryl group that is unsubstituted or substituted with at least one R₁₀ₐ, and R₁₀ₐ may be a C₃-C₆₀ carbocyclic group that is unsubstituted or substituted with a C₃-C₆₀ carbocyclic group. For example, in some embodiments, R₁₀ₐ may be: a C₆-C₆₀ aryl group that is unsubstituted or substituted with a C₃-C₁₀ cycloalkyl group or a C₆-C₆₀ aryl group; or a C₃-C₁₀ cycloalkyl group that is unsubstituted or substituted with a C₃-C₁₀ cycloalkyl group or a C₆-C₆₀ aryl group.

In one or more embodiments, the compound represented by Formula 1 may include one of (e.g., selected from among) the following compounds:

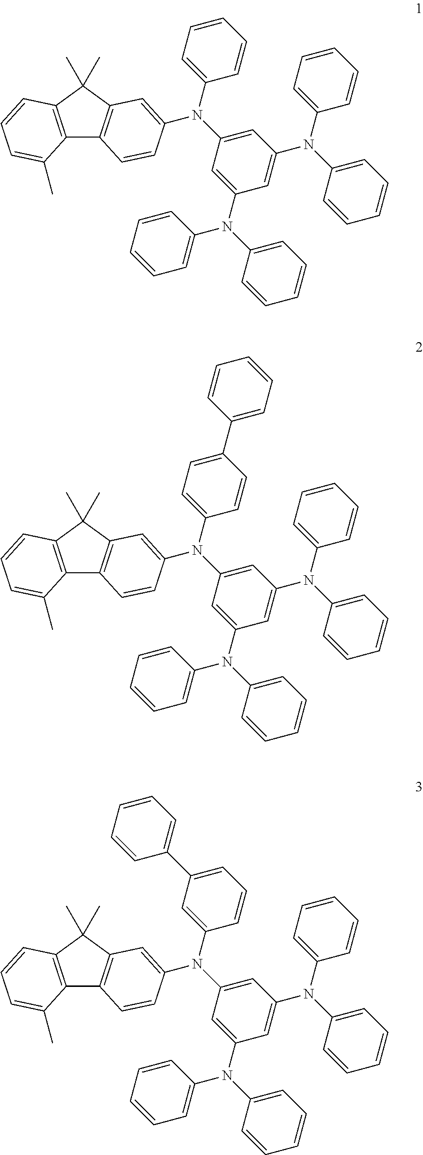 [SMILES: Cc1cccc2c1-c1ccc(N(c3ccccc3)c3cc(N(c4ccccc4)c4ccccc4)cc(N(c4ccccc4)c4ccccc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3cccc(-c4ccccc4)c3)c3cc(N(c4ccccc4)c4ccccc4)cc(N(c4ccccc4)c4ccccc4)c3)cc1C2(C)C]

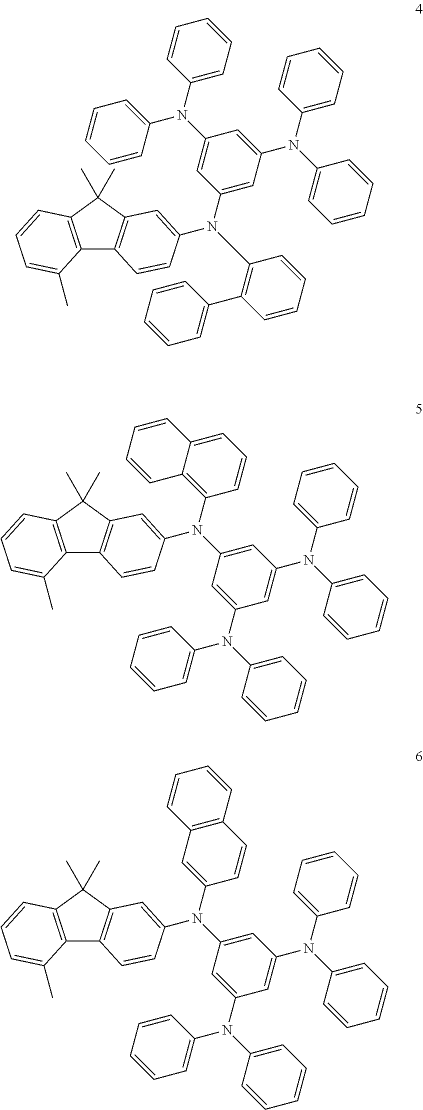 [SMILES: Cc1cccc2c1-c1ccc(N(c3cc(N(c4ccccc4)c4ccccc4)cc(N(c4ccccc4)c4ccccc4)c3)c3ccccc3-c3ccccc3)cc1C2(C)C ; Cc1cccc2c1-c1ccc(N(c3cc(N(c4ccccc4)c4ccccc4)cc(N(c4ccccc4)c4ccccc4)c3)c3cccc4ccccc34)cc1C2(C)C ; Cc1cccc2c1-c1ccc(N(c3cc(N(c4ccccc4)c4ccccc4)cc(N(c4ccccc4)c4ccccc4)c3)c3ccc4ccccc4c3)cc1C2(C)C]

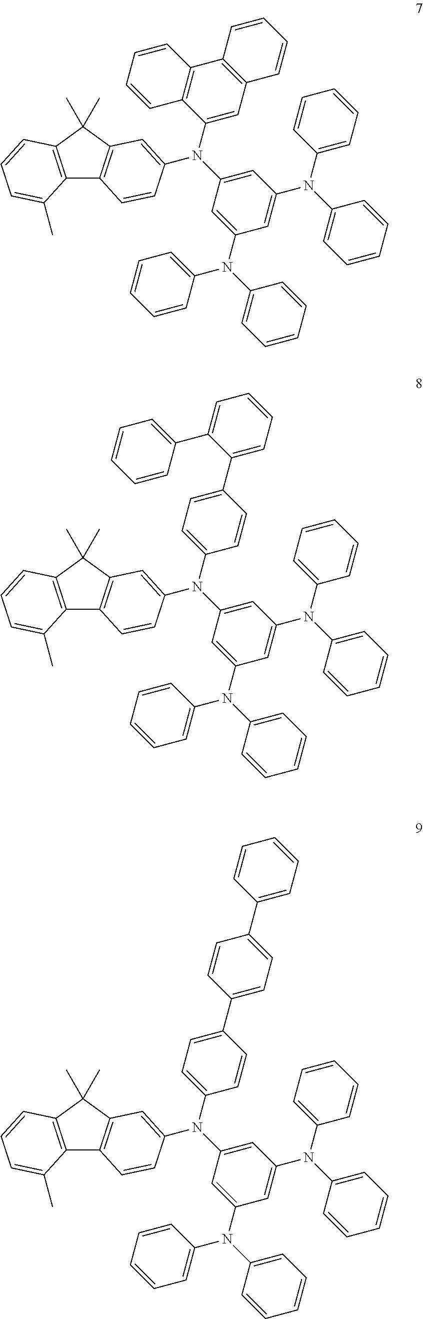 [SMILES: Cc1cccc2c1-c1ccc(N(c3cc(N(c4ccccc4)c4ccccc4)cc(N(c4ccccc4)c4ccccc4)c3)c3cc4ccccc4c4ccccc34)cc1C2(C)C ; Cc1cccc2c1-c1ccc(N(c3ccc(-c4ccccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4ccc(-c5ccccc5)cc4)cc3)c3cc(N(c4ccccc4)c4ccccc4)cc(N(c4ccccc4)c4ccccc4)c3)cc1C2(C)C]

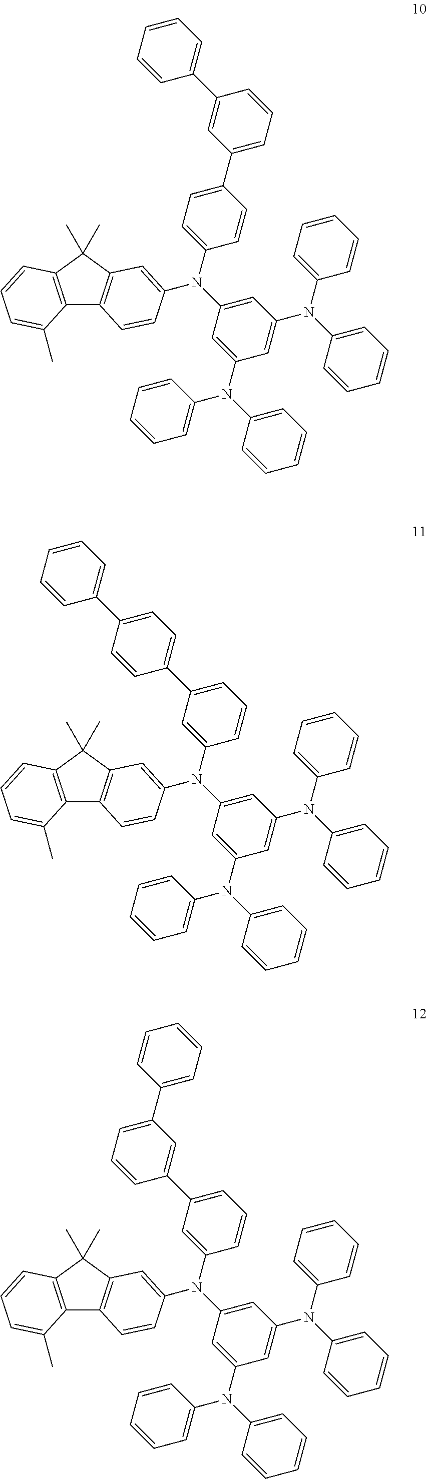 [SMILES: Cc1cccc2c1-c1ccc(N(c3ccc(-c4cccc(-c5ccccc5)c4)cc3)c3cc(N(c4ccccc4)c4ccccc4)cc(N(c4ccccc4)c4ccccc4)c3)cc1C2(C)C ; Cc1cccc2c1-c1ccc(N(c3cccc(-c4ccc(-c5ccccc5)cc4)c3)c3cc(N(c4ccccc4)c4ccccc4)cc(N(c4ccccc4)c4ccccc4)c3)cc1C2(C)C ; Cc1cccc2c1-c1ccc(N(c3cccc(-c4cccc(-c5ccccc5)c4)c3)c3cc(N(c4ccccc4)c4ccccc4)cc(N(c4ccccc4)c4ccccc4)c3)cc1C2(C)C]

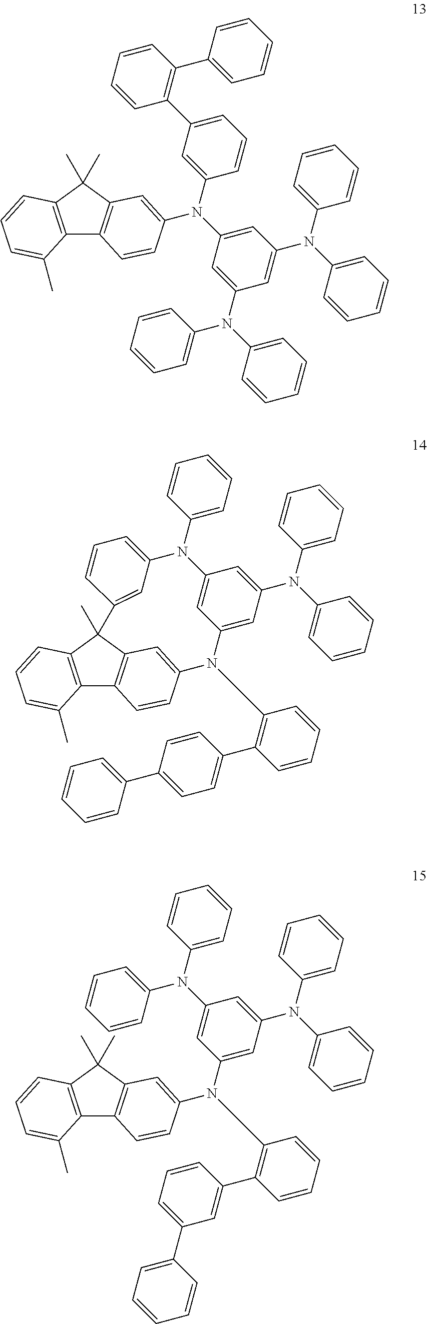 [SMILES: Cc1cccc2c1-c1ccc(N(c3cccc(-c4ccccc4-c4ccccc4)c3)c3cc(N(c4ccccc4)c4ccccc4)cc(N(c4ccccc4)c4ccccc4)c3)cc1C2(C)C ; Cc1cccc2c1-c1ccc3cc1C2(C)c1cccc(c1)N(c1ccccc1)c1cc(N(c2ccccc2)c2ccccc2)cc(c1)N3c1ccccc1-c1ccc(-c2ccccc2)cc1 ; Cc1cccc2c1-c1ccc(N(c3cc(N(c4ccccc4)c4ccccc4)cc(N(c4ccccc4)c4ccccc4)c3)c3ccccc3-c3cccc(-c4ccccc4)c3)cc1C2(C)C]

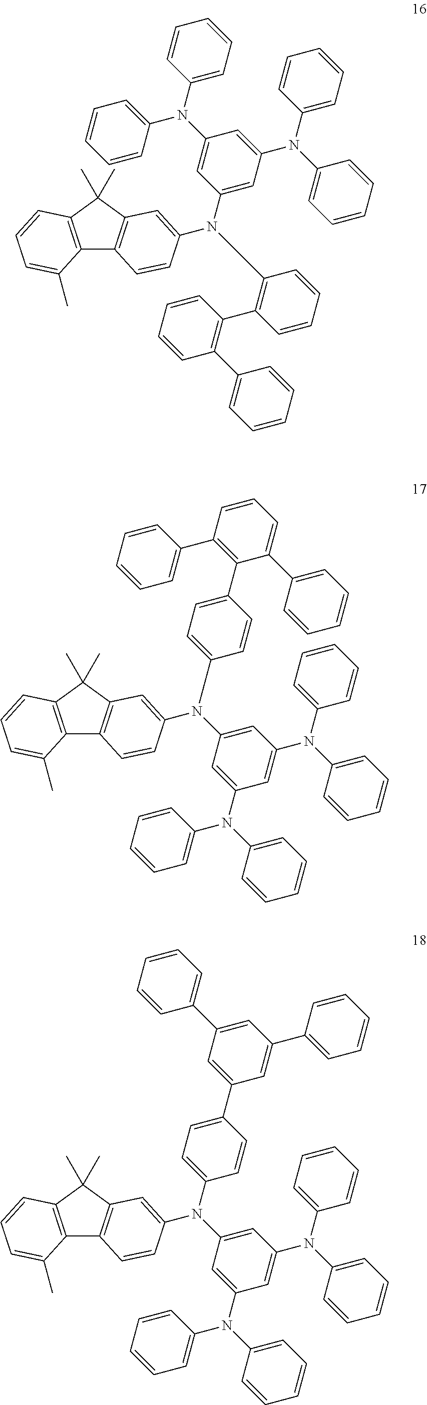 [SMILES: Cc1cccc2c1-c1ccc(N(c3cc(N(c4ccccc4)c4ccccc4)cc(N(c4ccccc4)c4ccccc4)c3)c3ccccc3-c3ccccc3-c3ccccc3)cc1C2(C)C ; Cc1cccc2c1-c1ccc(N(c3ccc(-c4c(-c5ccccc5)cccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4cc(-c5ccccc5)cc(-c5ccccc5)c4)cc3)c3cc(N(c4ccccc4)c4ccccc4)cc(N(c4ccccc4)c4ccccc4)c3)cc1C2(C)C]

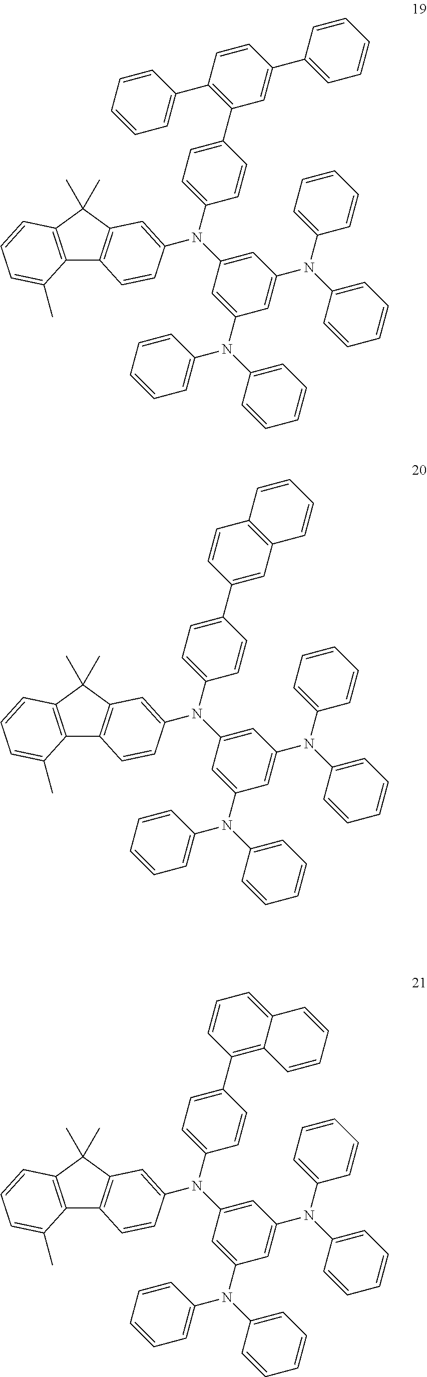 [SMILES: Cc1cccc2c1-c1ccc(N(c3ccc(-c4cc(-c5ccccc5)ccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4ccc5ccccc5c4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4cccc5ccccc45)cc3)c3cc(N(c4ccccc4)c4ccccc4)cc(N(c4ccccc4)c4ccccc4)c3)cc1C2(C)C]

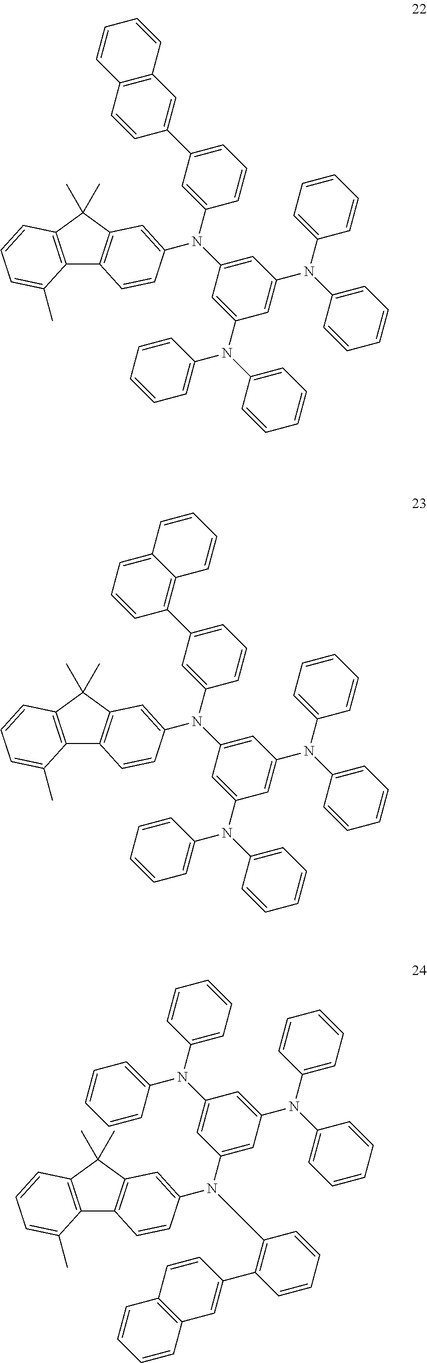 [SMILES: Cc1cccc2c1-c1ccc(N(c3cccc(-c4ccc5ccccc5c4)c3)c3cc(N(c4ccccc4)c4ccccc4)cc(N(c4ccccc4)c4ccccc4)c3)cc1C2(C)C ; Cc1cccc2c1-c1ccc(N(c3cccc(-c4cccc5ccccc45)c3)c3cc(N(c4ccccc4)c4ccccc4)cc(N(c4ccccc4)c4ccccc4)c3)cc1C2(C)C ; Cc1cccc2c1-c1ccc(N(c3cc(N(c4ccccc4)c4ccccc4)cc(N(c4ccccc4)c4ccccc4)c3)c3ccccc3-c3ccc4ccccc4c3)cc1C2(C)C]

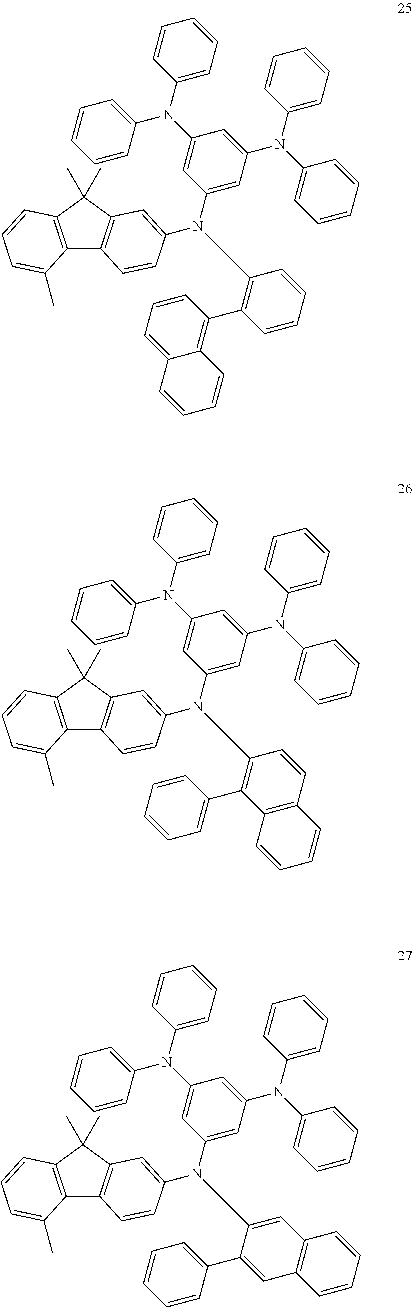 [SMILES: Cc1cccc2c1-c1ccc(N(c3cc(N(c4ccccc4)c4ccccc4)cc(N(c4ccccc4)c4ccccc4)c3)c3ccccc3-c3cccc4ccccc34)cc1C2(C)C ; Cc1cccc2c1-c1ccc(N(c3cc(N(c4ccccc4)c4ccccc4)cc(N(c4ccccc4)c4ccccc4)c3)c3ccc4ccccc4c3-c3ccccc3)cc1C2(C)C ; Cc1cccc2c1-c1ccc(N(c3cc(N(c4ccccc4)c4ccccc4)cc(N(c4ccccc4)c4ccccc4)c3)c3cc4ccccc4cc3-c3ccccc3)cc1C2(C)C]

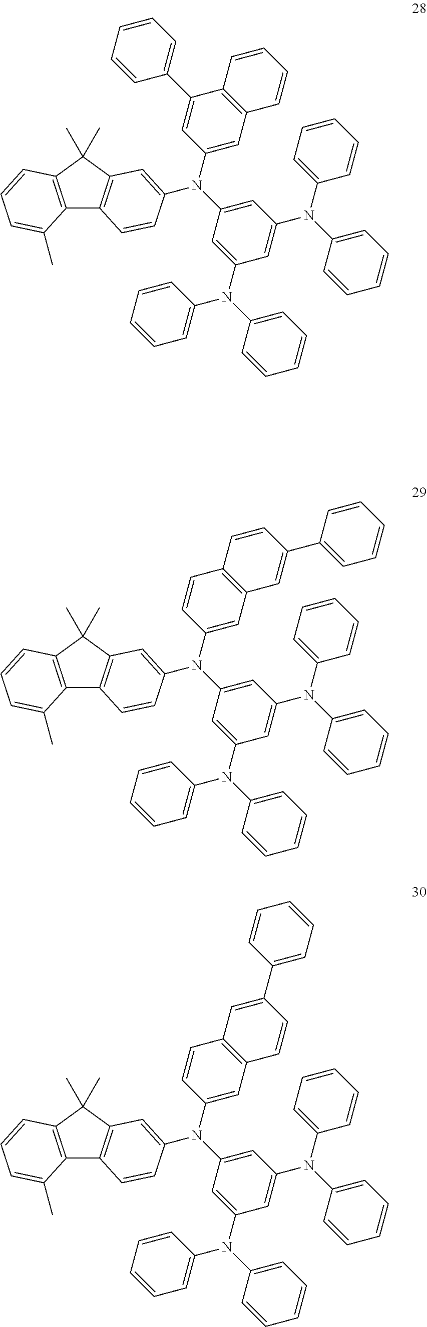 [SMILES: Cc1cccc2c1-c1ccc(N(c3cc(N(c4ccccc4)c4ccccc4)cc(N(c4ccccc4)c4ccccc4)c3)c3cc(-c4ccccc4)c4ccccc4c3)cc1C2(C)C ; Cc1cccc2c1-c1ccc(N(c3cc(N(c4ccccc4)c4ccccc4)cc(N(c4ccccc4)c4ccccc4)c3)c3ccc4ccc(-c5ccccc5)cc4c3)cc1C2(C)C ; Cc1cccc2c1-c1ccc(N(c3cc(N(c4ccccc4)c4ccccc4)cc(N(c4ccccc4)c4ccccc4)c3)c3ccc4cc(-c5ccccc5)ccc4c3)cc1C2(C)C]

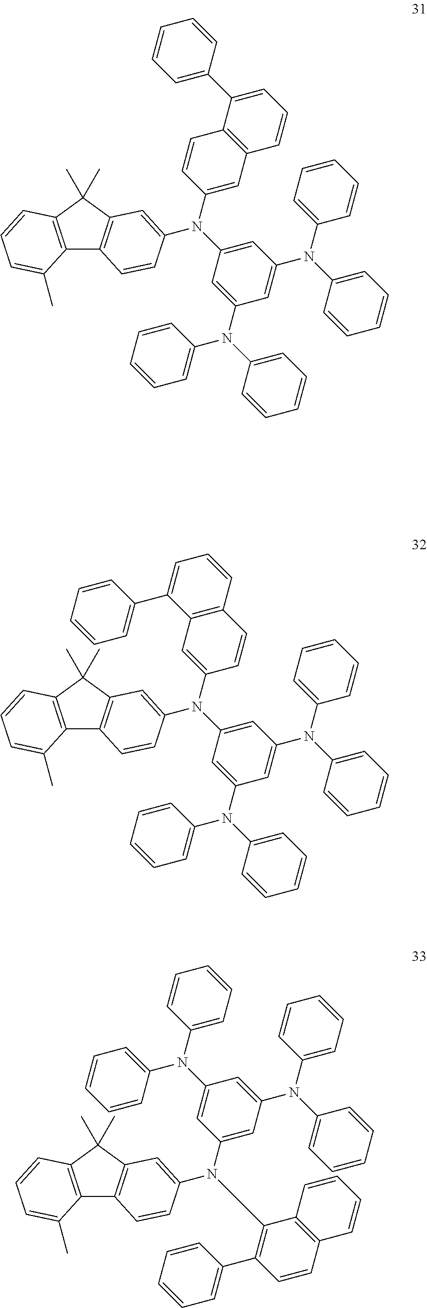 [SMILES: Cc1cccc2c1-c1ccc(N(c3cc(N(c4ccccc4)c4ccccc4)cc(N(c4ccccc4)c4ccccc4)c3)c3ccc4c(-c5ccccc5)cccc4c3)cc1C2(C)C ; Cc1cccc2c1-c1ccc(N(c3cc(N(c4ccccc4)c4ccccc4)cc(N(c4ccccc4)c4ccccc4)c3)c3ccc4cccc(-c5ccccc5)c4c3)cc1C2(C)C ; Cc1cccc2c1-c1ccc(N(c3cc(N(c4ccccc4)c4ccccc4)cc(N(c4ccccc4)c4ccccc4)c3)c3c(-c4ccccc4)ccc4ccccc34)cc1C2(C)C]

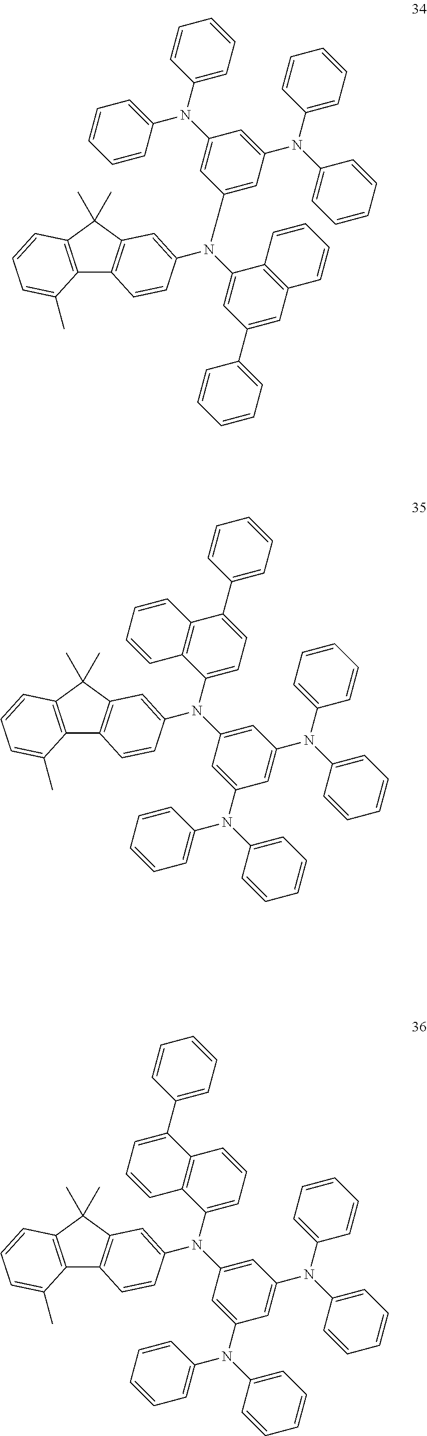 [SMILES: Cc1cccc2c1-c1ccc(N(c3cc(N(c4ccccc4)c4ccccc4)cc(N(c4ccccc4)c4ccccc4)c3)c3cc(-c4ccccc4)cc4ccccc34)cc1C2(C)C ; Cc1cccc2c1-c1ccc(N(c3cc(N(c4ccccc4)c4ccccc4)cc(N(c4ccccc4)c4ccccc4)c3)c3ccc(-c4ccccc4)c4ccccc34)cc1C2(C)C ; Cc1cccc2c1-c1ccc(N(c3cc(N(c4ccccc4)c4ccccc4)cc(N(c4ccccc4)c4ccccc4)c3)c3cccc4c(-c5ccccc5)cccc34)cc1C2(C)C]

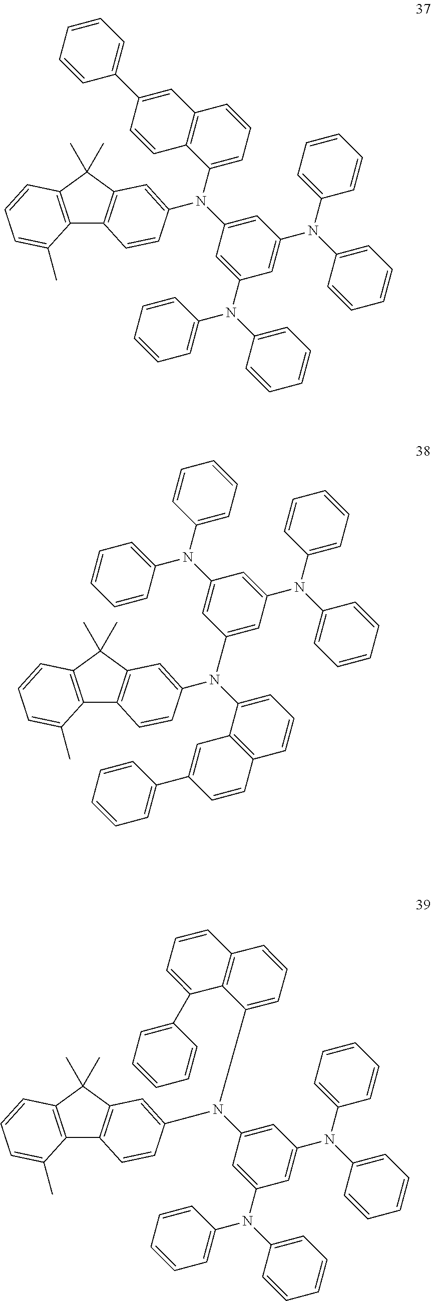 [SMILES: Cc1cccc2c1-c1ccc(N(c3cc(N(c4ccccc4)c4ccccc4)cc(N(c4ccccc4)c4ccccc4)c3)c3cccc4cc(-c5ccccc5)ccc34)cc1C2(C)C ; Cc1cccc2c1-c1ccc(N(c3cc(N(c4ccccc4)c4ccccc4)cc(N(c4ccccc4)c4ccccc4)c3)c3cccc4ccc(-c5ccccc5)cc34)cc1C2(C)C ; Cc1cccc2c1-c1ccc(N(c3cc(N(c4ccccc4)c4ccccc4)cc(N(c4ccccc4)c4ccccc4)c3)c3cccc4cccc(-c5ccccc5)c34)cc1C2(C)C]

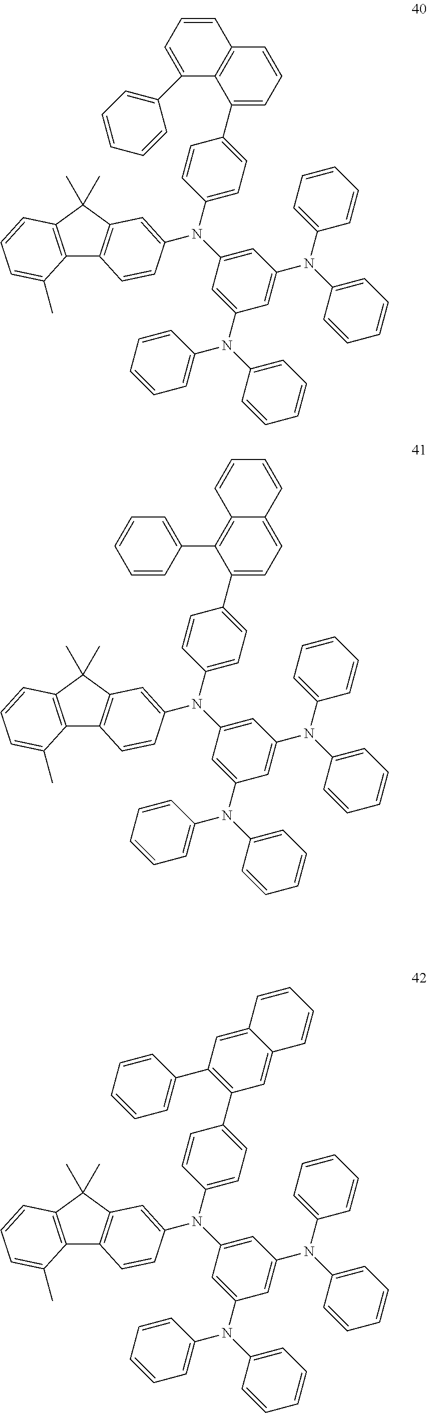

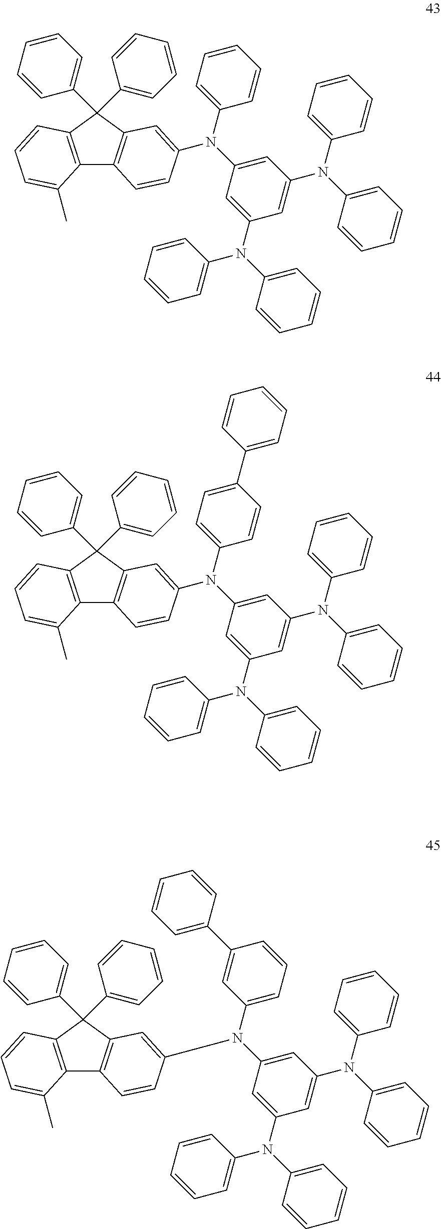 [SMILES: Cc1cccc2c1-c1ccc(N(c3ccccc3)c3cc(N(c4ccccc4)c4ccccc4)cc(N(c4ccccc4)c4ccccc4)c3)cc1C2(c1ccccc1)c1ccccc1 ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(c1ccccc1)c1ccccc1 ; Cc1cccc2c1-c1ccc(N(c3cccc(-c4ccccc4)c3)c3cc(N(c4ccccc4)c4ccccc4)cc(N(c4ccccc4)c4ccccc4)c3)cc1C2(c1ccccc1)c1ccccc1]

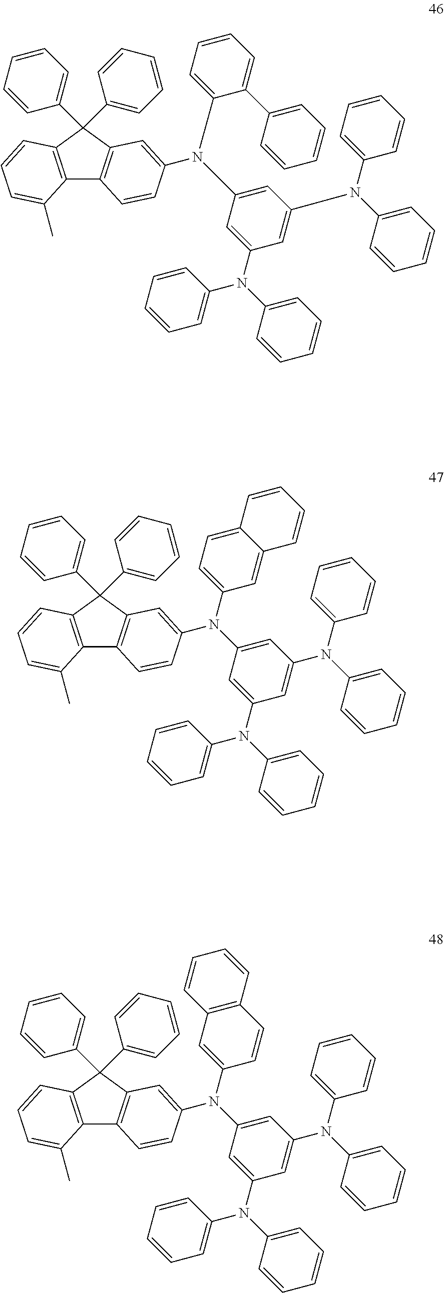 [SMILES: Cc1cccc2c1-c1ccc(N(c3cc(N(c4ccccc4)c4ccccc4)cc(N(c4ccccc4)c4ccccc4)c3)c3ccccc3-c3ccccc3)cc1C2(c1ccccc1)c1ccccc1 ; Cc1cccc2c1-c1ccc(N(c3cc(N(c4ccccc4)c4ccccc4)cc(N(c4ccccc4)c4ccccc4)c3)c3ccc4ccccc4c3)cc1C2(c1ccccc1)c1ccccc1 ; Cc1cccc2c1-c1ccc(N(c3cc(N(c4ccccc4)c4ccccc4)cc(N(c4ccccc4)c4ccccc4)c3)c3ccc4ccccc4c3)cc1C2(c1ccccc1)c1ccccc1]

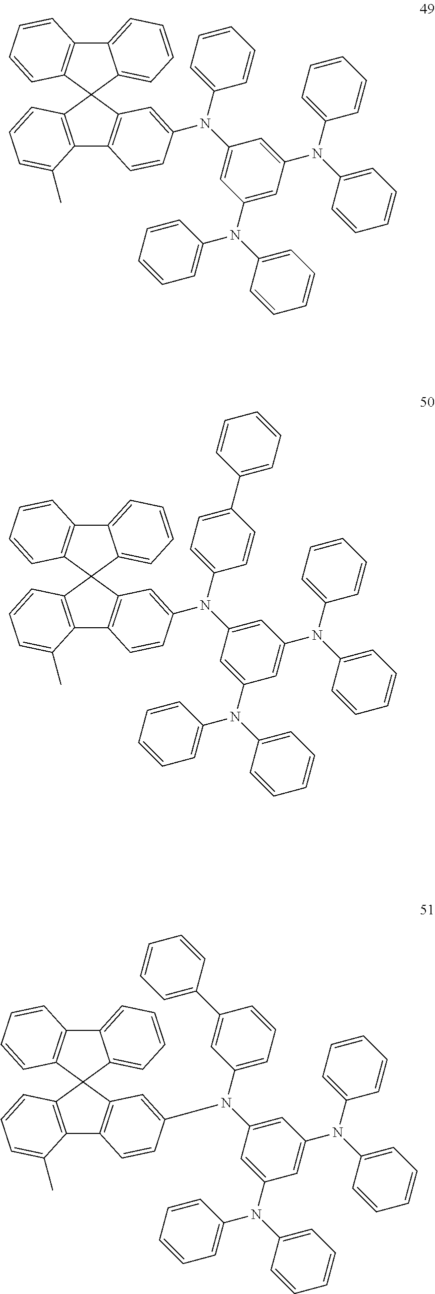 [SMILES: Cc1cccc2c1-c1ccc(N(c3ccccc3)c3cc(N(c4ccccc4)c4ccccc4)cc(N(c4ccccc4)c4ccccc4)c3)cc1C21c2ccccc2-c2ccccc21 ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C21c2ccccc2-c2ccccc21 ; Cc1cccc2c1-c1ccc(N(c3cccc(-c4ccccc4)c3)c3cc(N(c4ccccc4)c4ccccc4)cc(N(c4ccccc4)c4ccccc4)c3)cc1C21c2ccccc2-c2ccccc21]

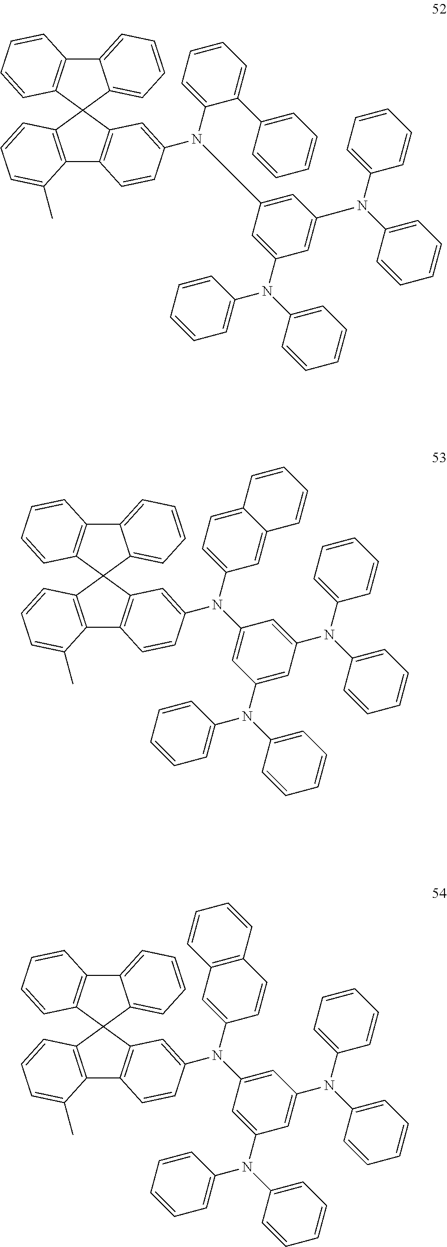 [SMILES: Cc1cccc2c1-c1ccc(N(c3cc(N(c4ccccc4)c4ccccc4)cc(N(c4ccccc4)c4ccccc4)c3)c3ccccc3-c3ccccc3)cc1C21c2ccccc2-c2ccccc21 ; Cc1cccc2c1-c1ccc(N(c3cc(N(c4ccccc4)c4ccccc4)cc(N(c4ccccc4)c4ccccc4)c3)c3ccc4ccccc4c3)cc1C21c2ccccc2-c2ccccc21 ; Cc1cccc2c1-c1ccc(N(c3cc(N(c4ccccc4)c4ccccc4)cc(N(c4ccccc4)c4ccccc4)c3)c3ccc4ccccc4c3)cc1C21c2ccccc2-c2ccccc21]

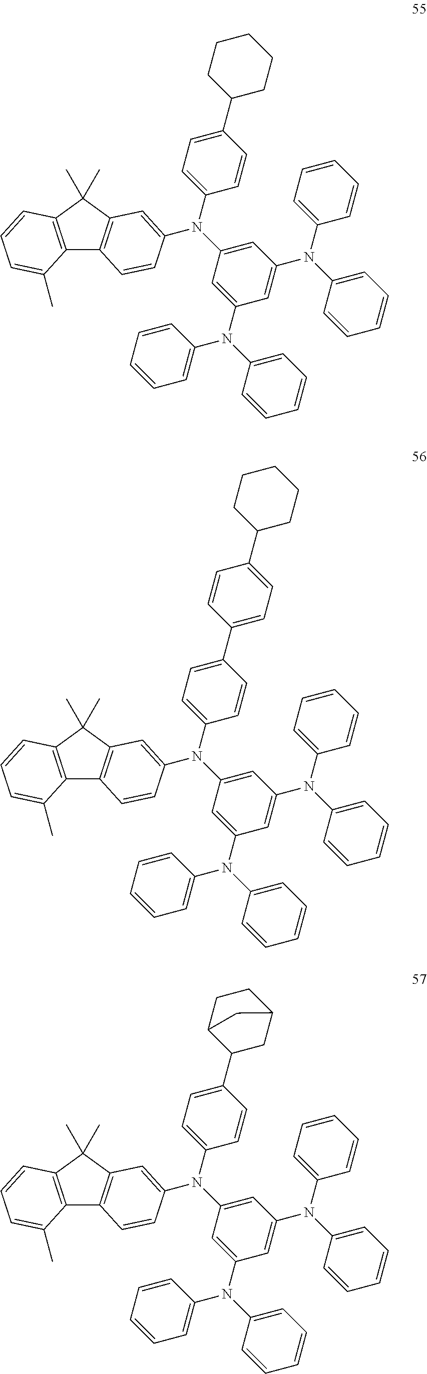

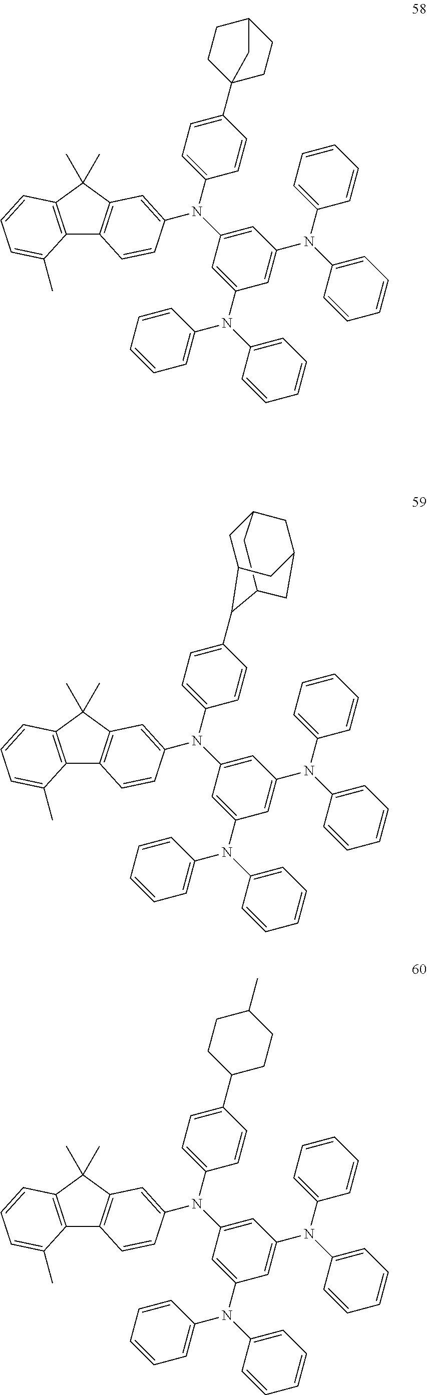 [SMILES: Cc1cccc2c1-c1ccc(N(c3ccc(C45CCC(CC4)C5)cc3)c3cc(N(c4ccccc4)c4ccccc4)cc(N(c4ccccc4)c4ccccc4)c3)cc1C2(C)C ; Cc1cccc2c1-c1ccc(N(c3ccc(C4C5CC6CC(C5)CC4C6)cc3)c3cc(N(c4ccccc4)c4ccccc4)cc(N(c4ccccc4)c4ccccc4)c3)cc1C2(C)C ; Cc1cccc2c1-c1ccc(N(c3ccc(C4CCC(C)CC4)cc3)c3cc(N(c4ccccc4)c4ccccc4)cc(N(c4ccccc4)c4ccccc4)c3)cc1C2(C)C]

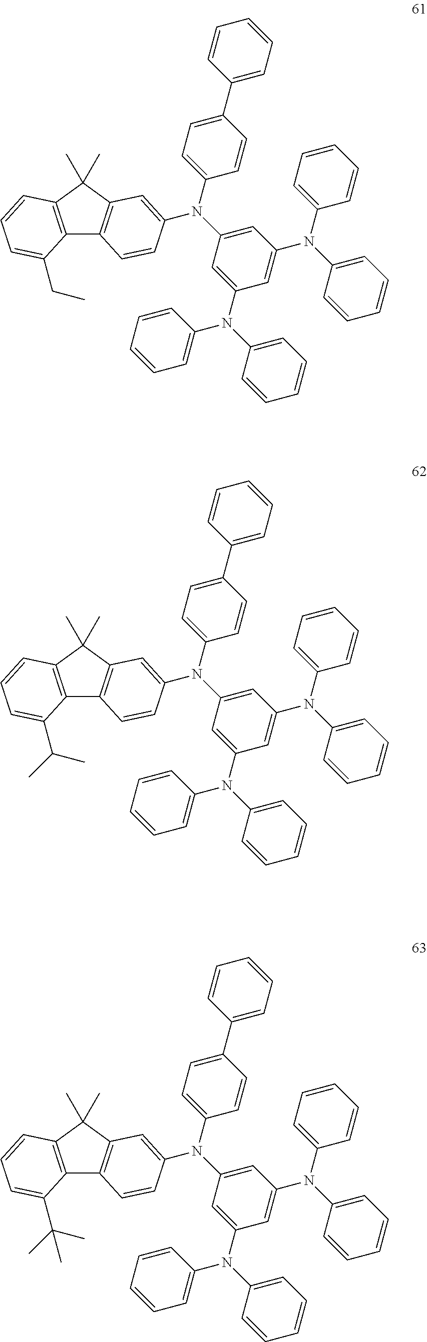 [SMILES: CCc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; CC(C)c1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; CC(C)(C)c1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C]

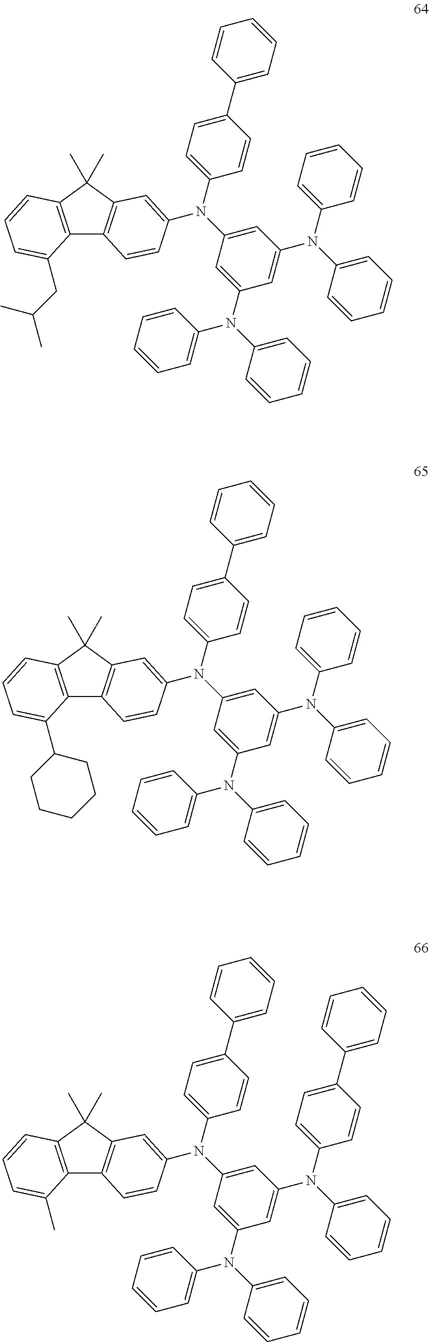 [SMILES: CC(C)Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; CC1(C)c2cc(N(c3ccc(-c4ccccc4)cc3)c3cc(N(c4ccccc4)c4ccccc4)cc(N(c4ccccc4)c4ccccc4)c3)ccc2-c2c(C3CCCCC3)cccc21 ; Cc1cccc2c1-c1ccc(N(c3ccc(-c4ccccc4)cc3)c3cc(N(c4ccccc4)c4ccccc4)cc(N(c4ccccc4)c4ccc(-c5ccccc5)cc4)c3)cc1C2(C)C]

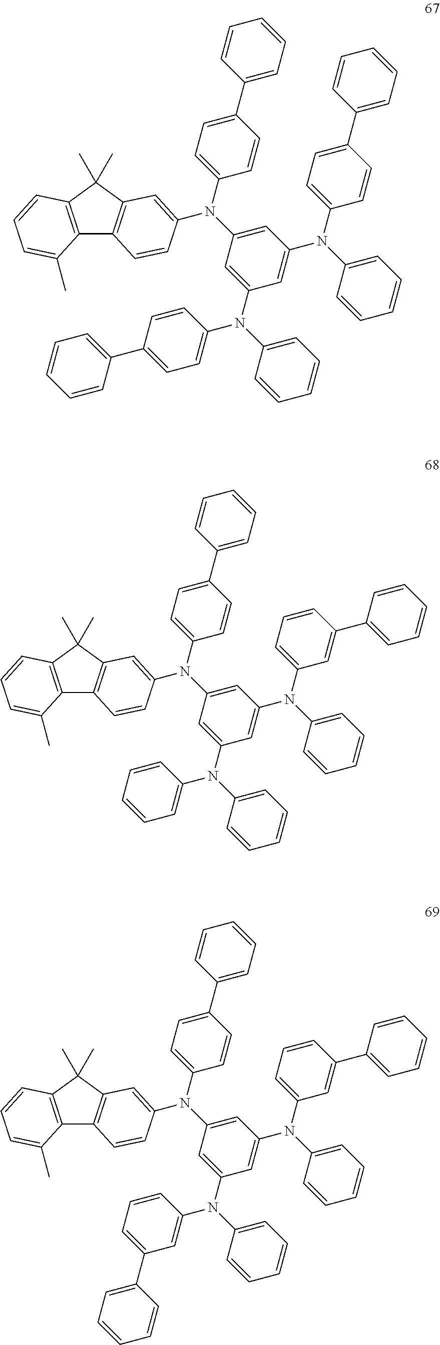 [SMILES: Cc1cccc2c1-c1ccc(N(c3ccc(-c4ccccc4)cc3)c3cc(N(c4ccccc4)c4ccc(-c5ccccc5)cc4)cc(N(c4ccccc4)c4ccc(-c5ccccc5)cc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4cccc(-c5ccccc5)c4)c3)cc1C2(C)C ; Cc1cccc2c1-c1ccc(N(c3ccc(-c4ccccc4)cc3)c3cc(N(c4ccccc4)c4cccc(-c5ccccc5)c4)cc(N(c4ccccc4)c4cccc(-c5ccccc5)c4)c3)cc1C2(C)C]

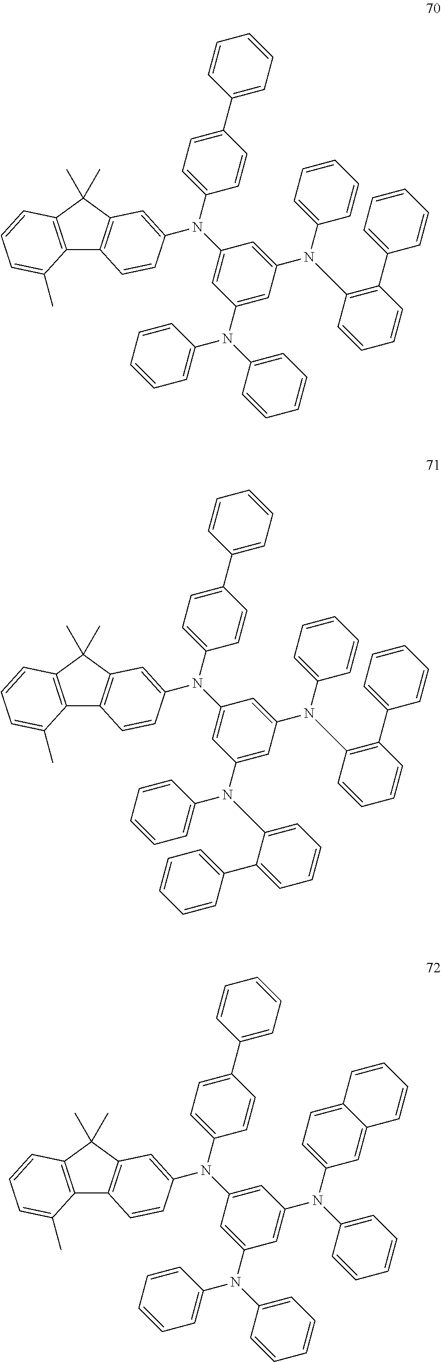 [SMILES: Cc1cccc2c1-c1ccc(N(c3ccc(-c4ccccc4)cc3)c3cc(N(c4ccccc4)c4ccccc4)cc(N(c4ccccc4)c4ccccc4-c4ccccc4)c3)cc1C2(C)C ; Cc1cccc2c1-c1ccc(N(c3ccc(-c4ccccc4)cc3)c3cc(N(c4ccccc4)c4ccccc4-c4ccccc4)cc(N(c4ccccc4)c4ccccc4-c4ccccc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4ccc5ccccc5c4)c3)cc1C2(C)C]

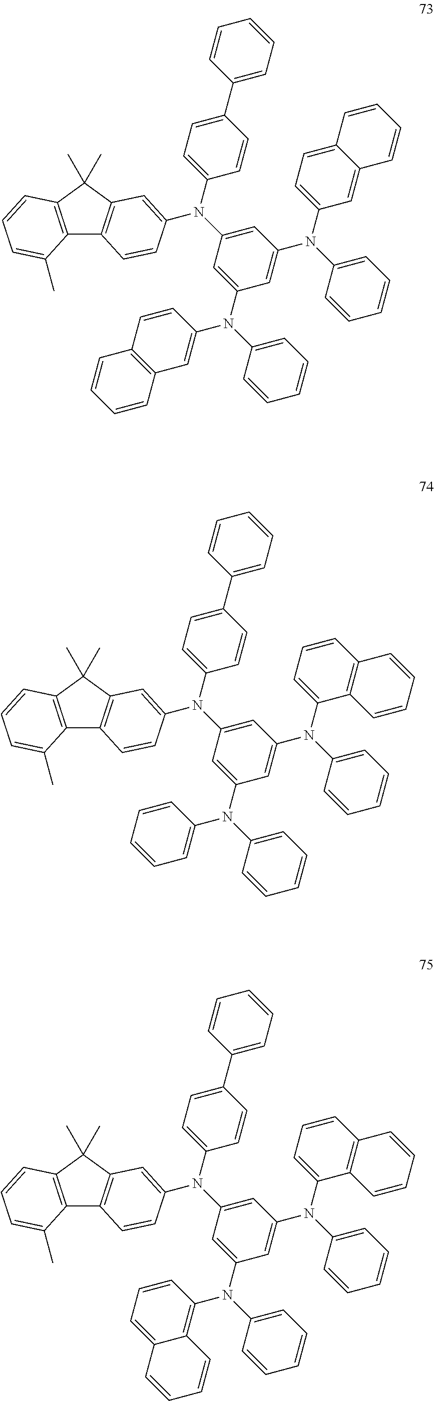 [SMILES: Cc1cccc2c1-c1ccc(N(c3ccc(-c4ccccc4)cc3)c3cc(N(c4ccccc4)c4ccc5ccccc5c4)cc(N(c4ccccc4)c4ccc5ccccc5c4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4cccc5ccccc45)c3)cc1C2(C)C ; Cc1cccc2c1-c1ccc(N(c3ccc(-c4ccccc4)cc3)c3cc(N(c4ccccc4)c4cccc5ccccc45)cc(N(c4ccccc4)c4cccc5ccccc45)c3)cc1C2(C)C]

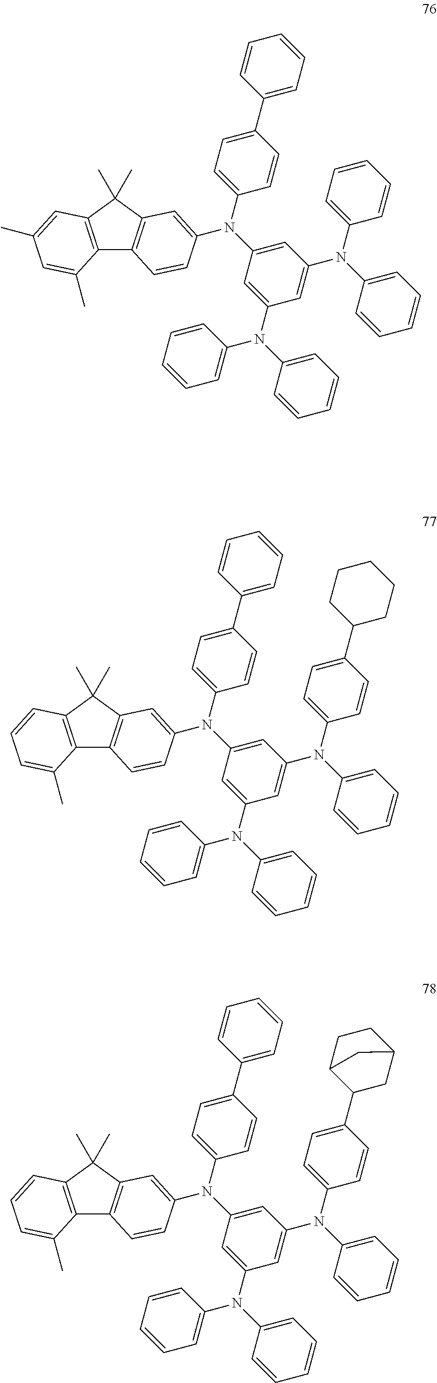 [SMILES: Cc1cc(C)c2c(c1)C(C)(C)c1cc(N(c3ccc(-c4ccccc4)cc3)c3cc(N(c4ccccc4)c4ccccc4)cc(N(c4ccccc4)c4ccccc4)c3)ccc1-2 ; Cc1cccc2c1-c1ccc(N(c3ccc(-c4ccccc4)cc3)c3cc(N(c4ccccc4)c4ccccc4)cc(N(c4ccccc4)c4ccc(C5CCCCC5)cc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4ccc(C5CC6CCC5C6)cc4)c3)cc1C2(C)C]

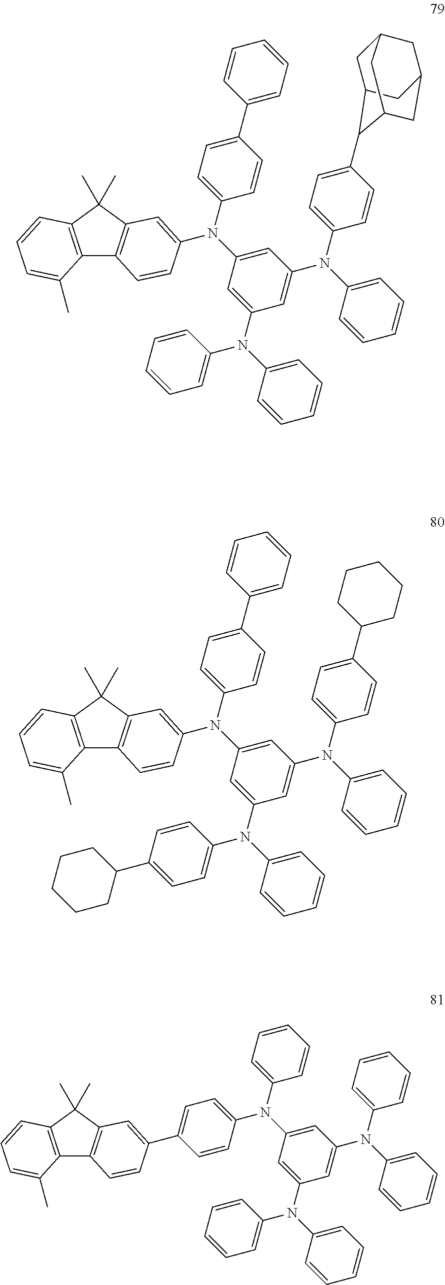 [SMILES: Cc1cccc2c1-c1ccc(N(c3ccc(-c4ccccc4)cc3)c3cc(N(c4ccccc4)c4ccccc4)cc(N(c4ccccc4)c4ccc(C5C6CC7CC(C6)CC5C7)cc4)c3)cc1C2(C)C ; Cc1cccc2c1-c1ccc(N(c3ccc(-c4ccccc4)cc3)c3cc(N(c4ccccc4)c4ccc(C5CCCCC5)cc4)cc(N(c4ccccc4)c4ccc(C5CCCCC5)cc4)c3)cc1C2(C)C ; Cc1cccc2c1-c1ccc(-c3ccc(N(c4ccccc4)c4cc(N(c5ccccc5)c5ccccc5)cc(N(c5ccccc5)c5ccccc5)c4)cc3)cc1C2(C)C]

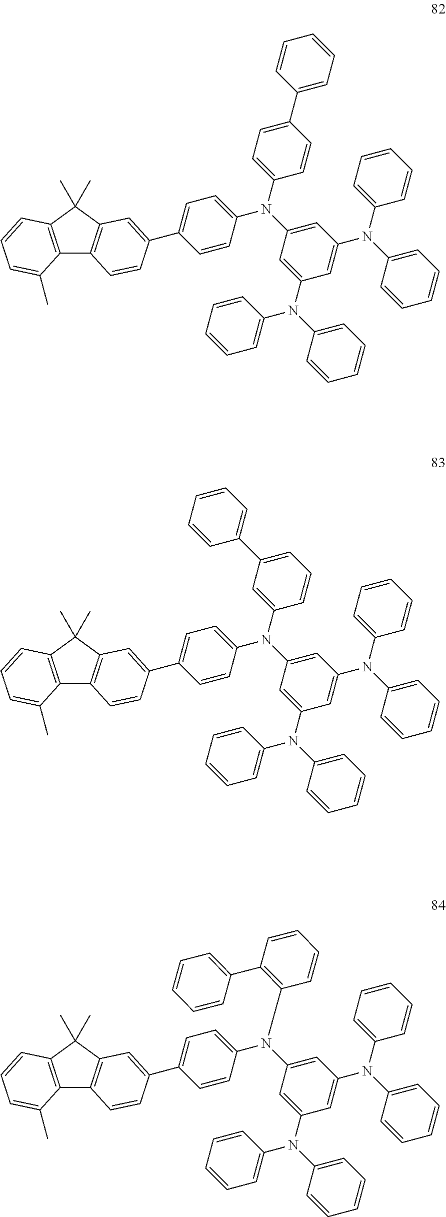 [SMILES: Cc1cccc2c1-c1ccc(-c3ccc(N(c4ccc(-c5ccccc5)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4cccc(-c5ccccc5)c4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4cc(N(c5ccccc5)c5ccccc5)cc(N(c5ccccc5)c5ccccc5)c4)c4ccccc4-c4ccccc4)cc3)cc1C2(C)C]

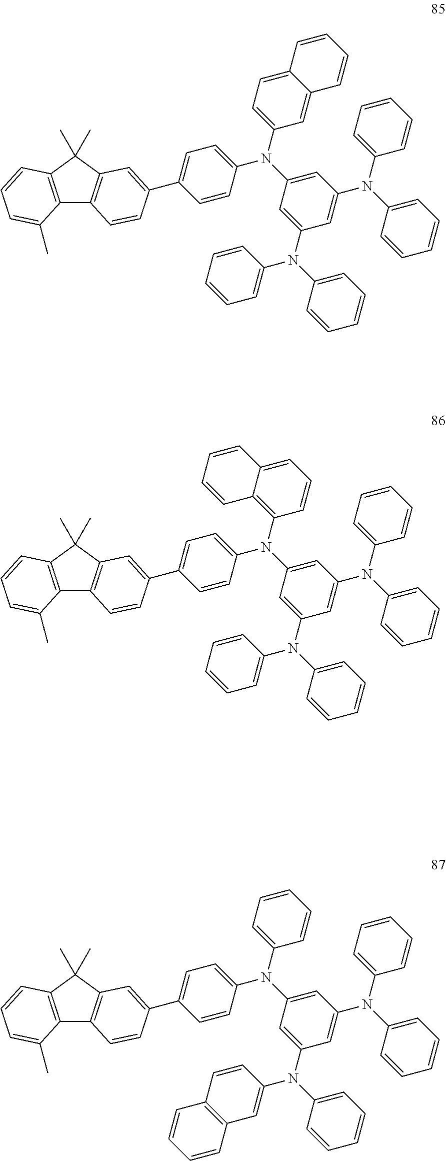 [SMILES: Cc1cccc2c1-c1ccc(-c3ccc(N(c4cc(N(c5ccccc5)c5ccccc5)cc(N(c5ccccc5)c5ccccc5)c4)c4ccc5ccccc5c4)cc3)cc1C2(C)C ; Cc1cccc2c1-c1ccc(-c3ccc(N(c4cc(N(c5ccccc5)c5ccccc5)cc(N(c5ccccc5)c5ccccc5)c4)c4cccc5ccccc45)cc3)cc1C2(C)C ; Cc1cccc2c1-c1ccc(-c3ccc(N(c4ccccc4)c4cc(N(c5ccccc5)c5ccccc5)cc(N(c5ccccc5)c5ccc6ccccc6c5)c4)cc3)cc1C2(C)C]

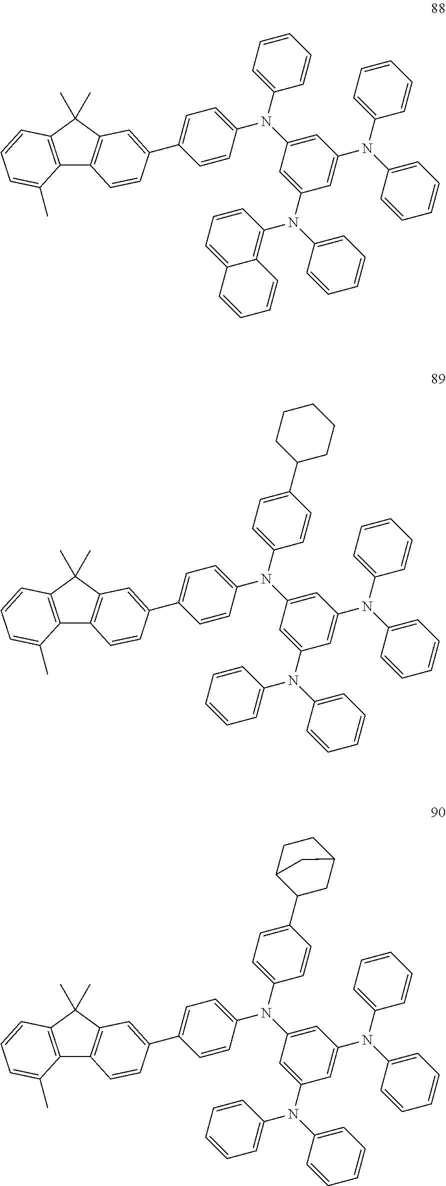 [SMILES: Cc1cccc2c1-c1ccc(-c3ccc(N(c4ccccc4)c4cc(N(c5ccccc5)c5ccccc5)cc(N(c5ccccc5)c5cccc6ccccc56)c4)cc3)cc1C2(C)C ; Cc1cccc2c1-c1ccc(-c3ccc(N(c4ccc(C5CCCCC5)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4ccc(C5CC6CCC5C6)cc4)c4cc(N(c5ccccc5)c5ccccc5)cc(N(c5ccccc5)c5ccccc5)c4)cc3)cc1C2(C)C]

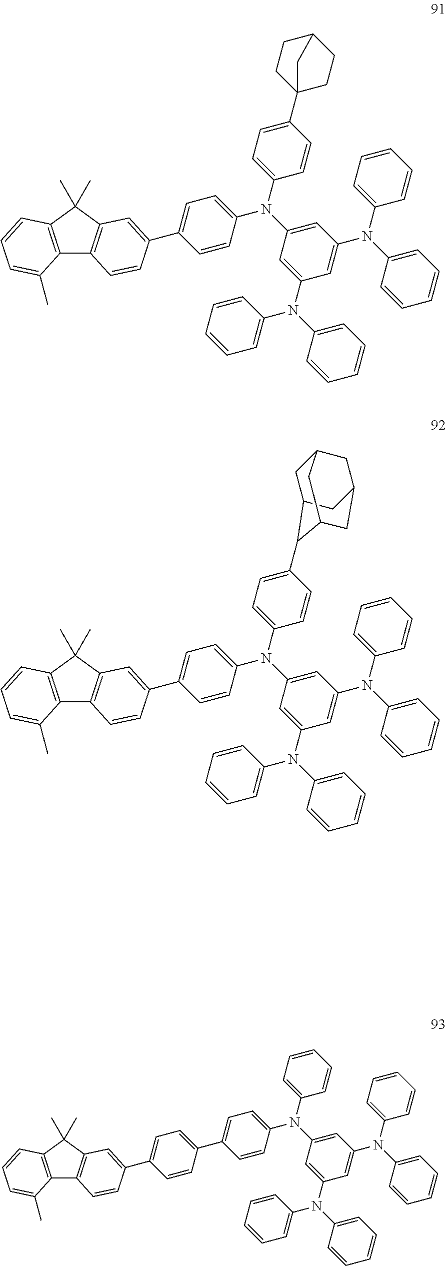 [SMILES: Cc1cccc2c1-c1ccc(-c3ccc(N(c4ccc(C56CCC(CC5)C6)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4ccc(C5C6CC7CC(C6)CC5C7)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(-c4ccc(N(c5ccccc5)c5cc(N(c6ccccc6)c6ccccc6)cc(N(c6ccccc6)c6ccccc6)c5)cc4)cc3)cc1C2(C)C]

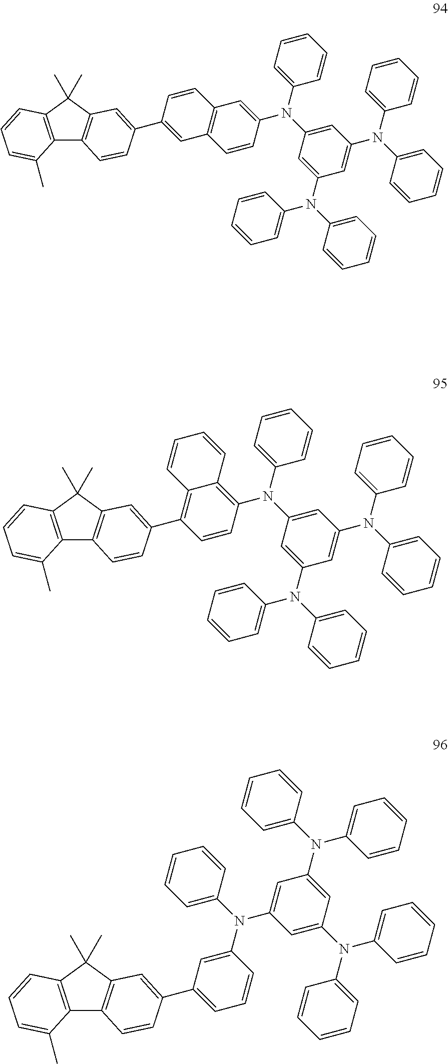 [SMILES: Cc1cccc2c1-c1ccc(-c3ccc4cc(N(c5ccccc5)c5cc(N(c6ccccc6)c6ccccc6)cc(N(c6ccccc6)c6ccccc6)c5)ccc4c3)cc1C2(C)C ; Cc1cccc2c1-c1ccc(-c3ccc(N(c4ccccc4)c4cc(N(c5ccccc5)c5ccccc5)cc(N(c5ccccc5)c5ccccc5)c4)c4ccccc34)cc1C2(C)C ; Cc1cccc2c1-c1ccc(-c3cccc(N(c4ccccc4)c4cc(N(c5ccccc5)c5ccccc5)cc(N(c5ccccc5)c5ccccc5)c4)c3)cc1C2(C)C]

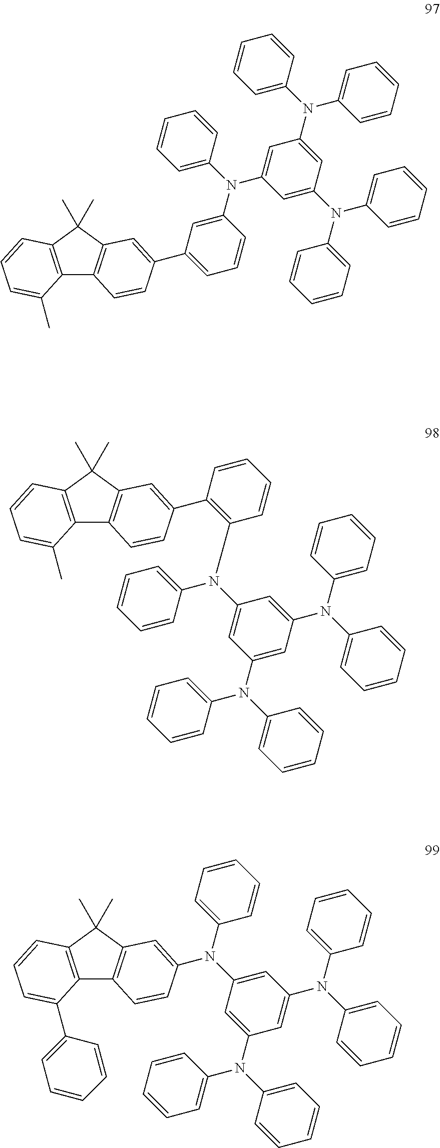 [SMILES: Cc1cccc2c1-c1ccc(-c3cccc(N(c4ccccc4)c4cc(N(c5ccccc5)c5ccccc5)cc(N(c5ccccc5)c5ccccc5)c4)c3)cc1C2(C)C ; Cc1cccc2c1-c1ccc(-c3ccccc3N(c3ccccc3)c3cc(N(c4ccccc4)c4ccccc4)cc(N(c4ccccc4)c4ccccc4)c3)cc1C2(C)C ; CC1(C)c2cc(N(c3ccccc3)c3cc(N(c4ccccc4)c4ccccc4)cc(N(c4ccccc4)c4ccccc4)c3)ccc2-c2c(-c3ccccc3)cccc21]

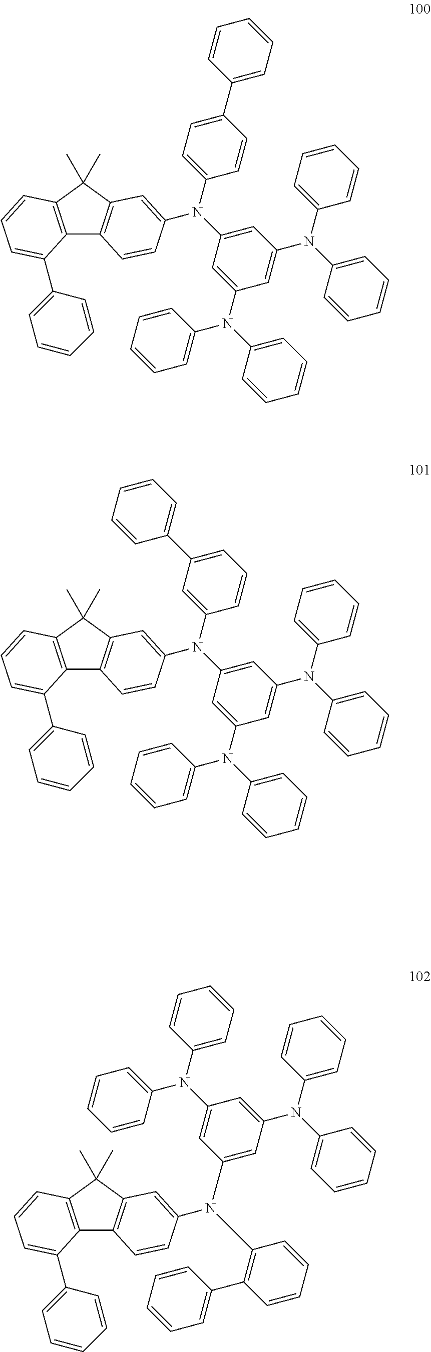 [SMILES: CC1(C)c2cc(N(c3ccc(-c4ccccc4)cc3)c3cc(N(c4ccccc4)c4ccccc4)cc(N(c4ccccc4)c4ccccc4)c3)ccc2-c2c(-c3ccccc3)cccc21 ; CC1(C)c2cc(N(c3cccc(-c4ccccc4)c3)c3cc(N(c4ccccc4)c4ccccc4)cc(N(c4ccccc4)c4ccccc4)c3)ccc2-c2c(-c3ccccc3)cccc21 ; CC1(C)c2cc(N(c3cc(N(c4ccccc4)c4ccccc4)cc(N(c4ccccc4)c4ccccc4)c3)c3ccccc3-c3ccccc3)ccc2-c2c(-c3ccccc3)cccc21]

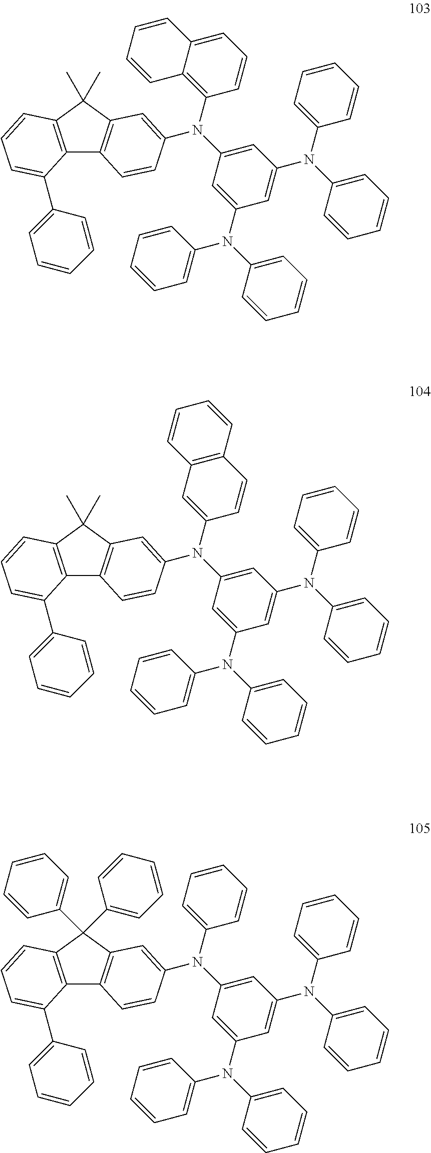 [SMILES: CC1(C)c2cc(N(c3cc(N(c4ccccc4)c4ccccc4)cc(N(c4ccccc4)c4ccccc4)c3)c3cccc4ccccc34)ccc2-c2c(-c3ccccc3)cccc21 ; CC1(C)c2cc(N(c3cc(N(c4ccccc4)c4ccccc4)cc(N(c4ccccc4)c4ccccc4)c3)c3ccc4ccccc4c3)ccc2-c2c(-c3ccccc3)cccc21 ; c1ccc(-c2cccc3c2-c2ccc(N(c4ccccc4)c4cc(N(c5ccccc5)c5ccccc5)cc(N(c5ccccc5)c5ccccc5)c4)cc2C3(c2ccccc2)c2ccccc2)cc1]

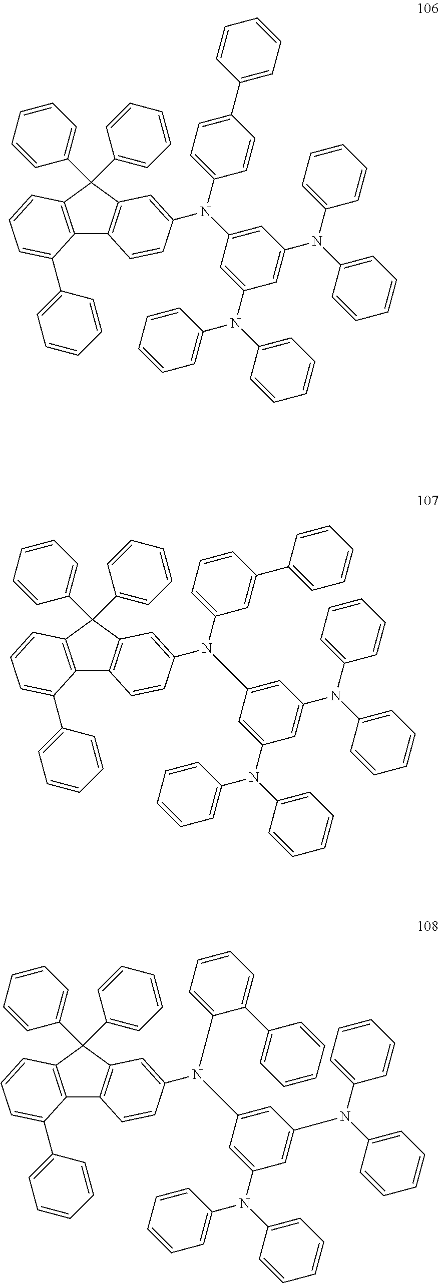 [SMILES: c1ccc(-c2ccc(N(c3cc(N(c4ccccc4)c4ccccc4)cc(N(c4ccccc4)c4ccccc4)c3)c3ccc4c(c3)C(c3ccccc3)(c3ccccc3)c3cccc(-c5ccccc5)c3-4)cc2)cc1 ; c1ccc(-c2cccc(N(c3cc(N(c4ccccc4)c4ccccc4)cc(N(c4ccccc4)c4ccccc4)c3)c3ccc4c(c3)C(c3ccccc3)(c3ccccc3)c3cccc(-c5ccccc5)c3-4)c2)cc1 ; c1ccc(-c2ccccc2N(c2cc(N(c3ccccc3)c3ccccc3)cc(N(c3ccccc3)c3ccccc3)c2)c2ccc3c(c2)C(c2ccccc2)(c2ccccc2)c2cccc(-c4ccccc4)c2-3)cc1]

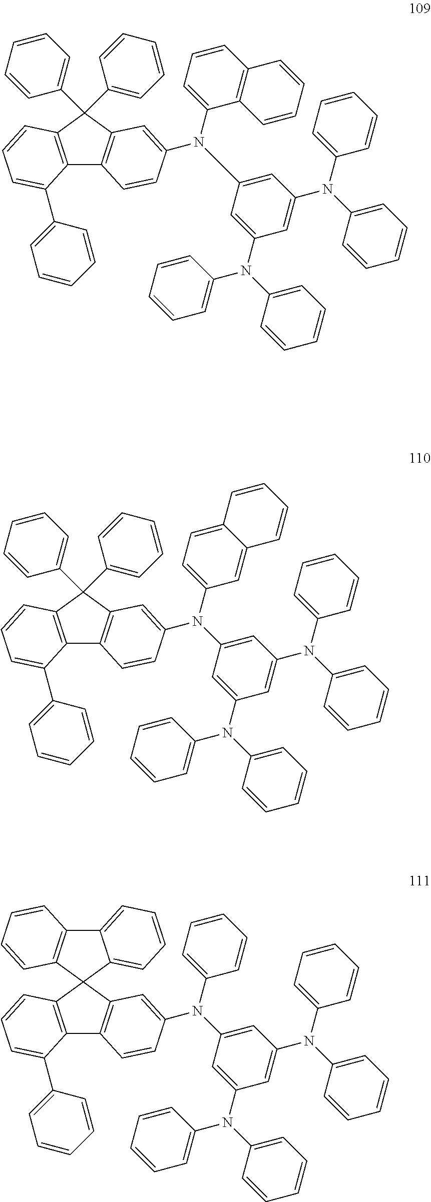 [SMILES: c1ccc(-c2cccc3c2-c2ccc(N(c4cc(N(c5ccccc5)c5ccccc5)cc(N(c5ccccc5)c5ccccc5)c4)c4cccc5ccccc45)cc2C3(c2ccccc2)c2ccccc2)cc1 ; c1ccc(-c2cccc3c2-c2ccc(N(c4cc(N(c5ccccc5)c5ccccc5)cc(N(c5ccccc5)c5ccccc5)c4)c4ccc5ccccc5c4)cc2C3(c2ccccc2)c2ccccc2)cc1 ; c1ccc(-c2cccc3c2-c2ccc(N(c4ccccc4)c4cc(N(c5ccccc5)c5ccccc5)cc(N(c5ccccc5)c5ccccc5)c4)cc2C32c3ccccc3-c3ccccc32)cc1]

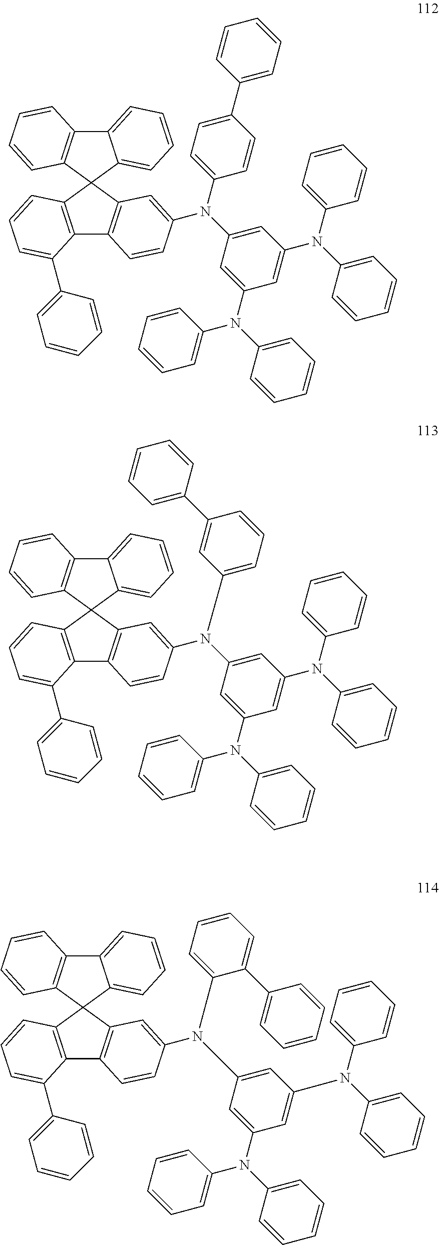 [SMILES: c1ccc(-c2ccc(N(c3cc(N(c4ccccc4)c4ccccc4)cc(N(c4ccccc4)c4ccccc4)c3)c3ccc4c(c3)C3(c5ccccc5-c5ccccc53)c3cccc(-c5ccccc5)c3-4)cc2)cc1 ; c1ccc(-c2cccc(N(c3cc(N(c4ccccc4)c4ccccc4)cc(N(c4ccccc4)c4ccccc4)c3)c3ccc4c(c3)C3(c5ccccc5-c5ccccc53)c3cccc(-c5ccccc5)c3-4)c2)cc1 ; c1ccc(-c2ccccc2N(c2cc(N(c3ccccc3)c3ccccc3)cc(N(c3ccccc3)c3ccccc3)c2)c2ccc3c(c2)C2(c4ccccc4-c4ccccc42)c2cccc(-c4ccccc4)c2-3)cc1]

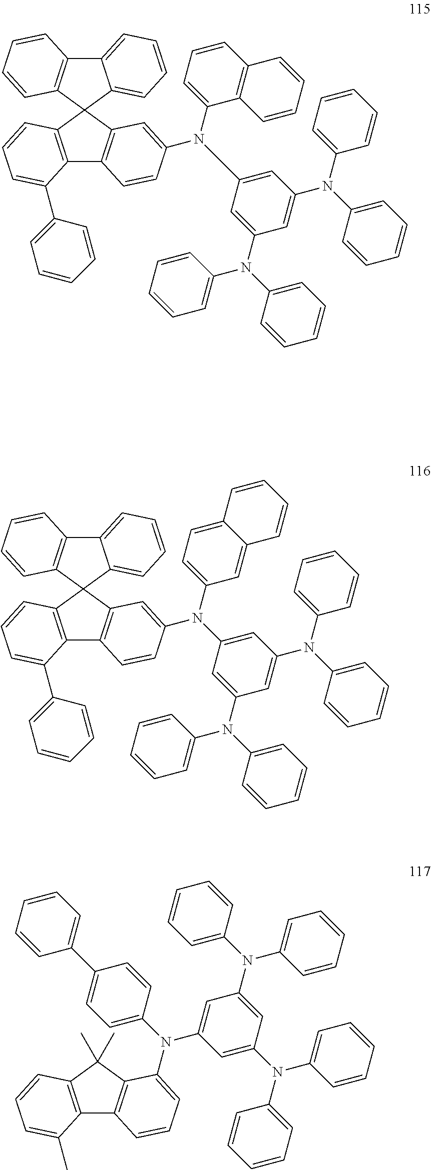 [SMILES: c1ccc(-c2cccc3c2-c2ccc(N(c4cc(N(c5ccccc5)c5ccccc5)cc(N(c5ccccc5)c5ccccc5)c4)c4cccc5ccccc45)cc2C32c3ccccc3-c3ccccc32)cc1 ; c1ccc(-c2cccc3c2-c2ccc(N(c4cc(N(c5ccccc5)c5ccccc5)cc(N(c5ccccc5)c5ccccc5)c4)c4ccc5ccccc5c4)cc2C32c3ccccc3-c3ccccc32)cc1 ; Cc1cccc2c1-c1cccc(N(c3ccc(-c4ccccc4)cc3)c3cc(N(c4ccccc4)c4ccccc4)cc(N(c4ccccc4)c4ccccc4)c3)c1C2(C)C]

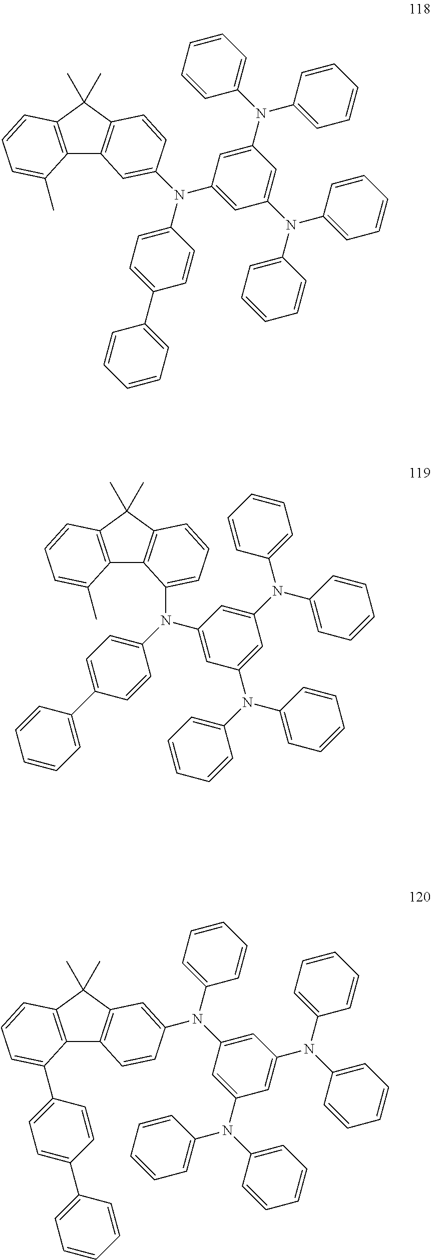 [SMILES: Cc1cccc2c1-c1cc(N(c3ccc(-c4ccccc4)cc3)c3cc(N(c4ccccc4)c4ccccc4)cc(N(c4ccccc4)c4ccccc4)c3)ccc1C2(C)C ; Cc1cccc2c1-c1c(N(c3ccc(-c4ccccc4)cc3)c3cc(N(c4ccccc4)c4ccccc4)cc(N(c4ccccc4)c4ccccc4)c3)cccc1C2(C)C ; CC1(C)c2cc(N(c3ccccc3)c3cc(N(c4ccccc4)c4ccccc4)cc(N(c4ccccc4)c4ccccc4)c3)ccc2-c2c(-c3ccc(-c4ccccc4)cc3)cccc21]

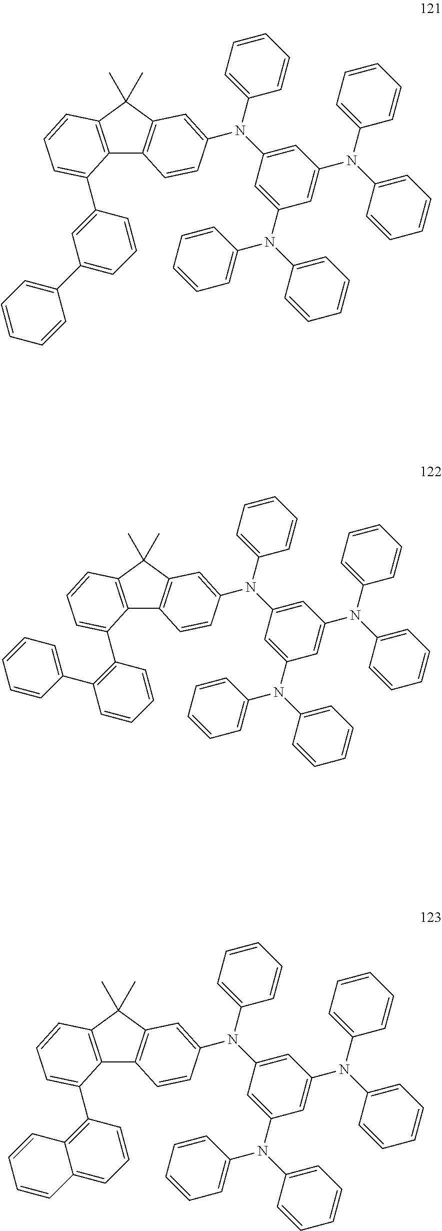 [SMILES: CC1(C)c2cc(N(c3ccccc3)c3cc(N(c4ccccc4)c4ccccc4)cc(N(c4ccccc4)c4ccccc4)c3)ccc2-c2c(-c3cccc(-c4ccccc4)c3)cccc21 ; CC1(C)c2cc(N(c3ccccc3)c3cc(N(c4ccccc4)c4ccccc4)cc(N(c4ccccc4)c4ccccc4)c3)ccc2-c2c(-c3ccccc3-c3ccccc3)cccc21 ; CC1(C)c2cc(N(c3ccccc3)c3cc(N(c4ccccc4)c4ccccc4)cc(N(c4ccccc4)c4ccccc4)c3)ccc2-c2c(-c3cccc4ccccc34)cccc21]

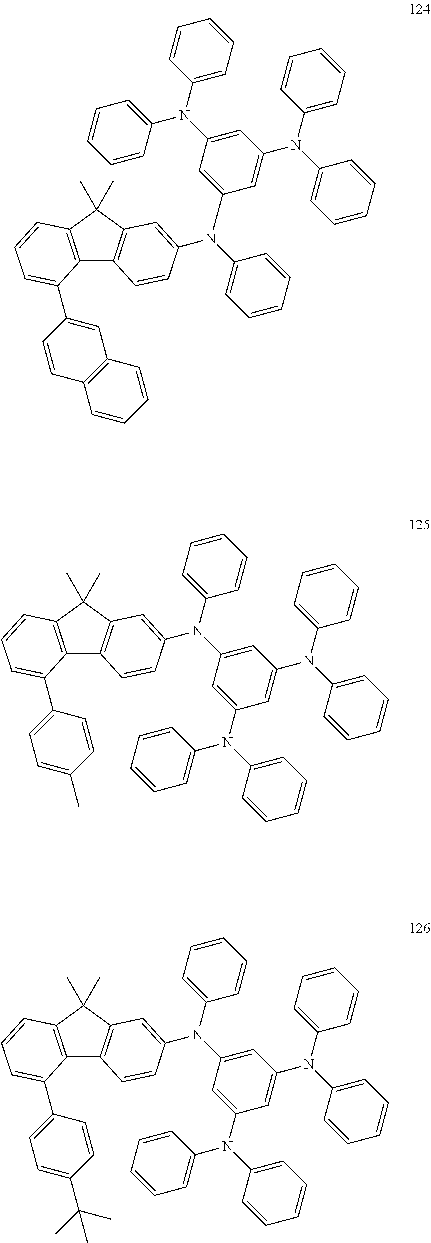 [SMILES: CC1(C)c2cc(N(c3ccccc3)c3cc(N(c4ccccc4)c4ccccc4)cc(N(c4ccccc4)c4ccccc4)c3)ccc2-c2c(-c3ccc4ccccc4c3)cccc21 ; Cc1ccc(-c2cccc3c2-c2ccc(N(c4ccccc4)c4cc(N(c5ccccc5)c5ccccc5)cc(N(c5ccccc5)c5ccccc5)c4)cc2C3(C)C)cc1 ; CC(C)(C)c1ccc(-c2cccc3c2-c2ccc(N(c4ccccc4)c4cc(N(c5ccccc5)c5ccccc5)cc(N(c5ccccc5)c5ccccc5)c4)cc2C3(C)C)cc1]

A light-emitting device according to one or more aspects of the present disclosure may include:
a first electrode;
a second electrode facing (e.g., opposite to and facing) the first electrode; and
an interlayer between the first electrode and the second electrode and including an emission layer,
wherein the interlayer may include the compound represented by Formula 1.

In one or more embodiments,
an electronic apparatus may include the light-emitting device.

In one or more embodiments, the electronic apparatus may further include a thin-film transistor,
wherein the thin-film transistor includes a source electrode and a drain electrode, and
the first electrode of the light-emitting device may be electrically connected to the source electrode or the drain electrode of the thin-film transistor.

The term “interlayer” as utilized herein refers to a single layer and/or all layers between the first electrode and the second electrode of the light-emitting device.

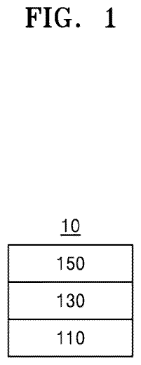

### Description of FIG. 1

FIG. 1 is a schematic cross-sectional view of a light-emitting device 10 according to one or more embodiments of the present disclosure. The light-emitting device 10 may include a first electrode 110, an interlayer 130, and a second electrode 150.

Hereinafter, a structure of the light-emitting device 10 according to one or more embodiments and a method of manufacturing the light-emitting device 10 are described in more detail with reference to FIG. 1.

### First Electrode 110

In FIG. 1, in one or more embodiments, a substrate may be additionally provided and arranged under the first electrode 110 and/or on the second electrode 150. As the substrate, a glass substrate or a plastic substrate may be utilized. In one or more embodiments, the substrate may be a flexible substrate and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure including (e.g., consisting of) a single layer or a multilayer structure including a plurality of layers. In some embodiments, the first electrode 110 may have a three-layer structure of ITO/Ag/ITO.

### Interlayer 130

The interlayer 130 is disposed above (e.g., on) the first electrode 110. The interlayer 130 may include an emission layer.

In one or more embodiments, the interlayer 130 may further include a hole transport region between the first electrode 110 and the emission layer, and an electron transport region between the emission layer and the second electrode 150.

In one or more embodiments, the interlayer 130 may further include, in addition to one or more suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer between the two or more emitting units. When the interlayer 130 includes emitting units and a charge generation layer as described above, the light-emitting device 10 may be a tandem light-emitting device.

### Hole Transport Region in Interlayer 130

The hole transport region may have: i) a single-layer structure including (e.g., consisting of) a single layer including (e.g., consisting of) a single material, ii) a single-layer structure including (e.g., consisting of) a single layer including (e.g., consisting of) a plurality of materials that are different from each other, or iii) a multilayer structure including a plurality of layers including a plurality of materials that are different from each other.

The hole transport region may include a hole transport layer, a hole injection layer, an emission auxiliary layer, an electron blocking layer, or any combination thereof.

In one or more embodiments, the hole transport region may have a multilayer structure including a hole injection layer/hole transport layer structure, a hole injection layer/hole transport layer/emission auxiliary layer structure, a hole transport layer/emission auxiliary layer structure, or a hole injection layer/hole transport layer/electron blocking layer structure, the constituent layers of each structure being stacked sequentially stacked from the first electrode 110 in the stated order.

In one or more embodiments, the hole transport region may include a compound represented by Formula 201, a compound represented by Formula 202, or any combination thereof:

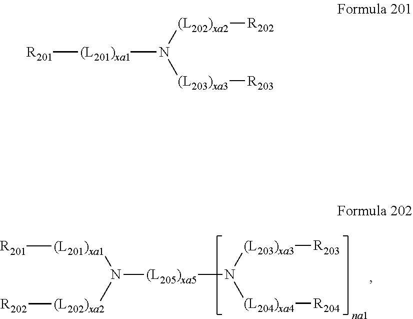

wherein, in Formulae 201 and 202,
L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
L₂₀₅ may be *—O—*′, *—S—*′, *—N(Q₂₀₁)-*′, a C₁-C₂₀ alkylene group that is unsubstituted or substituted with at least one R₁₀ₐ, a C₂-C₂₀ alkenylene group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
R₂₀₁ and R₂₀₂ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ, to form a C₈-C₆₀ polycyclic group (for example, a carbazole group) that is unsubstituted or substituted with at least one R₁₀ₐ (for example, see Compound HT16),
R₂₀₃ and R₂₀₄ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ, to form a C₈-C₆₀ polycyclic group that is unsubstituted or substituted with at least one R₁₀ₐ, and
na1 may be an integer from 1 to 4.

In one or more embodiments, each of Formulae 201 and 202 may include at least one selected from among groups represented by Formulae CY201 to CY217:

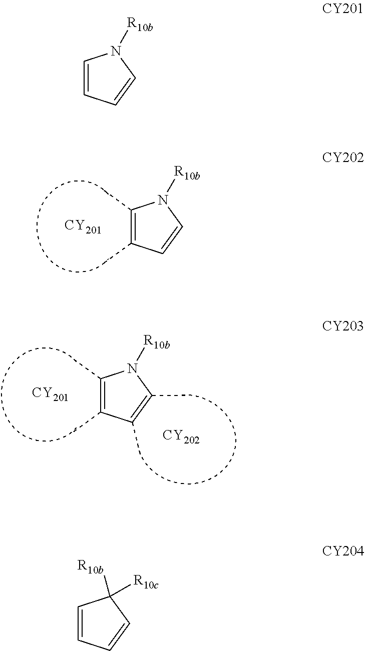

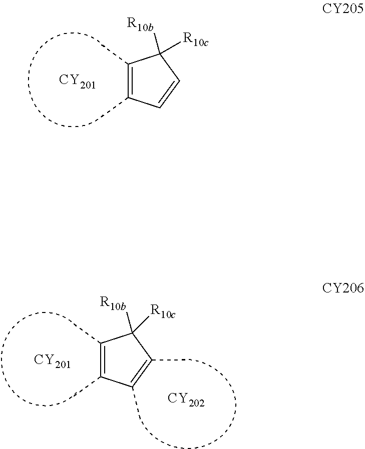

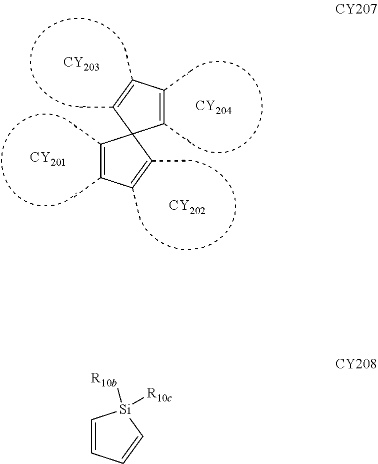

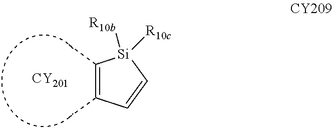

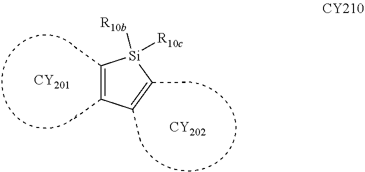

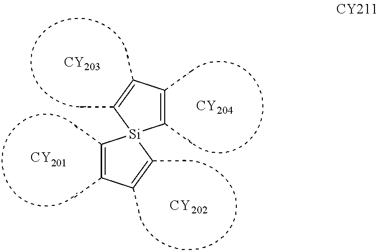

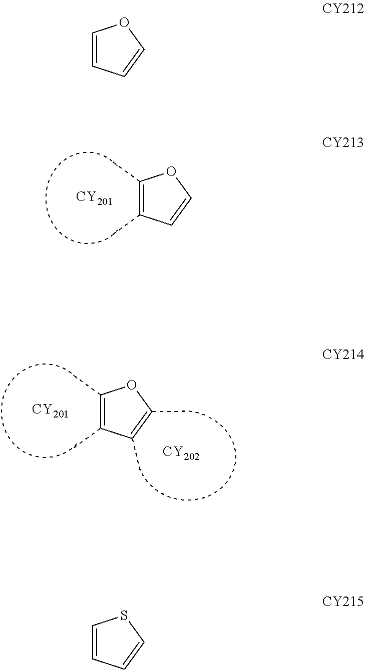

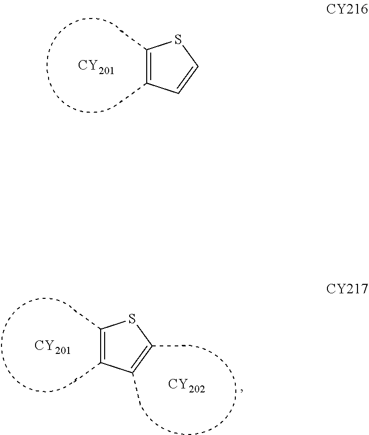

wherein, in Formulae CY201 to CY217, R_{10b}, and R_{10c} may each be the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from among the groups represented by Formulae CY201 to CY203.

In one or more embodiments, Formula 201 may include at least one selected from among the groups represented by Formulae CY201 to CY203 and at least one selected from among the groups represented by Formulae CY204 to CY217.

In one or more embodiments, in Formula 201, xa1 may be 1, R₂₀₁ may be a group represented by one selected from among Formulae CY201 to CY203, xa2 may be 0, and R₂₀₂ may be a group represented by one selected from among Formulae CY204 to CY207.

In one or more embodiments, each of Formulae 201 and 202 may not include (e.g., may exclude) any of the groups represented by Formulae CY201 to CY203.

In one or more embodiments, each of Formulae 201 and 202 may not include (e.g., may exclude) any of the groups represented by Formulae CY201 to CY203 and may include at least one selected from among the groups represented by Formulae CY204 to CY217.

In one or more embodiments, each of Formulae 201 and 202 may not include (e.g., may exclude) any of the groups represented by Formulae CY201 to CY217. In present disclosure, “not include a or any ‘component’” “exclude a or any ‘component’”, “‘component’-free”, and/or the like refers to that the “component” not being added, selected or utilized as a component in the composition/formula/structure, but, in some embodiments, the “component” of less than a suitable amount may still be included due to other impurities and/or external factors.

In one or more embodiments, the hole transport region may include at least one selected from among Compounds HT1 to HT46, 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N,N′-di(naphthalen-1-yl)-N,N′-diphenyl-benzidine (NPB(NPD)), β-NPB, N,N′-bis(3-methylphenyl)-N,N′-diphenyl-[1,1′-biphenyl]-4,4′-diamine (TPD), Spiro-TPD, Spiro-NPB, methylated NPB, 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzene sulfonic acid (PANI/DBSA), poly(3,4-ethylene dioxythiophene)/poly(4-styrene sulfonate) (PEDOT/PSS), polyaniline/camphor sulfonic acid (PANI/CSA), polyaniline/poly(4-styrene sulfonate) (PANI/PSS), or any combination thereof:

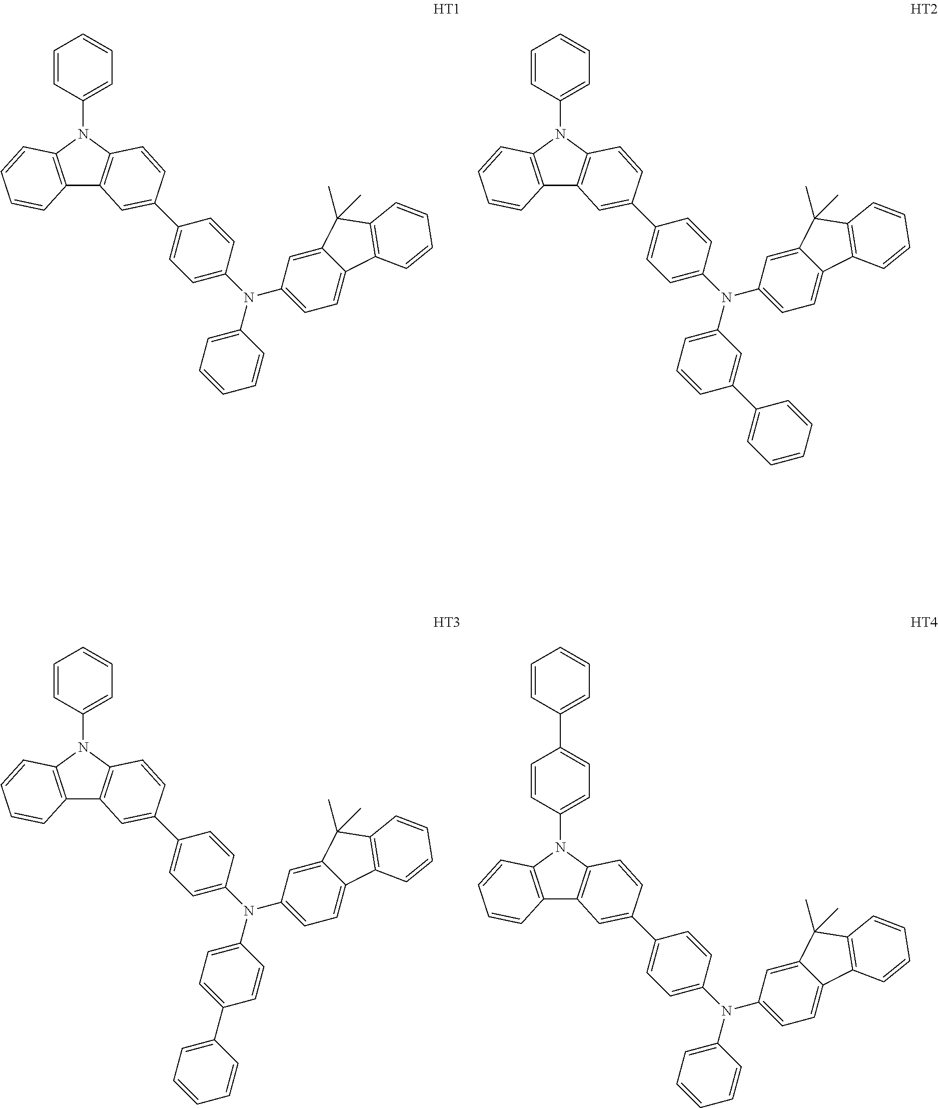 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

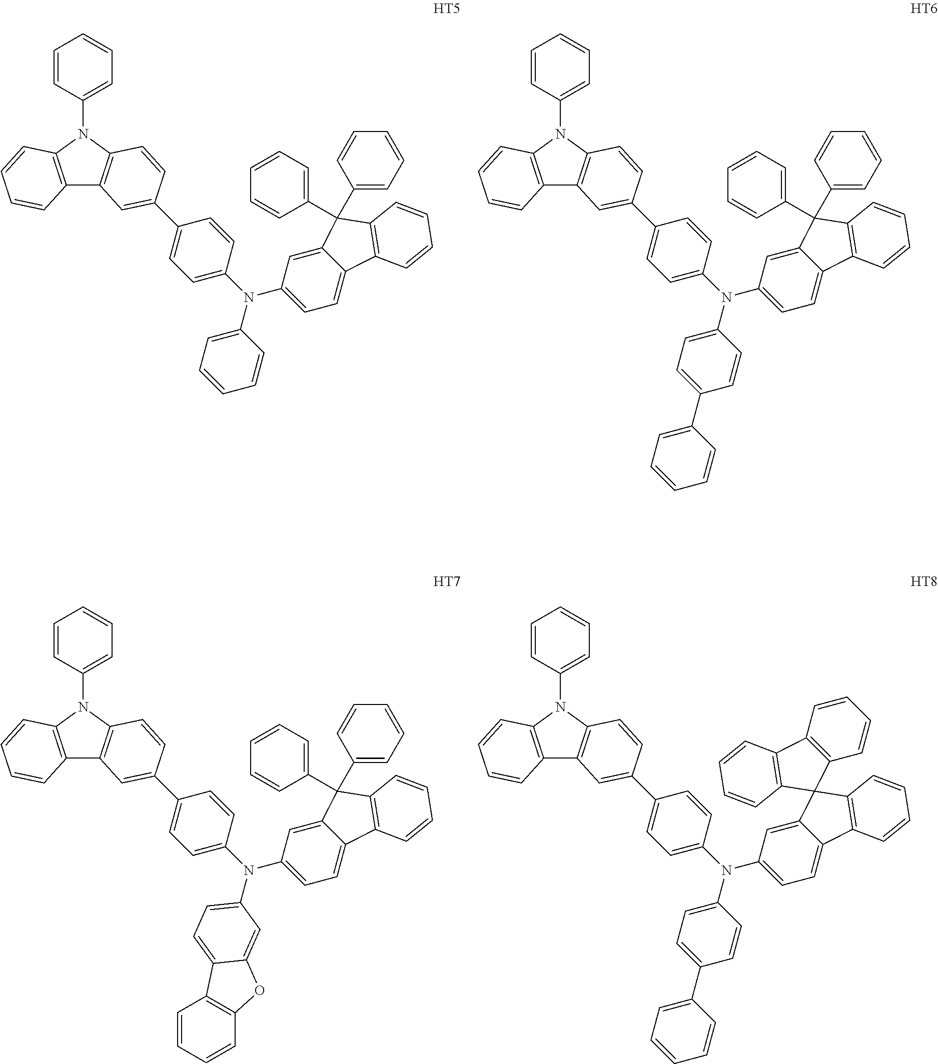 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

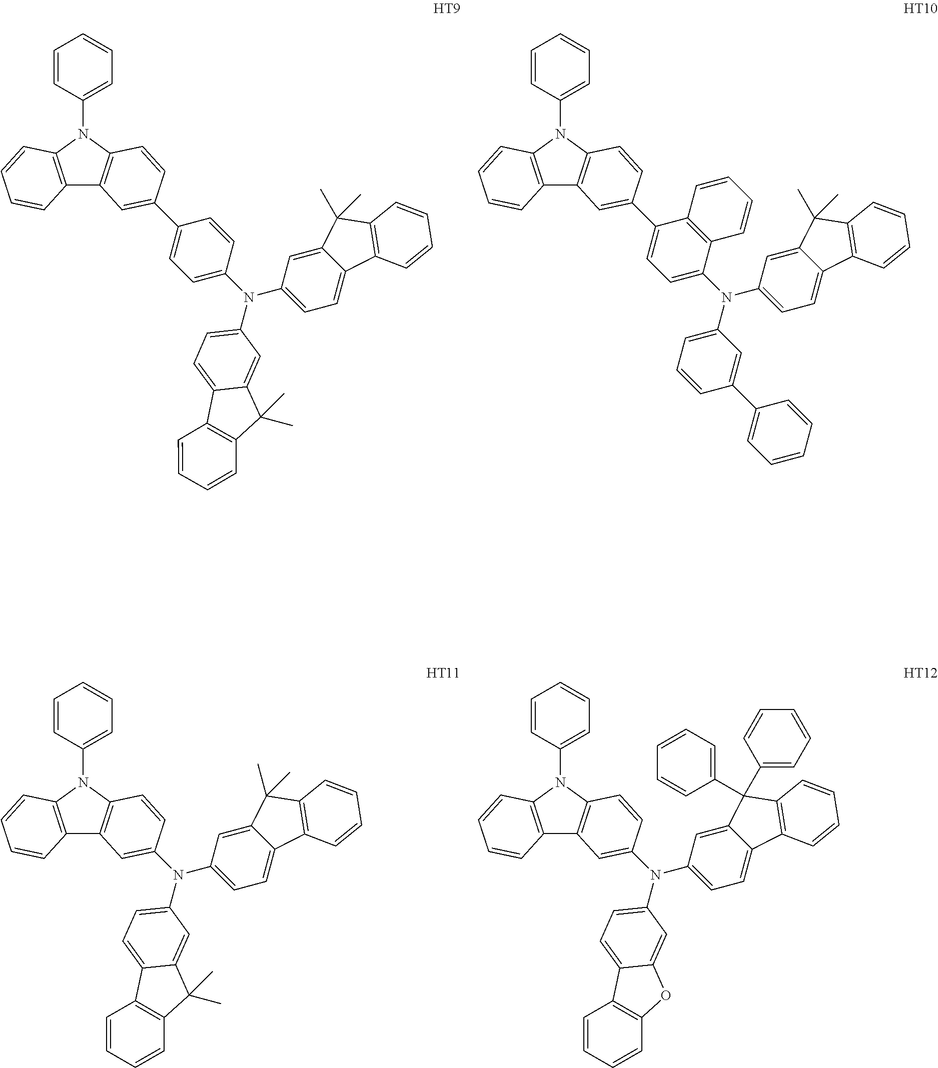 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

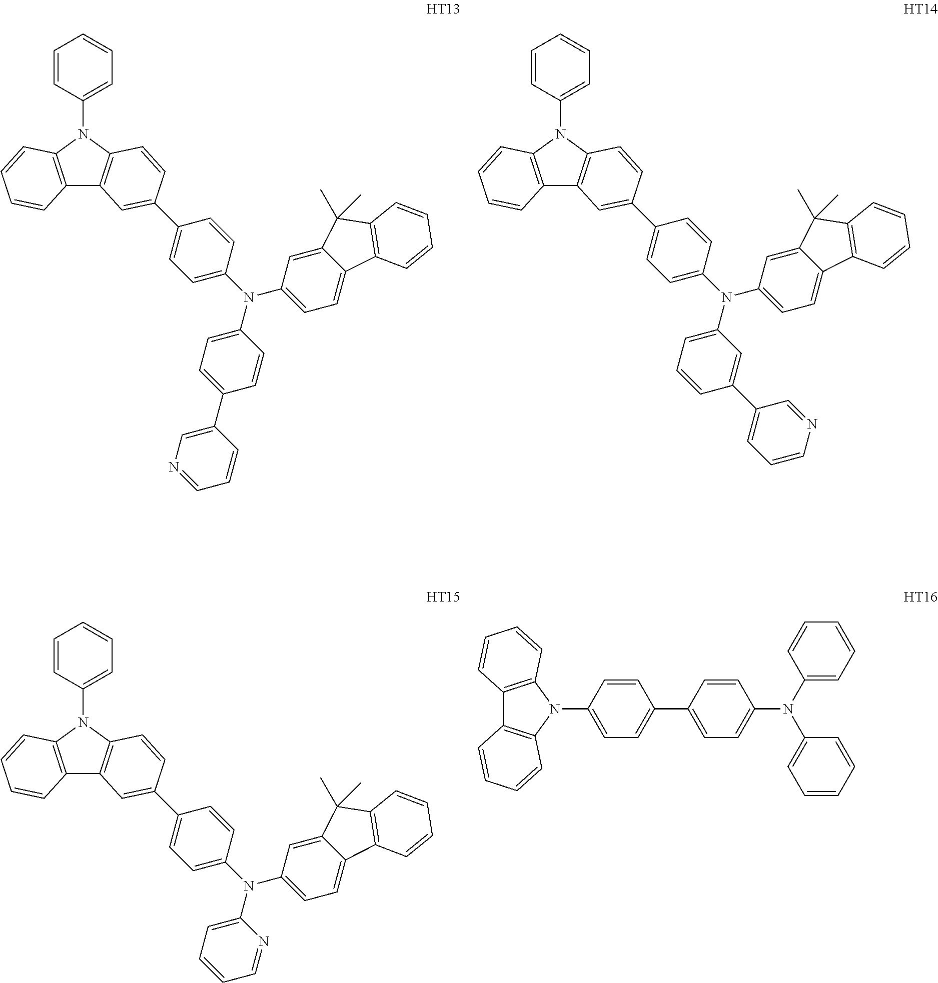 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

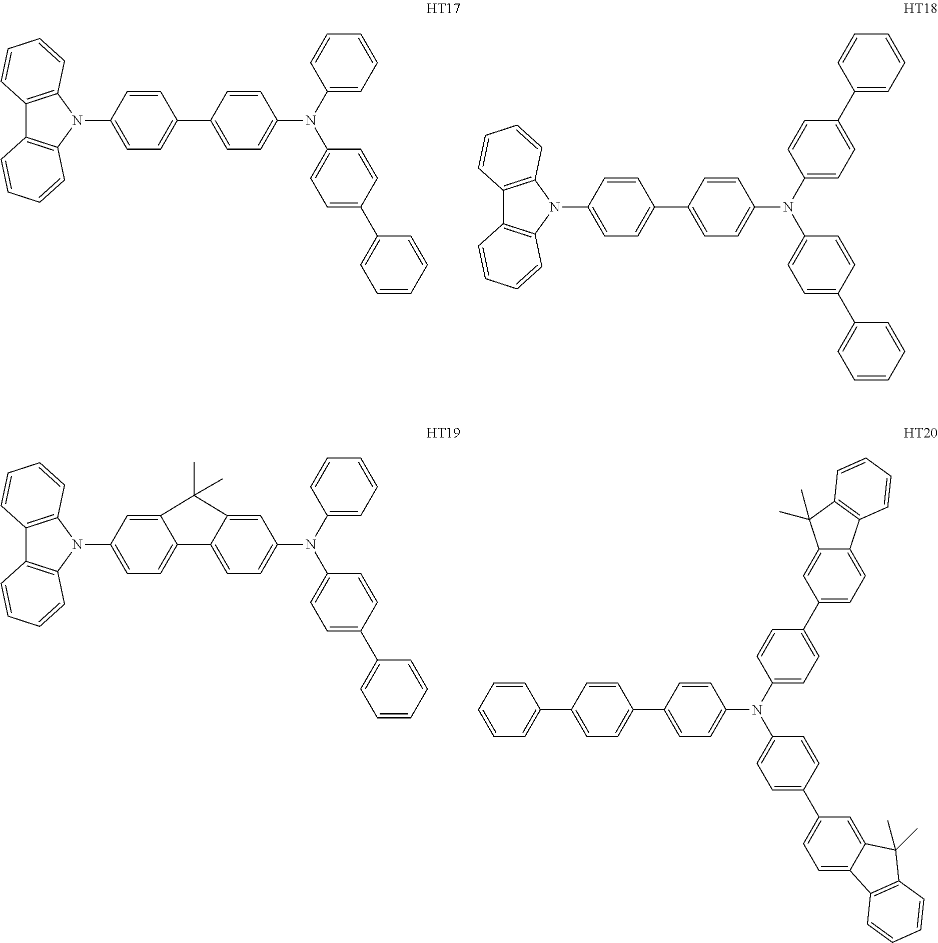 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

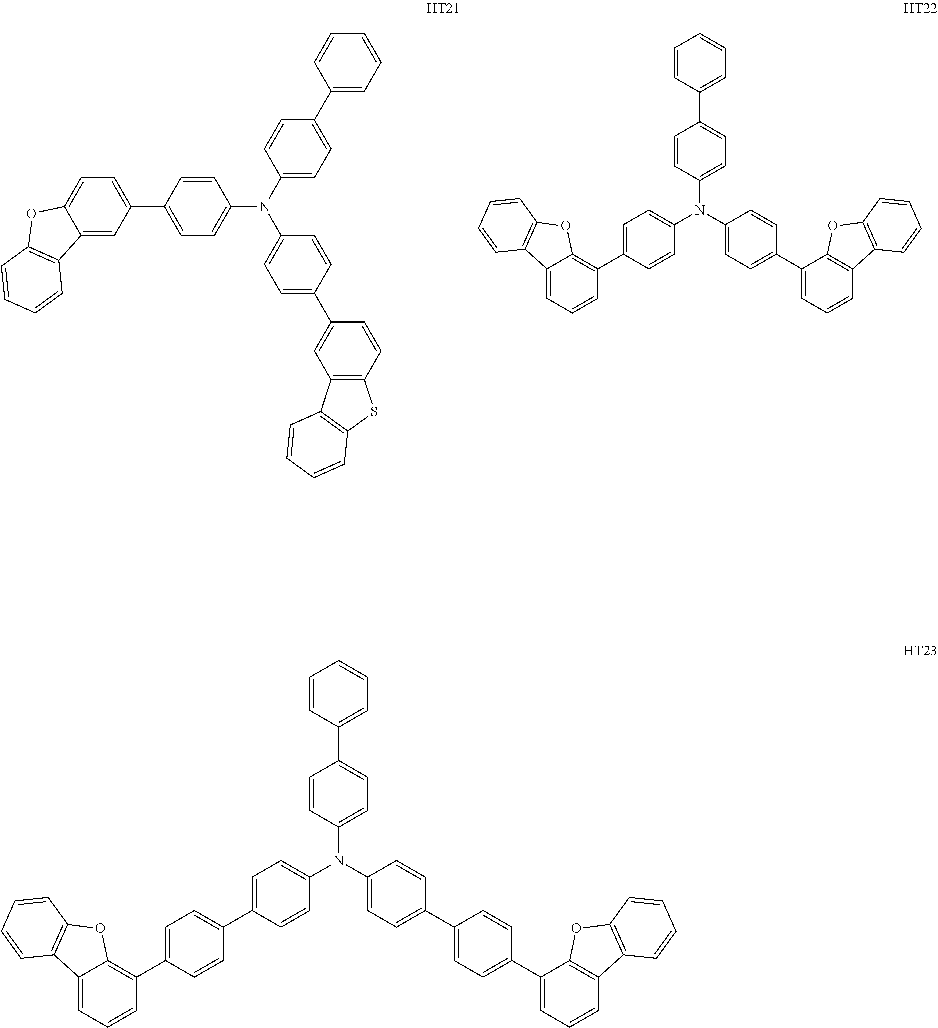 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

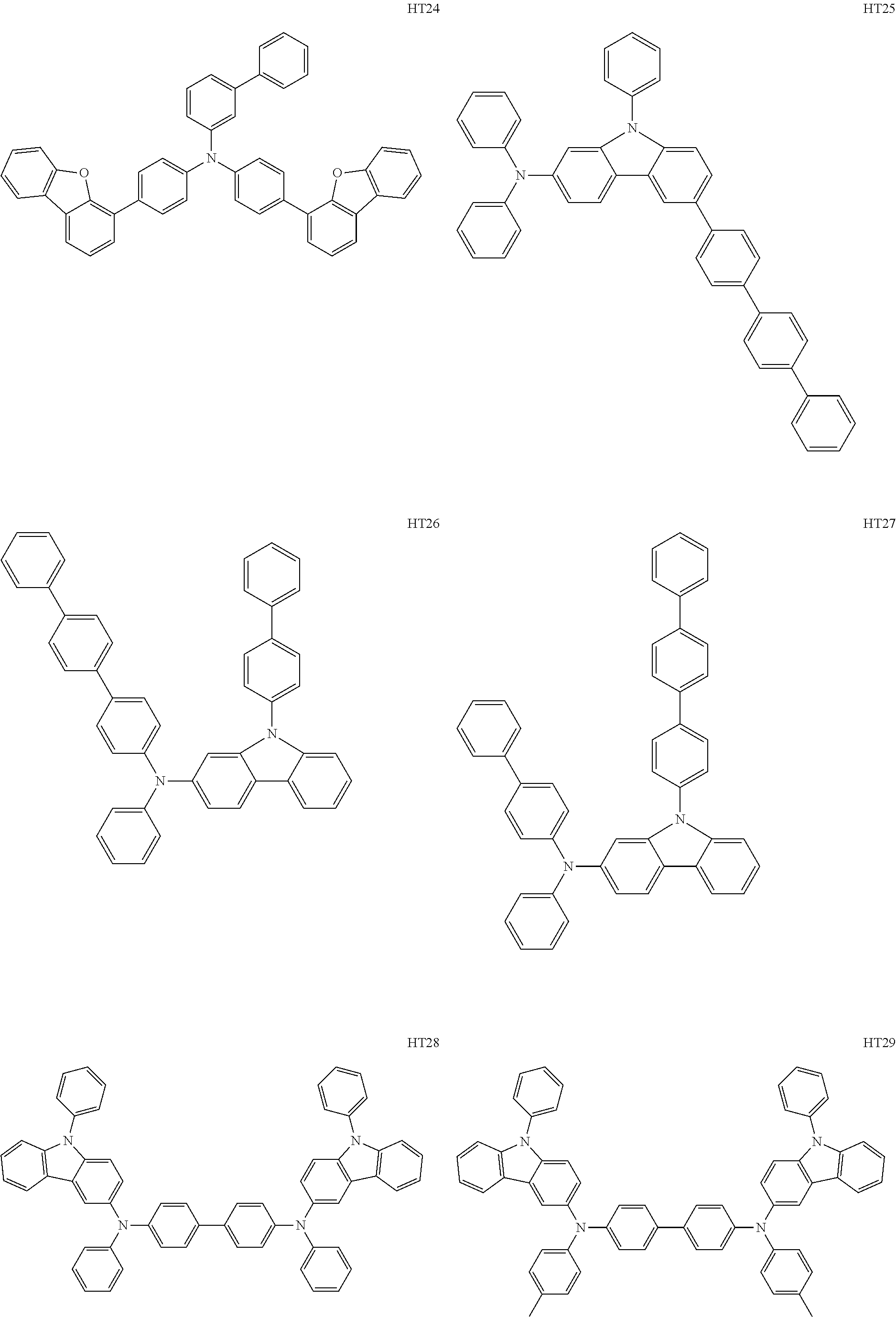

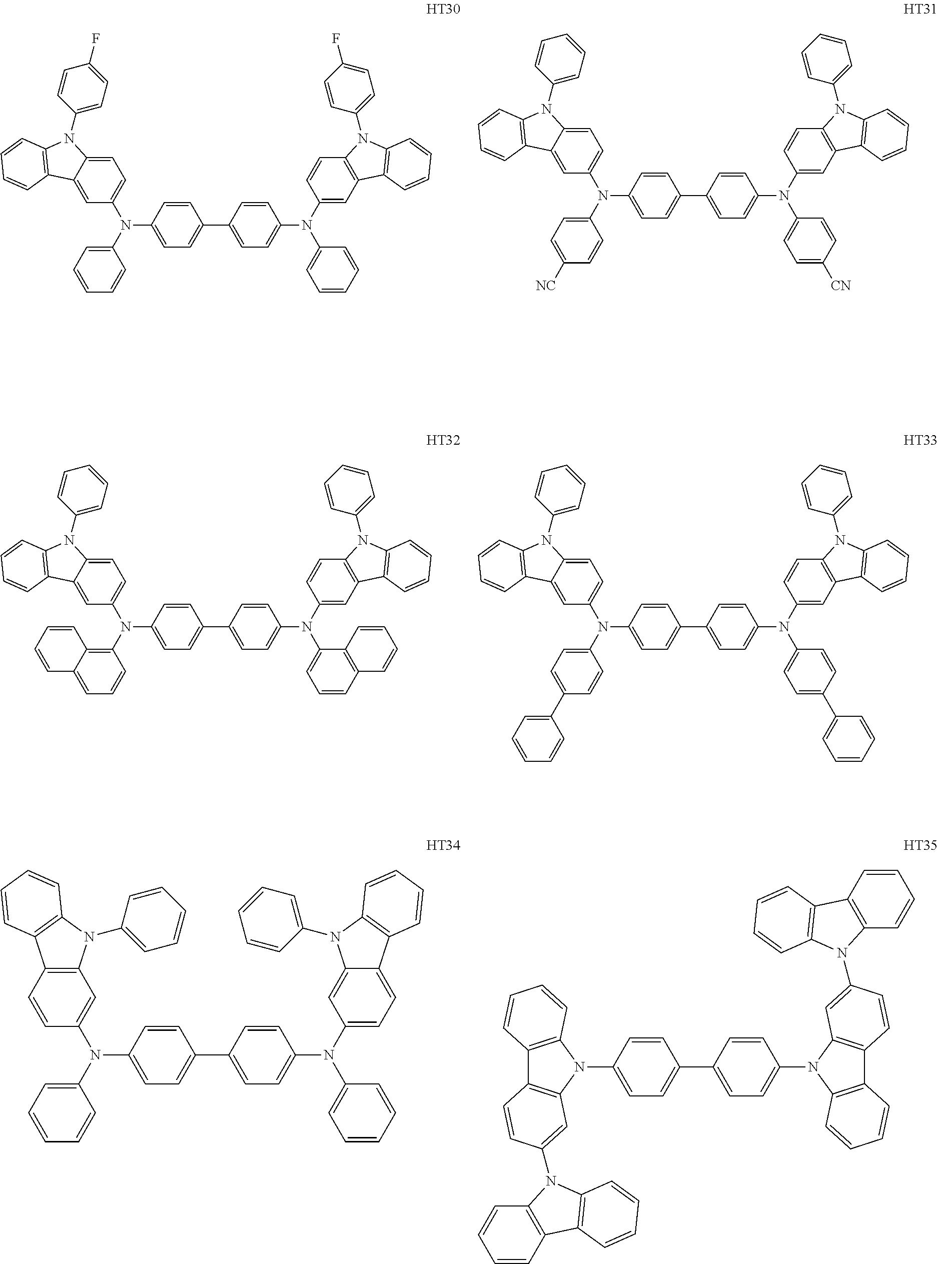

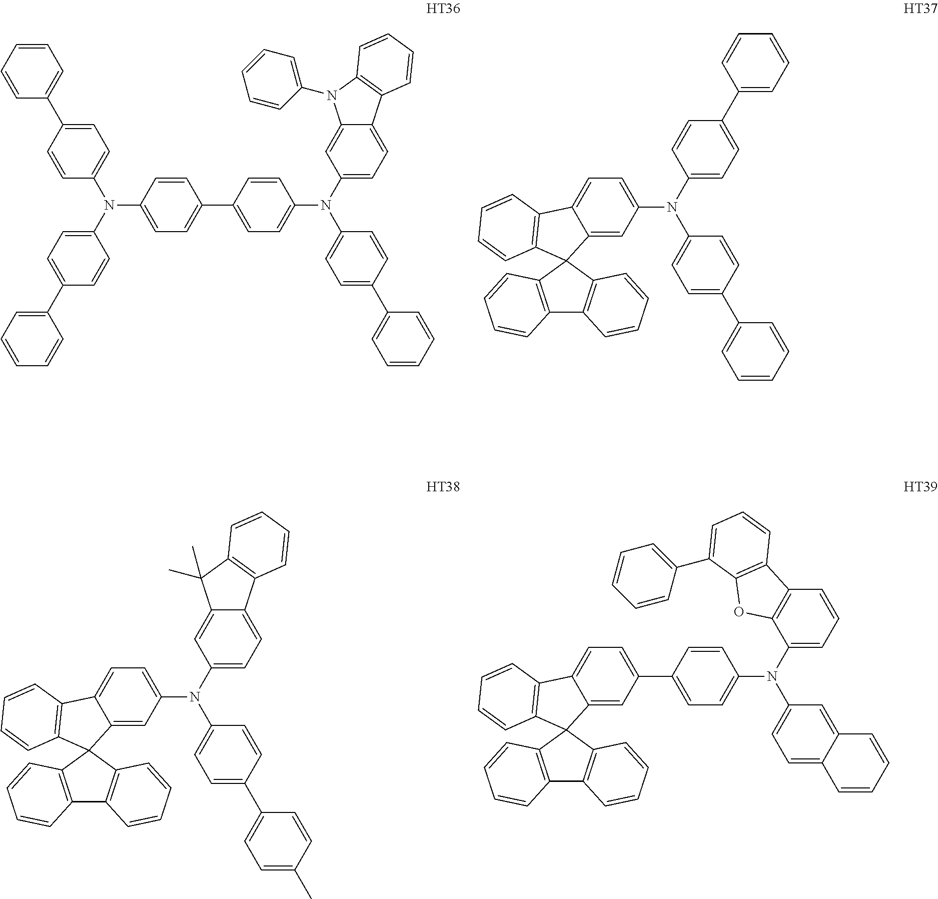 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

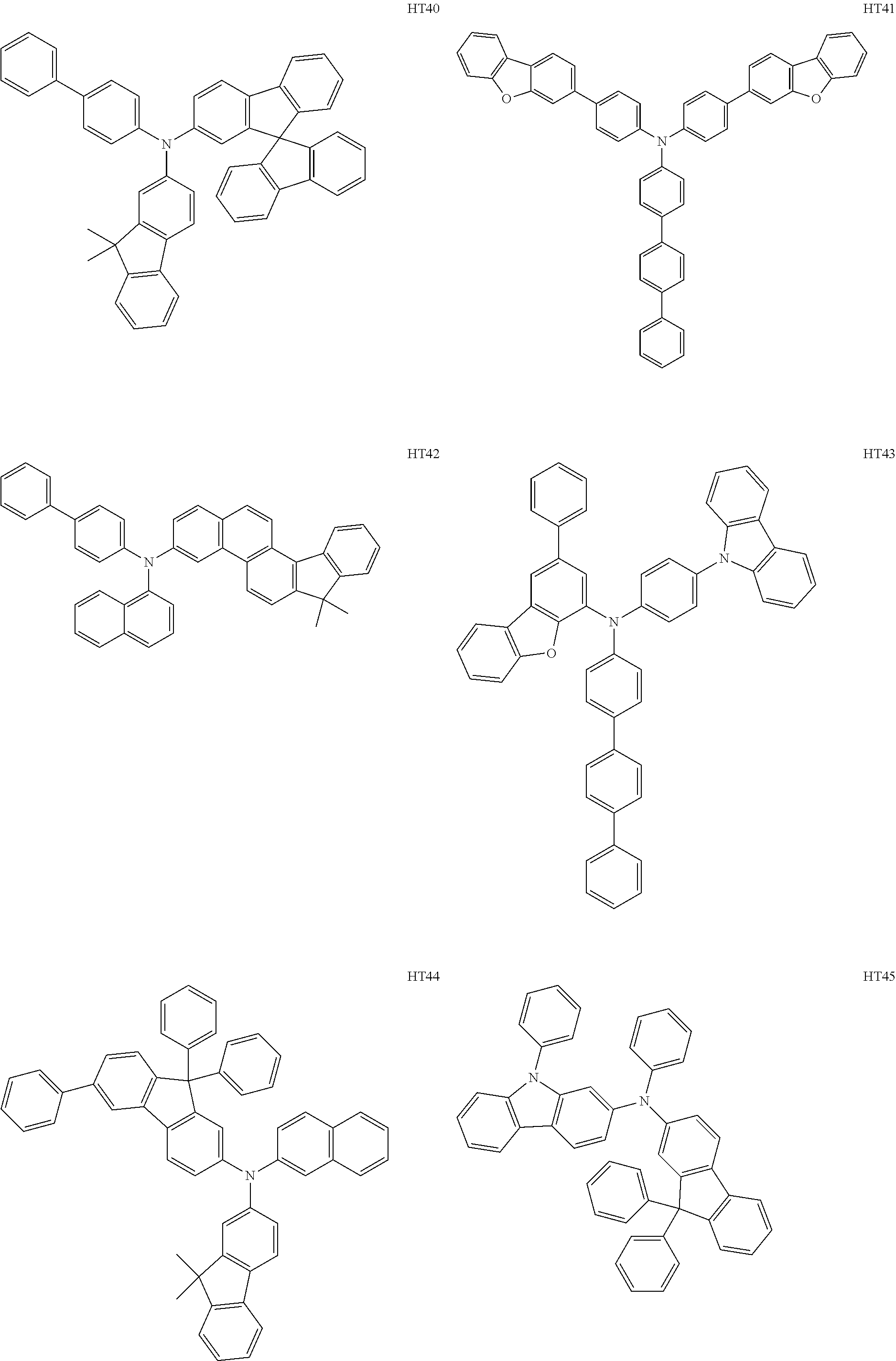

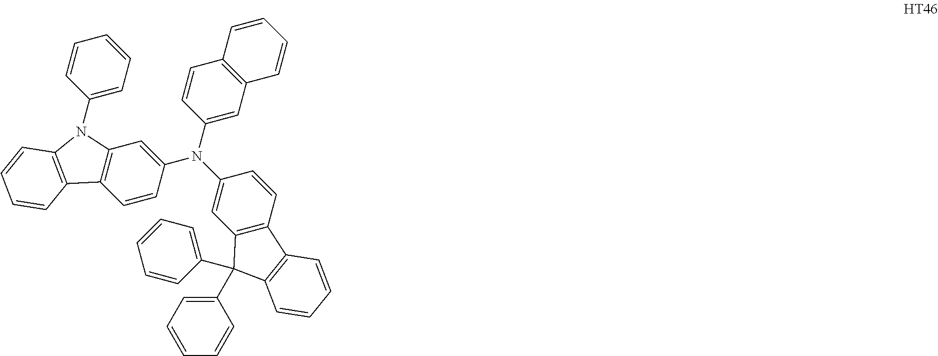 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

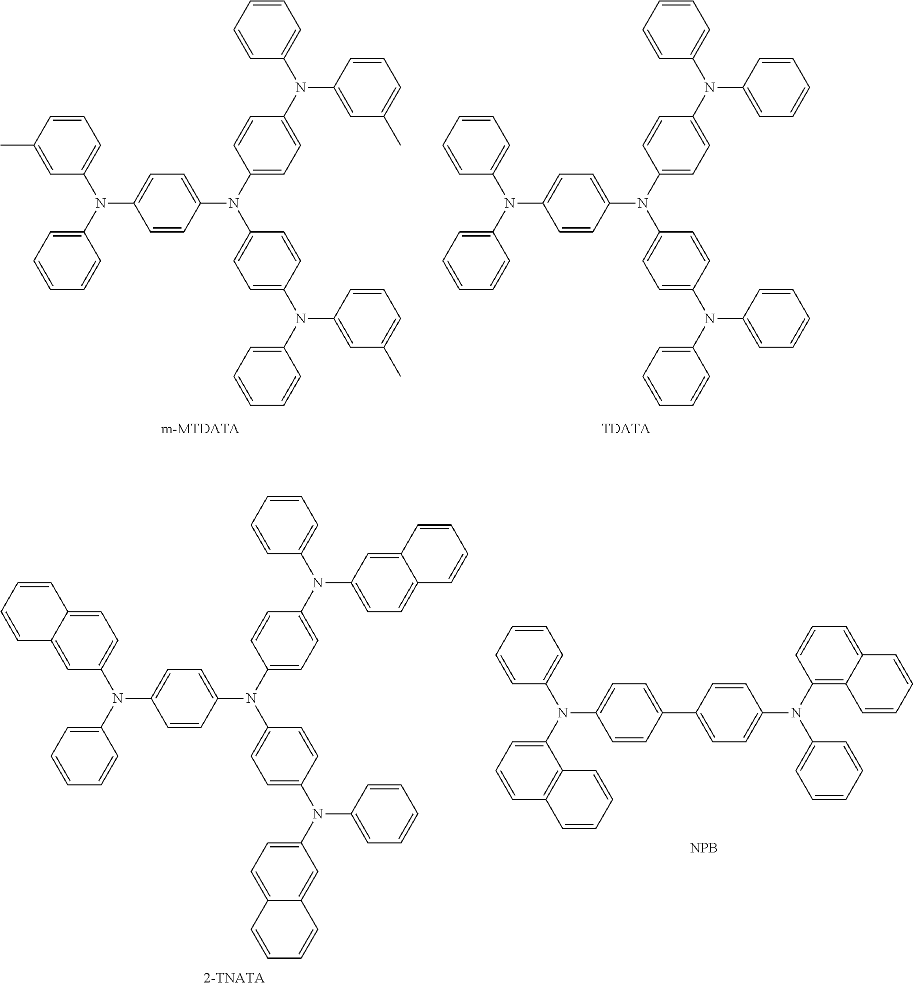

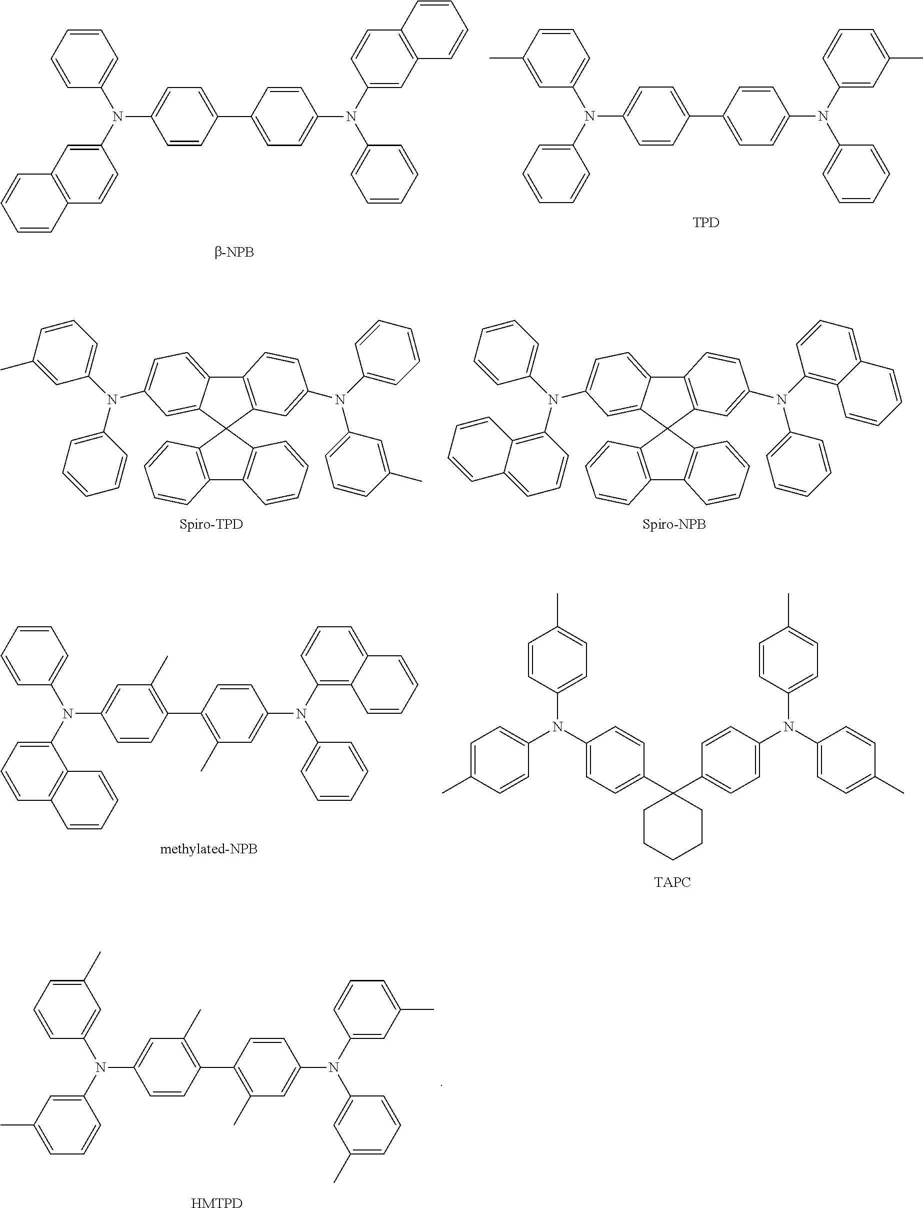

In one or more embodiments, the hole transport layer may include the compound represented by Formula 1.

A thickness of the hole transport region may be about 50 Å to about 10,000 Å, for example, about 100 Å to about 4,000 Å. When the hole transport region includes a hole injection layer, a hole transport layer, or any combination thereof, a thickness of the hole injection layer may be about 100 Å to about 9,000 Å, for example, about 100 Å to about 1,000 Å, and a thickness of the hole transport layer may be about 50 Å to about 2,000 Å, for example, about 100 Å to about 1,500 Å. When the thicknesses of the hole transport region, the hole injection layer, and the hole transport layer are within the ranges described above, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may block or reduce the leakage of electrons from the emission layer to the hole transport region. Materials that may be included in the hole transport region may be included in the emission auxiliary layer and the electron blocking layer.

p-Dopant

In one or more embodiments, the hole transport region may further include, in addition to one or more of these aforementioned materials, a charge-generation material for the improvement of conductive properties. The charge-generation material may be substantially uniformly or non-uniformly dispersed in the hole transport region (for example, in the form of a single layer including (e.g., consisting of) a charge-generation material).

The charge-generation material may be, for example, a p-dopant.

For example, in one or more embodiments, a lowest unoccupied molecular orbital (LUMO) energy level of the p-dopant may be less than or equal to −3.5 eV.

In one or more embodiments, the p-dopant may include a quinone derivative, a cyano group-containing compound, a compound including an element EL1 and an element EL2, or any combination thereof.

Non-limiting examples of the quinone derivative may include tetracyanoquinodimethane (TCNQ) and 2,3,5,6-tetrafluoro-7,7,8,8-tetracyanoquinodimethane (F4-TCNQ).

Non-limiting examples of the cyano group-containing compound may include dipyrazino[2,3-f: 2′,3′-h]quinoxaline-2,3,6,7,10,11-hexacarbonitrile (HAT-CN) and a compound represented by Formula 221.

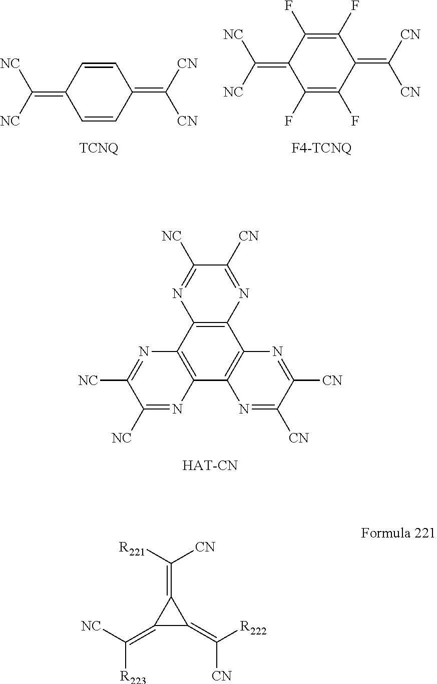

In Formula 221,
R₂₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, or any combination thereof, and the element EL2 may be a non-metal, a metalloid, or any combination thereof.

Non-limiting examples of the metal may include an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and/or a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Non-limiting examples of the metalloid may include silicon (Si), antimony (Sb), and/or tellurium (Te).

Non-limiting examples of the non-metal may include oxygen (O) and/or a halogen (for example, F, Cl, Br, I, etc.).

Non-limiting examples of the compound including the element EL1 and the element EL2 may include a metal oxide, a metal halide (for example, a metal fluoride, a metal chloride, a metal bromide, a metal iodide, etc.), a metalloid halide (for example, a metalloid fluoride, a metalloid chloride, a metalloid bromide, a metalloid iodide, etc.), a metal telluride, or any combination thereof.

Non-limiting examples of the metal oxide may include a tungsten oxide (for example, WO, W₂O₃, WO₂, WO₃, W₂O₅, etc.), a vanadium oxide (for example, VO, V₂O₃, VO₂, V₂O₅, etc.), a molybdenum oxide (MoO, Mo₂O₃, MoO₂, MoO₃, Mo₂O₅, etc.), and/or a rhenium oxide (for example, ReO₃, etc.).

Non-limiting examples of the metal halide may include an alkali metal halide, an alkaline earth metal halide, a transition metal halide, a post-transition metal halide, and/or a lanthanide metal halide.

Non-limiting examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and/or CsI.

Non-limiting examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and/or BaI₂.

Non-limiting examples of the transition metal halide may include a titanium halide (for example, TiF₄, TiC₄, TiBr₄, TiI₄, etc.), a zirconium halide (for example, ZrF₄, ZrCl₄, ZrBr₄, ZrI₄, etc.), a hafnium halide (for example, HfF₄, HfCl₄, HfBr₄, HfI₄, etc.), a vanadium halide (for example, VF₃, VCl₃, VBr₃, VI₃, etc.), a niobium halide (for example, NbF₃, NbCl₃, NbBr₃, NbI₃, etc.), a tantalum halide (for example, TaF₃, TaCl₃, TaBr₃, TaI₃, etc.), a chromium halide (for example, CrF₃, CrO₃, CrBr₃, CrI₃, etc.), a molybdenum halide (for example, MoF₃, MoCl₃, MoBr₃, MoI₃, etc.), a tungsten halide (for example, WF₃, WCI₃, WBr₃, WI₃, etc.), a manganese halide (for example, MnF₂, MnCl₂, MnBr₂, MnI₂, etc.), a technetium halide (for example, TcF₂, TcCl₂, TcBr₂, TcI₂, etc.), a rhenium halide (for example, ReF₂, ReCl₂, ReBr₂, ReI₂, etc.), a ferrous halide (for example, FeF₂, FeCl₂, FeBr₂, FeI₂, etc.), a ruthenium halide (for example, RuF₂, RuCl₂, RuBr₂, RuI₂, etc.), an osmium halide (for example, OsF₂, OsCl₂, OsBr₂, OsI₂, etc.), a cobalt halide (for example, CoF₂, COCl₂, CoBr₂, CoI₂, etc.), a rhodium halide (for example, RhF₂, RhCl₂, RhBr₂, RhI₂, etc.), an iridium halide (for example, IrF₂, IrCl₂, IrBr₂, IrI₂, etc.), a nickel halide (for example, NiF₂, NiCl₂, NiBr₂, NiI₂, etc.), a palladium halide (for example, PdF₂, PdCl₂, PdBr₂, PdI₂, etc.), a platinum halide (for example, PtF₂, PtCl₂, PtBr₂, PtI₂, etc.), a cuprous halide (for example, CuF, CuCl, CuBr, CuI, etc.), a silver halide (for example, AgF, AgCl, AgBr, AgI, etc.), and/or a gold halide (for example, AuF, AuCl, AuBr, AuI, etc.).

Non-limiting examples of the post-transition metal halide may include a zinc halide (for example, ZnF₂, ZnCl₂, ZnBr₂, ZnI₂, etc.), an indium halide (for example, InI₃, etc.), and/or a tin halide (for example, SnI₂, etc.).

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, and/or SmI₃.

Non-limiting examples of the metalloid halide may include an antimony halide (for example, SbCl₅, etc.).

Non-limiting examples of the metal telluride may include an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (for example, ZnTe, etc.), and/or a lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers selected from among a red emission layer, a green emission layer, and a blue emission layer, in which the two or more layers contact each other or are separated from each other, to emit white light (e.g., combined white light). In one or more embodiments, the emission layer may include two or more materials selected from among a red light-emitting material, a green light-emitting material, and a blue light-emitting material, in which the two or more materials are mixed with each other in a single layer, to emit white light (e.g., combined white light).

The emission layer may include a host and a dopant. The dopant may include a phosphorescent dopant, a fluorescent dopant, or any combination thereof.

An amount of the dopant in the emission layer may be from about 0.01 part by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act as a host or a dopant in the emission layer.

In one or more embodiments, the emission layer may include two hosts. In one or more embodiments, the emission layer may include a hole transporting host and an electron transporting host.

In one or more embodiments, the emission layer may include two dopants. In one or more embodiments, the emission layer may include a phosphorescent dopant and a fluorescent dopant.

In one or more embodiments, the fluorescent dopant may include a delayed fluorescence material.

A thickness of the emission layer may be about 100 Å to about 1,000 Å, for example, about 200 Å to about 600 Å. When the thickness of the emission layer is within the range described above, excellent or suitable luminescence characteristics may be obtained without a substantial increase in driving voltage.

### Host

The hole transporting host may be a compound having strong hole properties. The expression “a compound having strong hole properties” refers to a compound that is easy to accept and/or transport holes (e.g., having a relatively high hole mobility), and such properties may be obtained by including a hole-receiving moiety (also, referred to as a hole transporting moiety).

Such a compound including a hole-receiving moiety may include, for example, a π electron-rich heteroaromatic compound (e.g., a carbazole derivative or an indole derivative), or an aromatic amine compound.

The electron transporting host may be a compound having strong electron properties. The expression “a compound having strong electron properties” refers to a compound that is easy to accept and/or transport electrons (e.g., having a relatively high electron mobility), and such properties may be obtained by including an electron-receiving moiety (also, referred to as an electron transporting moiety).

Such a compound including an electron-receiving moiety may include, for example, a π electron-deficient heteroaromatic compound. For example, the electron-receiving moiety may include a nitrogen-containing heteroaromatic compound (e.g., a π electron-deficient nitrogen-containing heteroaromatic compound).

The emission layer of the light-emitting device according to one or more embodiments may include a hole transporting host and an electron transporting host as a mixed host. For example, in some embodiments, the host may be a premixed host of a hole transporting host and an electron transporting host.

When a compound includes only a hole transporting moiety or only an electron transporting moiety, it is clear whether the nature of the compound has hole transporting properties or electron transporting properties.

In some embodiments, a compound may include both (e.g., simultaneously) a hole transporting moiety and an electron transporting moiety. In these embodiments, a simple comparison between the total number of the hole transporting moieties and the total number of the electron transporting moieties in the compound may be a criterion for predicting whether the compound is a hole transporting compound or an electron transporting compound, but cannot be an absolute criterion. One of the reasons why such a simple comparison may not be an absolute criterion is that one hole transporting moiety and one electron transporting moiety do not respectively have exactly the same ability to attract and/or transport holes and electrons.

Therefore, a relatively reliable way to determine whether a compound having a certain structure is a hole transporting compound or an electron transporting compound is to directly implement the compound in a device.

In one or more embodiments, the host may include a compound represented by Formula 301:

[Ar₃₀₁]_{xb11}-[(L₃₀₁)_{xb1}-R₃₀₁]_{xb21}, Formula 301
wherein, in Formula 301,
Ar₃₀₁ and L₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be the same as described with respect to Q₁.

In some embodiments, when xb11 in Formula 301 is 2 or more, two or more of Ar₃₀₁ (s) may be linked to each other via a single bond.

In one or more embodiments, the host may include a compound represented by Formula 301-1, a compound represented by Formula 301-2, or any combination thereof:

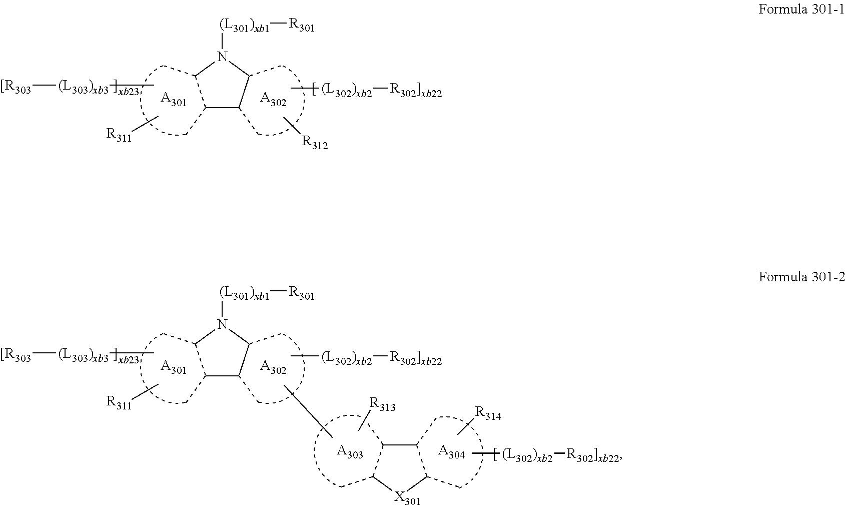

wherein, in Formulae 301-1 and 301-2,
ring A₃₀₁ to ring A₃₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described in the present disclosure,
L₃₀₂ to L₃₀₄ may each independently be the same as described with respect to L₃₀₁,
xb2 to xb4 may each independently be the same as described with respect to xb1, and
R₃₀₂ to R₃₀₅ and R₃₁₁ to R₃₁₄ may each be the same as described with respect to R₃₀₁.

In one or more embodiments, the host may include an alkaline earth metal complex, a post-transition metal complex, or any combination thereof. In some embodiments, the host may include a Be complex (for example, Compound H55), an Mg complex, a Zn complex, or any combination thereof.

In one or more embodiments, the host may include at least one selected from among Compounds H1 to H126, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(9H-carbazol-9-yl)benzene (mCP), 1,3,5-tri(carbazol-9-yl)benzene (TCP), or any combination thereof:

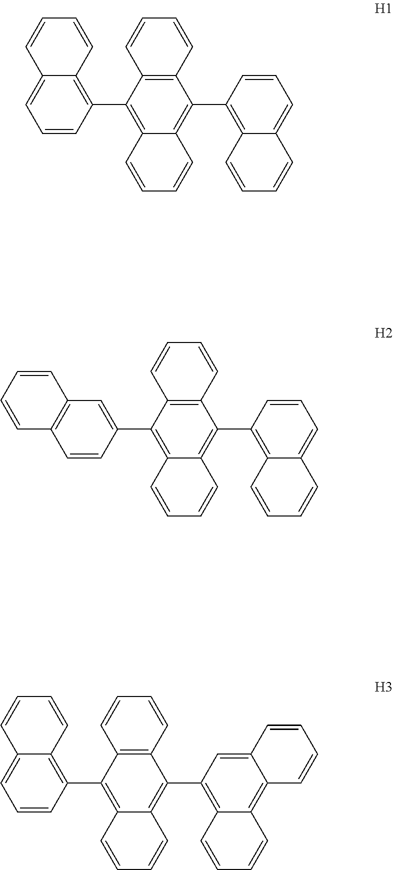

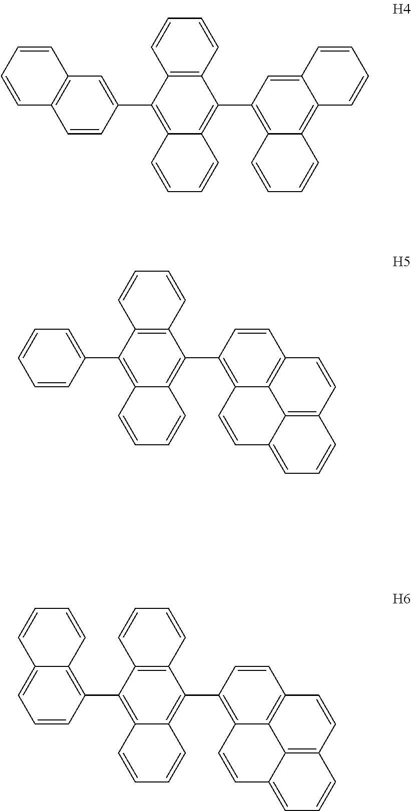

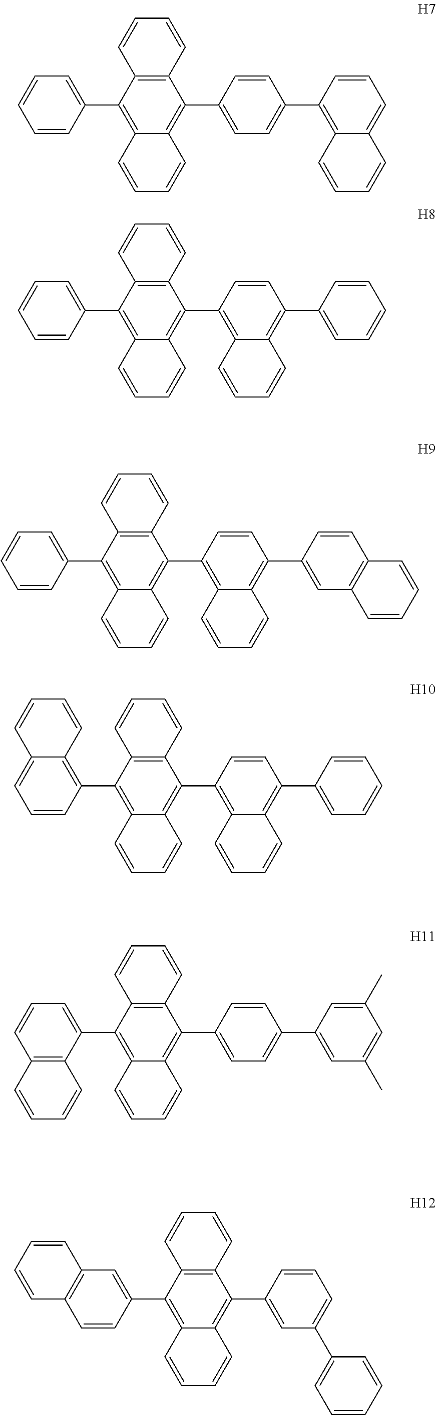

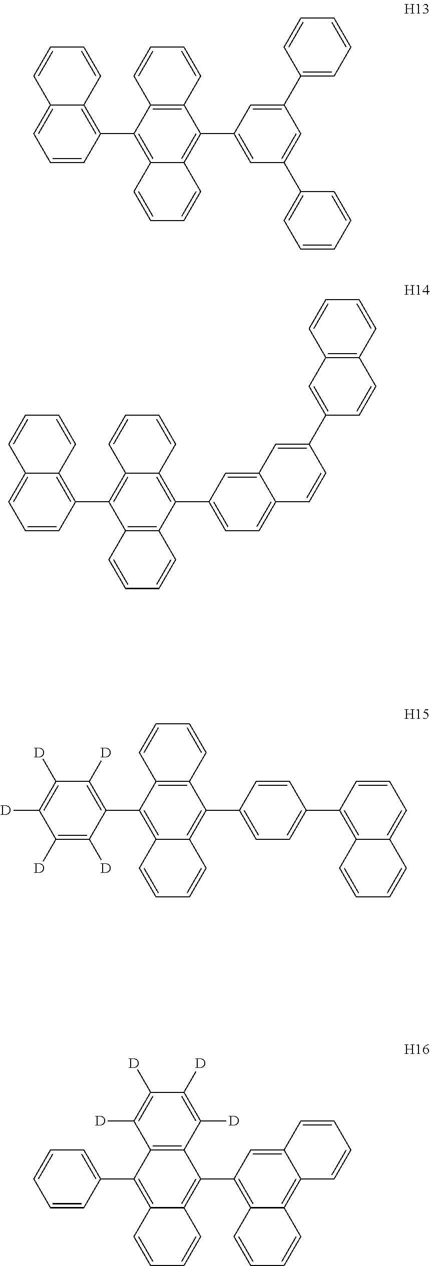

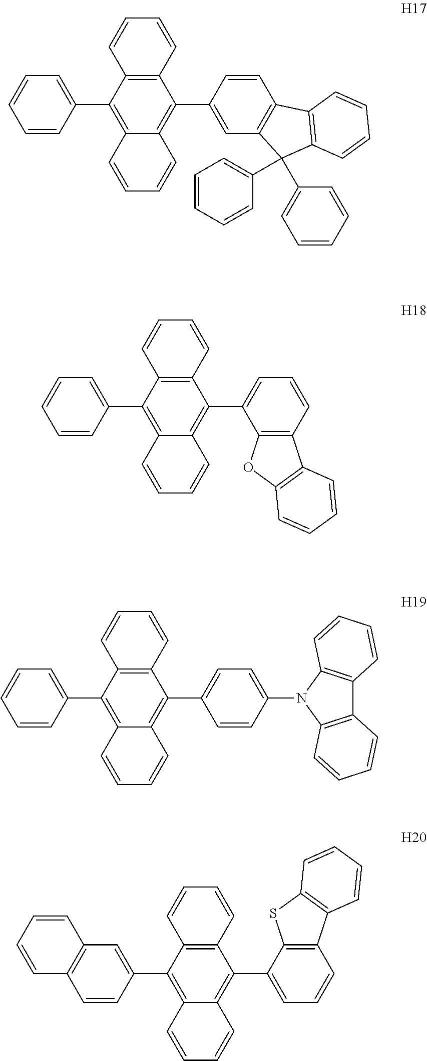

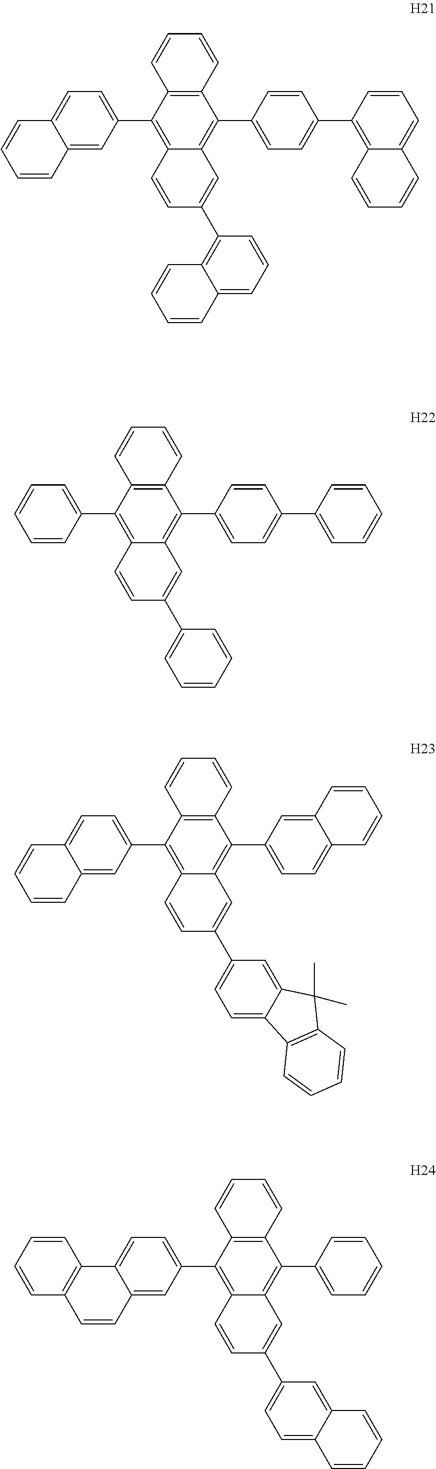

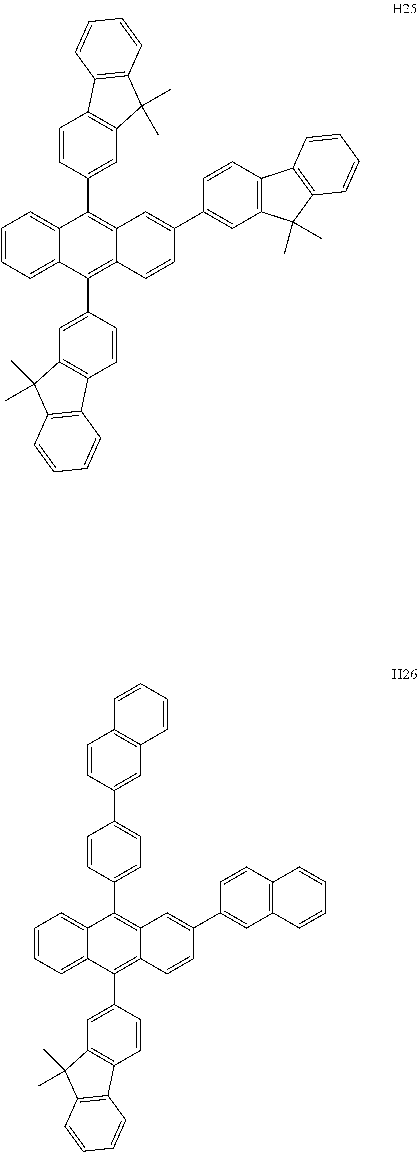

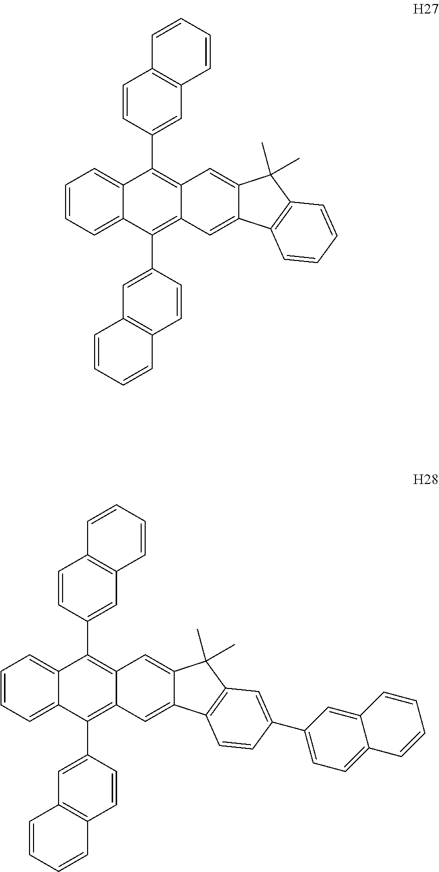

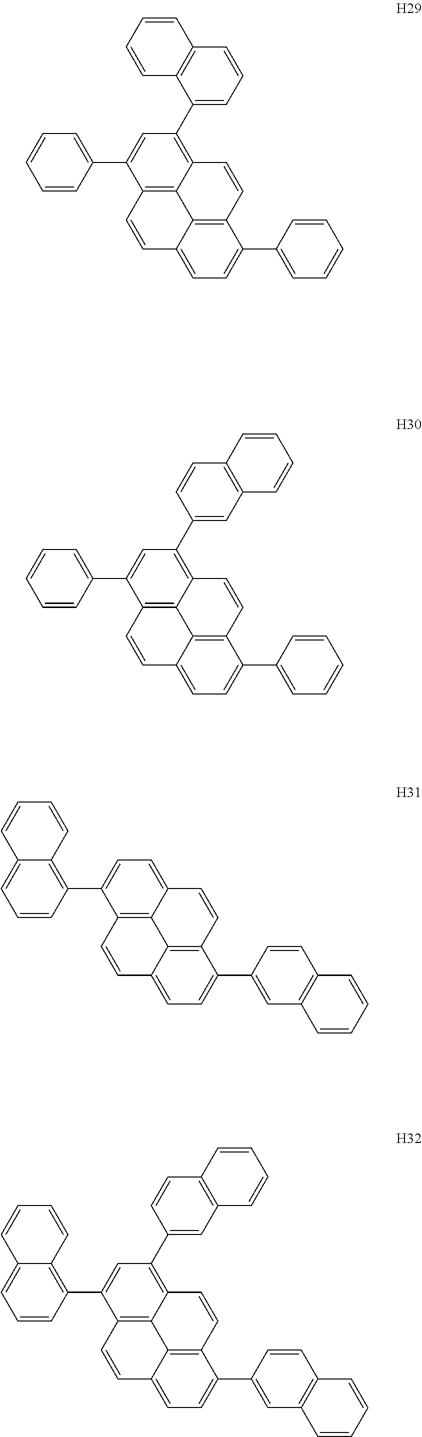

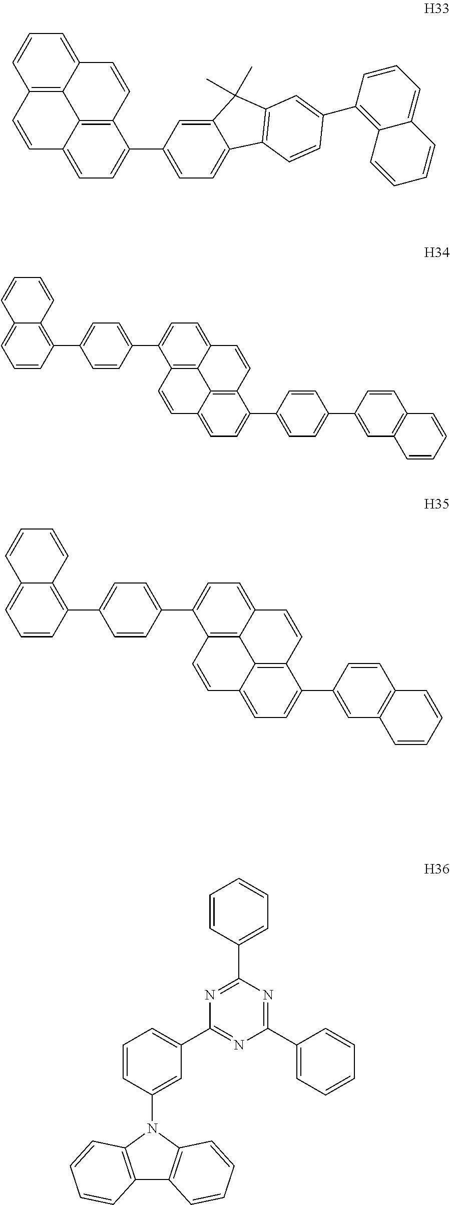

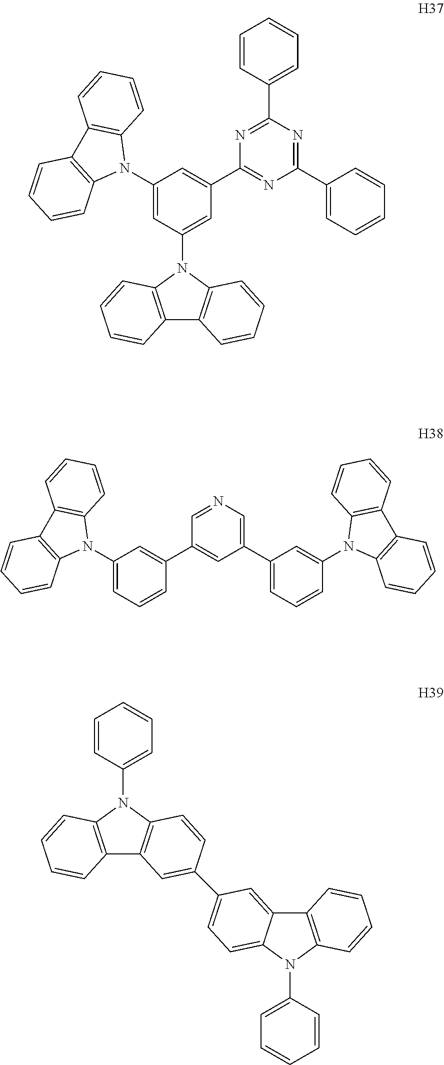 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

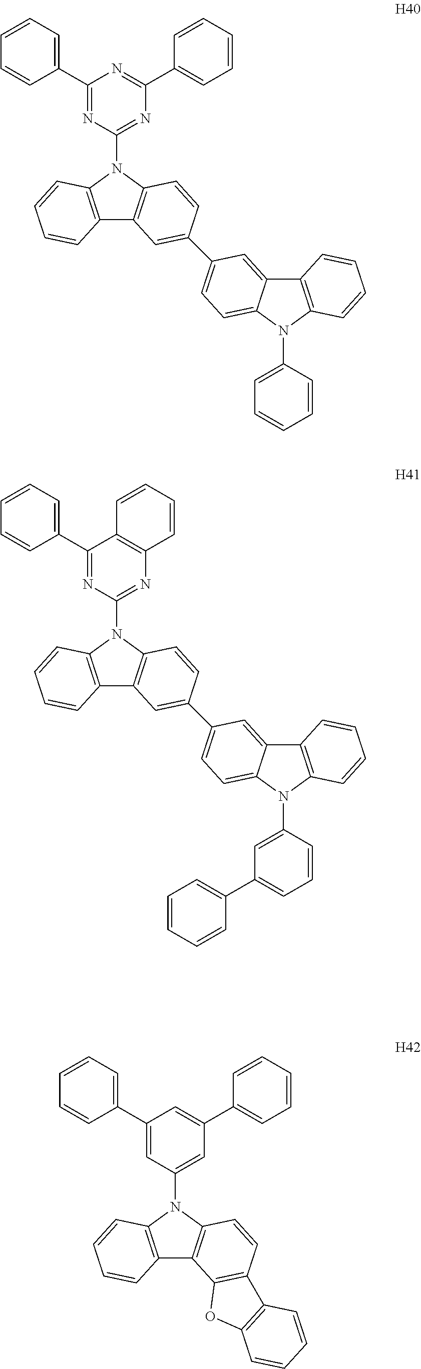 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

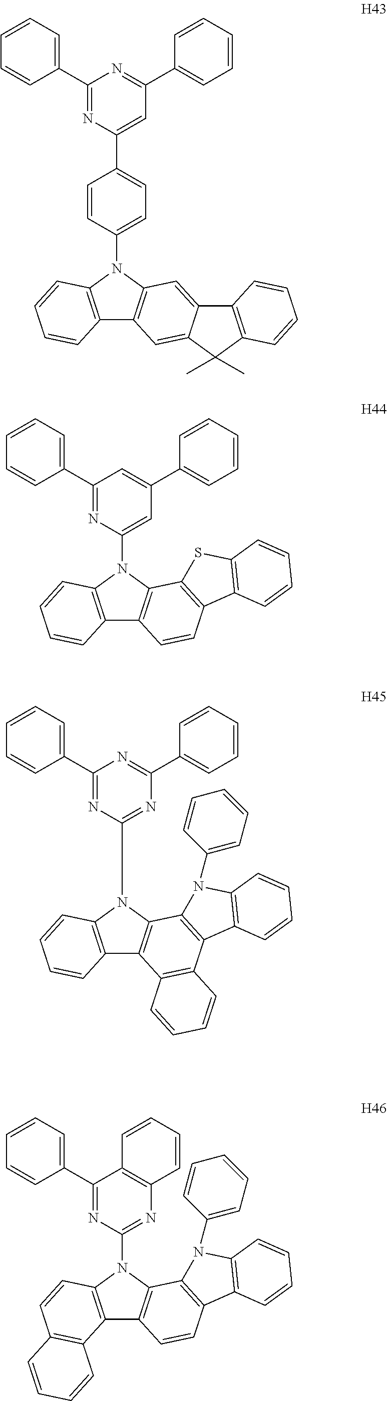 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

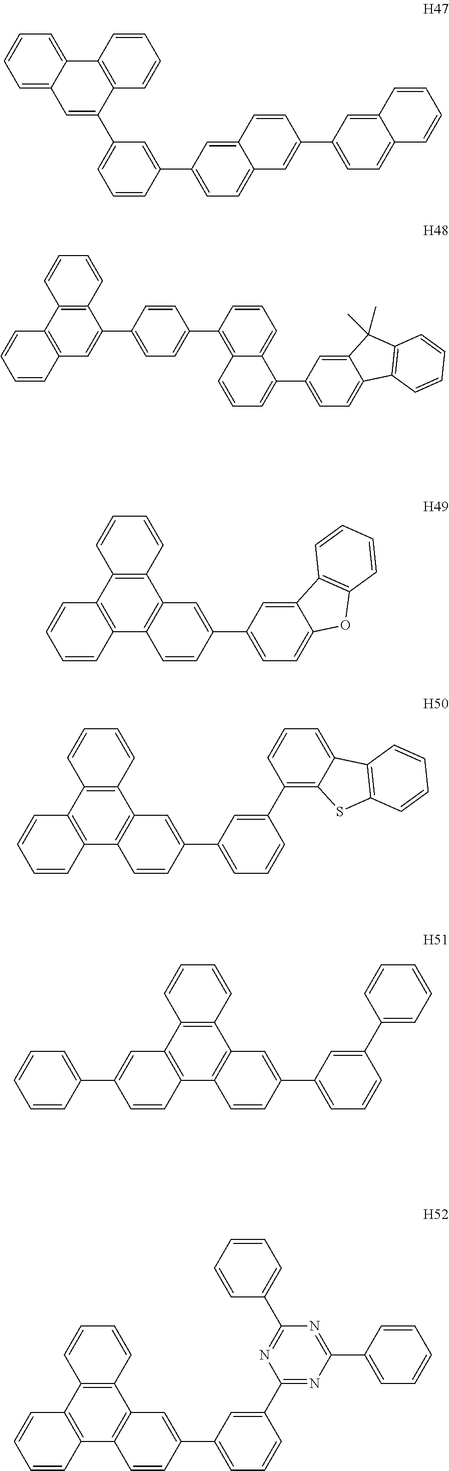

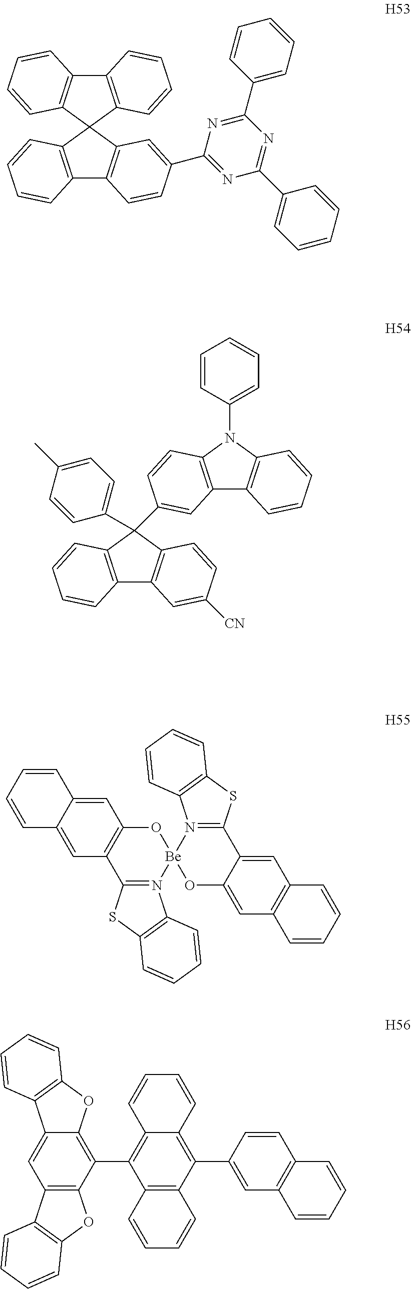

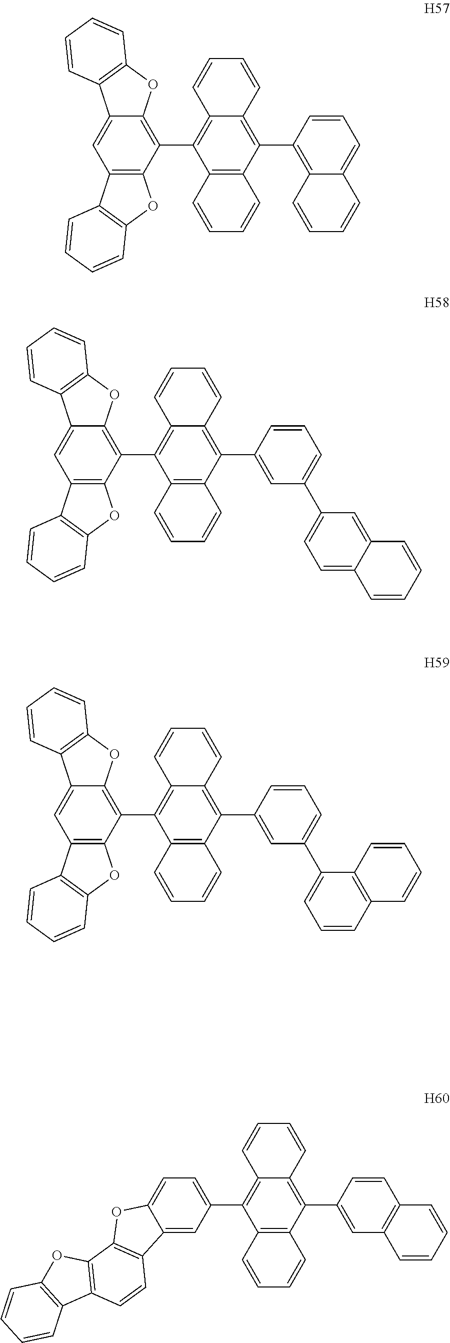 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

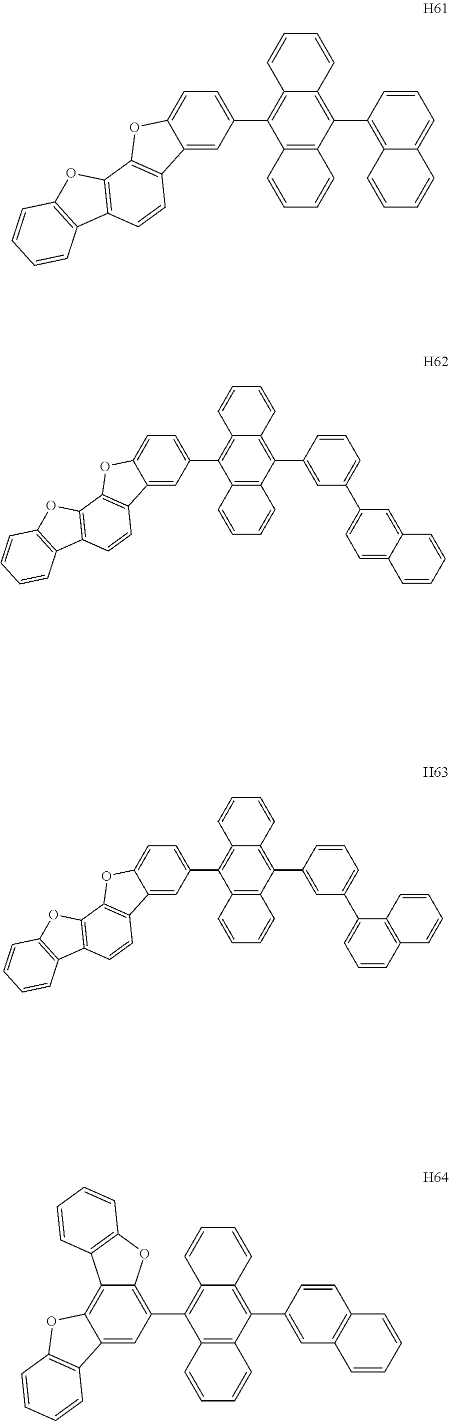 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

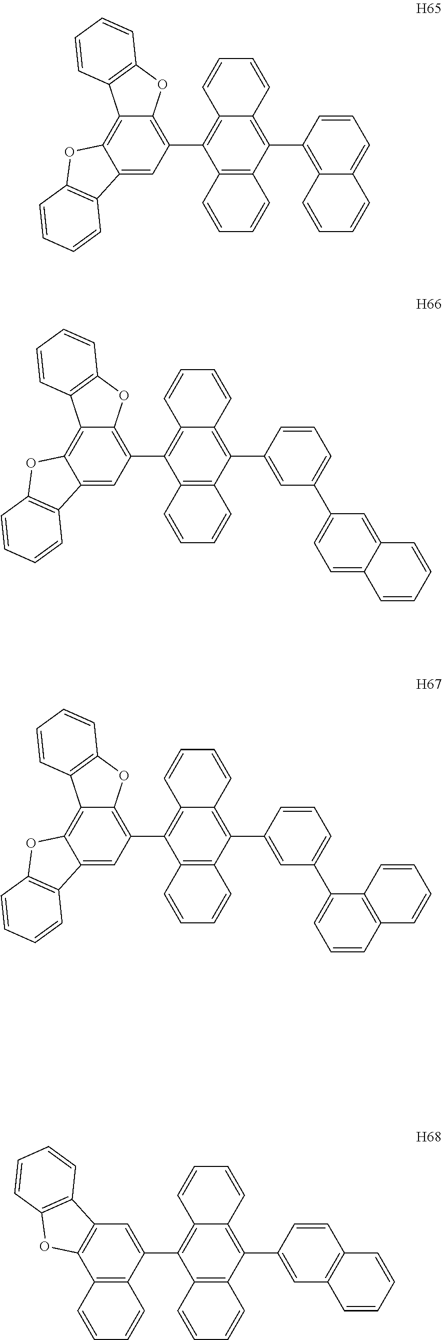 [SMILES: c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

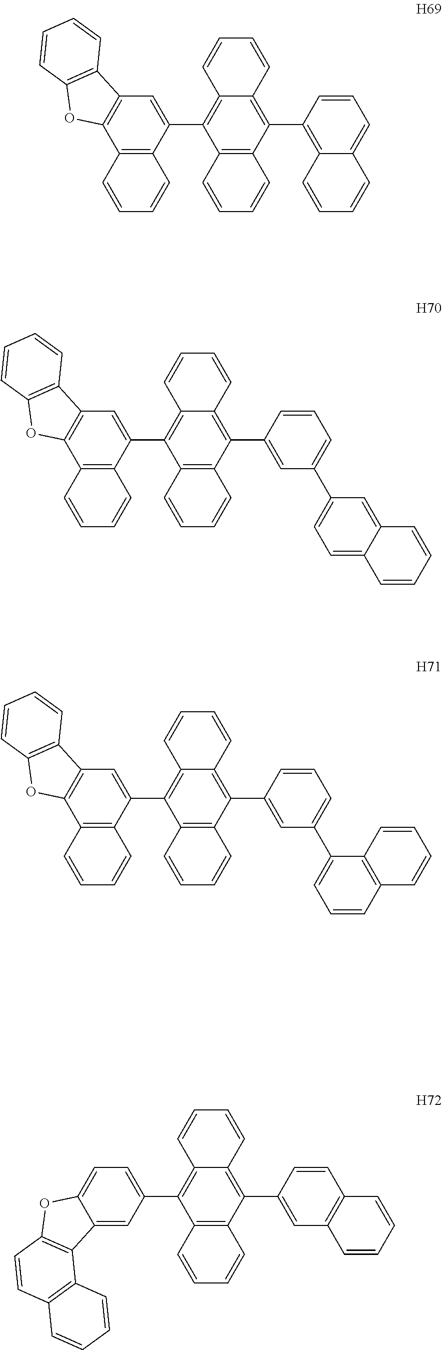 [SMILES: c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

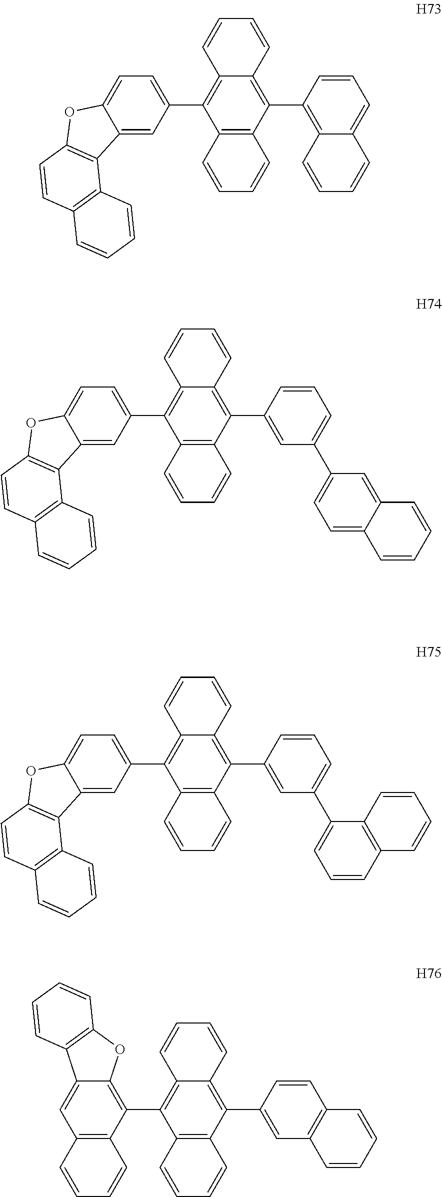 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1]

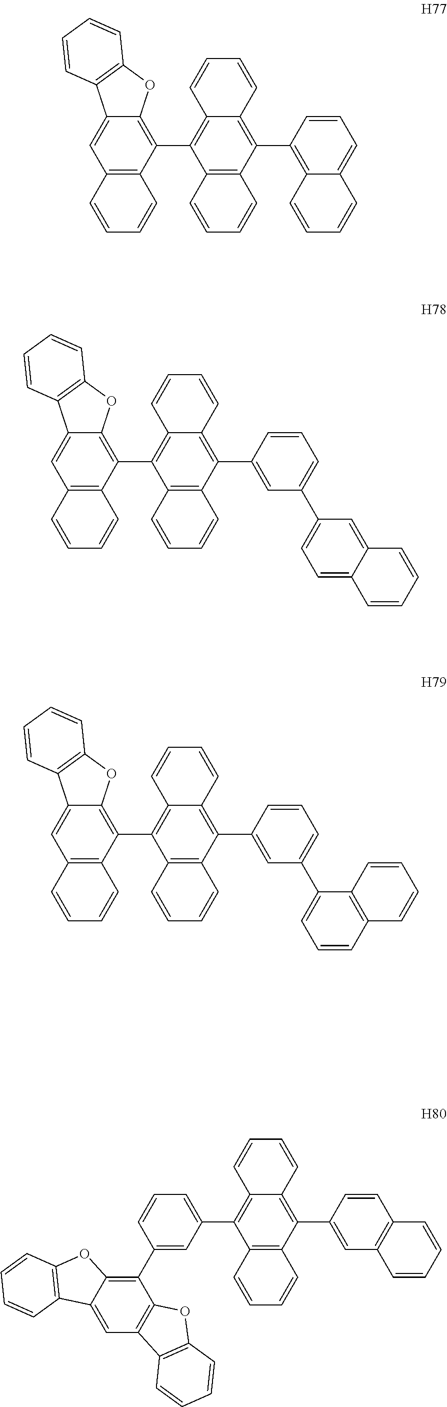 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

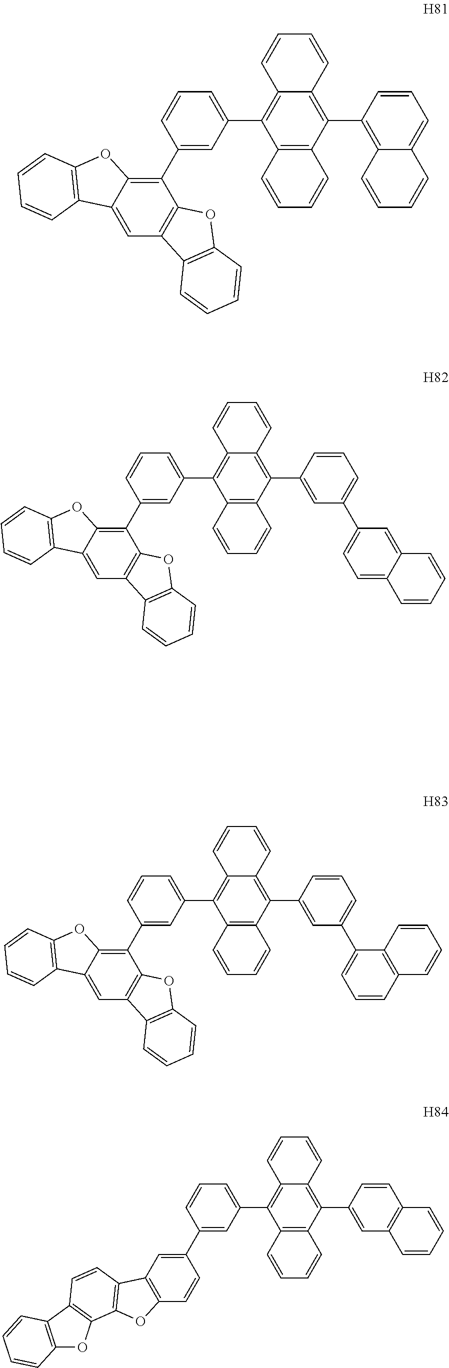 [SMILES: c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1]

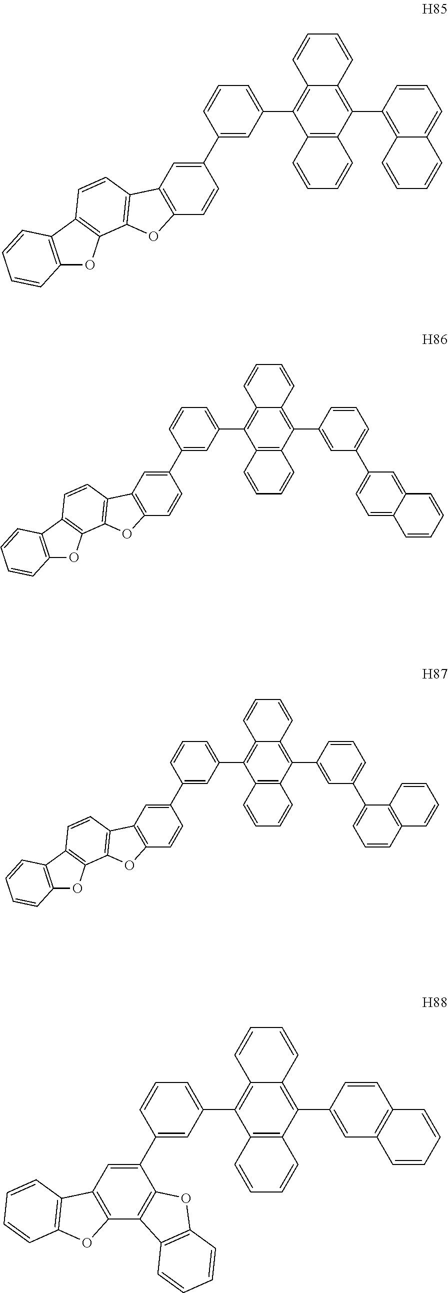 [SMILES: c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1]

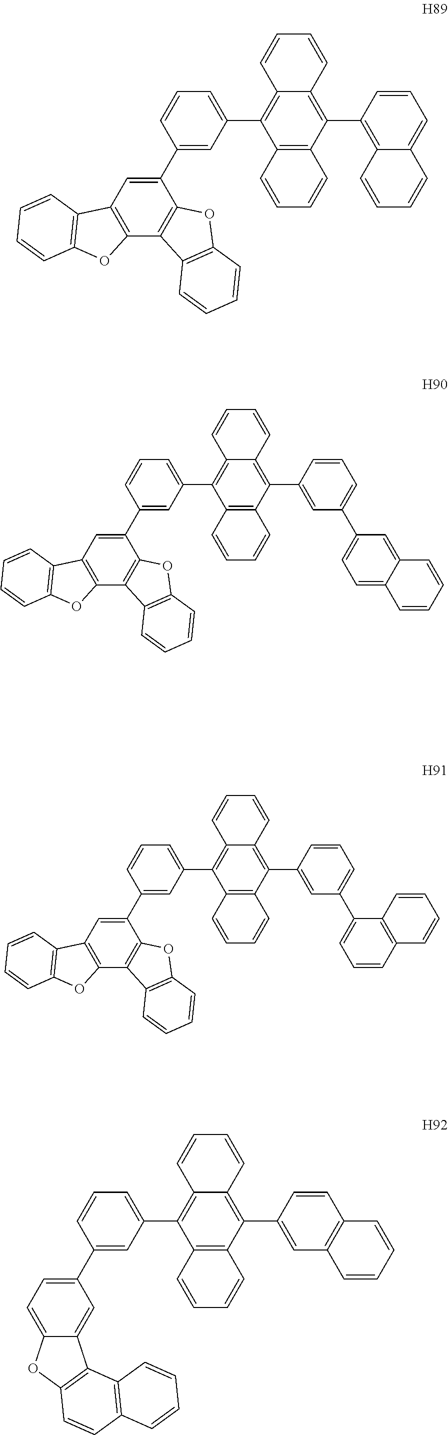 [SMILES: c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1]

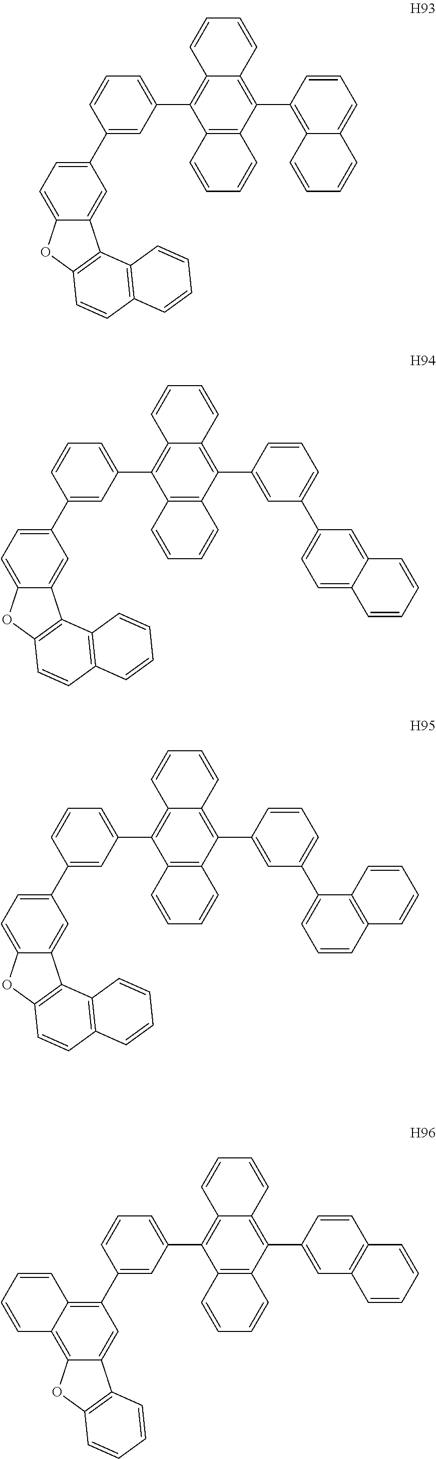 [SMILES: c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1]

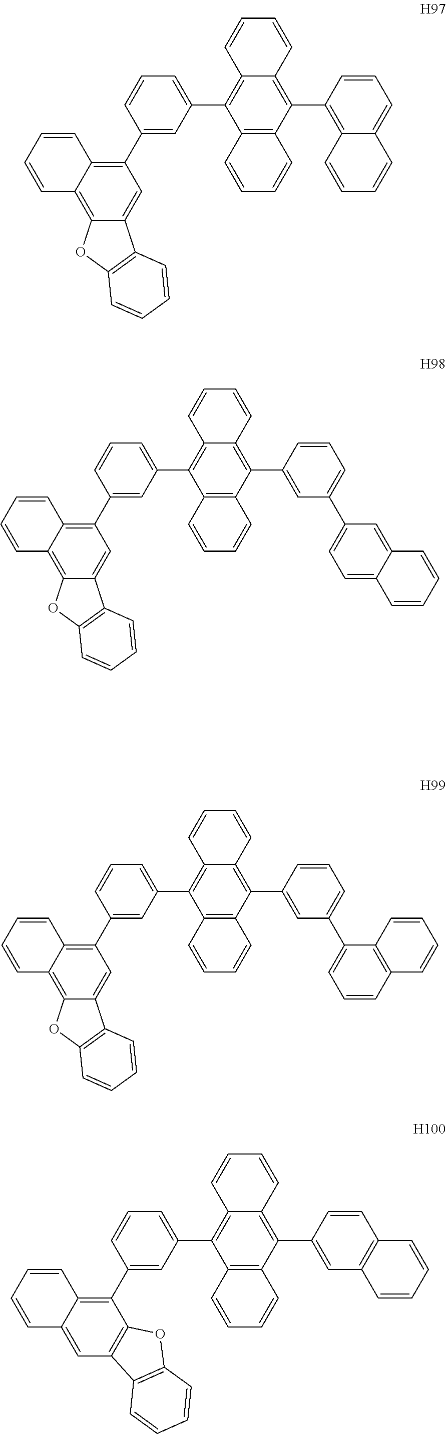 [SMILES: c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1]

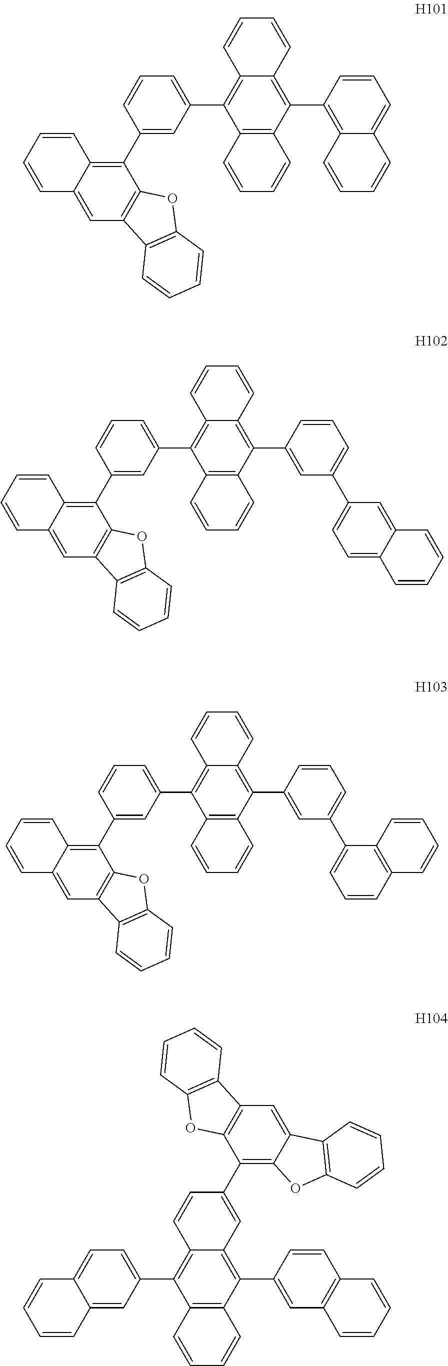 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

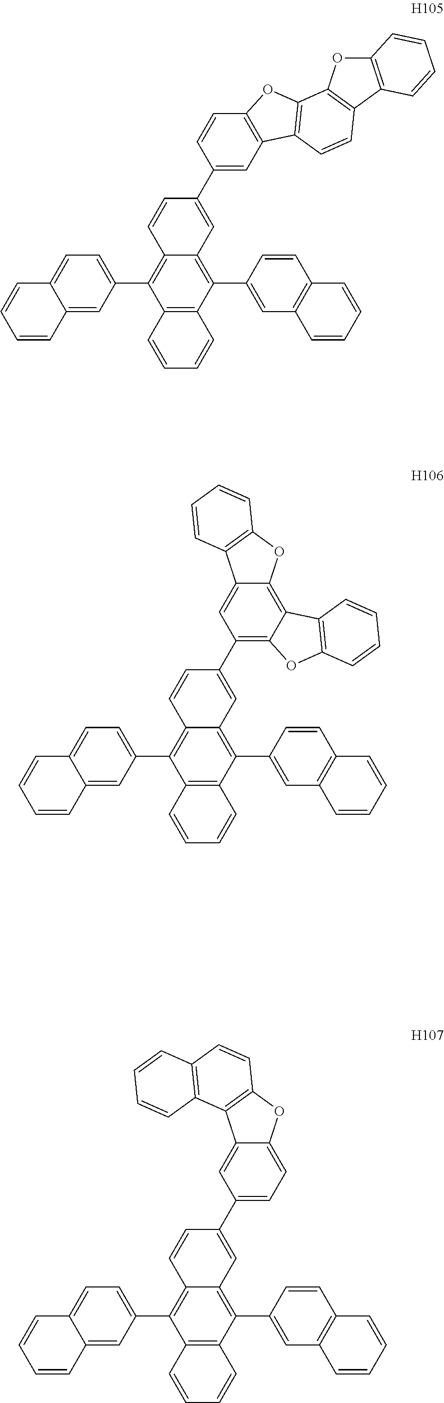 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

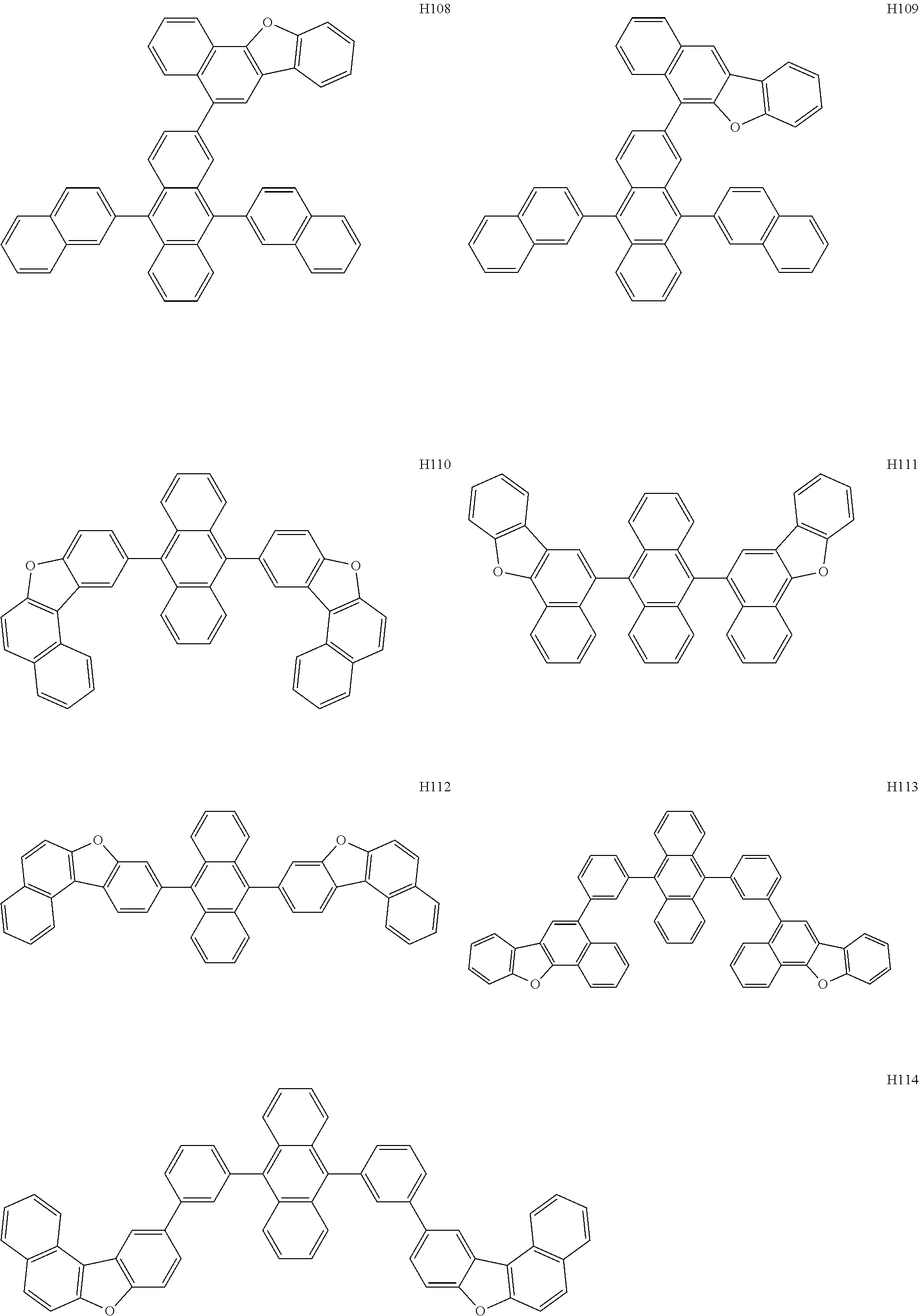

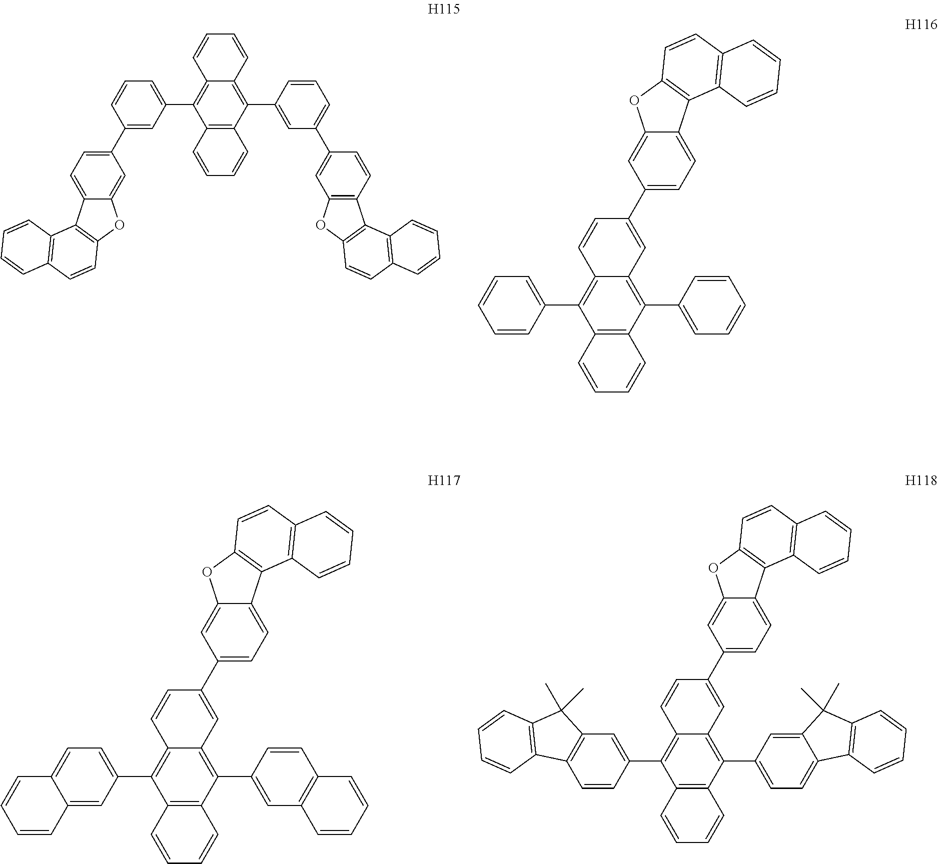 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

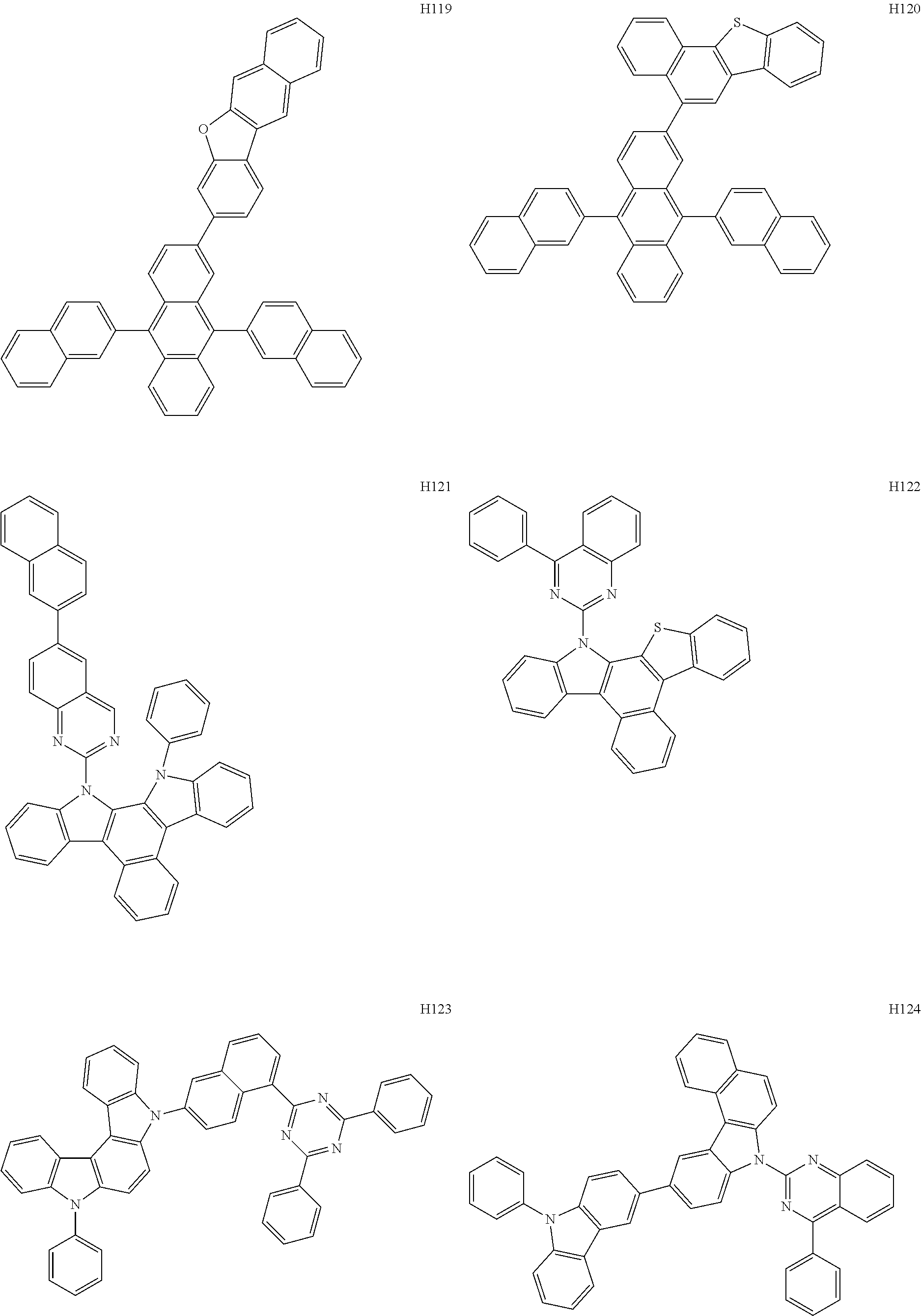

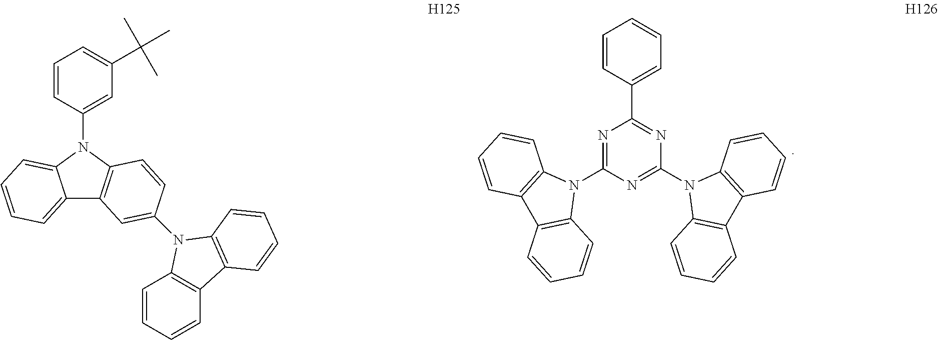 [SMILES: CC(C)(C)c1cccc(-n2c3ccccc3c3cc(-n4c5ccccc5c5ccccc54)ccc32)c1 ; c1ccc(-c2nc(-n3c4ccccc4c4ccccc43)nc(-n3c4ccccc4c4ccccc43)n2)cc1]

### Phosphorescent Dopant

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In one or more embodiments, the phosphorescent dopant may include an organometallic compound represented by Formula 401:

M(L₄₀₁)_{xc1}(L₄₀₂)_{xc2} Formula 401

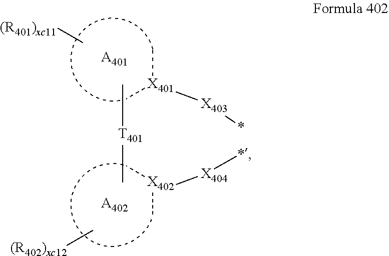

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 is 1, 2, or 3, wherein, when xc1 is 2 or more, two or more of L₄₀₁ (s) may be identical to or different from each other,
L₄₀₂ may be an organic ligand, and xc2 may be 0, 1, 2, 3, or 4, wherein, when xc2 is 2 or more, two or more of L₄₀₂ (s) may be identical to or different from each other,
X₄₀₁ and X₄₀₂ may each independently be nitrogen or carbon,
ring A₄₀₁ and ring A₄₀₂ may each independently a C₃-C₆₀ carbocyclic group or a C₁-C₆₀ heterocyclic group,
T₄₀₁ may be a single bond, —O—, —S—, —C(═O)—, —N(Q₄₁₁)-, —C(Q₄₁₁)(Q₄₁₂)-, —C(Q₄₁₁)=C(Q₄₁₂)-, —C(Q₄₁₁)═, or ═C═,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃) (Q₄₁₄),
Q₄₁₁ to Q₄₁₄ may each independently be the same as described with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group that is unsubstituted or substituted with at least one R₁₀ₐ, a C₁-C₂₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described with respect to Q₁,
xc11 and xc12 may each independently be an integer from 0 to 10, and
* and *′ in Formula 402 each indicate a binding site to M in Formula 401.

In one or more embodiments, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁ (s) among two or more of L₄₀₁ (s) may be optionally linked together via T₄₀₂, which is a linking group, and/or two ring A₄₀₂ (s) may be optionally linked together via T₄₀₃, which is a linking group (see Compounds PD1 to PD4 and PD7). T₄₀₂ and T₄₀₃ may each be the same as described with respect to T₄₀₁.

L₄₀₂ in Formula 401 may be an organic ligand. In one or more embodiments, L₄₀₂ may include a halogen, a diketone group (for example, an acetylacetonate group), a carboxylic acid group (for example, a picolinate group), —C(═O), an isonitrile group, a —CN group, a phosphorus-containing group (for example, a phosphine group, a phosphite group, etc.), or any combination thereof.

In the ligand of the organometallic compound, adjacent substituents may be optionally bonded together to form a ring.

In one or more embodiments, the phosphorescent dopant may include, for example, one selected from among Compounds PD1 to PD40 or any combination thereof:

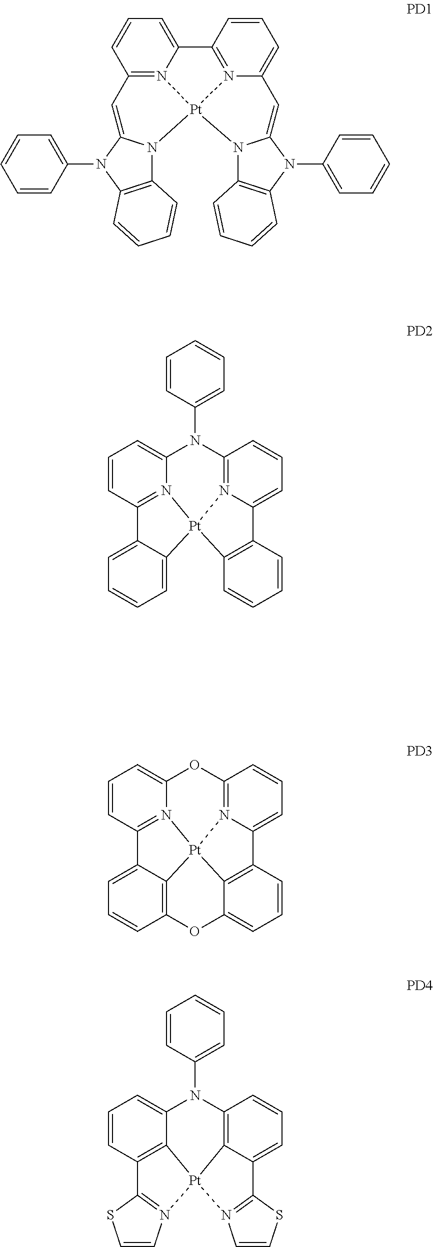

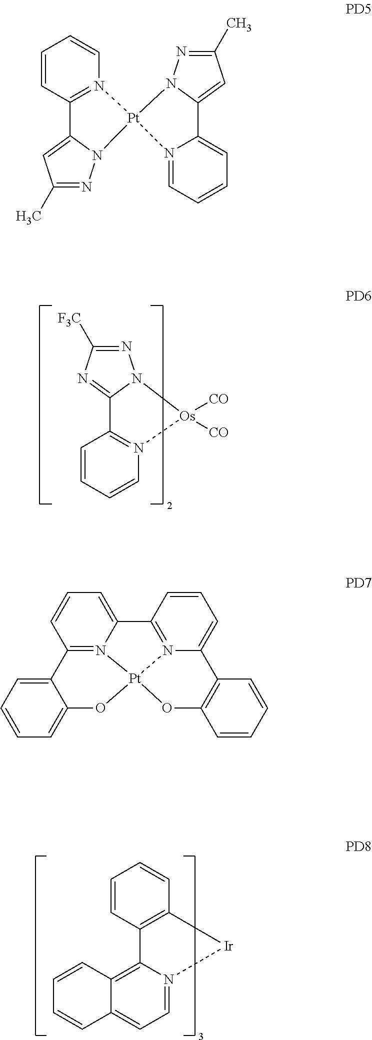

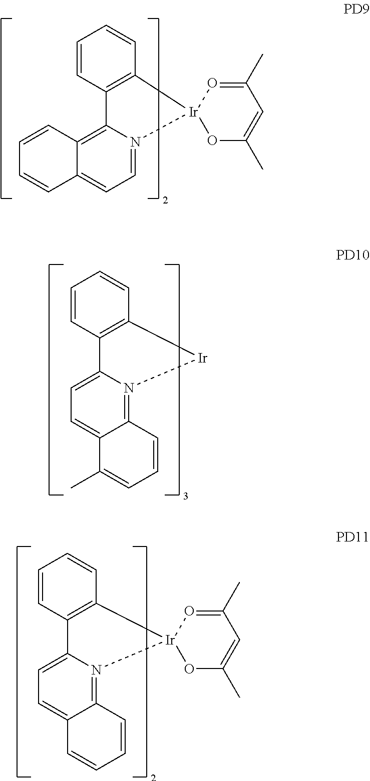

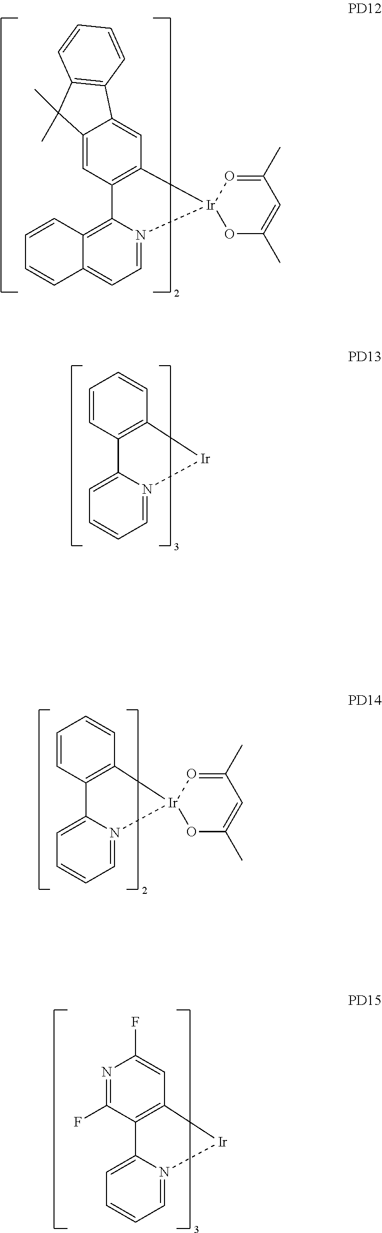

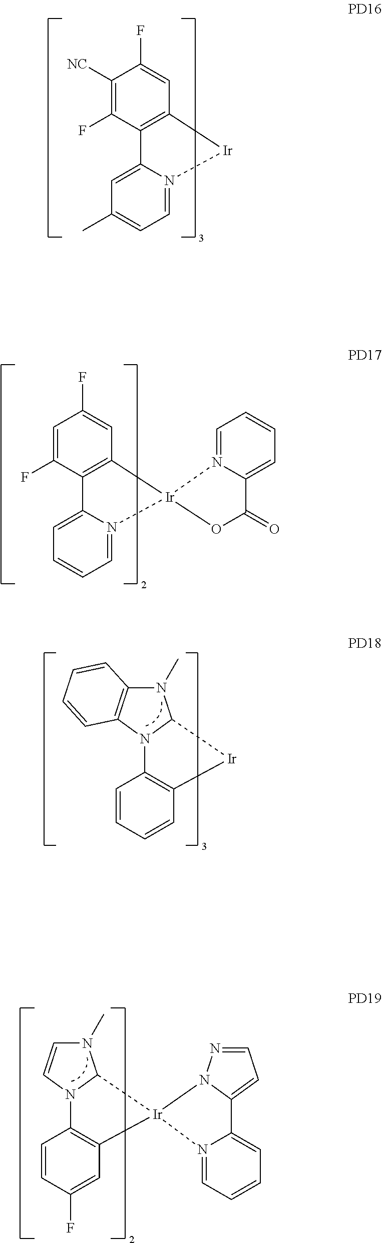

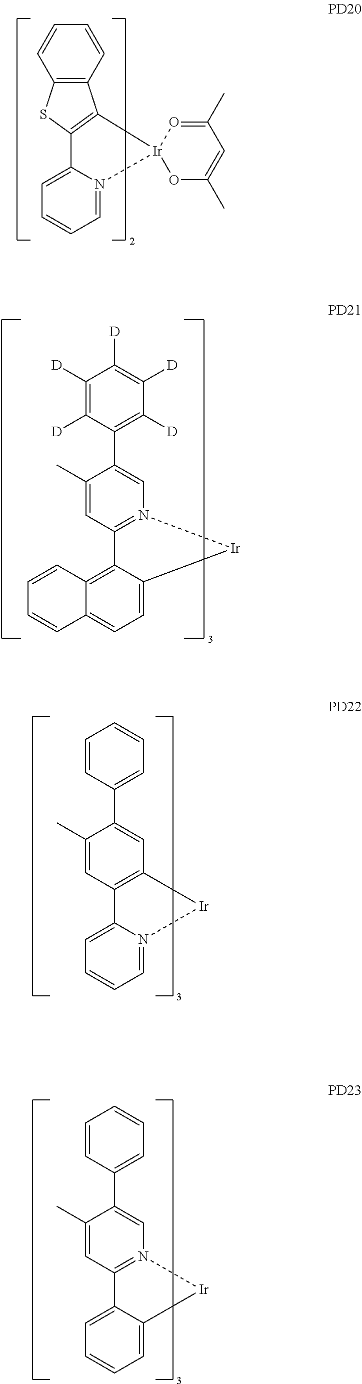

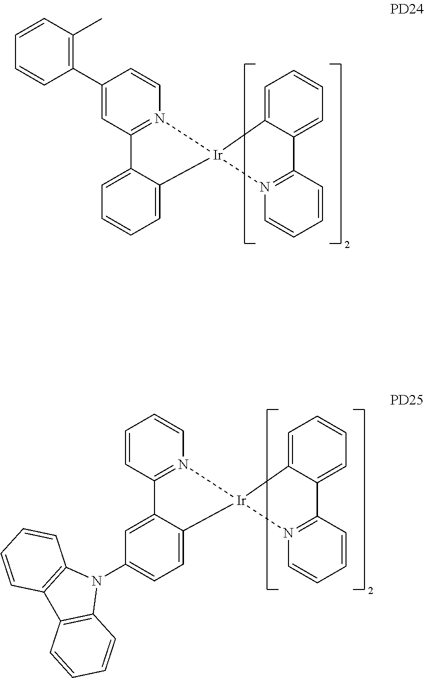

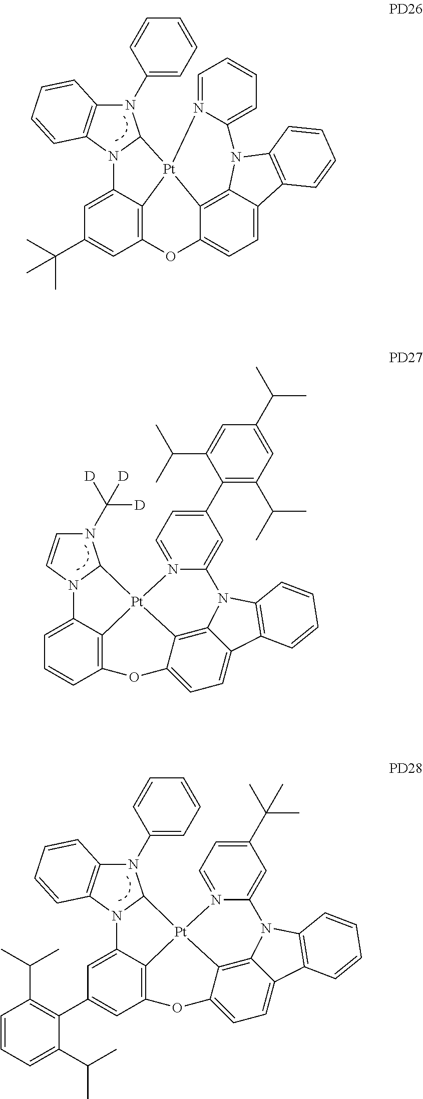

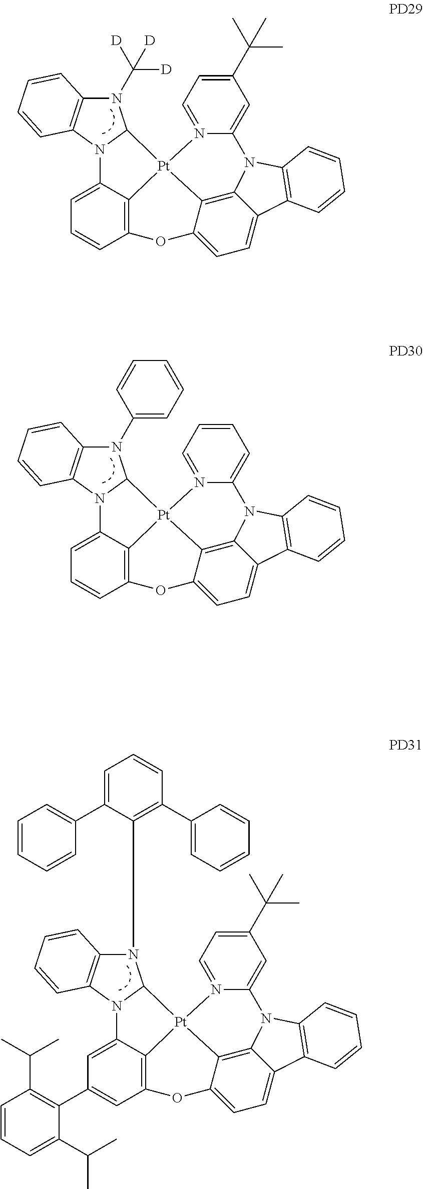

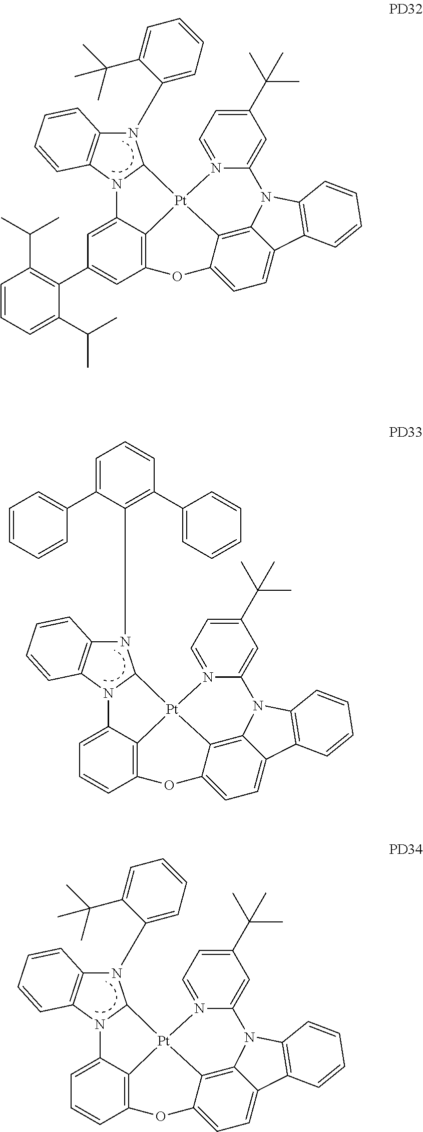

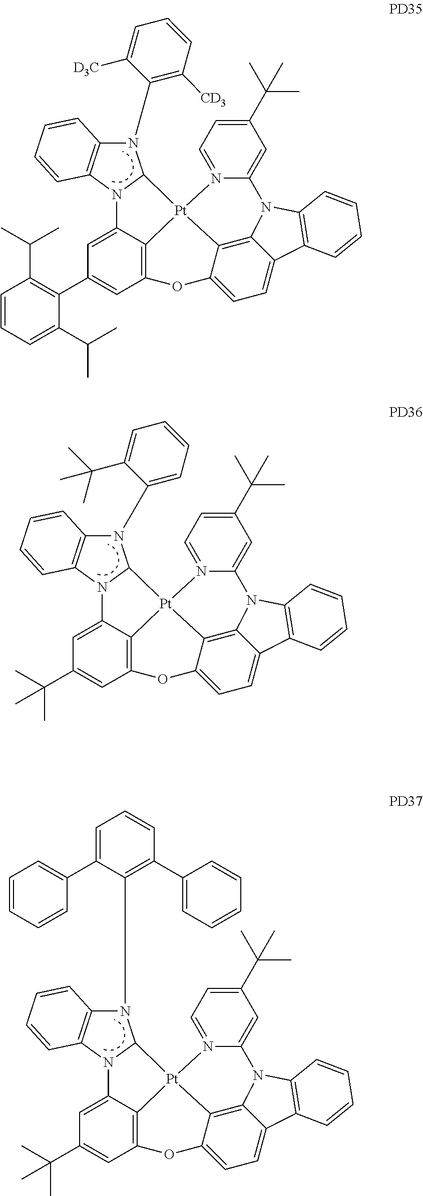

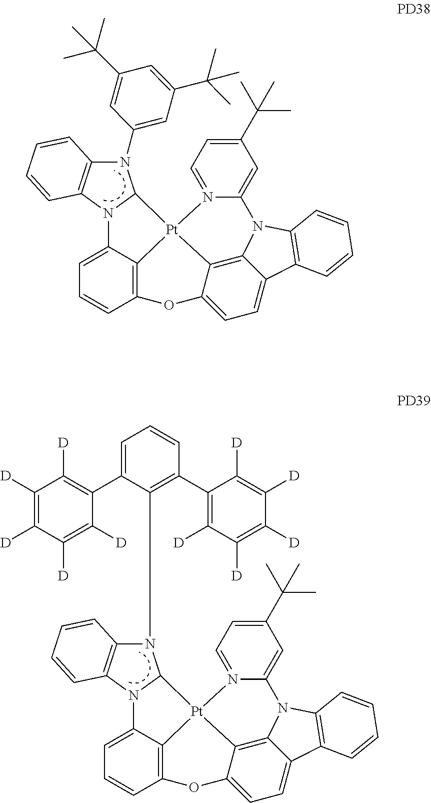

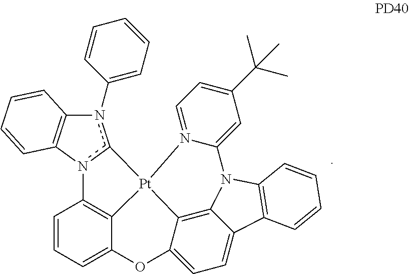

### Fluorescent Dopant

The fluorescent dopant may include an amine group-containing compound, a styryl group-containing compound, or any combination thereof.

For example, in one or more embodiments, the fluorescent dopant may include a compound represented by Formula 501:

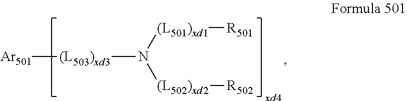

wherein, in Formula 501,
Ar₅₀₁, L₅₀₁ to L₅₀₃, R₅₀₁, and R₅₀₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, etc.) in which three or more monocyclic groups are condensed together.

In some embodiments, xd4 in Formula 501 may be 2.

In one or more embodiments, the fluorescent dopant may include: at least one selected from among Compounds FD1 to FD37; 4,4′-bis(2,2-diphenylvinyl)-1,1′-biphenyl (DPVBi); 4,4′-bis[4-(N,N-diphenylamino)styryl]biphenyl (DPAVBi); or any combination thereof:

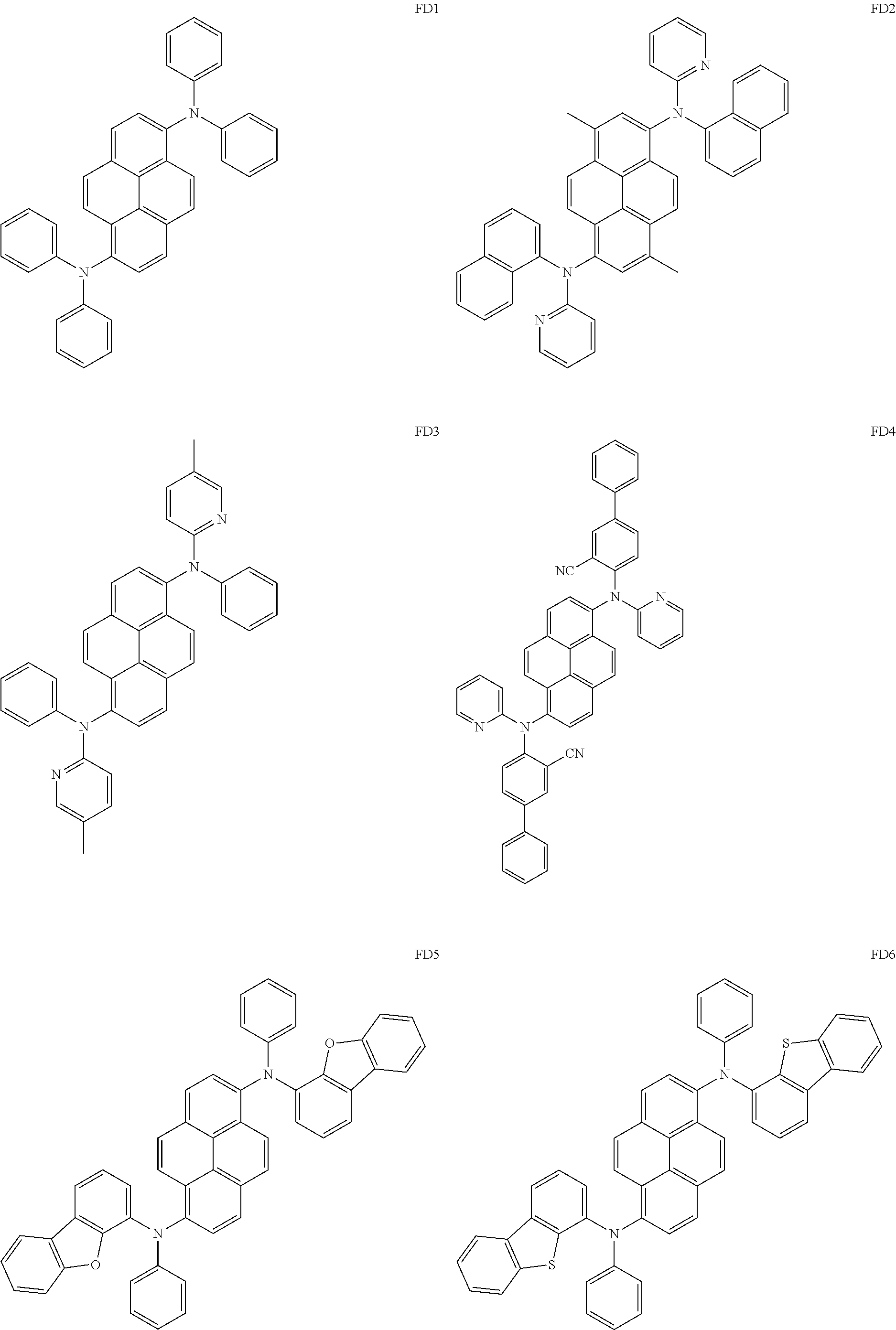

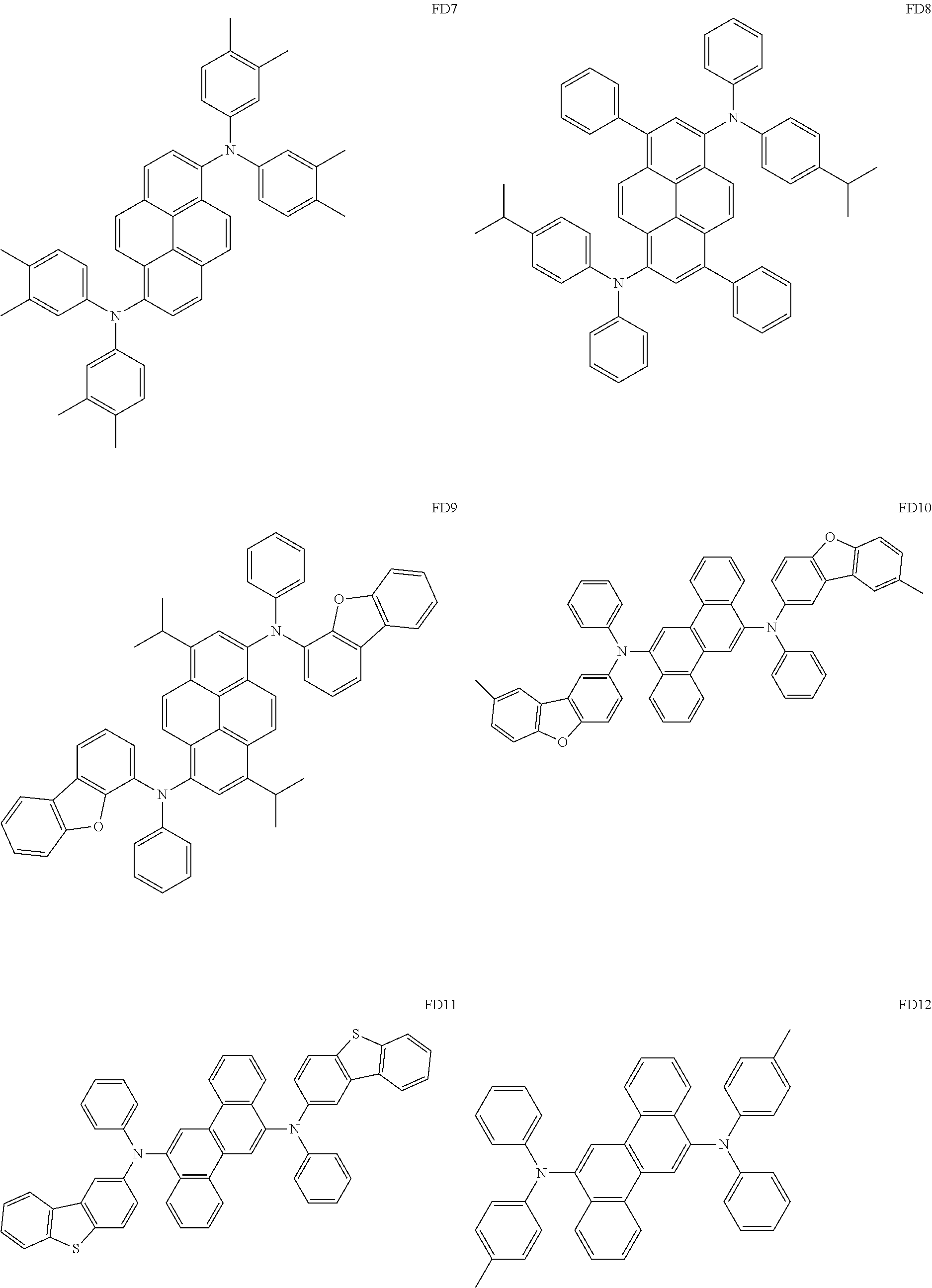

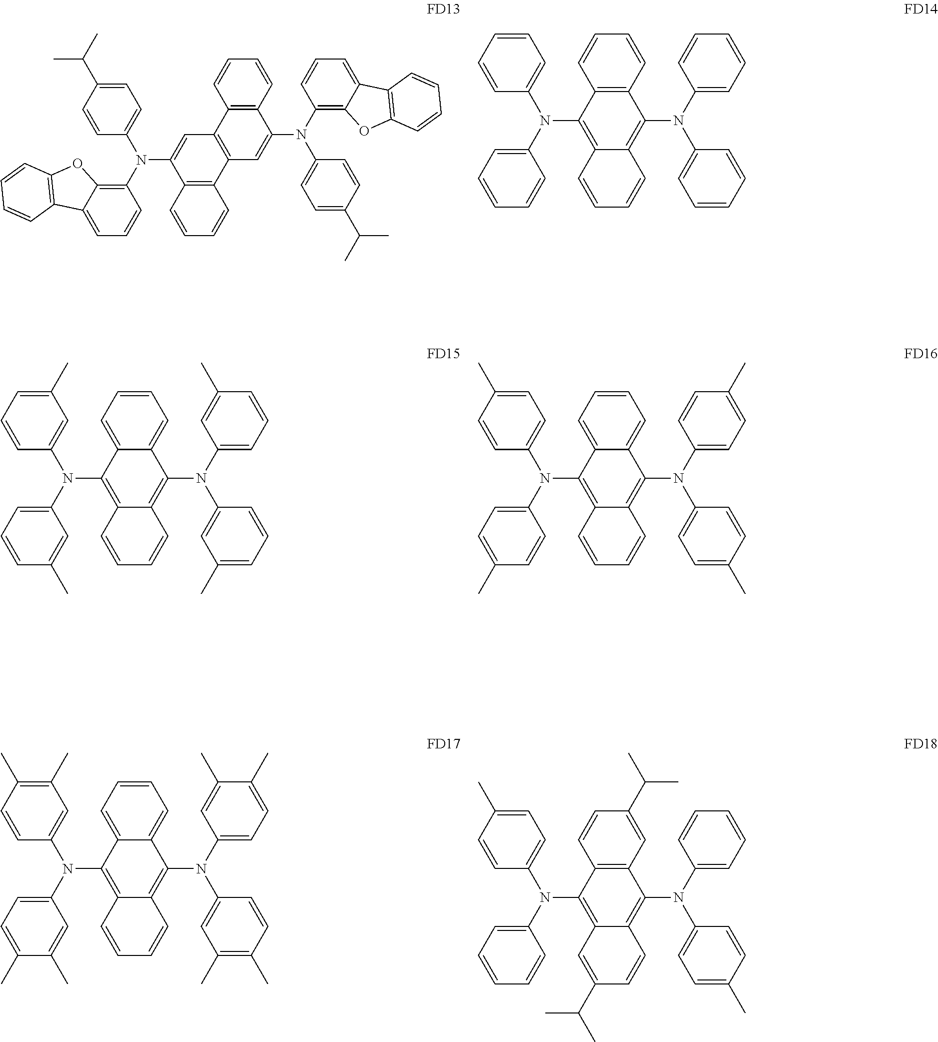

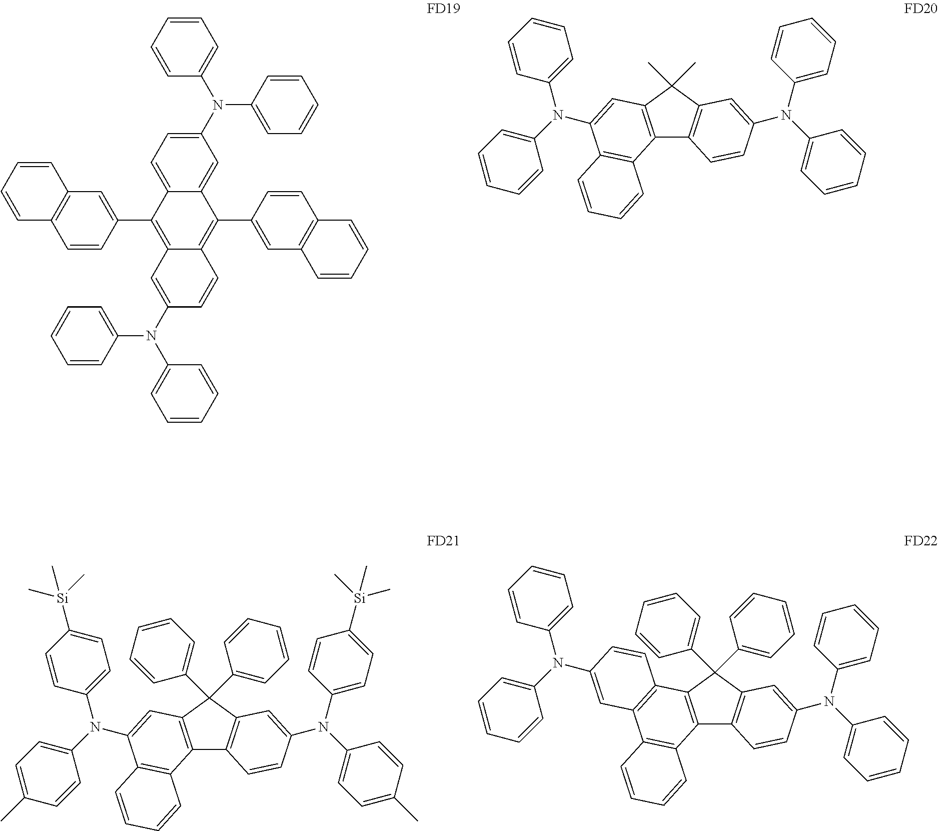

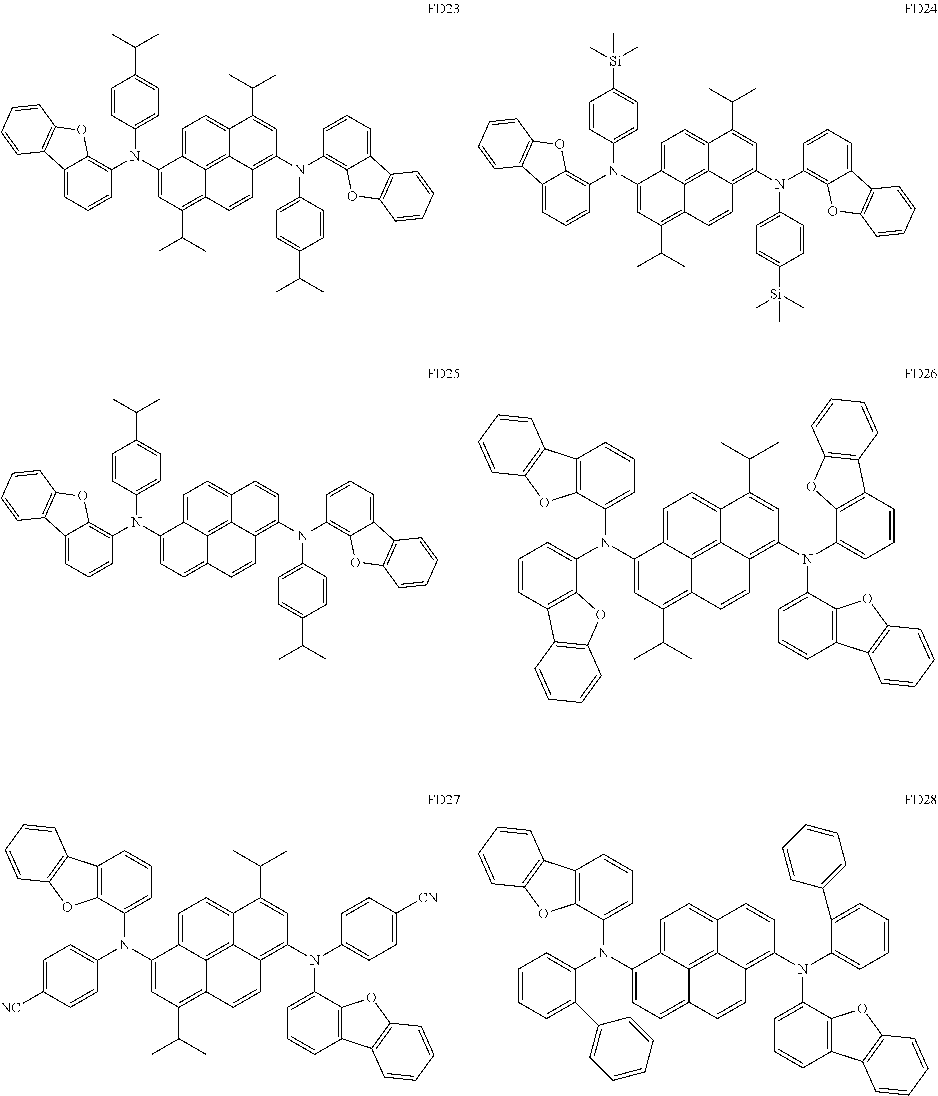

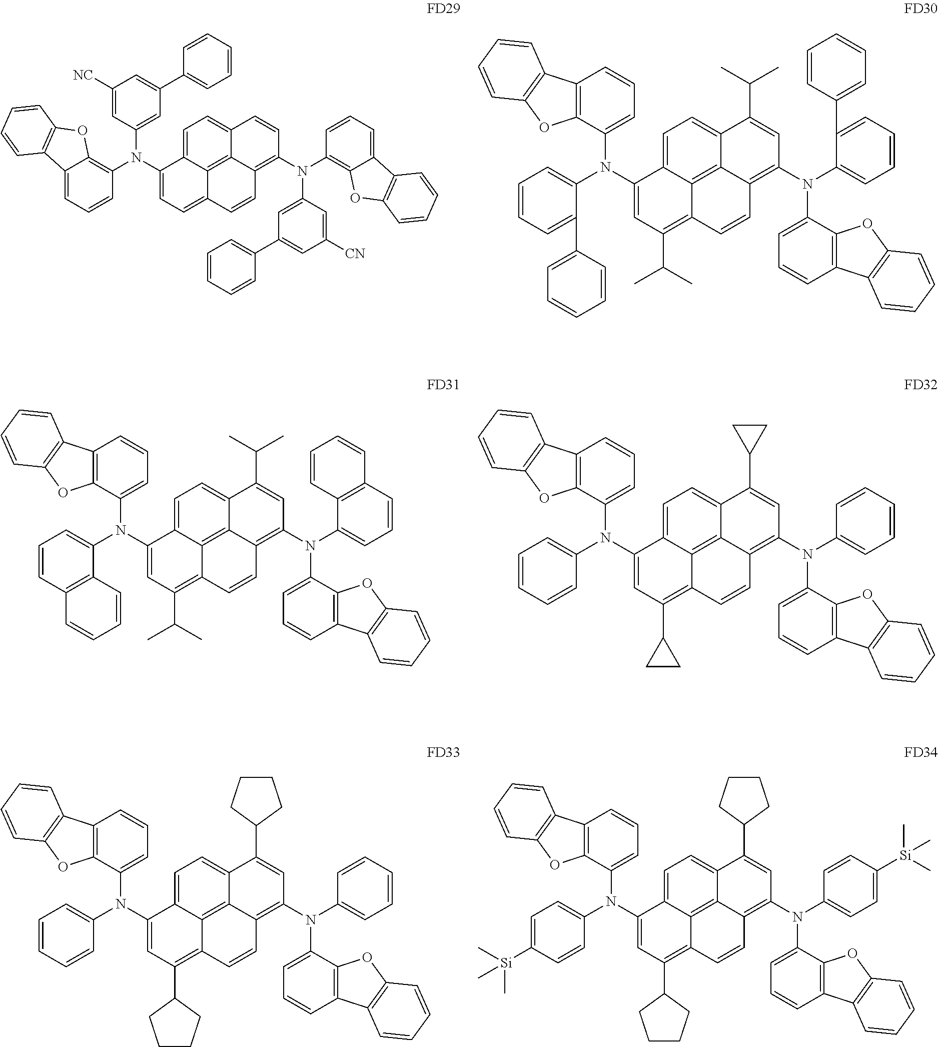

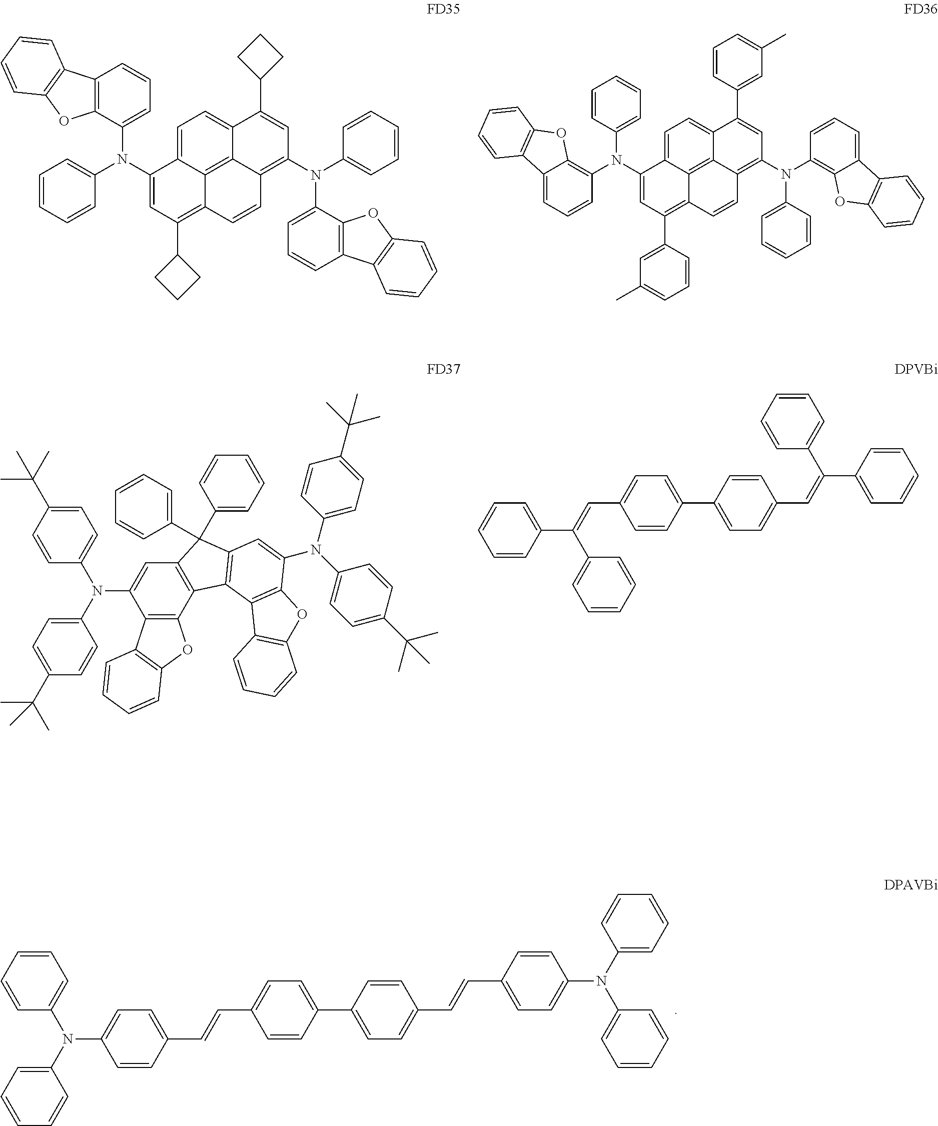

### Delayed Fluorescence Material

In one or more embodiments, the emission layer may include a delayed fluorescence material.

Herein, the delayed fluorescence material may be selected from compounds capable of emitting delayed fluorescence based on a delayed fluorescence emission mechanism.

The delayed fluorescence material included in the emission layer may act as a host or a dopant depending on the type or kind of other materials included in the emission layer.

In one or more embodiments, a difference between a triplet energy level (eV) of the delayed fluorescence material and a singlet energy level (eV) of the delayed fluorescence material may be at least about 0 eV and not more than about 0.5 eV. When the difference between the triplet energy level (eV) of the delayed fluorescence material and the singlet energy level (eV) of the delayed fluorescence material is satisfied within the range above, up-conversion from the triplet state to the singlet state of the delayed fluorescence materials may effectively occur, and thus, the light-emitting device 10 may have improved luminescence efficiency.

In one or more embodiments, the delayed fluorescence material may include: i) a material including at least one electron donor (for example, a π electron-rich C₃-C₆₀ cyclic group such as a carbazole group) and at least one electron acceptor (for example, a sulfoxide group, a cyano group, a π electron-deficient nitrogen-containing C₁-C₆₀ cyclic group, and/or the like), and/or ii) a material including a C₈-C₆₀ polycyclic group including at least two cyclic groups that are condensed with each other while sharing boron (B).

Non-limiting examples of the delayed fluorescence material may include at least one selected from among Compounds DF1 to DF14:

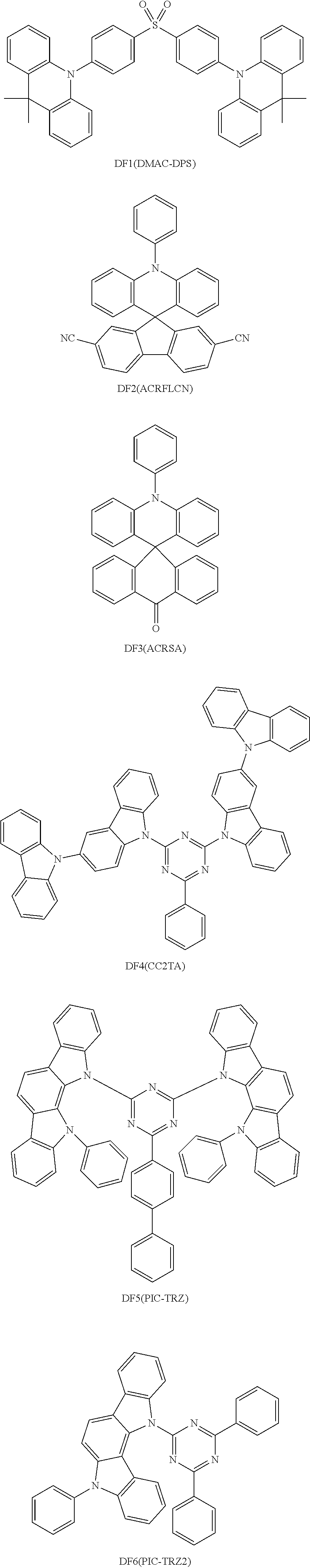

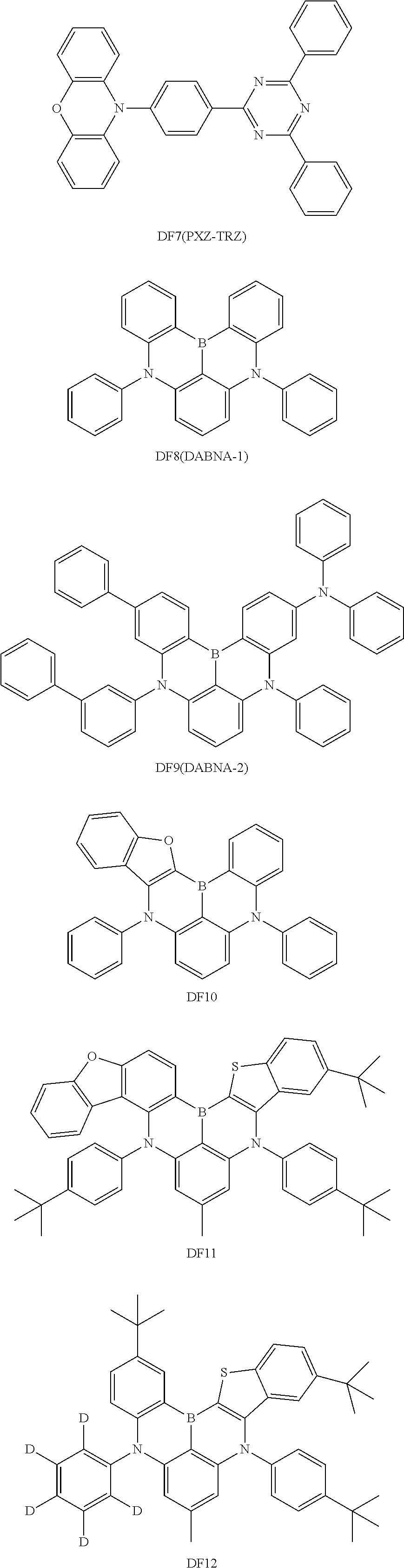

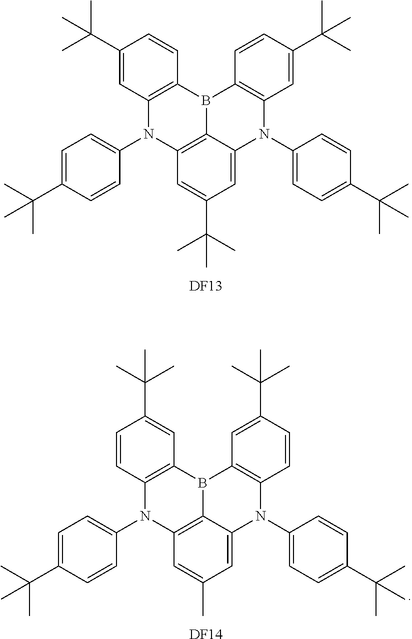

### Electron Transport Region in Interlayer 130

The electron transport region may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron transport region may include a hole blocking layer, an electron transport layer, an electron injection layer, or any combination thereof.

For example, in one or more embodiments, the electron transport region may have a structure including an electron transport layer/electron injection layer structure or a hole blocking layer/electron transport layer/electron injection layer structure, wherein in each structure, constituting layers are sequentially stacked from the emission layer in the stated order.

In one or more embodiments, the electron transport region (for example, a hole blocking layer or an electron transport layer in the electron transport region) may include a metal-free compound including at least one π electron-deficient nitrogen-containing C₁-C₆₀ cyclic group.

In one or more embodiments, the electron transport region may include a compound represented by Formula 601.

[Ar₆₀₁]ₓₑ₁₁-[(L₆₀₁)ₓₑ₁-R₆₀₁]ₓₑ₂₁ Formula 601

In Formula 601,
Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In one or more embodiments, when xe11 in Formula 601 is 2 or more, two or more of Ar₆₀₁ (s) may be linked together via a single bond.

In one or more embodiments, Ar₆₀₁ in Formula 601 may be a substituted or unsubstituted anthracene group.

In one or more embodiments, the electron transport region may include a compound represented by Formula 601-1:

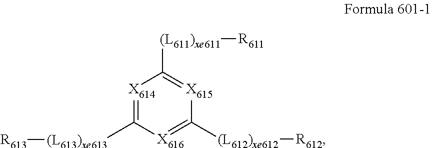

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each independently be the same as described with respect to L₆₀₁,
xe611 to xe613 may each independently be the same as described with respect to xe1,
R₆₁₁ to R₆₁₃ may each independently be the same as described with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In some embodiments, xe1 and xe611 to xe613 in Formulae 601 and 601-1 may each independently be 0, 1, or 2.

In one or more embodiments, the electron transport region may include: at least one selected from among Compounds ET1 to ET45; 2,9-dimethyl-4,7-diphenyl-1,10-phenanthroline (BCP); 4,7-diphenyl-1,10-phenanthroline (Bphen); tris(8-hydroxyquinolinato)aluminum (Alq₃), bis(2-methyl-8-quinolinolato-N1,O8)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), or any combination thereof:

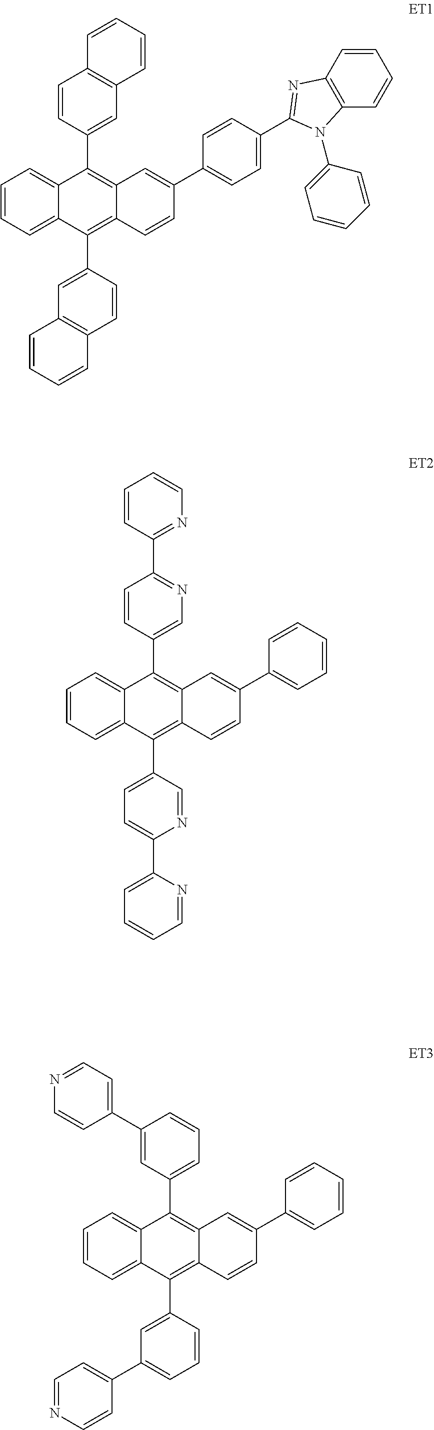 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

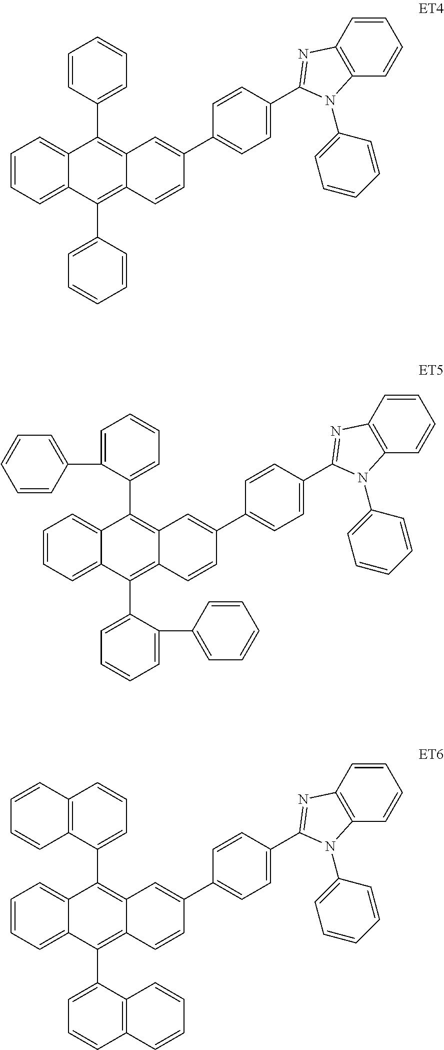 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

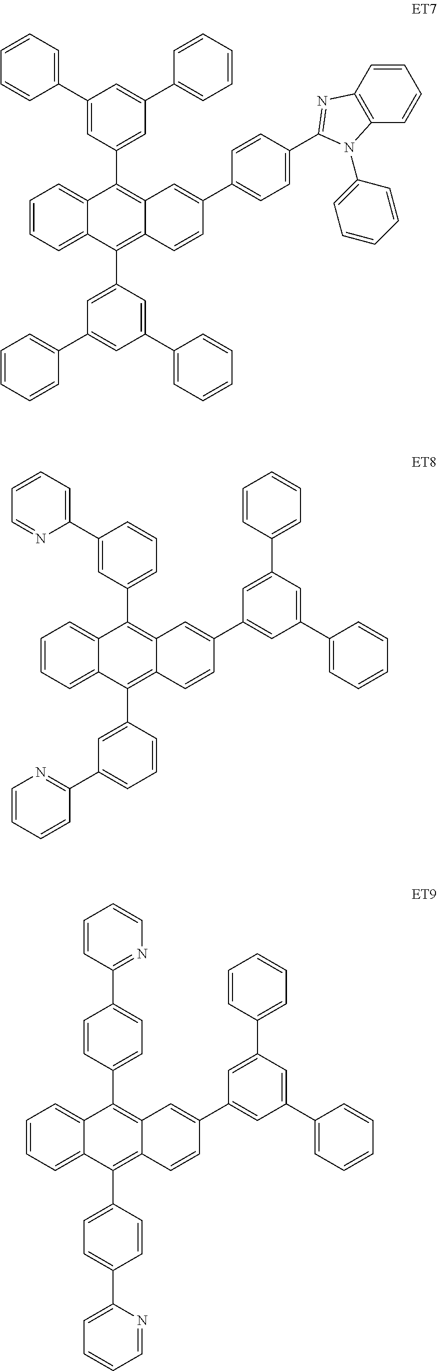 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

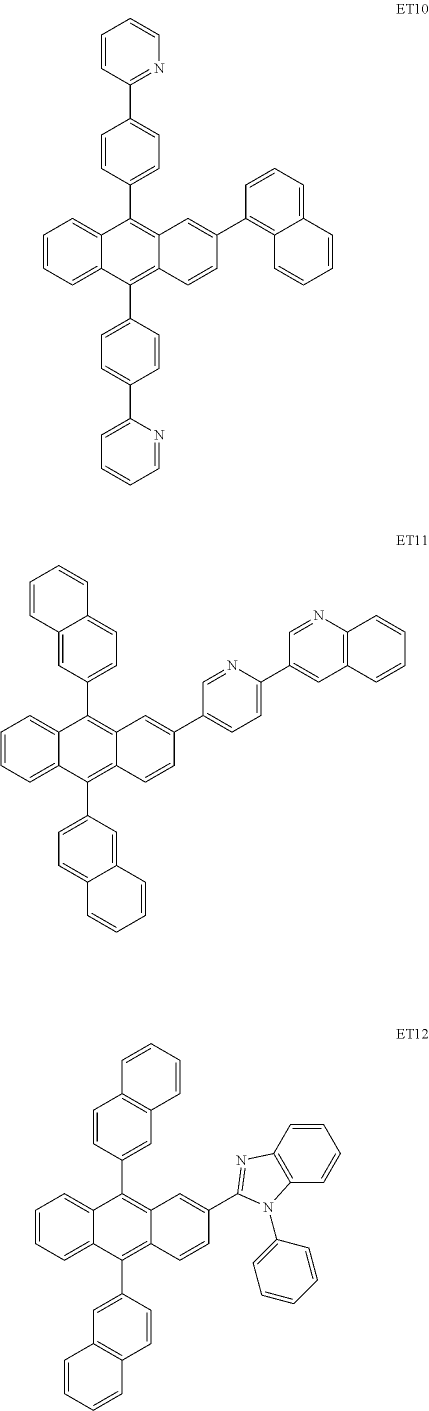 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

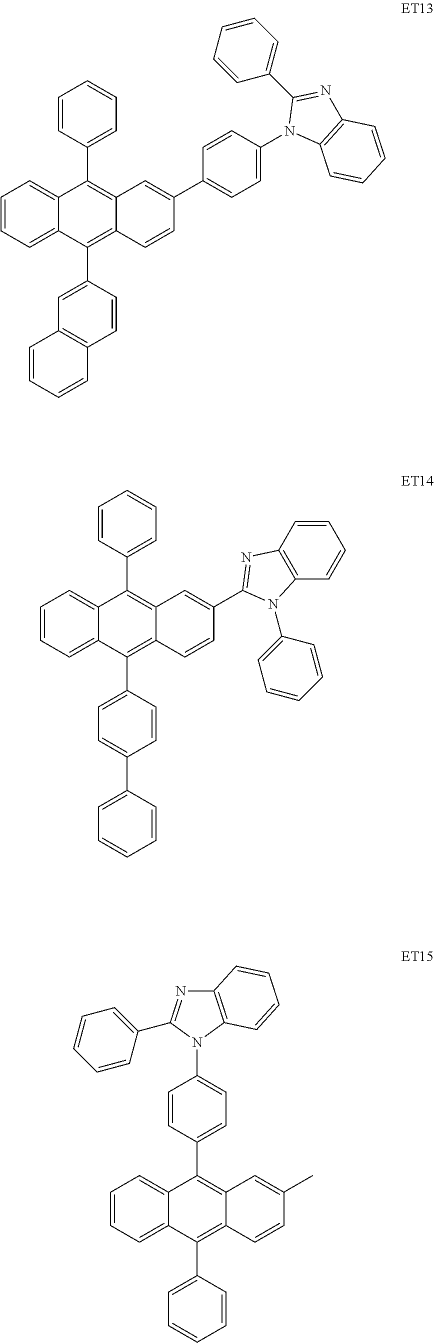 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

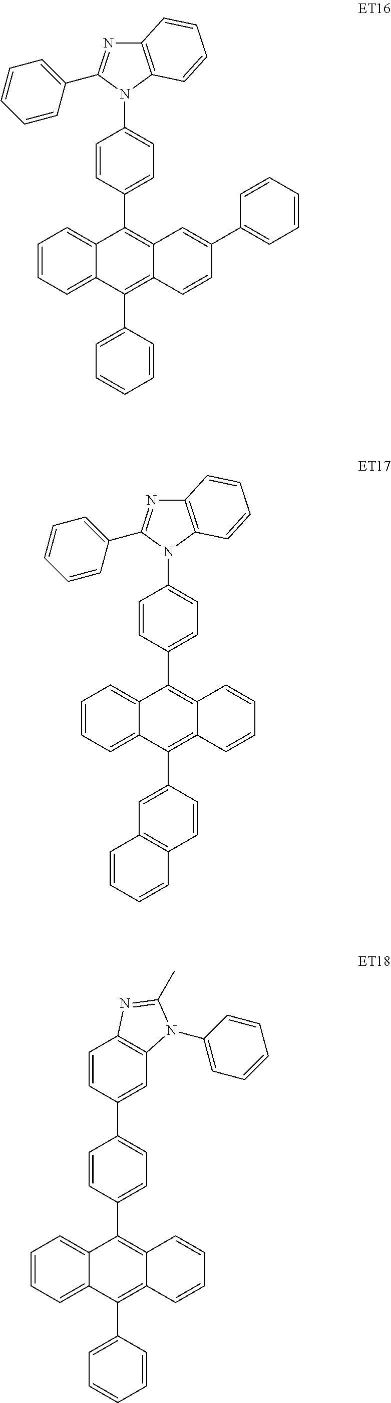 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

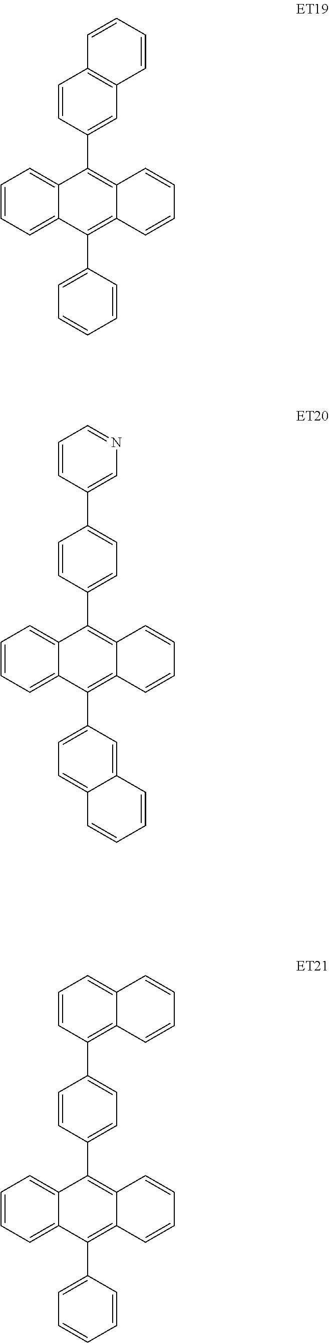

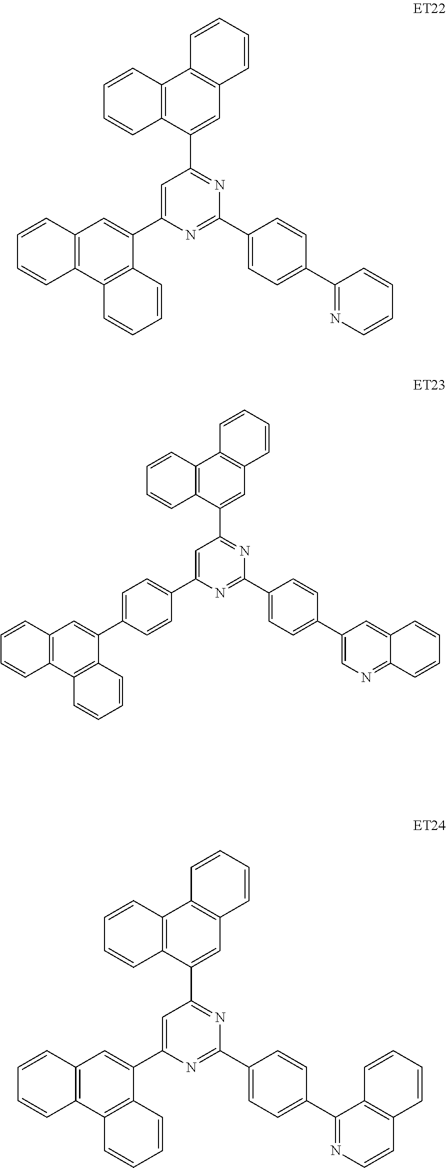 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

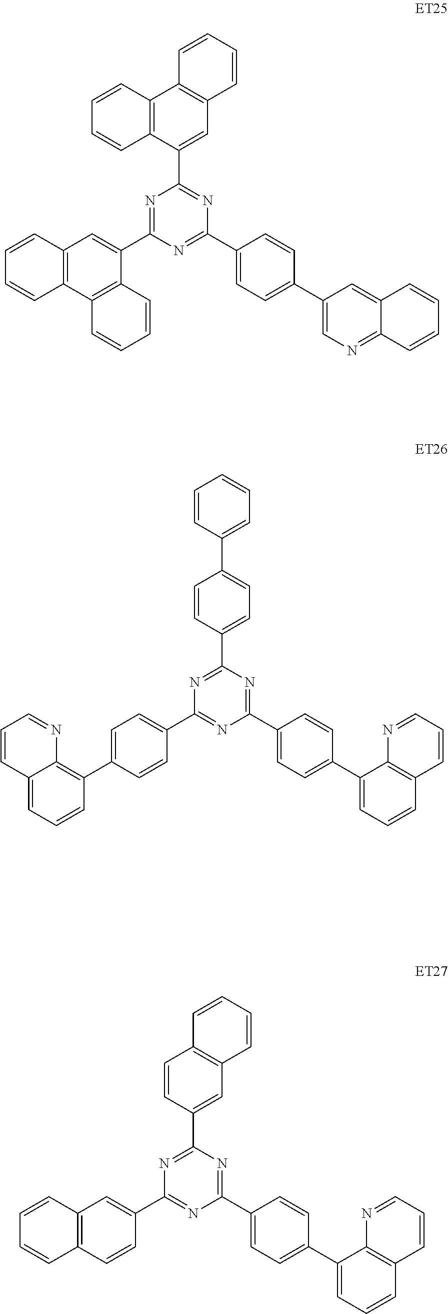 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

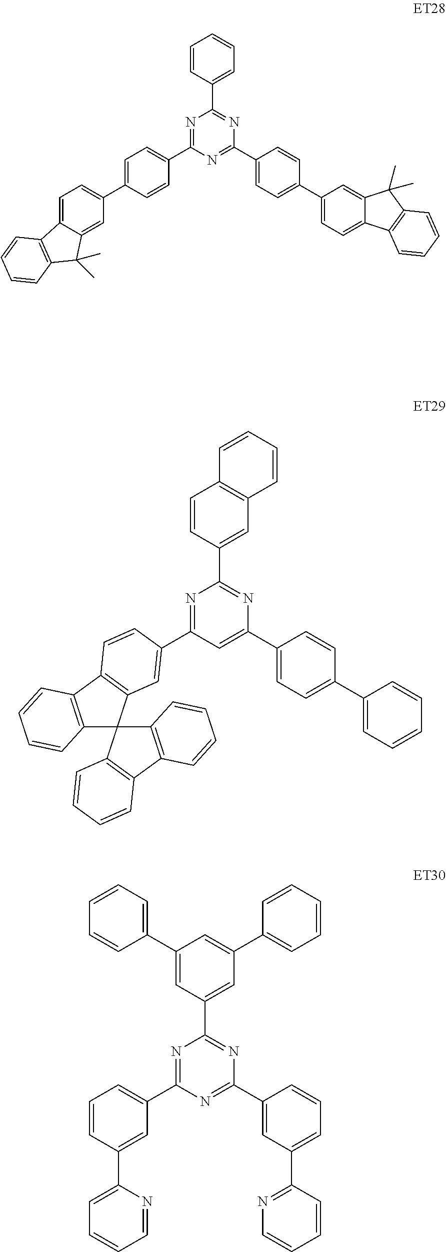 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

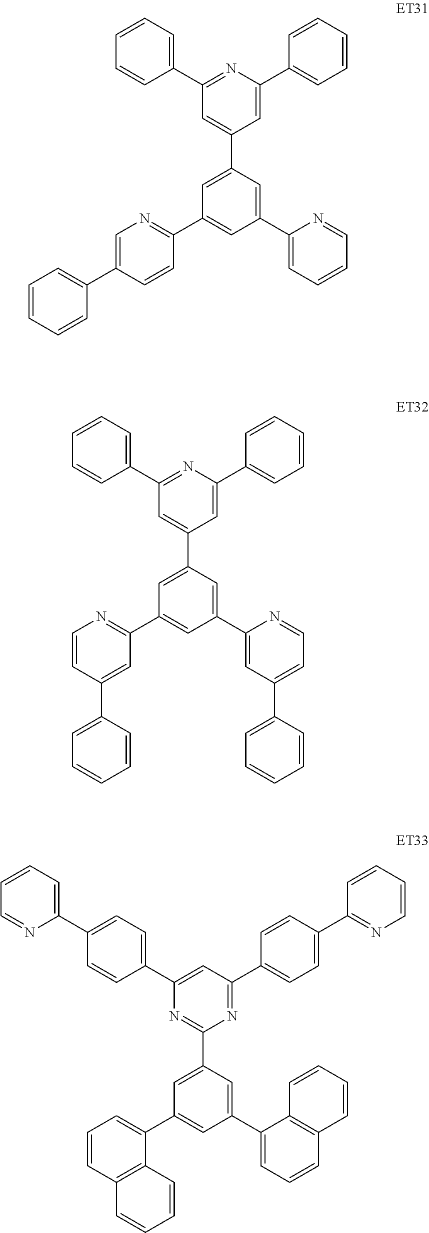 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

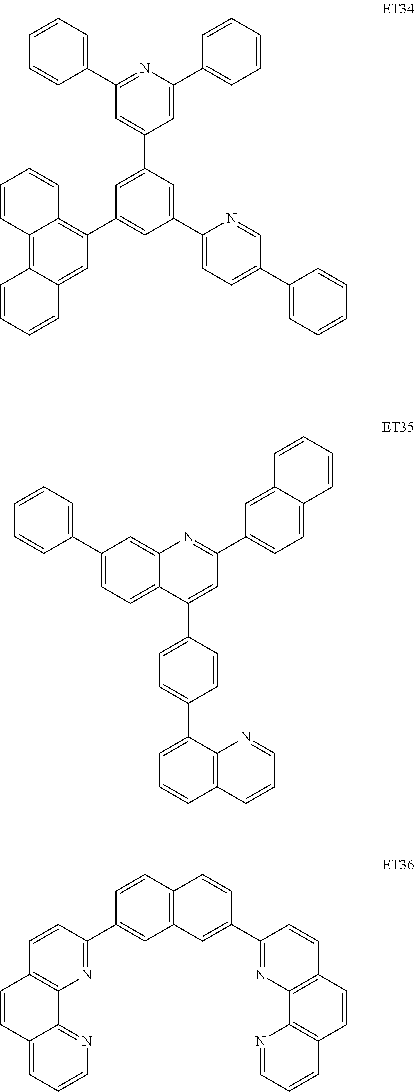 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

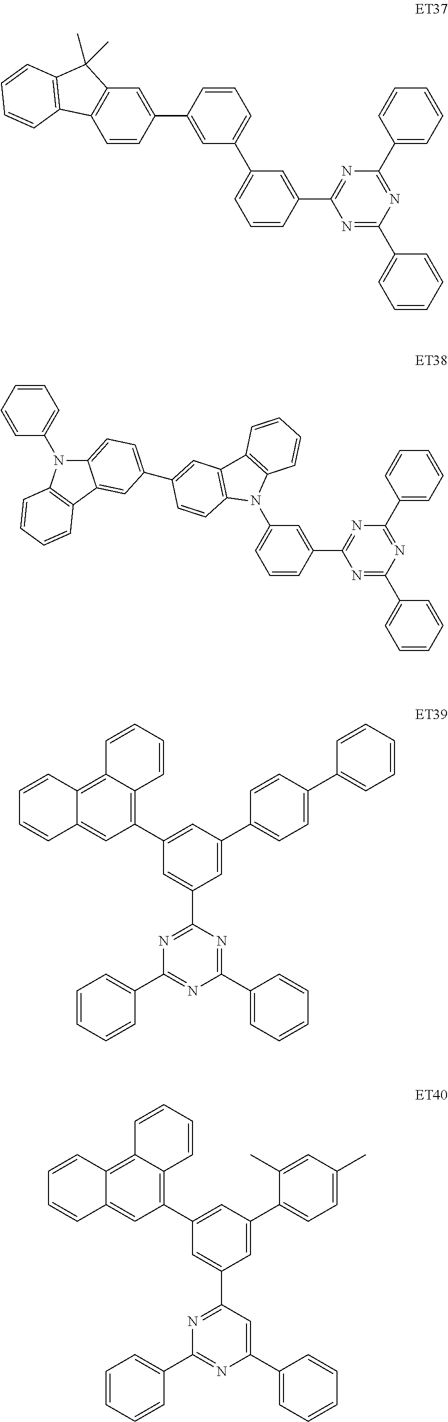 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

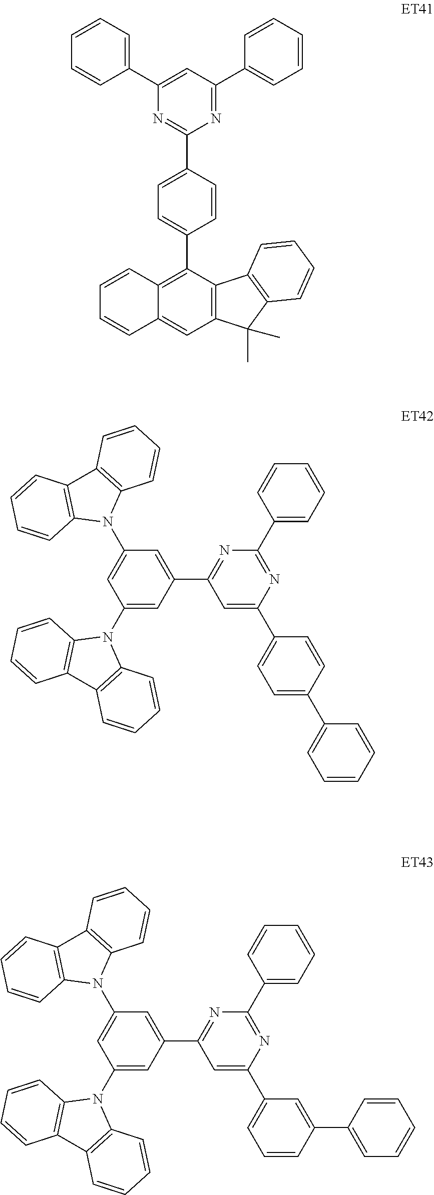 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

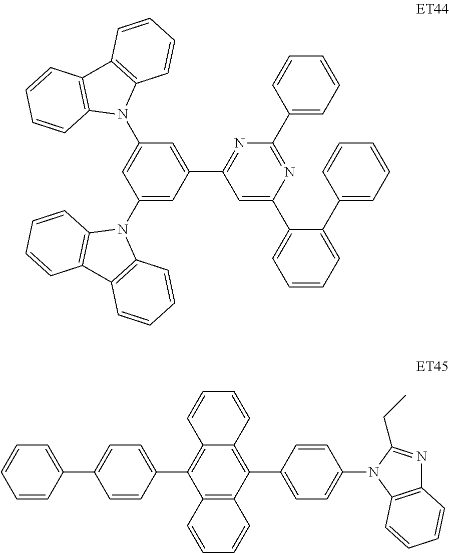 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

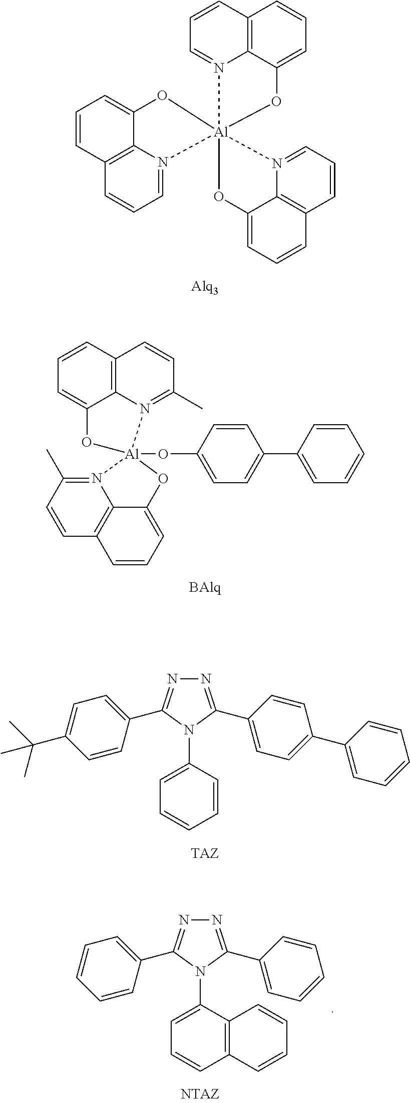

A thickness of the electron transport region may be about 100 Å to about 5,000 Å, for example, about 160 Å to about 4,000 Å. When the electron transport region includes a hole blocking layer, an electron transport layer, or any combination thereof, a thickness of the hole blocking layer may be about 20 Å to about 1,000 Å, for example, about 30 Å to about 300 Å. A thickness of the electron transport layer may be about 100 Å to about 1,000 Å, for example, about 150 Å to about 500 Å. When the thicknesses of the hole blocking layer and/or the electron transport layer are within these ranges as described above, satisfactory electron transporting characteristics may be obtained without a substantial increase in driving voltage.

In one or more embodiments, the electron transport region (for example, the electron transport layer in the electron transport region) may further include, in addition to one or more of the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In one or more embodiments, the metal-containing material may include a Li complex. The Li complex may include, for example, Compound ET-D1 (LiQ) or ET-D2:

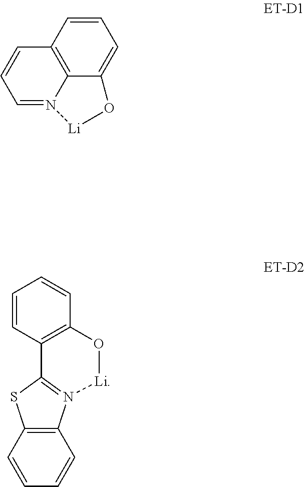

In one or more embodiments, the electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including multiple different materials, or iii) a multilayer structure including multiple layers including multiple different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, etc.), or tellurides of the alkali metal, the alkaline earth metal, and the rare earth metal, respectively, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (x is a real number satisfying 0<x<1), or BaₓCa₁₋ₓO (x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal tellurides. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of metal ions of the alkali metal, one of metal ions of the alkaline earth metal, and one of metal ions of the rare earth metal, respectively, and ii) a ligand bonded to the metal ion (e.g., the respective metal ion), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In some embodiments, the electron injection layer may further include an organic material (for example, a compound represented by Formula 601).

In one or more embodiments, the electron injection layer may include (e.g., consist of) i) an alkali metal-containing compound (for example, alkali metal halide), or ii) a) an alkali metal-containing compound (for example, alkali metal halide); and b) an alkali metal, an alkaline earth metal, a rare earth metal, or any combination thereof. In some embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be substantially uniformly or non-uniformly dispersed in a matrix including the organic material.

A thickness of the electron injection layer may be about 1 Å to about 100 Å, or, for example, about 3 Å to about 90 Å. When the thickness of the electron injection layer is within the range as described above, satisfactory electron injection characteristics may be obtained without a substantial increase in driving voltage.

### Second Electrode 150

The second electrode 150 may be arranged on the interlayer 130. The second electrode 150 may be a cathode, which is an electron injection electrode, and as a material for forming the second electrode 150, a metal, an alloy, an electrically conductive compound, or any combination thereof, each having a low-work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layer structure or a multilayer structure including a plurality of layers.

### Capping Layer

A first capping layer may be arranged outside (e.g., on) the first electrode 110, and/or a second capping layer may be arranged outside (e.g., on) the second electrode 150. In one or more embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

In some embodiments, light generated in the emission layer of the interlayer 130 of the light-emitting device 10 may be extracted toward the outside through the first electrode 110 which is a semi-transmissive electrode or a transmissive electrode, and the first capping layer. In some embodiments, light generated in the emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, such that the luminescence efficiency of the light-emitting device 10 may be increased.

Each of the first capping layer and the second capping layer may include a material having a refractive index of 1.6 or more (e.g., at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may (e.g., the first capping layer and the second capping layer may each independently) include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one of the first capping layer or the second capping layer may (e.g., the first capping layer and the second capping layer may each independently) include an amine group-containing compound.

In one or more embodiments, at least one of the first capping layer or the second capping layer may (e.g., the first capping layer and the second capping layer may each independently) include a compound represented by Formula 201, a compound represented by Formula 202, or any combination thereof.

In one or more embodiments, at least one of the first capping layer or the second capping layer may (e.g., the first capping layer and the second capping layer may each independently) include at least one selected from among Compounds HT28 to HT33, at least one selected from among Compounds CP1 to CP6, β-NPB, or any combination thereof:

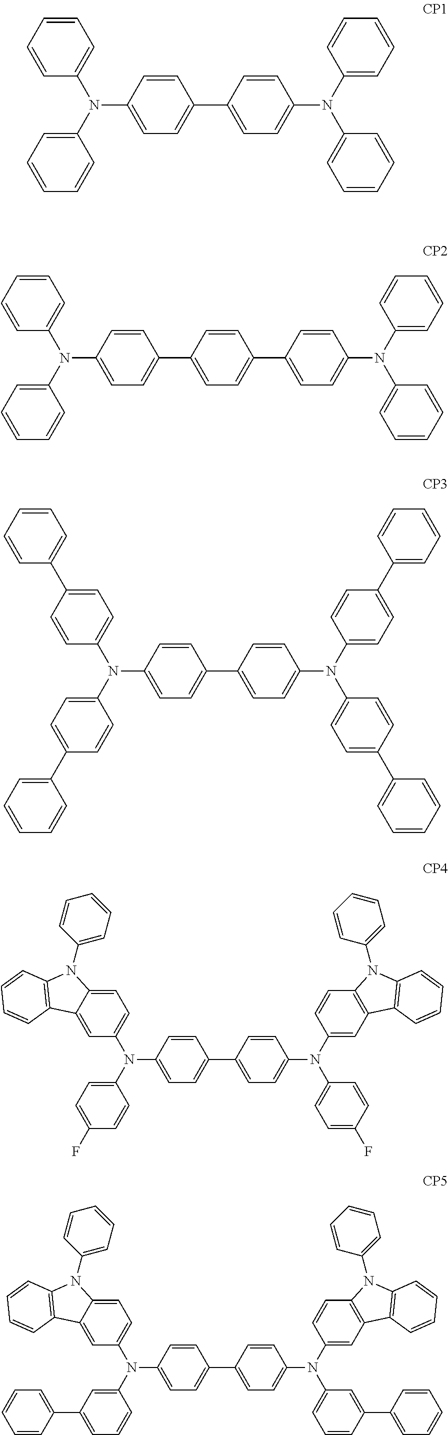 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

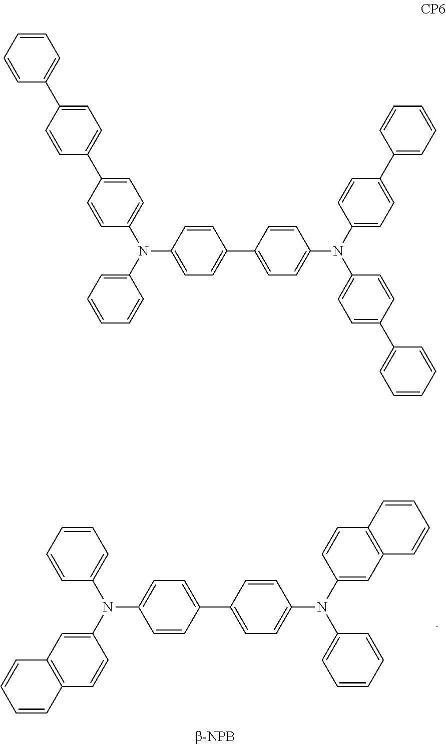 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in one or more suitable electronic apparatuses. For example, in one or more embodiments, the electronic apparatus including the light-emitting device may be a light-emitting apparatus, an authentication apparatus, and/or the like.

In one or mor embodiments, the electronic apparatus (for example, a light-emitting apparatus) may further include, in addition to the light-emitting device, i) a color filter, ii) a color conversion layer, or iii) a color filter and a color conversion layer. The color filter and/or the color conversion layer may be arranged in at least one travel direction of light emitted from the light-emitting device. For example, in some embodiments, the light emitted from the light-emitting device may be blue light or white light (e.g., combined white light). A detailed description of the light-emitting device is provided above.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area configured to emit first color light, a second area configured to emit second color light, and/or a third area configured to emit third color light, wherein the first color light, the second color light, and/or the third color light may have different maximum emission wavelengths. In one or more embodiments, the first color light may be red light, the second color light may be green light, and the third color light may be blue light. In one or more embodiments, the plurality of color filter areas (or the plurality of color conversion areas) may include quantum dots. In some embodiments, the first area may include red quantum dots to emit red light, the second area may include green quantum dots to emit green light, and the third area may not include (e.g., may exclude) quantum dots. In one or more embodiments, the first area, the second area, and/or the third area may each further include a scatterer.

In one or more embodiments, the light-emitting device may be to emit first light, the first area may be to absorb the first light to emit first-first color light, the second area may be to absorb the first light to emit second-first color light, and the third area may be to absorb the first light to emit third-first color light. In some embodiments, the first-first color light, the second-first color light, and the third-first color light may have different maximum emission wavelengths. In some embodiments, the first light may be blue light, the first-first color light may be red light, the second-first color light may be green light, and the third-first color light may be blue light.

In one or more embodiments, the electronic apparatus may further include a thin-film transistor, in addition to the light-emitting device as described above. The thin-film transistor may include a source electrode, a drain electrode, and an active layer, wherein one selected from among the source electrode and the drain electrode may be electrically connected to the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

In one or more embodiments, various functional layers may be additionally arranged on the sealing portion, in addition to the color filter and/or the color conversion layer, according to the utilization of the electronic apparatus. Non-limiting examples of the functional layers may include a touch screen layer and/or a polarizing layer. The touch screen layer may be a pressure-sensitive touch screen layer, a capacitive touch screen layer, or an infrared touch screen layer.

The authentication apparatus may be, for example, a biometric authentication apparatus that authenticates an individual by utilizing biometric information of a living body (for example, fingertips, pupils, etc.). The authentication apparatus may further include, in addition to the light-emitting device as described above, a biometric information collector.

The electronic apparatus may be applied to one or more of displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Equipment

The light-emitting device may be included in one or more suitable electronic equipment.

In one or more embodiments, the electronic equipment including the light-emitting device may be at least one selected from among a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including multiple displays tiled together, a theater or stadium screen, a phototherapy device, a signboard, and combinations thereof.

Because the light-emitting device has excellent or suitable effects in terms of luminescence efficiency long lifespan, the electronic equipment including the light-emitting device may have desirable characteristics with high luminance, high resolution, and low power consumption.

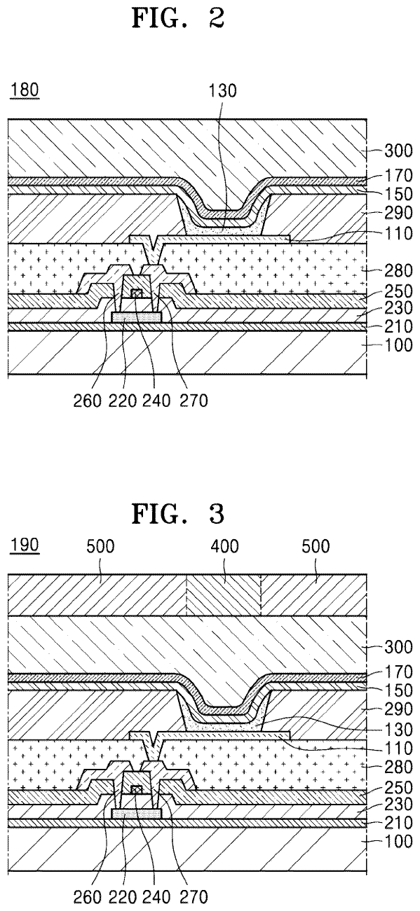

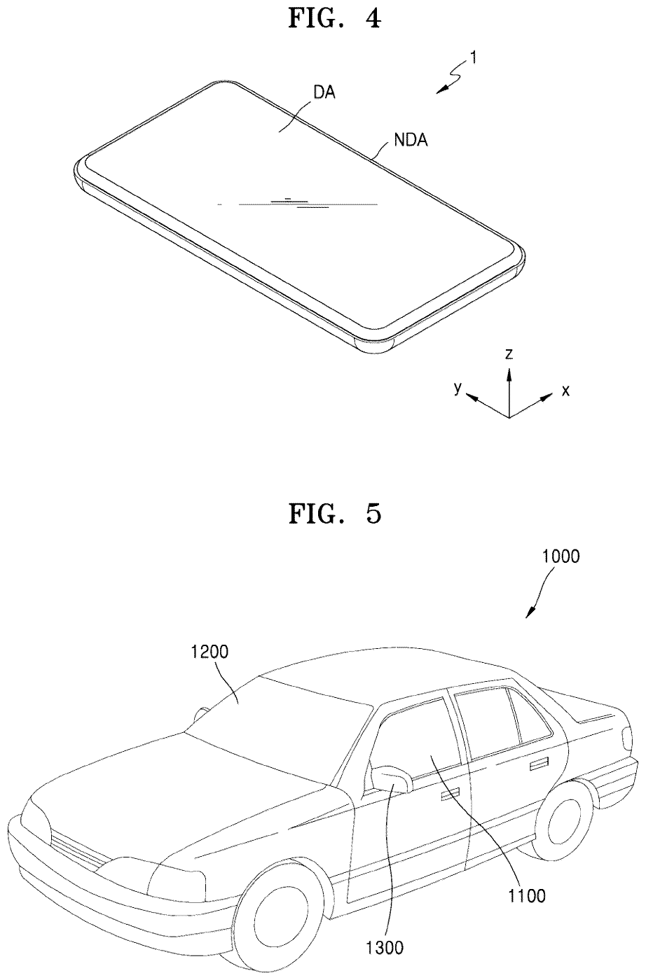

### Description of FIG. 2 and FIG. 3

FIG. 2 is a cross-sectional view of an electronic apparatus 180 according to one or more embodiments of the present disclosure.

The electronic apparatus 180 of FIG. 2 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on the substrate 100. The buffer layer 210 may prevent or reduce penetration of impurities through the substrate 100 and may provide a flat surface on the substrate 100.

The TFT may be arranged on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be arranged between the gate electrode 240 and the source electrode 260 and between the gate electrode 240 and the drain electrode 270, to insulate from one another.

The source electrode 260 and the drain electrode 270 may be on the interlayer insulating film 250. The interlayer insulating film 250 and the gate insulating film 230 may be formed to expose the source region and the drain region of the activation layer 220, and the source electrode 260 and the drain electrode 270 may be arranged in contact with the exposed portions of the source region and the drain region of the activation layer 220, respectively.

The TFT may be electrically connected to the light-emitting device to drive the light-emitting device, and may be covered and protected by a passivation layer 280. The passivation layer 280 may include an inorganic insulating film, an organic insulating film, or any combination thereof. The light-emitting device may be provided on the passivation layer 280. The light-emitting device may include a first electrode 110, an interlayer 130, and a second electrode 150.

The first electrode 110 may be on the passivation layer 280. The passivation layer 280 may be arranged to expose a portion of the drain electrode 270, not fully covering the drain electrode 270, and the first electrode 110 may be arranged to be connected to the exposed portion of the drain electrode 270.

A pixel-defining film 290 including an insulating material may be on the first electrode 110. The pixel-defining film 290 may expose a certain region of the first electrode 110, and the interlayer 130 may be formed in the exposed region of the first electrode 110. The pixel-defining film 290 may be a polyimide-based organic film or a polyacrylic organic film. In some embodiments, at least some layers of the interlayer 130 may extend beyond the upper portion of the pixel-defining film 290 to be arranged in the form of a common layer.

The second electrode 150 may be on the interlayer 130, and a capping layer 170 may be additionally formed on the second electrode 150. The capping layer 170 may be formed to cover the second electrode 150.

The encapsulation portion 300 may be on the capping layer 170. The encapsulation portion 300 may be disposed on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, and/or the like), an epoxy-based resin (for example, aliphatic glycidyl ether (AGE), and/or the like), or any combination thereof; or a combination of the inorganic film and the organic film.

FIG. 3 is a cross-sectional view of an electronic apparatus 190 according to one or more embodiments.

The electronic apparatus 190 of FIG. 3 is substantially the same as the electronic apparatus 180 of FIG. 2, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus 190 of FIG. 3 may be a tandem light-emitting device.

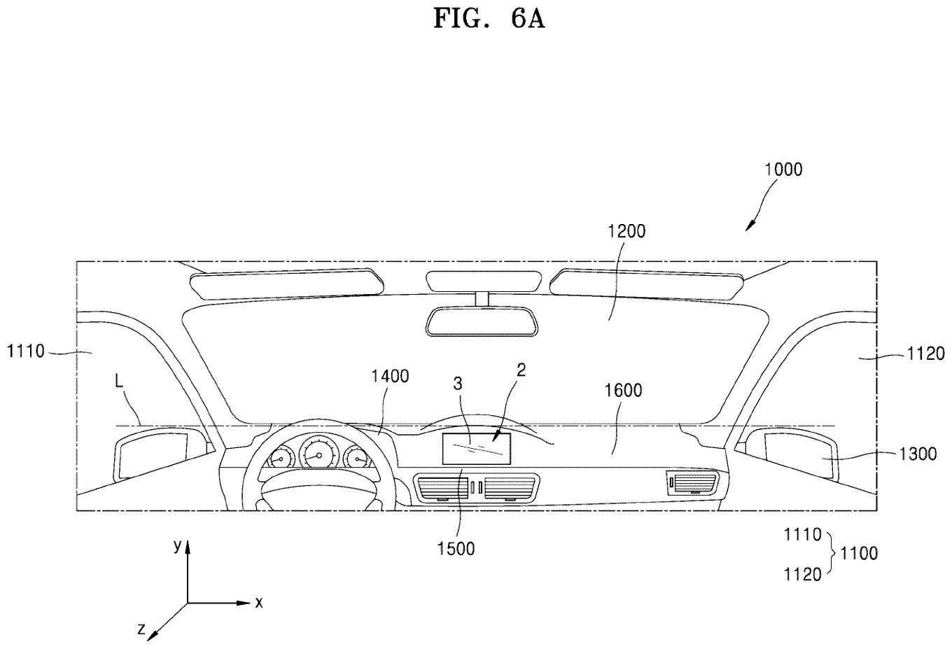

### Description of FIG. 4

FIG. 4 is a schematic perspective view of electronic equipment 1 including a light-emitting device according to one or more embodiments of the present disclosure. The electronic equipment 1 may be, as an apparatus that displays a moving image or a still image, a portable electronic equipment, such as a mobile phone, a smartphone, a tablet personal computer (PC), a mobile communication terminal, an electronic notebook, an electronic book, a portable multimedia player (PMP), a navigation, or a ultra-mobile PC (UMPC), as well as one or more suitable products, such as a television, a laptop, a monitor, a billboard, or an Internet of things (IOT). The electronic equipment 1 may be such a product above or a part thereof. In some embodiments, the electronic equipment 1 may be a wearable device, such as a smart watch, a watch phone, a glasses-type or kind display, or a head mounted display (HMD), or a part of the wearable device. However, embodiments of the present disclosure are not limited thereto. In one or more embodiments, the electronic equipment 1 may be a dashboard of a vehicle, a center information display (CID) arranged on a center fascia or dashboard of a vehicle, a room mirror display instead of a side-view mirror of a vehicle, an entertainment for the back seat of a vehicle, or a display arranged on the back of the front seat of a vehicle, a head up display (HUD) installed on the front of a vehicle or projected on a front window glass, and/or a computer generated hologram augmented reality head up display (CGH AR HUD). FIG. 4 illustrates an embodiment in which the electronic equipment 1 is a smartphone for convenience of explanation.

The electronic equipment 1 may include a display area DA and a non-display area NDA outside the display area DA. A display apparatus of the electronic equipment 1 may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display devices arranged on the display area DA may be arranged. On the non-display area NDA, a pad, which is an area to which an electronic element or a printed circuit board, may be electrically connected may be arranged.

In the electronic equipment 1, a length in an x-axis direction and a length (e.g., a width) in a y-axis direction may be different from each other. In some embodiments, as shown in FIG. 4, the length in the x-axis direction may be less than the length (e.g., the width) in the y-axis direction. In some embodiments, the length in the x-axis direction may be substantially the same as the length (e.g., the width) in the y-axis direction. In some embodiments, the length in the x-axis direction may be greater than the length (e.g., the width) in the y-axis direction.

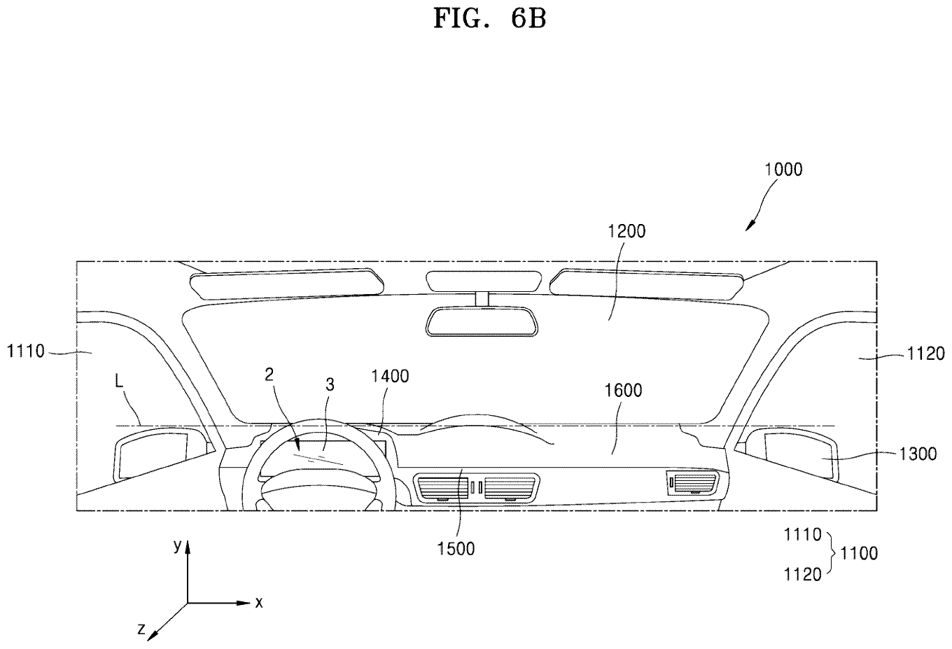

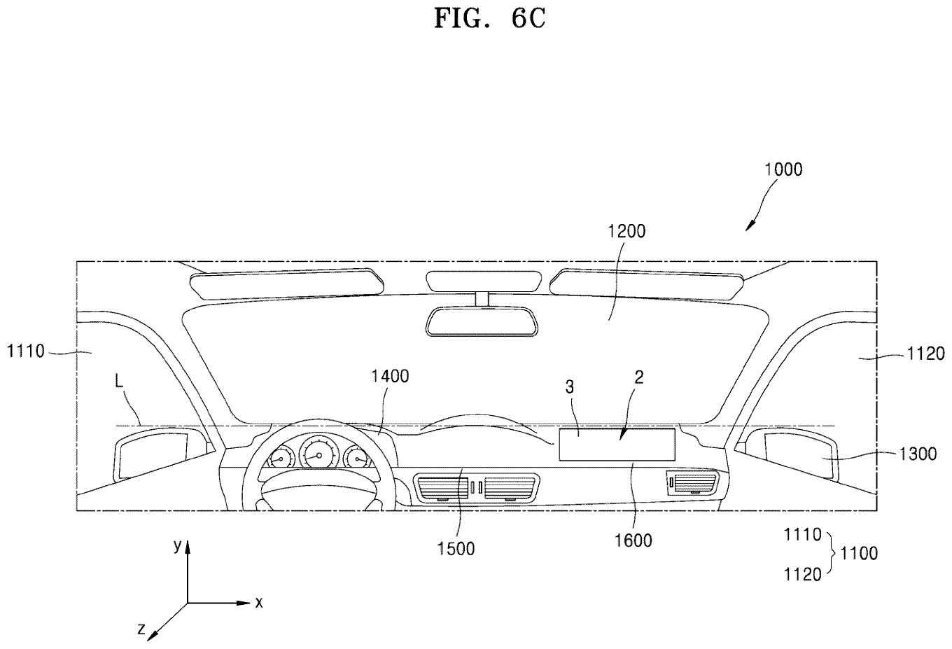

### Descriptions of FIGS. 5 and 6A to 6C

FIG. 5 is a schematic view of an exterior of a vehicle 1000 as electronic equipment including a light-emitting device, according to one or more embodiments. FIGS. 6A to 6C are each a schematic view of an interior of a vehicle 1000 according to one or more embodiments.

Referring to FIGS. 5, 6A, 6B, and 6C, the vehicle 1000 may refer to one or more suitable apparatuses for moving an object to be transported, such as a human, an object, or an animal, from a departure point to a destination point. The vehicle 1000 may include a vehicle traveling on a road or a track, a vessel moving over the sea or a river, an airplane flying in the sky utilizing the action of air, and/or the like.

In one or more embodiments, the vehicle 1000 may travel on a road or a track. The vehicle 1000 may move in a certain direction according to rotation of at least one wheel thereof. In some embodiments, the vehicle 1000 may include a three-wheeled or four-wheeled vehicle, a construction machine, a two-wheeled vehicle, a prime mover device, a bicycle, or a train running on a track.

The vehicle 1000 may include a vehicle body having an interior and an exterior, and a chassis in which mechanical apparatuses necessary for driving are installed as other parts except for the vehicle body. The exterior of the vehicle body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on a side of the vehicle 1000. In some embodiments, the side window glass 1100 may be installed on a door of the vehicle 1000. A plurality of side window glasses 1100 may be provided and may face each other. In some embodiments, the side window glass 1100 may include a first side window glass 1110 and a second side window glass 1120. In some embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in an x direction or a −x direction (the direction opposite the x-direction). In some embodiments, the first side window glass 1110 and the second side window glass 1120 may be spaced apart from each other in the x direction or the −x direction. In other words, an imaginary straight line L connecting the side window glasses 1100 may extend in the x direction or the −x direction. In some embodiments, an imaginary or virtual straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side-view mirror 1300 may provide a rear view of the vehicle 1000. The side-view mirror 1300 may be installed on the exterior of the vehicle body. In some embodiments, a plurality of side-view mirrors 1300 may be provided. Any one of the plurality of side-view mirrors 1300 may be arranged outside the first side window glass 1110. The other one of the plurality of side-view mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning light, a seat belt warning light, an odometer, a tachograph, an automatic shift selector indicator, a door open warning light, an engine oil warning light, and/or a low fuel warning light.

The center fascia 1500 may include a control panel on which a plurality of buttons for adjusting an audio device, an air conditioning device, and/or a heater of a seat are disposed. The center fascia 1500 may be arranged on one side of the cluster 1400.

The passenger seat dashboard 1600 may be spaced apart from the cluster 1400 with the center fascia 1500 arranged therebetween. In some embodiments, the cluster 1400 may be arranged to correspond to a driver seat, and the passenger seat dashboard 1600 may be arranged to correspond to a passenger seat. In some embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In some embodiments, the display apparatus 2 may be arranged between the side window glasses 1100 facing each other. The display apparatus 2 may be arranged on at least one of the cluster 1400, the center fascia 1500, or the passenger seat dashboard 1600.

The display apparatus 2 may include an organic light-emitting display, an inorganic electroluminescent display, a quantum dot display, and/or the like. Hereinafter, as the display apparatus 2 according to one or more embodiments, an organic light-emitting display apparatus including the light-emitting device will be described as an example, but one or more suitable types (kinds) of display apparatuses as described above may be utilized in embodiments.

Referring to FIG. 6A, in one or more embodiments, the display apparatus 2 may be arranged on the center fascia 1500. In some embodiments, the display apparatus 2 may display navigation information. In some embodiments, the display apparatus 2 may display audio, video, or information regarding vehicle settings.

Referring to FIG. 6B, in one or more embodiments, the display apparatus 2 may be arranged on the cluster 1400. In this case, the cluster 1400 may display driving information and/or the like through the display apparatus 2. In some embodiments, the cluster 1400 may be implemented digitally. The digital cluster 1400 may display vehicle information and driving information as images. In some embodiments, a needle and a gauge of a tachometer and one or more suitable warning light icons may be displayed by a digital signal.

Referring to FIG. 6C, in one or more embodiments, the display apparatus 2 may be arranged on the passenger seat dashboard 1600. The display apparatus 2 may be embedded in the passenger seat dashboard 1600 or arranged on the passenger seat dashboard 1600. In some embodiments, the display apparatus 2 arranged on the passenger seat dashboard 1600 may display an image related to information displayed on the cluster 1400 and/or information displayed on the center fascia 1500. In some embodiments, the display apparatus 2 arranged on the passenger seat dashboard 1600 may display information different from information displayed on the cluster 1400 and/or information displayed on the center fascia 1500.

### Manufacturing Method

The layers constituting the hole transport region, the emission layer, and the layers constituting the electron transport region may each be formed in a certain region by utilizing one or more suitable methods such as vacuum deposition, spin coating, casting, Langmuir-Blodgett (LB) deposition, ink-jet printing, laser-printing, laser-induced thermal imaging, and/or the like.

When the layers constituting the hole transport region, the emission layer, and the layers constituting the electron transport region are each formed by vacuum deposition, the deposition may be performed at a deposition temperature in a range of about 100° C. to about 500° C., at a vacuum degree in a range of about 10⁻⁸ torr to about 10⁻³ torr, and at a deposition speed in a range of about 0.01 Å/sec to about 100 Å/sec, depending on a material to be included in a layer to be formed and the structure of a layer to be formed.

### DEFINITION OF TERMS

The term “C₃-C₆₀ carbocyclic group” as utilized herein refers to a cyclic group including (e.g., consisting of) carbon only as a ring-forming atom and having three to sixty carbon atoms, and the term “C₁-C₆₀ heterocyclic group” as utilized herein refers to a cyclic group that has one to sixty carbon atoms and further includes, in addition to the carbon atoms, a heteroatom as a ring-forming atom. The C₃-C₆₀ carbocyclic group and the C₁-C₆₀ heterocyclic group may each be a monocyclic group including (e.g., consisting of) one (e.g., exactly one) ring or a polycyclic group in which two or more rings are condensed with each other. In one or more embodiments, the number of ring-forming atoms of the C₁-C₆₀ heterocyclic group may be 3 to 61.

The term “cyclic group” as utilized herein may include both (e.g., simultaneously) the C₃-C₆₀ carbocyclic group and the C₁-C₆₀ heterocyclic group.

The term “π electron-rich C₃-C₆₀ cyclic group” as utilized herein refers to a cyclic group that has three to sixty carbon atoms and does not include *—N═*′ as a ring-forming moiety, and the term “π electron-deficient nitrogen-containing C₁-C₆₀ cyclic group” as utilized herein refers to a heterocyclic group that has one to sixty carbon atoms and includes *—N═*′ as a ring-forming moiety.

In one or more embodiments,
the C₃-C₆₀ carbocyclic group may be i) Group T1 (e.g., one selected from among the groups in Group T1) or ii) a condensed cyclic group in which two or more of Group T1 (e.g., two or more selected from among the groups in Group T1) are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 (e.g., at least one of the groups in Group T2) and at least one Group T1 (e.g., at least one of the groups in Group T1) are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),
the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may be a furan group, a thiophene group, a 1H-pyrrole group, a silole group, or a borole group, and
Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as utilized herein may refer to a group condensed to any cyclic group, a monovalent group, or a polyvalent group (for example, a divalent group, a trivalent group, a tetravalent group, etc.) according to the structure of a formula for which the corresponding term is utilized. In one or more embodiments, the “benzene group” may be a benzo group, a phenyl group, a phenylene group, and/or the like, which may be easily understood by those of ordinary skill in the art according to the structure of a formula including the “benzene group.”

Depending on context, in the present disclosure, a divalent group may refer to or include a polyvalent group (e.g., trivalent, tetravalent, etc., and not just divalent) per, e.g., the structure of a formula in connection with which of the terms are utilized.

For example, non-limiting examples of a monovalent C₃-C₆₀ carbocyclic group and a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and non-limiting examples of a divalent C₃-C₆₀ carbocyclic group and a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as utilized herein refers to a linear or branched aliphatic hydrocarbon monovalent group that has one to sixty carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having substantially the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of a C₂-C₆₀ alkyl group, and non-limiting examples thereof may include an ethenyl group, a propenyl group, and a butenyl group. The term “C₂-C₆₀ alkenylene group” as utilized herein refers to a divalent group having substantially the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of a C₂-C₆₀ alkyl group, and non-limiting examples thereof may include an ethynyl group and a propynyl group. The term “C₂-C₆₀ alkynylene group” as utilized herein refers to a divalent group having substantially the same structure as the C₂-C₆₀ alkynyl group.

The term “C₁-C₆₀ alkoxy group” as utilized herein refers to a monovalent group represented by -OA₁₀₁ (wherein A₁₀₁ is a C₁-C₆₀ alkyl group), and non-limiting examples thereof may include a methoxy group, an ethoxy group, and an isopropyloxy group.

The term “C₃-C₁₀ cycloalkyl group” as utilized herein refers to a monovalent saturated hydrocarbon monocyclic group including 3 to 10 carbon atoms. Non-limiting examples of the C₃-C₁ cycloalkyl group as utilized herein may include a cyclopropyl group, a cyclobutyl group, a cyclopentyl group, a cyclohexyl group, a cycloheptyl group, a cyclooctyl group, an adamantyl group, a norbornyl (bicyclo[2.2.1]heptyl) group, a bicyclo[1.1.1]pentyl group, a bicyclo[2.1.1]hexyl group, or a bicyclo[2.2.2]octyl group. The term “C₃-C₁a cycloalkylene group” as utilized herein refers to a divalent group having substantially the same structure as the C₃-C₁₀ cycloalkyl group.

The term “C₁-C₁₀ heterocycloalkyl group” as utilized herein refers to a monovalent cyclic group that has one to ten carbon atoms and further includes, in addition to the carbon atoms, at least one heteroatom as a ring-forming atom, and non-limiting examples thereof may include a 1,2,3,4-oxatriazolidinyl group, a tetrahydrofuranyl group, and a tetrahydrothiophenyl group. The term “C₁-C₁₀ heterocycloalkylene group” as utilized herein refers to a divalent group having substantially the same structure as the C₁-C₁₀ heterocycloalkyl group.

The term “C₃-C₁₀ cycloalkenyl group” as utilized herein refers to a monovalent cyclic group that has three to ten carbon atoms and at least one carbon-carbon double bond in the ring thereof and no aromaticity, and non-limiting examples thereof may include a cyclopentenyl group, a cyclohexenyl group, and a cycloheptenyl group. The term “C₃-C₁₀ cycloalkenylene group” as utilized herein refers to a divalent group having substantially the same structure as the C₃-C₁₀ cycloalkenyl group.

The term “C₁-C₁₀ heterocycloalkenyl group” as utilized herein refers to a monovalent cyclic group that has one to ten carbon atoms, further includes, in addition to the carbon atoms, at least one heteroatom as a ring-forming atom, and has at least one double bond in the ring thereof. Non-limiting examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as utilized herein refers to a divalent group having substantially the same structure as the C₁-C₁₀ heterocycloalkenyl group.

The term “C₆-C₆₀ aryl group” as utilized herein refers to a monovalent group having a carbocyclic aromatic system of six to sixty carbon atoms, and the term “C₆-C₆₀ arylene group” as utilized herein refers to a divalent group having a carbocyclic aromatic system of six to sixty carbon atoms. Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the two or more rings may be condensed with each other.

The term “C₁-C₆₀ heteroaryl group” as utilized herein refers to a monovalent group having a heterocyclic aromatic system that has one to sixty carbon atoms and further includes, in addition to the carbon atoms, at least one heteroatom as a ring-forming atom. The term “C₁-C₆₀ heteroarylene group” as utilized herein refers to a divalent group having a heterocyclic aromatic system that has one to sixty carbon atoms and further includes, in addition to the carbon atoms, at least one heteroatom as a ring-forming atom. Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the two or more rings may be condensed with each other.

The term “monovalent non-aromatic condensed polycyclic group” as utilized herein refers to a monovalent group having two or more rings condensed with each other, only carbon atoms (for example, eight to sixty carbon atoms) as ring-forming atoms, and no aromaticity in its molecular structure when considered as a whole. Non-limiting examples of the monovalent non-aromatic condensed polycyclic group may include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as utilized herein refers to a divalent group having substantially the same structure as the monovalent non-aromatic condensed polycyclic group.

The term “monovalent non-aromatic condensed heteropolycyclic group” as utilized herein refers to a monovalent group that has two or more rings condensed with each other, further includes, in addition to carbon atoms (for example, one to sixty carbon atoms), at least one heteroatom as a ring-forming atom, and has no aromaticity in its molecular structure when considered as a whole. Non-limiting examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having substantially the same structure as the monovalent non-aromatic condensed heteropolycyclic group.

The term “C₆-C₆₀ aryloxy group” as utilized herein refers to -OA₁₀₂ (wherein A₁₀₂ is a C₆-C₆₀ aryl group), and the term “C₆-C₆₀ arylthio group” as utilized herein refers to -SA₁₀₃ (wherein A₁₀₃ is a C₆-C₆₀ aryl group).

The term “C₇-C₆₀ arylalkyl group” as utilized herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as utilized herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(021), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

The term “heteroatom” as utilized herein refers to any atom other than a carbon atom. Non-limiting examples of the heteroatom may include O, S, N, P, Si, B, Ge, Se, or any combination thereof.

The term “third-row transition metal” as utilized herein includes hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “tert-Bu” or “Bu^{t}” as utilized herein refers to a tert-butyl group, and the term “OMe” as utilized herein refers to a methoxy group.

The term “biphenyl group” as utilized herein refers to “a phenyl group that is substituted with a phenyl group.” In some embodiments, the “biphenyl group” may be a substituted phenyl group having a C₆-C₆₀ aryl group as a substituent.

The term “terphenyl group” as utilized herein refers to “a phenyl group substituted with a biphenyl group.” The “terphenyl group” is a substituted phenyl group having, as a substituent, a C₆-C₆₀ aryl group substituted with a C₆-C₆₀ aryl group.

The maximum number of carbon atoms in the substituent definition is an example. For example, the number 60 as the maximum number of carbon atoms in the C₁-C₆₀ alkyl group is an example, and the definition of an alkyl group is also equally applied to a C₁-C₂₀ alkyl group. The other cases may each independently be the same.

* and *′ as utilized herein, unless defined otherwise, each refer to a binding site to a neighboring atom in a corresponding formula.

Hereinafter, compounds and light-emitting devices according to one or more embodiments will be described in more detail with reference to the following Examples.

### EXAMPLES

### Synthesis of Compounds

### Synthesis Example 1: Synthesis of Compound 2

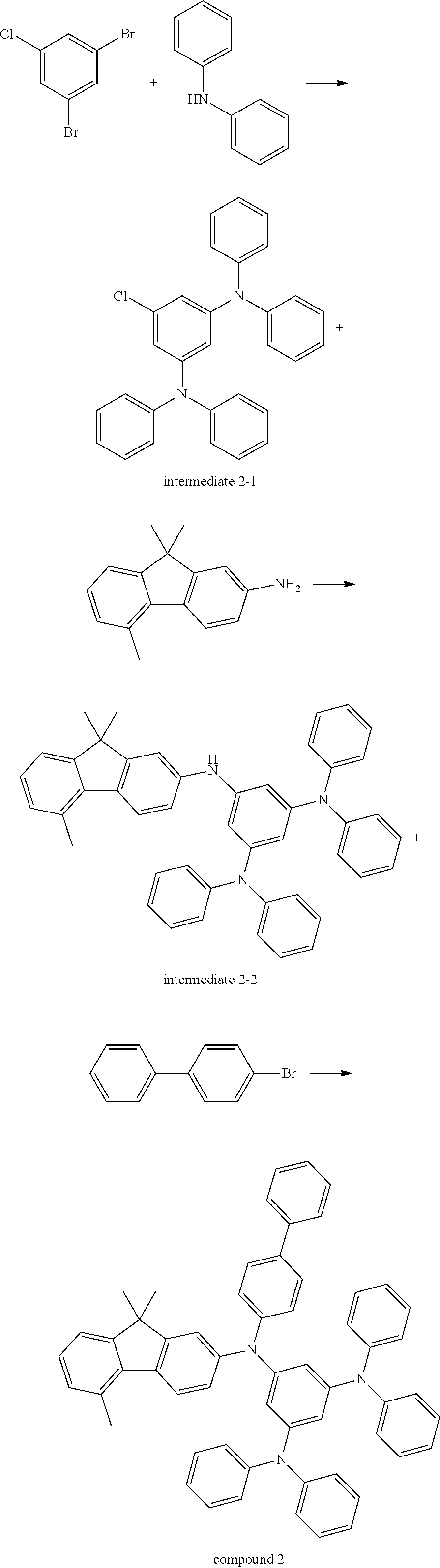

### Synthesis of Intermediate 2-1

2.70 g (10 mmol) of 1,3-dibromo-5-chlorobenzene, 3.49 g (20 mmol) of diphenylamine, 0.92 g (1 mmol) of tris(dibenzylideneacetone)dipalladium(0) (Pd₂(dba)₃), 0.48 g (2 mmol) of P(t-Bu)₃, and 5.76 g (60 mmol) of sodium tert-butoxide were dissolved in 60 mL of toluene and then stirred at 80° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 3.13 g (yield: 70%) of Intermediate 2-1. The resulting compound was identified by liquid chromatography-mass spectrometry (LC-MS). C₃₀H₂₃ClN₂:M⁺446.9

### Synthesis of Intermediate 2-2

4.47 g (10 mmol) of Intermediate 2-1, 2.68 g (12 mmol) of 5,9,9-trimethyl-9H-fluoren-2-amine, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of o-xylene and then stirred at 140° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 4.12 g (yield: 65%) of Intermediate 2-2. The resulting compound was identified by LC-MS. C₄₆H₃₉N₃:M⁺633.8

### Synthesis of Compound 2

6.34 g (10 mmol) of Intermediate 2-2, 3.49 g (10 mmol) of 4-bromo-1,1′-biphenyl, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of toluene and then stirred at 80° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 5.50 g (yield: 70%) of Compound 2. The resulting compound was identified by mass spectroscopy/fast atom bombardment (MS/FAB) and ¹H nuclear magnetic resonance spectroscopy (NMR).

### Synthesis Example 2: Synthesis of Compound 6

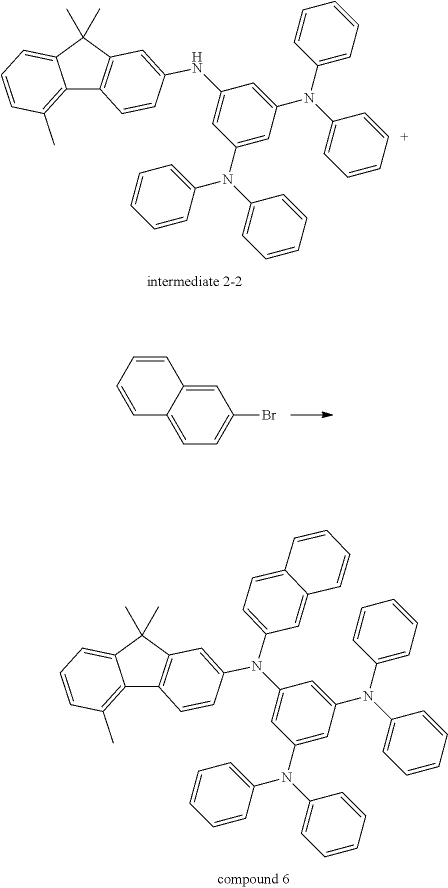 [SMILES: Cc1cccc2c1-c1ccc(Nc3cc(N(C4=CC=C[C+]=C4)c4ccccc4)cc(N(c4ccccc4)c4ccccc4)c3)cc1C2(C)C ; Brc1ccc2ccccc2c1 ; Cc1cccc2c1-c1ccc(N(c3cc(N(c4ccccc4)c4ccccc4)cc(N(c4ccccc4)c4ccccc4)c3)c3ccc4ccccc4c3)cc1C2(C)C]

Compound 6 was synthesized in substantially the same manner as in Synthesis of Compound 2, except that 2-bromonaphthalene was utilized instead of 4-bromo-1,1′-biphenyl. The resulting compound was identified by MS/FAB and ¹H NMR.

### Synthesis Example 3: Synthesis of Compound 17

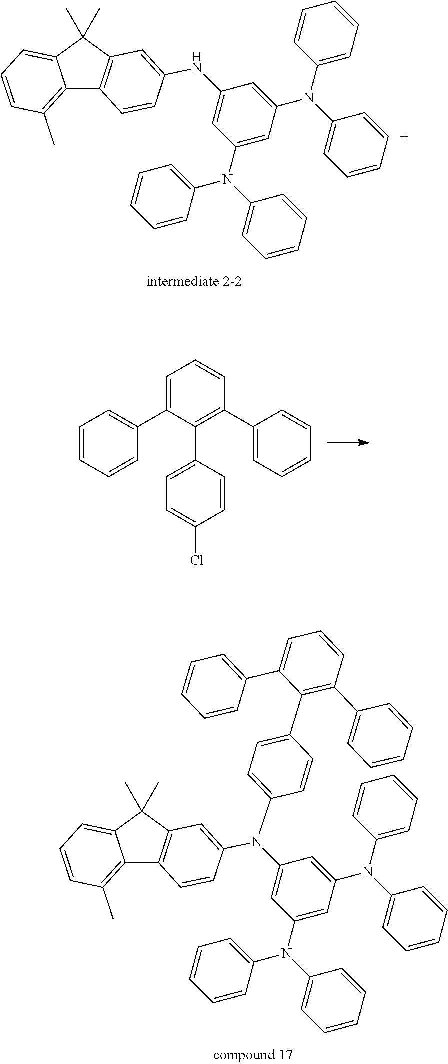 [SMILES: Cc1cccc2c1-c1ccc(Nc3cc(N(C4=CC=C[C+]=C4)c4ccccc4)cc(N(c4ccccc4)c4ccccc4)c3)cc1C2(C)C ; Clc1ccc(-c2c(-c3ccccc3)cccc2-c2ccccc2)cc1 ; Cc1cccc2c1-c1ccc(N(c3ccc(-c4c(-c5ccccc5)cccc4-c4ccccc4)cc3)c3cc(N(c4ccccc4)c4ccccc4)cc(N(c4ccccc4)c4ccccc4)c3)cc1C2(C)C]

Compound 17 was synthesized in substantially the same manner as in Synthesis of Compound 2, except that 4″-chloro-3′-phenyl-1,1′:2′,1″-terphenyl was utilized instead of 4-bromo-1,1′-biphenyl. The resulting compound was identified by MS/FAB and ¹H NMR.

### Synthesis Example 4: Synthesis of Compound 26

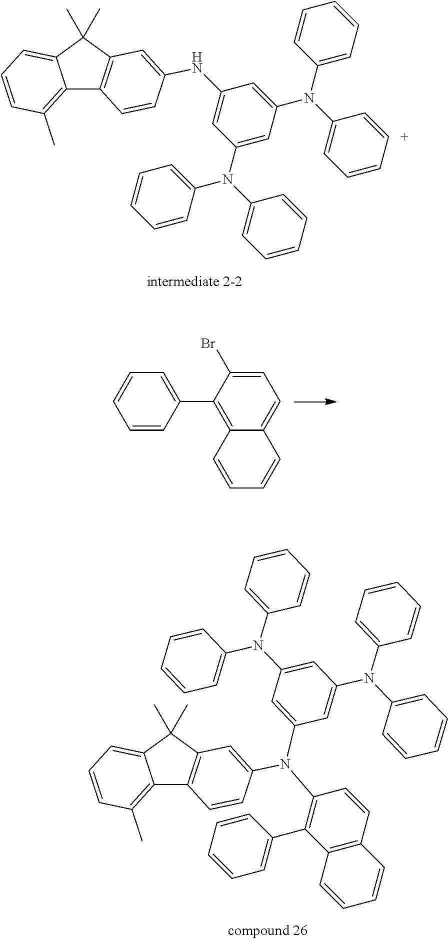 [SMILES: Cc1cccc2c1-c1ccc(Nc3cc(N(C4=CC=C[C+]=C4)c4ccccc4)cc(N(c4ccccc4)c4ccccc4)c3)cc1C2(C)C ; Brc1ccc2ccccc2c1-c1ccccc1 ; Cc1cccc2c1-c1ccc(N(c3cc(N(c4ccccc4)c4ccccc4)cc(N(c4ccccc4)c4ccccc4)c3)c3ccc4ccccc4c3-c3ccccc3)cc1C2(C)C]

Compound 26 was synthesized in substantially the same manner as in Synthesis of Compound 2, except that 2-bromo-1-phenylnaphthalene was utilized instead of 4-bromo-1,1′-biphenyl. The resulting compound was identified by MS/FAB and ¹H NMR.

### Synthesis Example 5: Synthesis of Compound 44

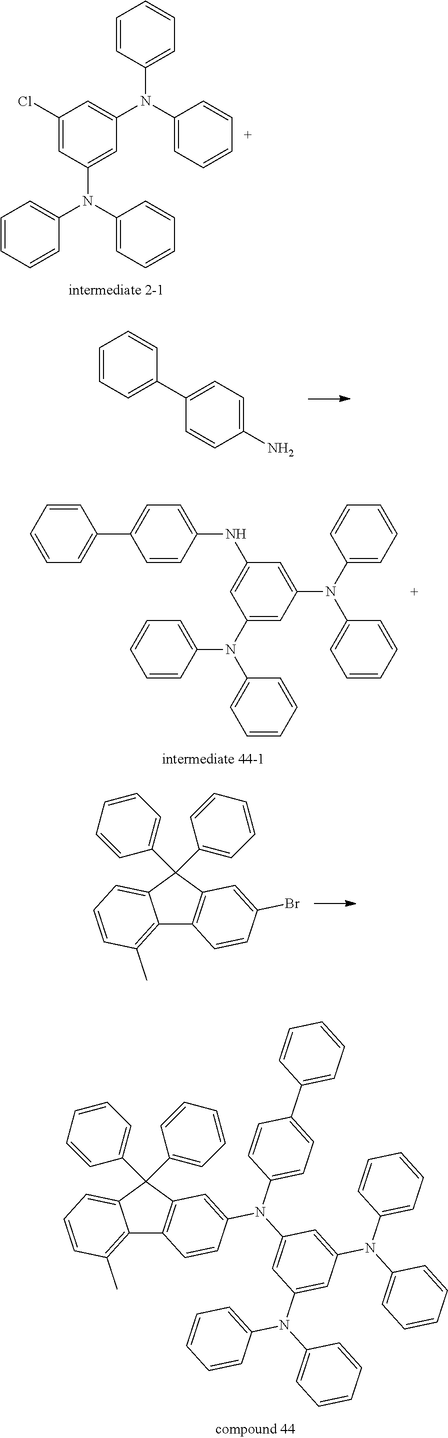 [SMILES: Clc1cc(N(c2ccccc2)c2ccccc2)cc(N(c2ccccc2)c2ccccc2)c1 ; Nc1ccc(-c2ccccc2)cc1 ; c1ccc(-c2ccc(Nc3cc(N(c4ccccc4)c4ccccc4)cc(N(c4ccccc4)c4ccccc4)c3)cc2)cc1 ; Cc1cccc2c1-c1ccc(Br)cc1C2(c1ccccc1)c1ccccc1 ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(c1ccccc1)c1ccccc1]

### Synthesis of Intermediate 44-1

4.47 g (10 mmol) of Intermediate 2-1, 2.03 g (12 mmol) of [1,1′-biphenyl]-4-amine, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of o-xylene and then stirred at 140° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 3.77 g (yield: 65%) of Intermediate 44-1. The resulting compound was identified by LC-MS:C₄₂H₃₃N₃:M⁺579.7

### Synthesis of Compound 44

5.80 g (10 mmol) of Intermediate 44-1, 4.11 g (10 mmol) of 2-bromo-5-methyl-9,9-diphenyl-9H-fluorene, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of toluene and then stirred at 80° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 6.37 g (yield: 70%) of Compound 44. The resulting compound was identified by MS/FAB and ¹H NMR.

### Synthesis Example 6: Synthesis of Compound 46

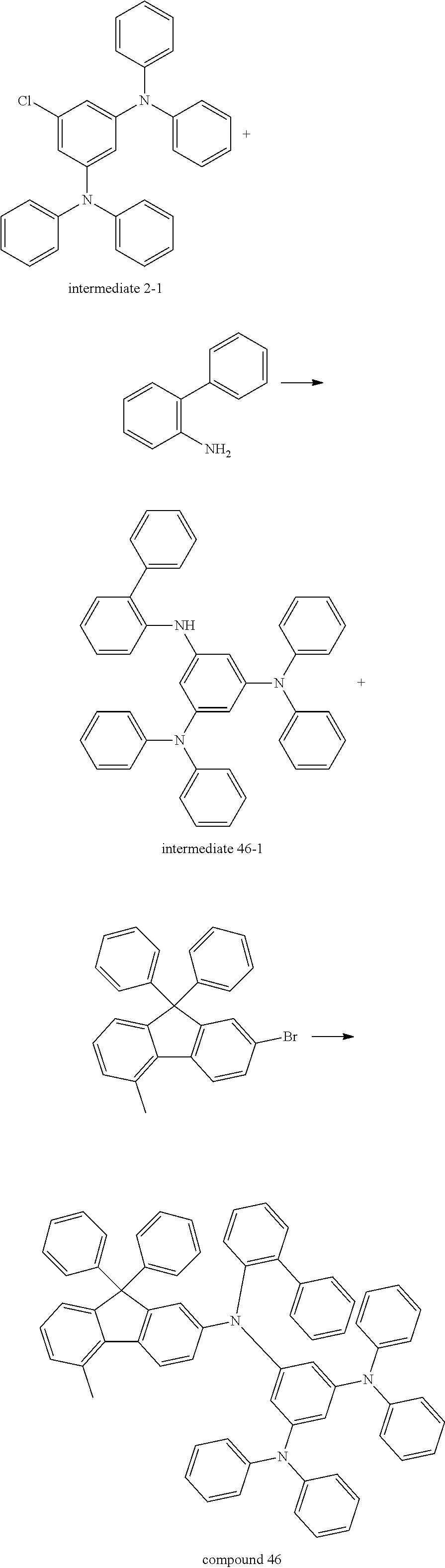 [SMILES: Clc1cc(N(c2ccccc2)c2ccccc2)cc(N(c2ccccc2)c2ccccc2)c1 ; Nc1ccccc1-c1ccccc1 ; c1ccc(-c2ccccc2Nc2cc(N(c3ccccc3)c3ccccc3)cc(N(c3ccccc3)c3ccccc3)c2)cc1 ; Cc1cccc2c1-c1ccc(Br)cc1C2(c1ccccc1)c1ccccc1 ; Cc1cccc2c1-c1ccc(N(c3cc(N(c4ccccc4)c4ccccc4)cc(N(c4ccccc4)c4ccccc4)c3)c3ccccc3-c3ccccc3)cc1C2(c1ccccc1)c1ccccc1]

Compound 46 was synthesized in substantially the same manner as in Synthesis of Compound 44, except that [1,1′-biphenyl]-2-amine was utilized instead of [1,1′-biphenyl]-4-amine. The resulting compound was identified by MS/FAB and ¹H NMR.

### Synthesis Example 7: Synthesis of Compound 56

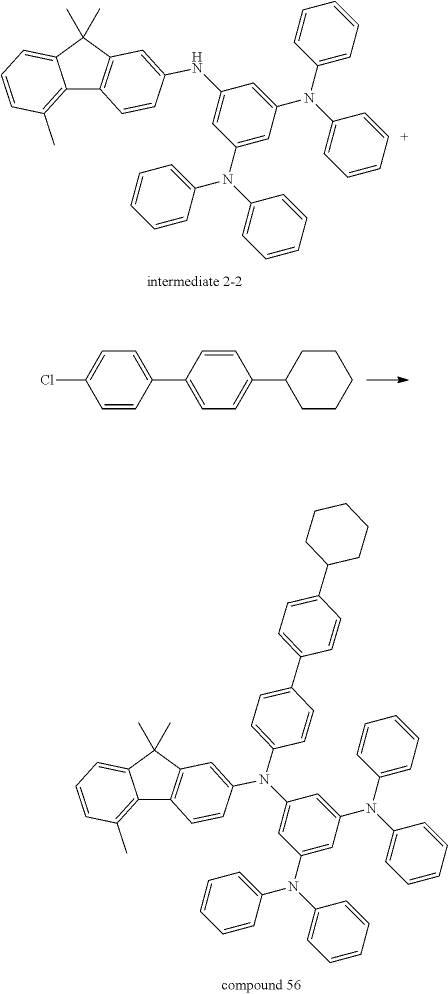 [SMILES: Cc1cccc2c1-c1ccc(Nc3cc(N(C4=CC=C[C+]=C4)c4ccccc4)cc(N(c4ccccc4)c4ccccc4)c3)cc1C2(C)C ; Clc1ccc(-c2ccc(C3CCCCC3)cc2)cc1 ; Cc1cccc2c1-c1ccc(N(c3ccc(-c4ccc(C5CCCCC5)cc4)cc3)c3cc(N(c4ccccc4)c4ccccc4)cc(N(c4ccccc4)c4ccccc4)c3)cc1C2(C)C]

Compound 56 was synthesized in substantially the same manner as in Synthesis of Compound 2, except that 4-chloro-4′-cyclohexyl-1,1′-biphenyl was utilized instead of 4-bromo-1,1′-biphenyl. The resulting compound was identified by MS/FAB and ¹H NMR.

### Synthesis Example 8: Synthesis of Compound 82

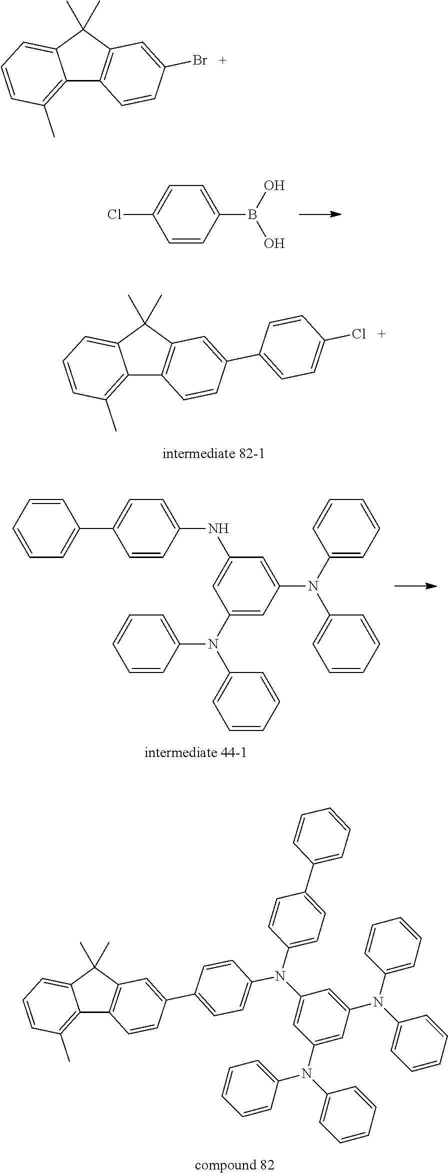 [SMILES: Cc1cccc2c1-c1ccc(Br)cc1C2(C)C ; OB(O)c1ccc(Cl)cc1 ; Cc1cccc2c1-c1ccc(-c3ccc(Cl)cc3)cc1C2(C)C ; c1ccc(-c2ccc(Nc3cc(N(c4ccccc4)c4ccccc4)cc(N(c4ccccc4)c4ccccc4)c3)cc2)cc1 ; Cc1cccc2c1-c1ccc(-c3ccc(N(c4ccc(-c5ccccc5)cc4)c4cc(N(c5ccccc5)c5ccccc5)cc(N(c5ccccc5)c5ccccc5)c4)cc3)cc1C2(C)C]

### Synthesis of Intermediate 82-1

2.87 g (10.0 mmol) of 2-bromo-5,9,9-trimethyl-9H-fluorene, 1.56 g (10.0 mmol) of (4-chlorophenyl)boronic acid, 0.58 g (0.5 mmol) of Pd(PPh₃)₄, and 4.14 g (30.0 mmol) of K₂CO₃ were dissolved in 60 mL of a mixed solution of tetrahydrofuran (THF)/H₂O (2/1) and then stirred at 80° C. for 16 hours. The reaction solution was cooled at room temperature, followed by three times of an extraction process utilizing 60 mL of water and 60 mL of diethyl ether.

The collected organic layer was dried over MgSO₄, the residue obtained by evaporating a solvent was separated and purified by silica gel chromatography, thereby obtaining 1.60 g (yield: 50%) of Intermediate 82-1. The resulting compound was identified by LC-MS. C₂₂H₁₉Cl:M⁺318.8

### Synthesis of Compound 82

3.19 g (10 mmol) of Intermediate 82-1, 5.80 g (10 mmol) of Intermediate 44-1, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of o-xylene and then stirred at 130° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 6.03 g (yield: 70%) of Compound 82. The resulting compound was identified by MS/FAB and ¹H NMR.

### Synthesis Example 9: Synthesis of Compound 89

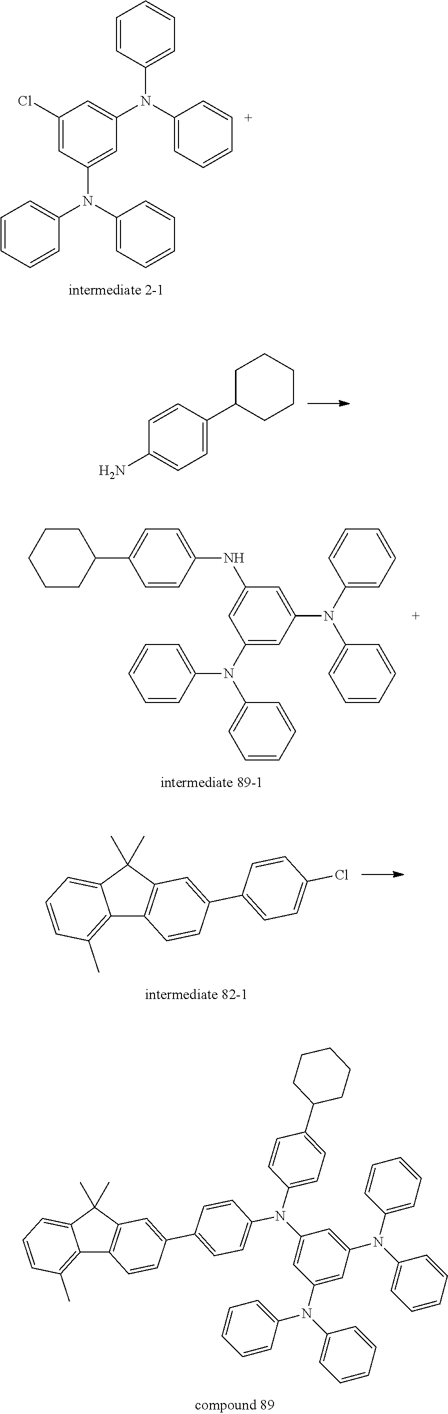 [SMILES: Clc1cc(N(c2ccccc2)c2ccccc2)cc(N(c2ccccc2)c2ccccc2)c1 ; Nc1ccc(C2CCCCC2)cc1 ; c1ccc(N(c2ccccc2)c2cc(Nc3ccc(C4CCCCC4)cc3)cc(N(c3ccccc3)c3ccccc3)c2)cc1 ; Cc1cccc2c1-c1ccc(-c3ccc(Cl)cc3)cc1C2(C)C ; Cc1cccc2c1-c1ccc(-c3ccc(N(c4ccc(C5CCCCC5)cc4)c4cc(N(c5ccccc5)c5ccccc5)cc(N(c5ccccc5)c5ccccc5)c4)cc3)cc1C2(C)C]

### Synthesis of Intermediate 89-1

4.47 g (10 mmol) of Intermediate 2-1, 2.10 g (12 mmol) of 4-cyclohexylaniline, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of o-xylene and then stirred at 140° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 3.81 g (yield: 65%) of Intermediate 89-1. The resulting compound was identified by LC-MS:C₄₂H₃₉N₃:M⁺585.7

### Synthesis of Compound 89

5.86 g (10 mmol) of Intermediate 89-1, 3.19 g (10 mmol) of Intermediate 82-1, 0.46 g (0.5 mmol) of tris(dibenzylideneacetone)dipalladium(0) (Pd₂(dba)₃), 0.24 g (1 mmol) of P(t-Bu)₃, and 2.88 g (30 mmol) of sodium tert-butoxide were dissolved in 60 mL of o-xylene and then stirred at 140° C. for 3 hours. The reaction solution was cooled at room temperature, and then, 40 mL of water added thereto, followed by three times of an extraction process utilizing 50 mL of ethyl ether.

The collected organic layer was dried over MgSO₄, the residue obtained by evaporating a solvent was separated and purified by silica gel chromatography, thereby obtaining 6.08 g (yield: 70%) of Compound 89. The resulting compound was identified by MS/FAB and ¹H NMR.

### Synthesis Example 10: Synthesis of Compound 106

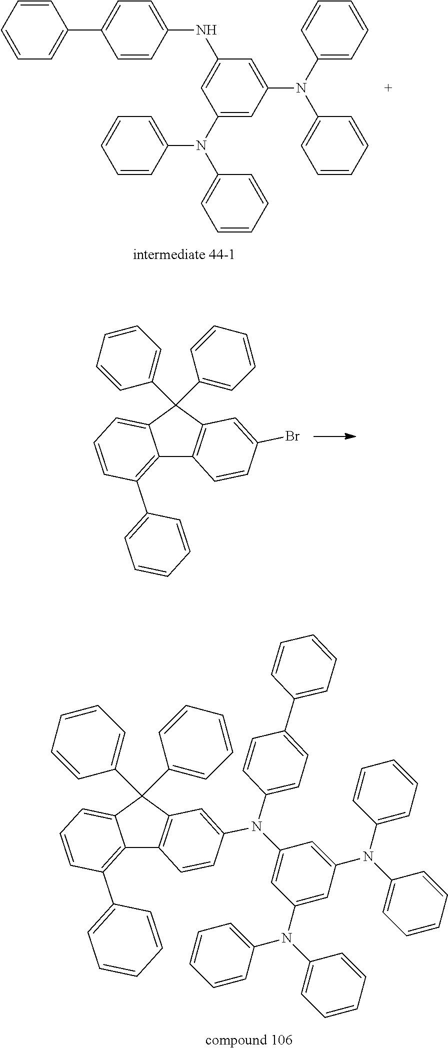 [SMILES: c1ccc(-c2ccc(Nc3cc(N(c4ccccc4)c4ccccc4)cc(N(c4ccccc4)c4ccccc4)c3)cc2)cc1 ; Brc1ccc2c(c1)C(c1ccccc1)(c1ccccc1)c1cccc(-c3ccccc3)c1-2 ; c1ccc(-c2ccc(N(c3cc(N(c4ccccc4)c4ccccc4)cc(N(c4ccccc4)c4ccccc4)c3)c3ccc4c(c3)C(c3ccccc3)(c3ccccc3)c3cccc(-c5ccccc5)c3-4)cc2)cc1]

Compound 106 was synthesized in substantially the same manner as in Synthesis of Compound 44, except that 2-bromo-5,9,9-triphenyl-9H-fluorene was utilized instead of 2-bromo-5-methyl-9,9-diphenyl-9H-fluorene. The resulting compound was identified by MS/FAB and ¹H NMR.

¹H NMR and MS/FAB of each of the compounds synthesized according to Synthesis Examples are shown in Table 1. Synthesis methods of other compounds in addition to the compound synthesized in Synthesis Examples may be easily recognized by those skilled in the art by referring to the synthesis paths and source materials.

TABLE 1

| Compound | | MS/FAB | |
|---|---|---|---|
| No. | ¹H NMR (CDCl₃, 400 MHz) | found | calc. |
| Compound | 7.86(d, 1H), 7.75(d, 2H), 7.55-7.33(m, 9H), | 785.26 | 785.38 |
| 2 | 7.26-7.00(m, 23H), 6.49(s, 3H), 2.57(s, 3H), | | |
| | 1.69(s, 6H) | | |
| Compound | 7.86(d, 1H), 7.78-7.71 (m, 2H), 7.54(d, 1H), | 759.24 | 759.36 |
| 6 | 7.45-7.33(m, 5H), 7.26-7.00(m, 24H), 6.49(s, | | |
| | 3H), 2.57(s, 3H), 1.69(s, 6H) | | |
| Compound | 8.06(d, 2H), 7.86-7.79(m, 6H), 7.55(d, 2H), | 937.22 | 937.44 |
| 17 | 7.46-7.33(m, 10H), 7.26-7.00(m, 23H), 6.49(s, | | |
| | 3H), 2.57(s, 3H), 1.69(s, 6H) | | |
| Compound | 8.80(d, 1H), 7.97(d, 1H), 7.89-7.86(d, 2H), 7.76(d, | 835.19 | 835.39 |
| 26 | 1H), 7.51-7.45(m, 6H), 7.33(s, 1H), 7.26-7.00(m, | | |
| | 25H), 6.49(s, 3H), 2.57(s, 3H), 1.69(s, 6H) | | |
| Compound | 7.86(d, 1H), 7.75(d, 2H), 7.55-7.00(m, 42H), 6.49(s, | 909.25 | 909.41 |
| 44 | 3H), 2.57(s, 3H) | | |
| Compound | 8.10 (d, 1H), 7.86(d, 1H), 7.45-7.00(m, 43H), 6.49(s, | 909.31 | 909.41 |
| 46 | 3H), 2.57(s, 3H) | | |
| Compound | 7.86(d, 1H), 7.55-7.33(m, 10H), 7.26-7.00(m, 23H), | 867.23 | 867.46 |
| 56 | 6.49(s, 3H), 2.72(m, 1H), 2.57(s, 3H), 1.85-1.43(m, | | |
| | 16H) | | |
| Compound | 8.09(d, 1H), 7.89(s, 1H), 7.78-7.75(m, 3H), | 861.23 | 861.41 |
| 82 | 7.55-7.37(m, 12H), 7.26-7.23(m, 10H), 7.08-7.00(m, | | |
| | 12H), 6.49(s, 3H), 2.57(s, 3H), 1.69(s, 6H) | | |
| Compound | 8.09(d, 1H), 7.89(s, 1H), 7.78(d, 1H), 7.55(d, 2H), | 867.23 | 867.46 |
| 89 | 7.45(d ,1H), 7.37(d, 2H), 7.26-7.18(m, 12H), | | |
| | 7.08-7.00(m, 14H), 6.49(s, 3H), 2.72(m, 1H), | | |
| | 2.57(s, 3H), 1.85-1.43(m, 16H) | | |
| Compound | 7.86(d, 1H), 7.79-7.75(m, 5H), 7.65(d, 1H), | 971.21 | 971.42 |
| 106 | 7.55-7.00(m, 43H), 6.49(s, 3H) | | |

### Manufacture of Light-Emitting Device

### Comparative Example 1

As an anode, a glass substrate (product of Corning Inc.) with a 15 Ω/cm² (1,200 Å) ITO electrode formed thereon was cut to a size of 50 mm×50 mm×0.7 mm, sonicated with isopropyl alcohol and then pure water each for 5 minutes, cleaned by irradiation of ultraviolet rays and exposure of ozone thereto for 30 minutes, and then mounted on a vacuum deposition apparatus.

NPD was deposited on the anode to form a hole injection layer having a thickness of 300 Å, HT3 was deposited on the hole injection layer to form a hole transport layer having a thickness of 200 Å, and then, CzSi was deposited on the hole transport layer to form an emission auxiliary layer having a thickness of 100 Å.

H125, H126, PD40, and DF14 were co-deposited on the emission auxiliary layer at a weight ratio of 42:42:15:1 to form an emission layer having a thickness of 200 Å, TSPO1 was deposited on the emission layer to form a hole blocking layer having a thickness of 200 Å, TPBI was deposited on the hole blocking layer to form an electron transport layer having a thickness of 300 Å, LiF was deposited on the electron transport layer to form an electron injection layer having a thickness of 10 Å, and Al was deposited on the electron injection layer to form a cathode having a thickness of 3,000 Å, thereby completing manufacture of a light-emitting device.

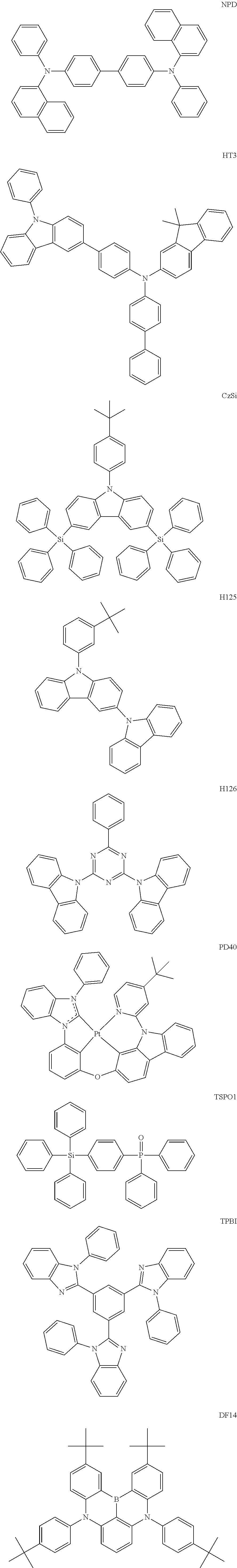

### Comparative Example 2

A light-emitting device was manufactured in substantially the same manner as in Comparative Example 1, except that instead of HT3, Compound 1000 was utilized in the hole transport layer.

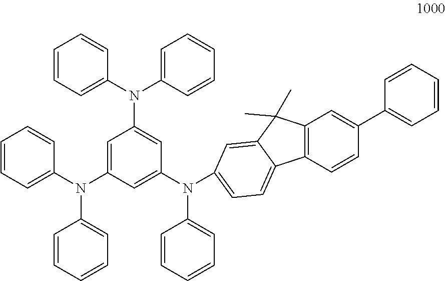 [SMILES: CC1(C)c2cc(-c3ccccc3)ccc2-c2ccc(N(c3ccccc3)c3cc(N(c4ccccc4)c4ccccc4)cc(N(c4ccccc4)c4ccccc4)c3)cc21]

### Examples 1 to 10

Light-emitting devices were each manufactured in substantially the same manner as in Comparative Example 1, except that instead of HT3, a compound in Table 2 was utilized in the respective hole transport layer.

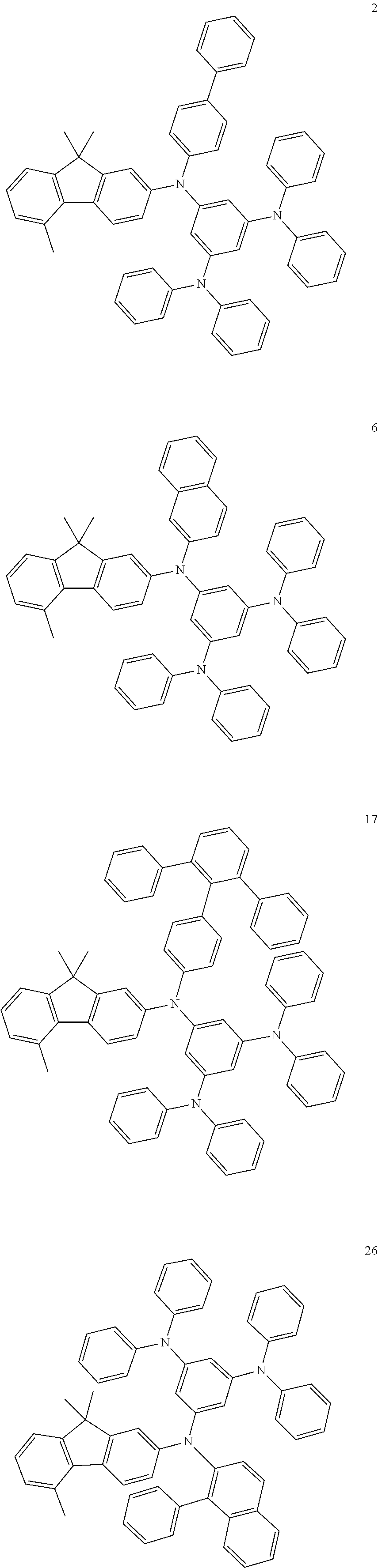 [SMILES: Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3cc(N(c4ccccc4)c4ccccc4)cc(N(c4ccccc4)c4ccccc4)c3)c3ccc4ccccc4c3)cc1C2(C)C ; Cc1cccc2c1-c1ccc(N(c3ccc(-c4c(-c5ccccc5)cccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3cc(N(c4ccccc4)c4ccccc4)cc(N(c4ccccc4)c4ccccc4)c3)c3ccc4ccccc4c3-c3ccccc3)cc1C2(C)C]

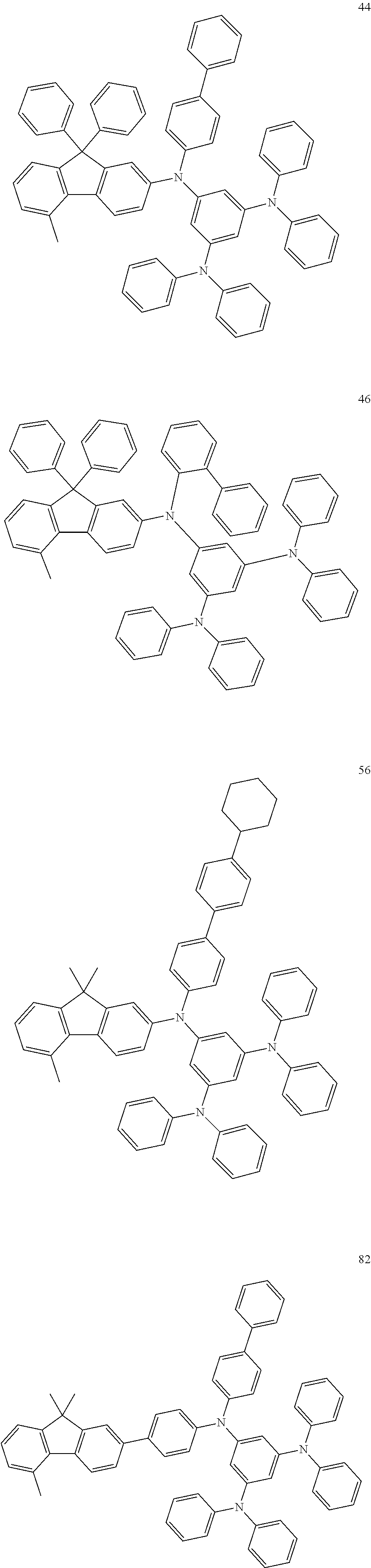 [SMILES: Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(c1ccccc1)c1ccccc1 ; Cc1cccc2c1-c1ccc(N(c3cc(N(c4ccccc4)c4ccccc4)cc(N(c4ccccc4)c4ccccc4)c3)c3ccccc3-c3ccccc3)cc1C2(c1ccccc1)c1ccccc1 ; Cc1cccc2c1-c1ccc(N(c3ccc(-c4ccc(C5CCCCC5)cc4)cc3)c3cc(N(c4ccccc4)c4ccccc4)cc(N(c4ccccc4)c4ccccc4)c3)cc1C2(C)C ; Cc1cccc2c1-c1ccc(-c3ccc(N(c4ccc(-c5ccccc5)cc4)c4cc(N(c5ccccc5)c5ccccc5)cc(N(c5ccccc5)c5ccccc5)c4)cc3)cc1C2(C)C]

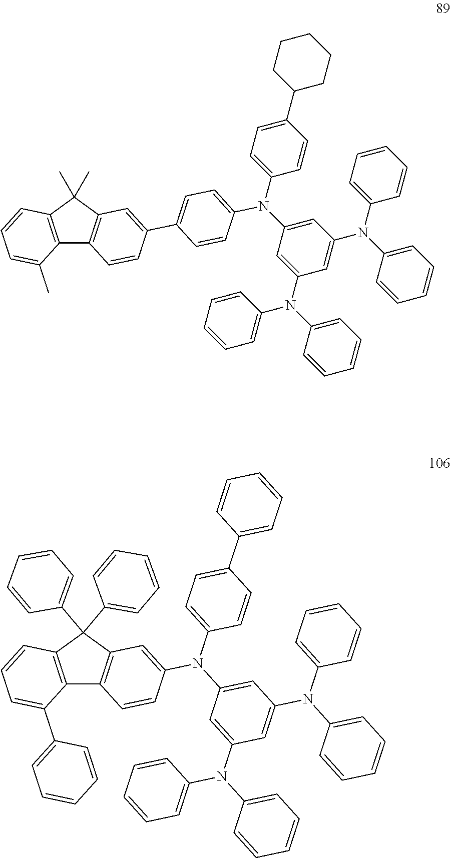 [SMILES: Cc1cccc2c1-c1ccc(-c3ccc(N(c4ccc(C5CCCCC5)cc4)c4cc(N(c5ccccc5)c5ccccc5)cc(N(c5ccccc5)c5ccccc5)c4)cc3)cc1C2(C)C ; c1ccc(-c2ccc(N(c3cc(N(c4ccccc4)c4ccccc4)cc(N(c4ccccc4)c4ccccc4)c3)c3ccc4c(c3)C(c3ccccc3)(c3ccccc3)c3cccc(-c5ccccc5)c3-4)cc2)cc1]

To evaluate the characteristics of each of the light-emitting devices of Examples and Comparative Examples above, driving voltage at a current density of 10 mA/cm², lifespan, and efficiency were measured. The driving voltage, lifespan, and efficiency of each of the organic light-emitting devices were measured utilizing a source meter (manufactured by Keithley Instrument Inc., 2400 series). The lifespan, T₉₅, of an organic light-emitting device was obtained by measuring a time taken, in hours, to reach 95% of its initial luminance. Lifespan was measured using ENC's lifespan measurement equipment (LTS96CH).

Results of evaluating the characteristics of each of the light-emitting devices are shown in Table 2.

TABLE 2

| | | emission | | | | | | |
|---|---|---|---|---|---|---|---|---|
| | | layer | Phospho- | Delayed | Driving | | Emission | Lifespan |
| | | (HT:ET = | rescence | fluorescence | voltage | Efficiency | wavelength | (T₉₅) |
| | HTL | 5:5) | (Sensitizer) | dopant | (V) | (cd/A) | (nm) | (hr) |
| Comparative | HT3 | H125/H126 | PD40 | DF14 | 5.6 | 18.8 | 463 | 95 |
| Example 1 | | | | | | | | |
| Comparative | 1000 | H125/H126 | PD40 | DF14 | 5.3 | 18.9 | 463 | 98 |
| Example 2 | | | | | | | | |
| Example 1 | 2 | H125/H126 | PD40 | DF14 | 4.9 | 20.1 | 463 | 121 |
| Example 2 | 6 | H125/H126 | PD40 | DF14 | 4.8 | 20.5 | 463 | 125 |
| Example 3 | 17 | H125/H126 | PD40 | DF14 | 4.8 | 20.8 | 463 | 132 |
| Example 4 | 26 | H125/H126 | PD40 | DF14 | 4.7 | 20.6 | 463 | 125 |
| Example 5 | 44 | H125/H126 | PD40 | DF14 | 4.8 | 20.9 | 463 | 129 |
| Example 6 | 46 | H125/H126 | PD40 | DF14 | 4.7 | 20.6 | 463 | 124 |
| Example 7 | 56 | H125/H126 | PD40 | DF14 | 4.9 | 21.0 | 463 | 135 |
| Example 8 | 82 | H125/H126 | PD40 | DF14 | 4.7 | 20.2 | 463 | 133 |
| Example 9 | 89 | H125/H126 | PD40 | DF14 | 4.8 | 21.0 | 463 | 130 |
| Example 10 | 106 | H125/H126 | PD40 | DF14 | 4.8 | 20.5 | 463 | 129 |

HT: hole transporting host;
ET: electron transporting host

Referring to Table 2, it may be confirmed that each of the light emitting devices of Examples has higher efficiency, lower driving voltage, and longer lifespan compared to the light emitting devices of Comparative Examples.

In particular, in the case of Comparative Example 2 in which Compound 1000 in which a 2-position of a fluorene group was substituted was utilized, it could be confirmed that a result was substantially worse or was not better than results of Examples of the disclosure.

A light-emitting device including the compound according to one or more embodiments has excellent or suitable efficiency and excellent or suitable lifespan.

In the present disclosure, it will be understood that the term “comprise(s),” “include(s),” or “have/has” specifies the presence of stated features, integers, steps, operations, elements, and/or components, but do not preclude the presence or addition of one or more other features, integers, steps, operations, elements, components, and/or groups thereof.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

The light-emitting device, the electronic apparatus, the display device/apparatus, the electronic equipment, the electronic device, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the appended claims and equivalents thereof.

What is claimed is:

1. A compound represented by Formula 1: 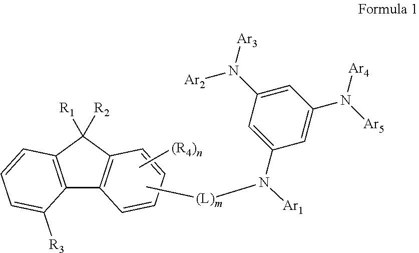
wherein, in Formula 1,
R₁, R₂, R₄, and Ar₁ to Ar₅ are each independently hydrogen, deuterium, a C₁-C₆₀ alkyl group that is unsubstituted or substituted with at least one R₁₀ₐ, a C₃-C₁₀ cycloalkyl group that is unsubstituted or substituted with at least one R₁₀ₐ, a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ,
R₃ is a C₁-C₆₀ alkyl group that is unsubstituted or substituted with at least one R₁₀ₐ, a C₃-C₁₀ cycloalkyl group that is unsubstituted or substituted with at least one R₁₀ₐ, or a C₆-C₆₀ aryl group that is unsubstituted or substituted with at least one R₁₀ₐ,
optionally, R₁ and R₂ are linked together to form a ring,
L is a single bond, a C₆-C₆₀ arylene group that is unsubstituted or substituted with at least one R₁₀ₐ, a C₁-C₆₀ heteroarylene group that is unsubstituted or substituted with at least one R₁₀ₐ, a divalent non-aromatic condensed polycyclic group that is unsubstituted or substituted with at least one R₁₀ₐ, or a divalent non-aromatic condensed heteropolycyclic group that is unsubstituted or substituted with at least one R₁₀ₐ,
m is an integer from 0 to 3, wherein, when m is 2 or more, each L is identical to or different from each other,
n is an integer from 0 to 3, wherein, when n is 2 or more, each R₄ is identical to or different from each other, and
wherein R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ being each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The compound of claim 1, wherein Ar₁ to Ar₅ are each independently a C₆-C₆₀ aryl group that is unsubstituted or substituted with at least one R₁₀ₐ.

3. The compound of claim 1, wherein Ar₁ to Ar₅ are each independently a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group, each unsubstituted or substituted with at least one R₁₀ₐ.

4. The compound of claim 1, wherein each R₄ is independently hydrogen or deuterium.

5. The compound of claim 1, wherein R₁ to R₃ are each independently a methyl group, an ethyl group, an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group, each unsubstituted or substituted with at least one R₁₀ₐ.

6. The compound of claim 1, wherein the compound represented by Formula 1 is represented by Formula 2: 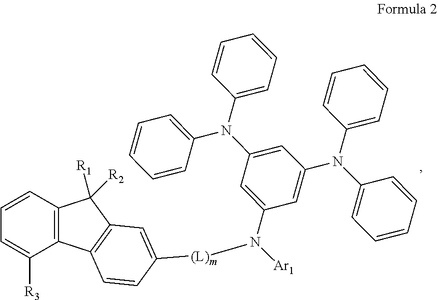
in Formula 2,
R₁ to R₃, Ar₁, L, and m each being the same as defined in Formula 1.

7. The compound of claim 6, wherein R₁ to R₃ are each independently a methyl group, a phenyl group, or a naphthyl group, each unsubstituted or substituted with at least one R₁₀ₐ.

8. The compound of claim 6, wherein Ar₁ is a C₆-C₆₀ aryl group that is unsubstituted or substituted with at least one R₁₀ₐ, and
R₁₀ₐ is a C₃-C₆₀ carbocyclic group that is unsubstituted or substituted with a C₃-C₆₀ carbocyclic group.

9. The compound of claim 1, comprising one selected from among compounds:

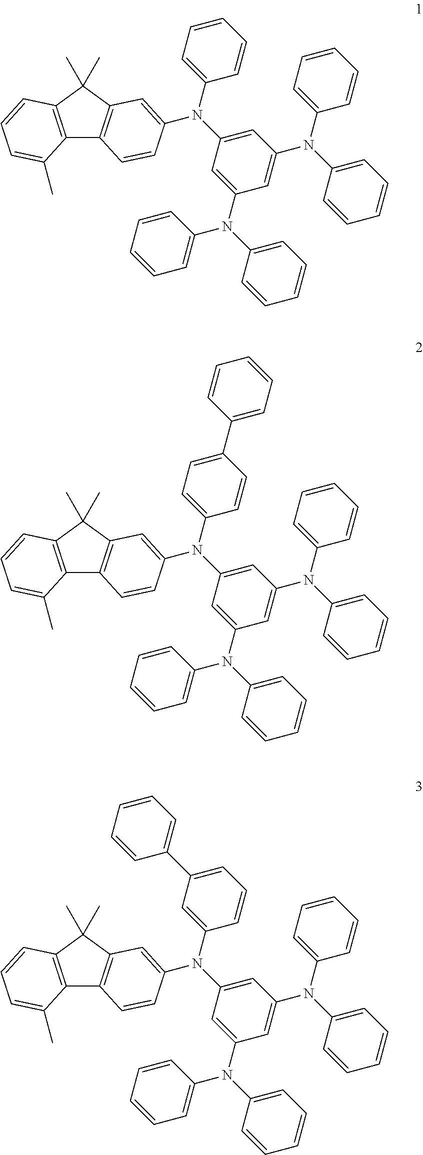 [SMILES: Cc1cccc2c1-c1ccc(N(c3ccccc3)c3cc(N(c4ccccc4)c4ccccc4)cc(N(c4ccccc4)c4ccccc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3cccc(-c4ccccc4)c3)c3cc(N(c4ccccc4)c4ccccc4)cc(N(c4ccccc4)c4ccccc4)c3)cc1C2(C)C]

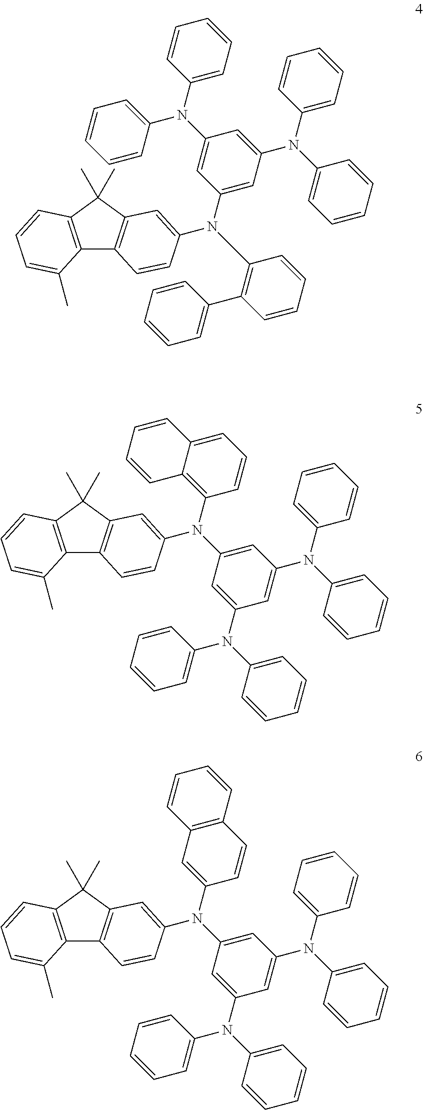 [SMILES: Cc1cccc2c1-c1ccc(N(c3cc(N(c4ccccc4)c4ccccc4)cc(N(c4ccccc4)c4ccccc4)c3)c3ccccc3-c3ccccc3)cc1C2(C)C ; Cc1cccc2c1-c1ccc(N(c3cc(N(c4ccccc4)c4ccccc4)cc(N(c4ccccc4)c4ccccc4)c3)c3cccc4ccccc34)cc1C2(C)C ; Cc1cccc2c1-c1ccc(N(c3cc(N(c4ccccc4)c4ccccc4)cc(N(c4ccccc4)c4ccccc4)c3)c3ccc4ccccc4c3)cc1C2(C)C]

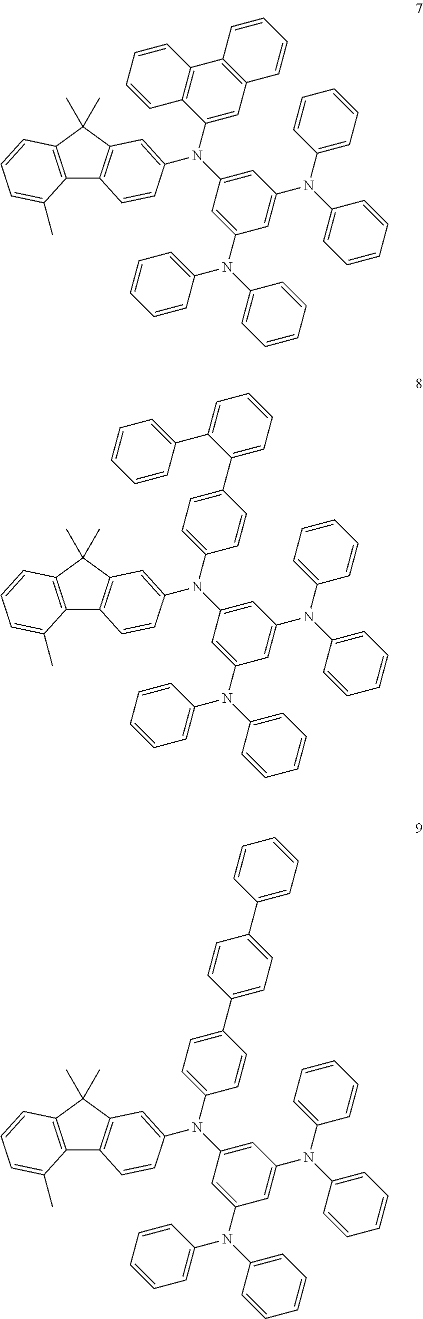 [SMILES: Cc1cccc2c1-c1ccc(N(c3cc(N(c4ccccc4)c4ccccc4)cc(N(c4ccccc4)c4ccccc4)c3)c3cc4ccccc4c4ccccc34)cc1C2(C)C ; Cc1cccc2c1-c1ccc(N(c3ccc(-c4ccccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4ccc(-c5ccccc5)cc4)cc3)c3cc(N(c4ccccc4)c4ccccc4)cc(N(c4ccccc4)c4ccccc4)c3)cc1C2(C)C]

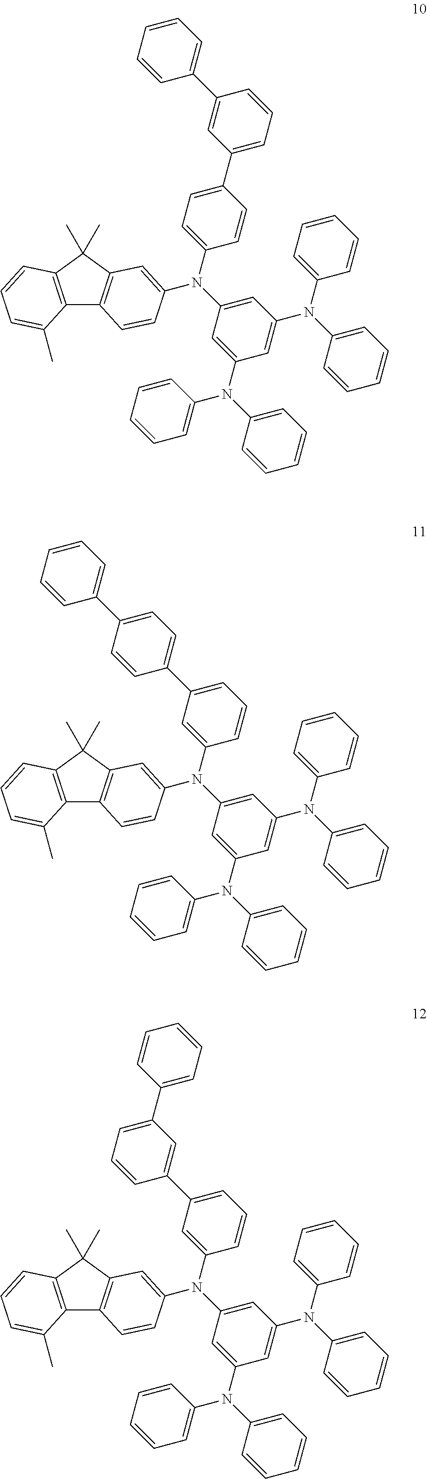 [SMILES: Cc1cccc2c1-c1ccc(N(c3ccc(-c4cccc(-c5ccccc5)c4)cc3)c3cc(N(c4ccccc4)c4ccccc4)cc(N(c4ccccc4)c4ccccc4)c3)cc1C2(C)C ; Cc1cccc2c1-c1ccc(N(c3cccc(-c4ccc(-c5ccccc5)cc4)c3)c3cc(N(c4ccccc4)c4ccccc4)cc(N(c4ccccc4)c4ccccc4)c3)cc1C2(C)C ; Cc1cccc2c1-c1ccc(N(c3cccc(-c4cccc(-c5ccccc5)c4)c3)c3cc(N(c4ccccc4)c4ccccc4)cc(N(c4ccccc4)c4ccccc4)c3)cc1C2(C)C]

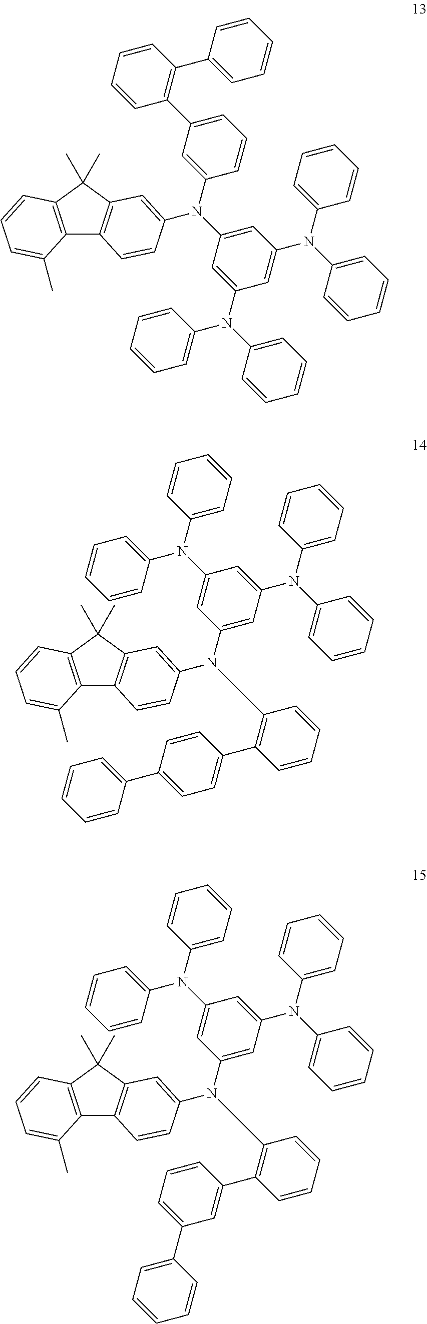 [SMILES: Cc1cccc2c1-c1ccc(N(c3cccc(-c4ccccc4-c4ccccc4)c3)c3cc(N(c4ccccc4)c4ccccc4)cc(N(c4ccccc4)c4ccccc4)c3)cc1C2(C)C ; Cc1cccc2c1-c1ccc(N(c3cc(N(c4ccccc4)c4ccccc4)cc(N(c4ccccc4)c4ccccc4)c3)c3ccccc3-c3ccc(-c4ccccc4)cc3)cc1C2(C)C ; Cc1cccc2c1-c1ccc(N(c3cc(N(c4ccccc4)c4ccccc4)cc(N(c4ccccc4)c4ccccc4)c3)c3ccccc3-c3cccc(-c4ccccc4)c3)cc1C2(C)C]

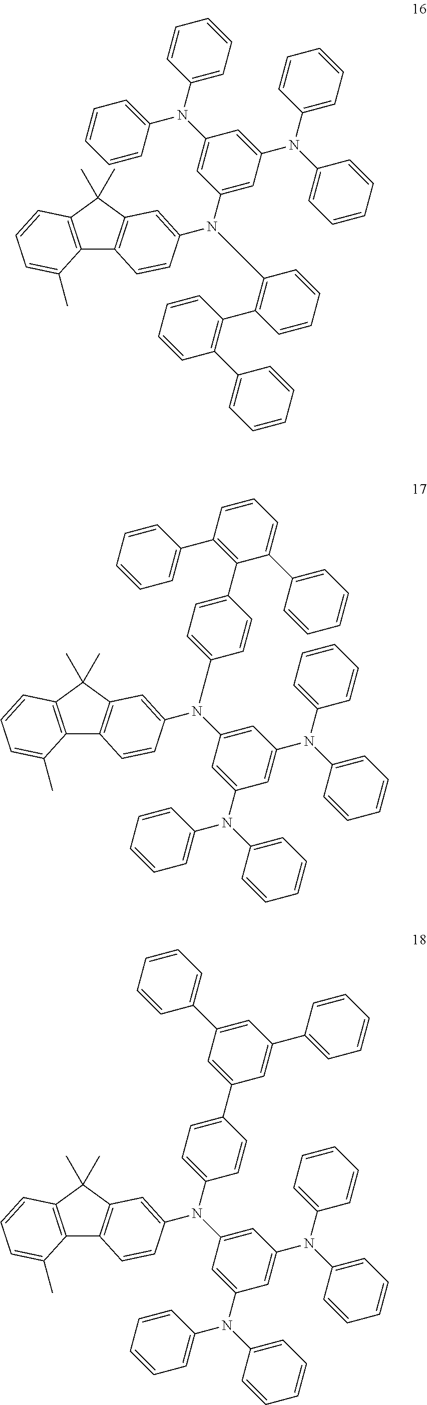 [SMILES: Cc1cccc2c1-c1ccc(N(c3cc(N(c4ccccc4)c4ccccc4)cc(N(c4ccccc4)c4ccccc4)c3)c3ccccc3-c3ccccc3-c3ccccc3)cc1C2(C)C ; Cc1cccc2c1-c1ccc(N(c3ccc(-c4c(-c5ccccc5)cccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4cc(-c5ccccc5)cc(-c5ccccc5)c4)cc3)c3cc(N(c4ccccc4)c4ccccc4)cc(N(c4ccccc4)c4ccccc4)c3)cc1C2(C)C]

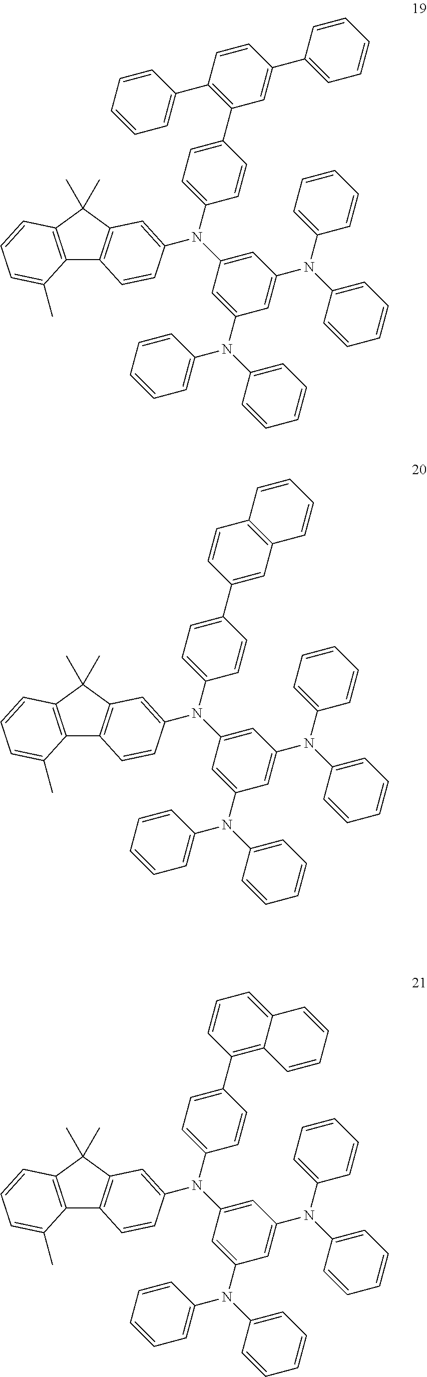 [SMILES: Cc1cccc2c1-c1ccc(N(c3ccc(-c4cc(-c5ccccc5)ccc4-c4ccccc4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4ccc5ccccc5c4)cc3)c3cc(N(c4ccccc4)c4ccccc4)cc(N(c4ccccc4)c4ccccc4)c3)cc1C2(C)C ; Cc1cccc2c1-c1ccc(N(c3ccc(-c4cccc5ccccc45)cc3)c3cc(N(c4ccccc4)c4ccccc4)cc(N(c4ccccc4)c4ccccc4)c3)cc1C2(C)C]

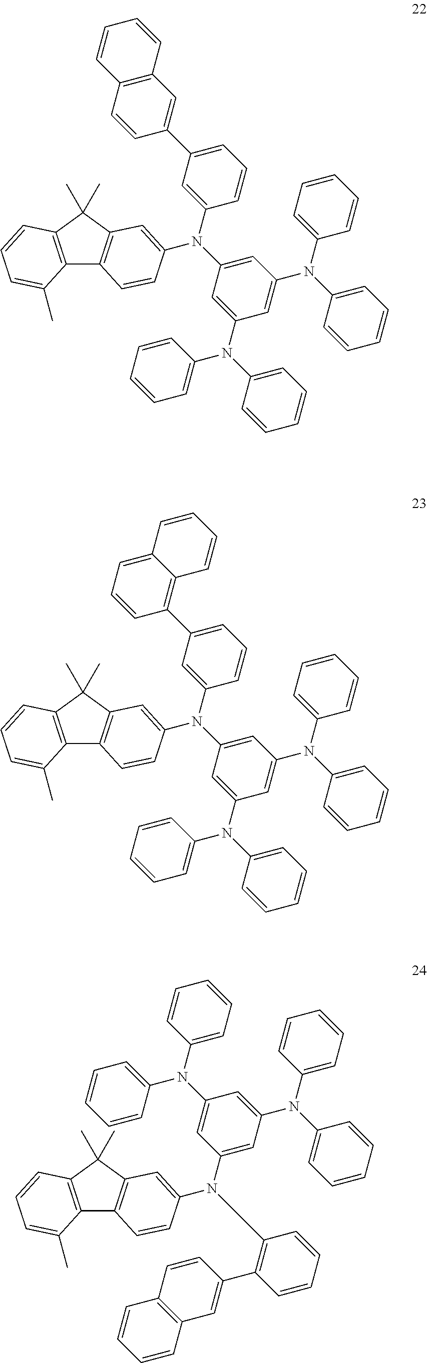 [SMILES: Cc1cccc2c1-c1ccc(N(c3cccc(-c4ccc5ccccc5c4)c3)c3cc(N(c4ccccc4)c4ccccc4)cc(N(c4ccccc4)c4ccccc4)c3)cc1C2(C)C ; Cc1cccc2c1-c1ccc(N(c3cccc(-c4cccc5ccccc45)c3)c3cc(N(c4ccccc4)c4ccccc4)cc(N(c4ccccc4)c4ccccc4)c3)cc1C2(C)C ; Cc1cccc2c1-c1ccc(N(c3cc(N(c4ccccc4)c4ccccc4)cc(N(c4ccccc4)c4ccccc4)c3)c3ccccc3-c3ccc4ccccc4c3)cc1C2(C)C]

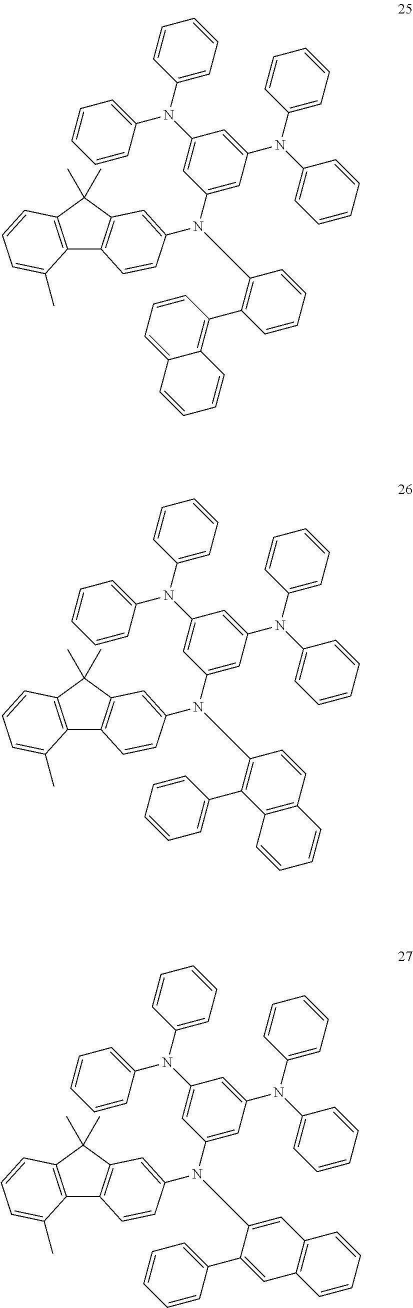 [SMILES: Cc1cccc2c1-c1ccc(N(c3cc(N(c4ccccc4)c4ccccc4)cc(N(c4ccccc4)c4ccccc4)c3)c3ccccc3-c3cccc4ccccc34)cc1C2(C)C ; Cc1cccc2c1-c1ccc(N(c3cc(N(c4ccccc4)c4ccccc4)cc(N(c4ccccc4)c4ccccc4)c3)c3ccc4ccccc4c3-c3ccccc3)cc1C2(C)C ; Cc1cccc2c1-c1ccc(N(c3cc(N(c4ccccc4)c4ccccc4)cc(N(c4ccccc4)c4ccccc4)c3)c3cc4ccccc4cc3-c3ccccc3)cc1C2(C)C]

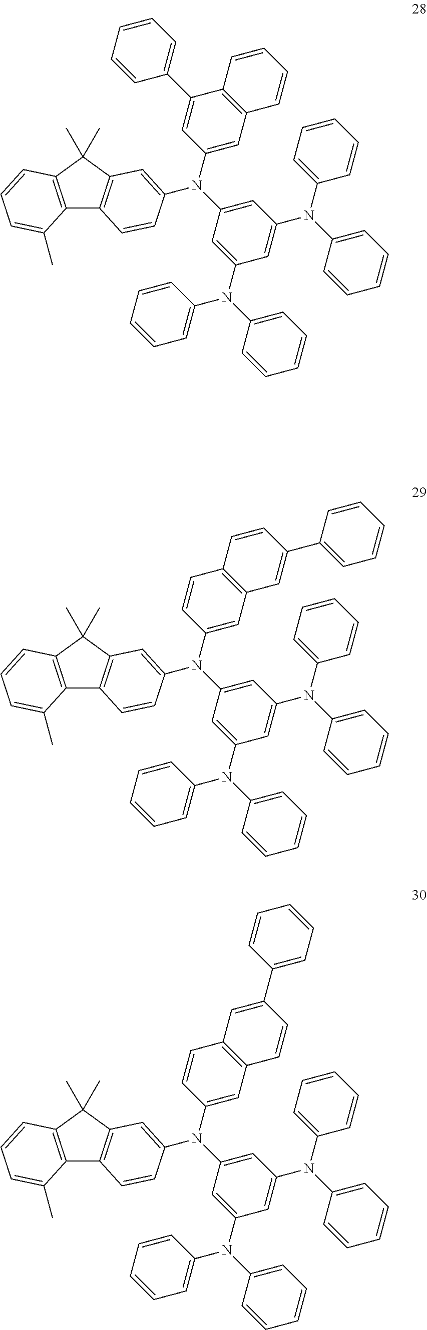 [SMILES: Cc1cccc2c1-c1ccc(N(c3cc(N(c4ccccc4)c4ccccc4)cc(N(c4ccccc4)c4ccccc4)c3)c3cc(-c4ccccc4)c4ccccc4c3)cc1C2(C)C ; Cc1cccc2c1-c1ccc(N(c3cc(N(c4ccccc4)c4ccccc4)cc(N(c4ccccc4)c4ccccc4)c3)c3ccc4ccc(-c5ccccc5)cc4c3)cc1C2(C)C ; Cc1cccc2c1-c1ccc(N(c3cc(N(c4ccccc4)c4ccccc4)cc(N(c4ccccc4)c4ccccc4)c3)c3ccc4cc(-c5ccccc5)ccc4c3)cc1C2(C)C]

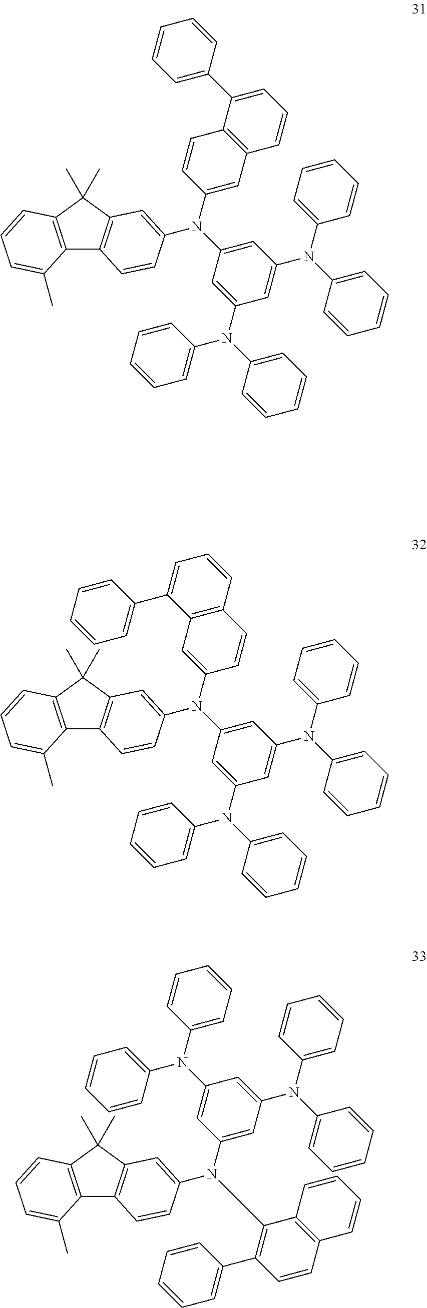 [SMILES: Cc1cccc2c1-c1ccc(N(c3cc(N(c4ccccc4)c4ccccc4)cc(N(c4ccccc4)c4ccccc4)c3)c3ccc4c(-c5ccccc5)cccc4c3)cc1C2(C)C ; Cc1cccc2c1-c1ccc(N(c3cc(N(c4ccccc4)c4ccccc4)cc(N(c4ccccc4)c4ccccc4)c3)c3ccc4cccc(-c5ccccc5)c4c3)cc1C2(C)C ; Cc1cccc2c1-c1ccc(N(c3cc(N(c4ccccc4)c4ccccc4)cc(N(c4ccccc4)c4ccccc4)c3)c3c(-c4ccccc4)ccc4ccccc34)cc1C2(C)C]

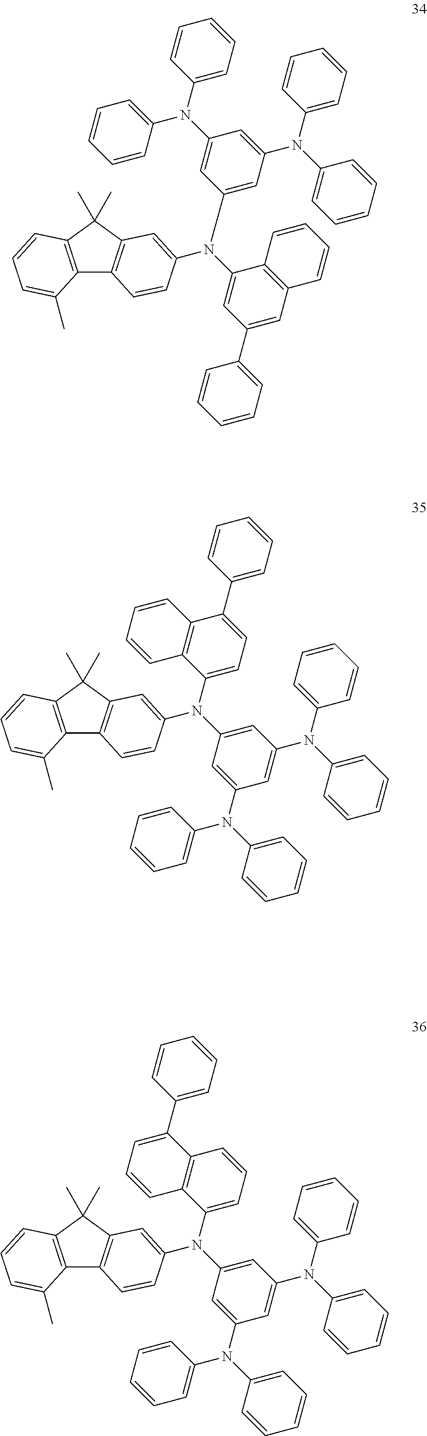 [SMILES: Cc1cccc2c1-c1ccc(N(c3cc(N(c4ccccc4)c4ccccc4)cc(N(c4ccccc4)c4ccccc4)c3)c3cc(-c4ccccc4)cc4ccccc34)cc1C2(C)C ; Cc1cccc2c1-c1ccc(N(c3cc(N(c4ccccc4)c4ccccc4)cc(N(c4ccccc4)c4ccccc4)c3)c3ccc(-c4ccccc4)c4ccccc34)cc1C2(C)C ; Cc1cccc2c1-c1ccc(N(c3cc(N(c4ccccc4)c4ccccc4)cc(N(c4ccccc4)c4ccccc4)c3)c3cccc4c(-c5ccccc5)cccc34)cc1C2(C)C]

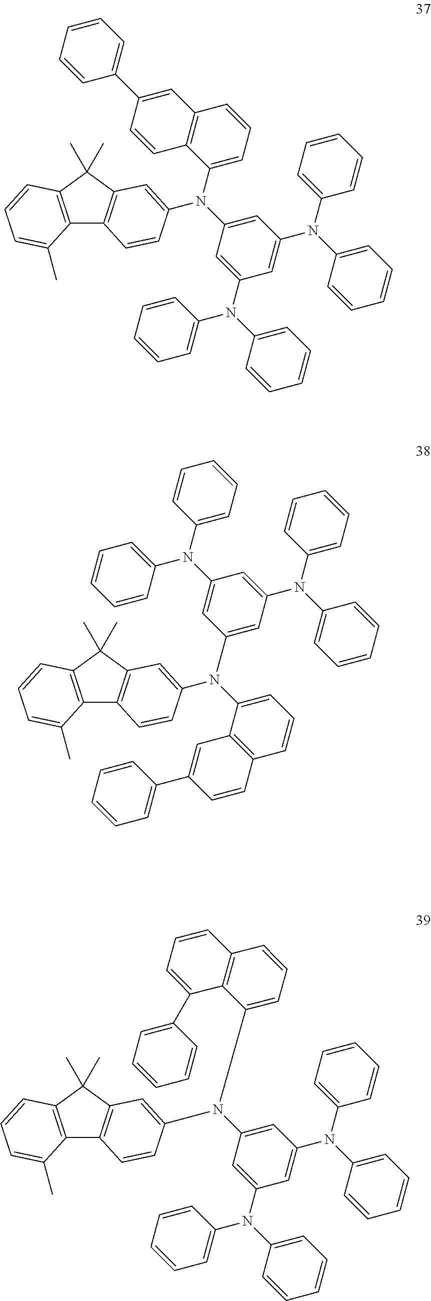 [SMILES: Cc1cccc2c1-c1ccc(N(c3cc(N(c4ccccc4)c4ccccc4)cc(N(c4ccccc4)c4ccccc4)c3)c3cccc4cc(-c5ccccc5)ccc34)cc1C2(C)C ; Cc1cccc2c1-c1ccc(N(c3cc(N(c4ccccc4)c4ccccc4)cc(N(c4ccccc4)c4ccccc4)c3)c3cccc4ccc(-c5ccccc5)cc34)cc1C2(C)C ; Cc1cccc2c1-c1ccc(N(c3cc(N(c4ccccc4)c4ccccc4)cc(N(c4ccccc4)c4ccccc4)c3)c3cccc4cccc(-c5ccccc5)c34)cc1C2(C)C]

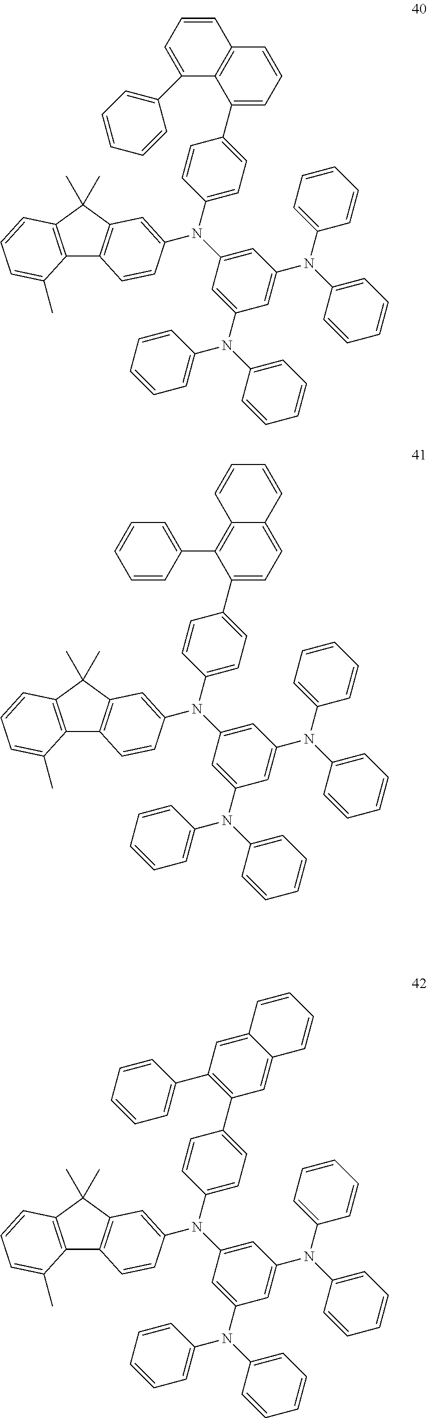

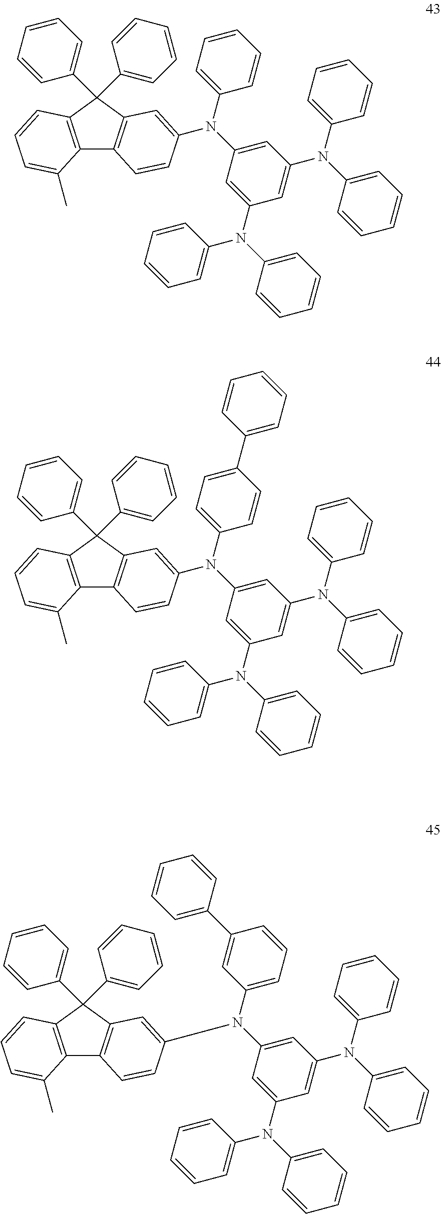 [SMILES: Cc1cccc2c1-c1ccc(N(c3ccccc3)c3cc(N(c4ccccc4)c4ccccc4)cc(N(c4ccccc4)c4ccccc4)c3)cc1C2(c1ccccc1)c1ccccc1 ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(c1ccccc1)c1ccccc1 ; Cc1cccc2c1-c1ccc(N(c3cccc(-c4ccccc4)c3)c3cc(N(c4ccccc4)c4ccccc4)cc(N(c4ccccc4)c4ccccc4)c3)cc1C2(c1ccccc1)c1ccccc1]

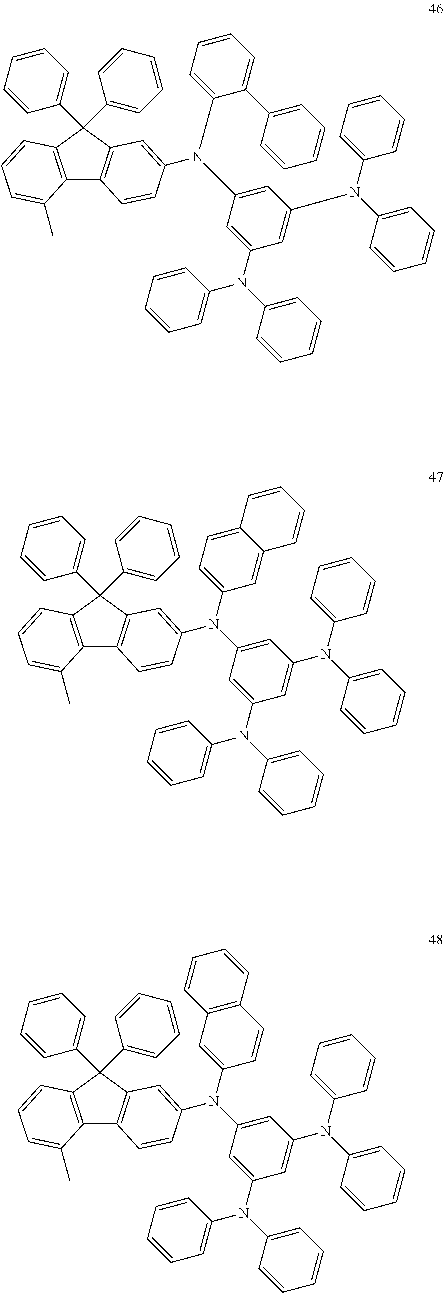 [SMILES: Cc1cccc2c1-c1ccc(N(c3cc(N(c4ccccc4)c4ccccc4)cc(N(c4ccccc4)c4ccccc4)c3)c3ccccc3-c3ccccc3)cc1C2(c1ccccc1)c1ccccc1 ; Cc1cccc2c1-c1ccc(N(c3cc(N(c4ccccc4)c4ccccc4)cc(N(c4ccccc4)c4ccccc4)c3)c3ccc4ccccc4c3)cc1C2(c1ccccc1)c1ccccc1 ; Cc1cccc2c1-c1ccc(N(c3cc(N(c4ccccc4)c4ccccc4)cc(N(c4ccccc4)c4ccccc4)c3)c3ccc4ccccc4c3)cc1C2(c1ccccc1)c1ccccc1]

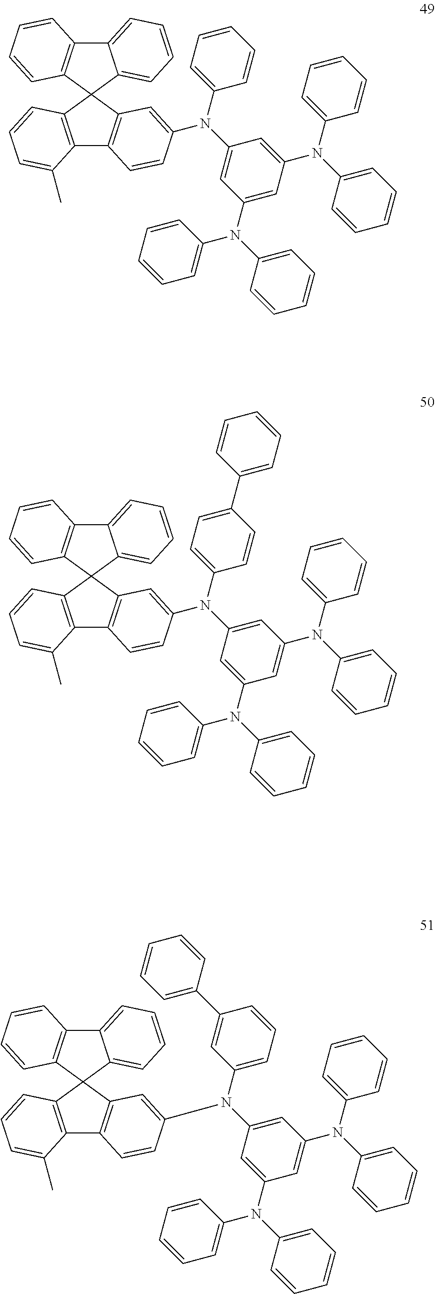 [SMILES: Cc1cccc2c1-c1ccc(N(c3ccccc3)c3cc(N(c4ccccc4)c4ccccc4)cc(N(c4ccccc4)c4ccccc4)c3)cc1C21c2ccccc2-c2ccccc21 ; Cc1cccc2c1-c1ccc(N(c3ccc(-c4ccccc4)cc3)c3cc(N(c4ccccc4)c4ccccc4)cc(N(c4ccccc4)c4ccccc4)c3)cc1C21c2ccccc2-c2ccccc21 ; Cc1cccc2c1-c1ccc(N(c3cccc(-c4ccccc4)c3)c3cc(N(c4ccccc4)c4ccccc4)cc(N(c4ccccc4)c4ccccc4)c3)cc1C21c2ccccc2-c2ccccc21]

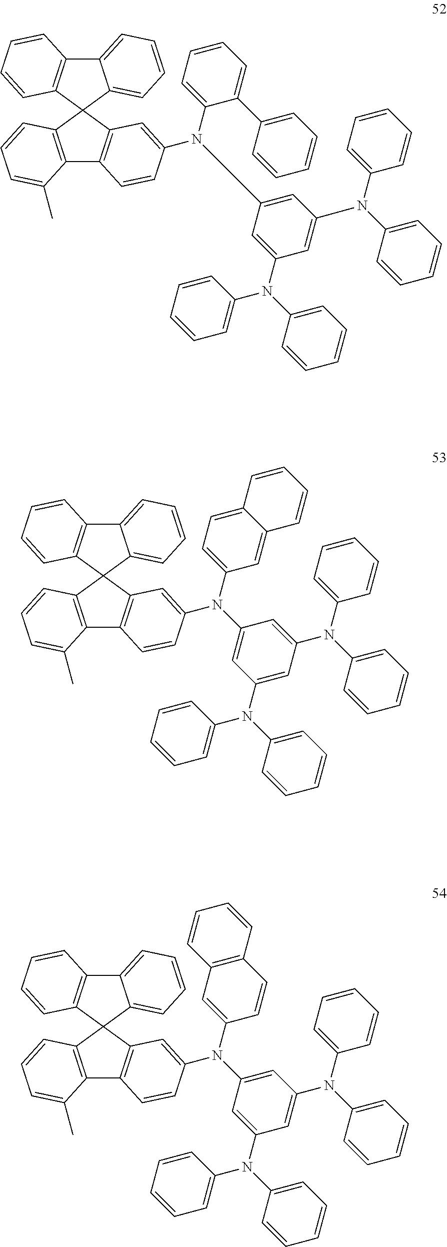 [SMILES: Cc1cccc2c1-c1ccc(N(c3cc(N(c4ccccc4)c4ccccc4)cc(N(c4ccccc4)c4ccccc4)c3)c3ccccc3-c3ccccc3)cc1C21c2ccccc2-c2ccccc21 ; Cc1cccc2c1-c1ccc(N(c3cc(N(c4ccccc4)c4ccccc4)cc(N(c4ccccc4)c4ccccc4)c3)c3ccc4ccccc4c3)cc1C21c2ccccc2-c2ccccc21 ; Cc1cccc2c1-c1ccc(N(c3cc(N(c4ccccc4)c4ccccc4)cc(N(c4ccccc4)c4ccccc4)c3)c3ccc4ccccc4c3)cc1C21c2ccccc2-c2ccccc21]

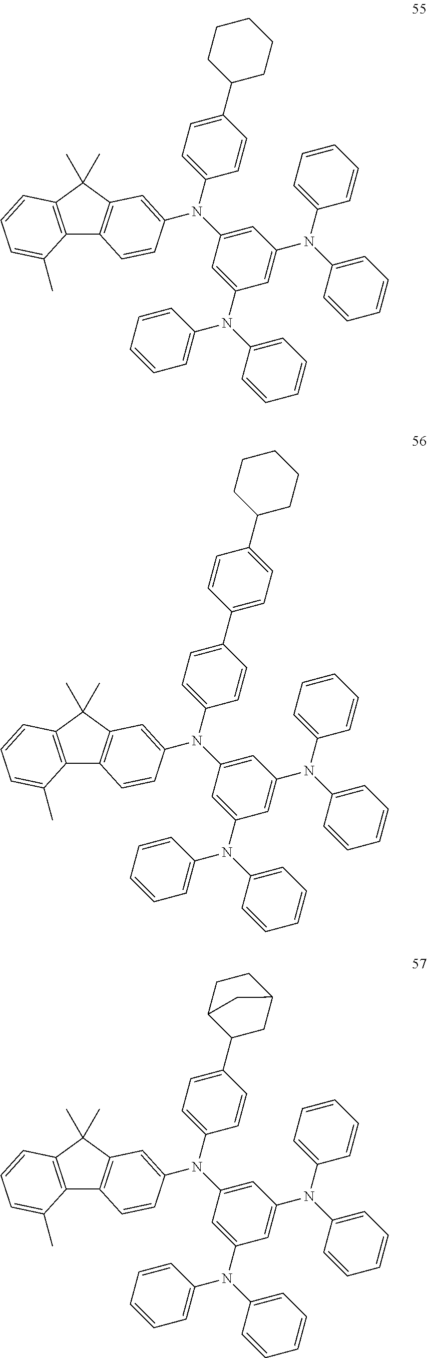

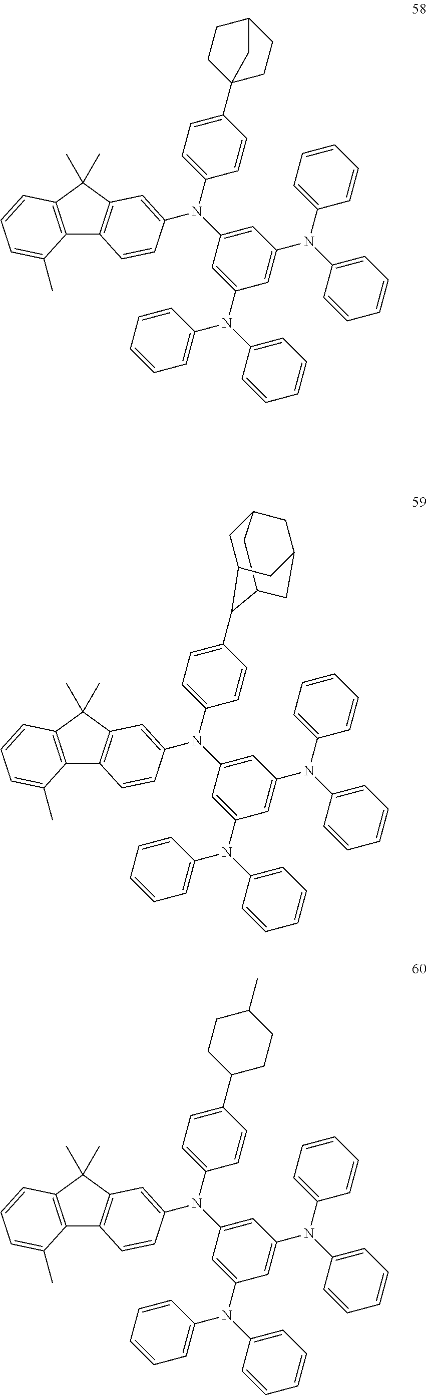 [SMILES: Cc1cccc2c1-c1ccc(N(c3ccc(C45CCC(CC4)C5)cc3)c3cc(N(c4ccccc4)c4ccccc4)cc(N(c4ccccc4)c4ccccc4)c3)cc1C2(C)C ; Cc1cccc2c1-c1ccc(N(c3ccc(C4C5CC6CC(C5)CC4C6)cc3)c3cc(N(c4ccccc4)c4ccccc4)cc(N(c4ccccc4)c4ccccc4)c3)cc1C2(C)C ; Cc1cccc2c1-c1ccc(N(c3ccc(C4CCC(C)CC4)cc3)c3cc(N(c4ccccc4)c4ccccc4)cc(N(c4ccccc4)c4ccccc4)c3)cc1C2(C)C]

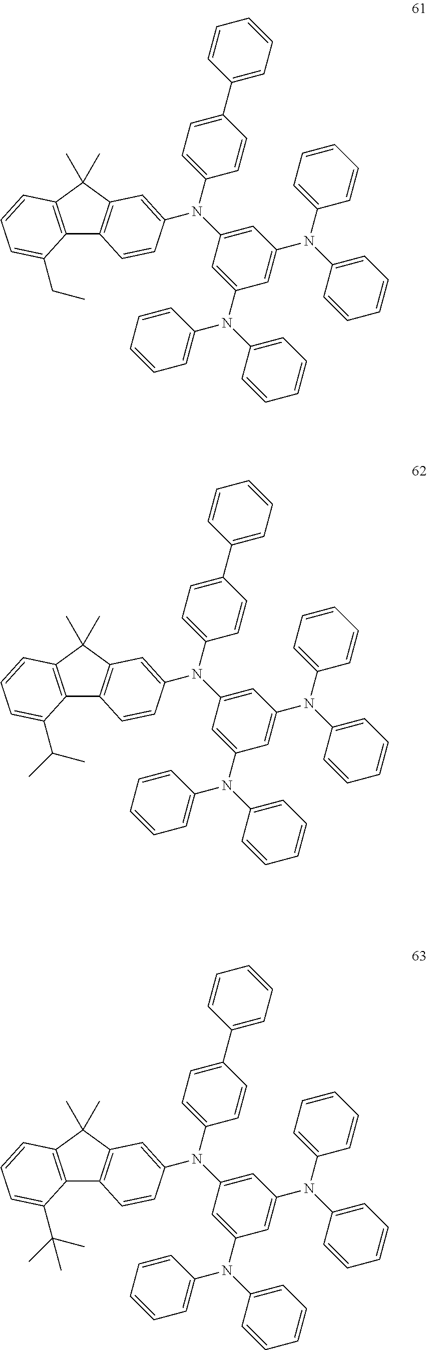 [SMILES: CCc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; CC(C)c1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; CC(C)(C)c1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C]

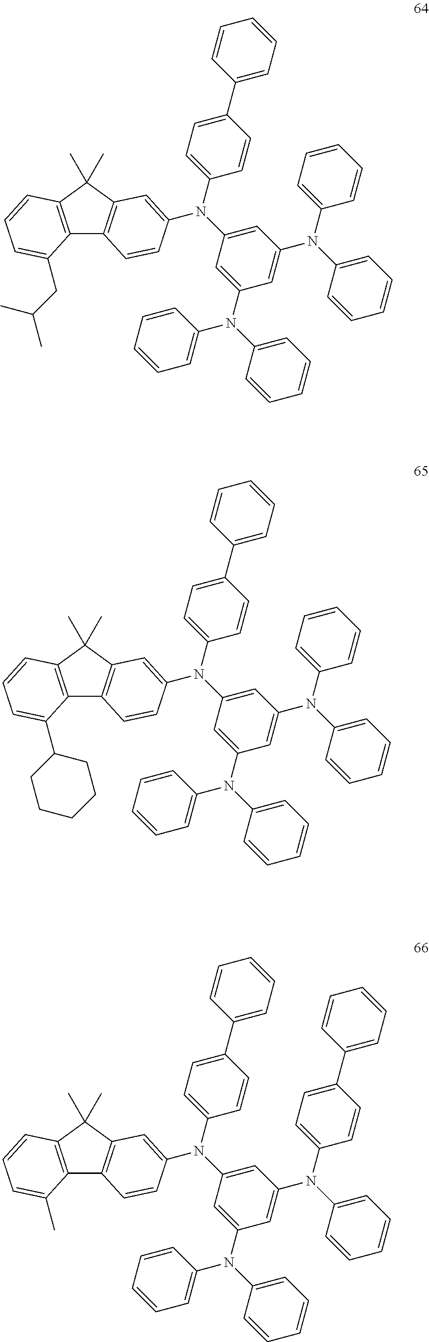 [SMILES: CC(C)Cc1cccc2c1-c1ccc(N(c3ccc(-c4ccccc4)cc3)c3cc(N(c4ccccc4)c4ccccc4)cc(N(c4ccccc4)c4ccccc4)c3)cc1C2(C)C ; CC1(C)c2cc(N(c3ccc(-c4ccccc4)cc3)c3cc(N(c4ccccc4)c4ccccc4)cc(N(c4ccccc4)c4ccccc4)c3)ccc2-c2c(C3CCCCC3)cccc21 ; Cc1cccc2c1-c1ccc(N(c3ccc(-c4ccccc4)cc3)c3cc(N(c4ccccc4)c4ccccc4)cc(N(c4ccccc4)c4ccc(-c5ccccc5)cc4)c3)cc1C2(C)C]

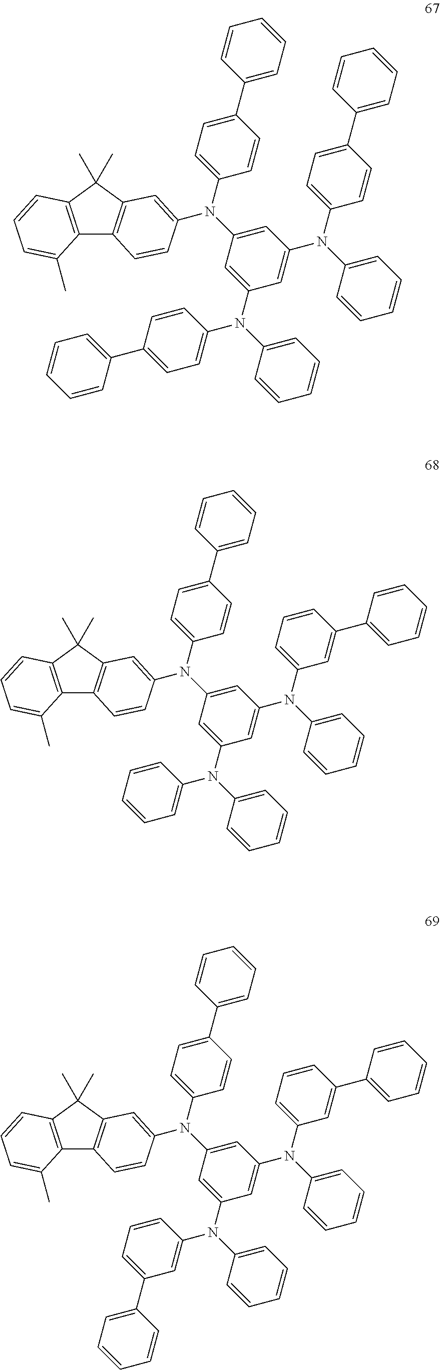 [SMILES: Cc1cccc2c1-c1ccc(N(c3ccc(-c4ccccc4)cc3)c3cc(N(c4ccccc4)c4ccc(-c5ccccc5)cc4)cc(N(c4ccccc4)c4ccc(-c5ccccc5)cc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4cccc(-c5ccccc5)c4)c3)cc1C2(C)C ; Cc1cccc2c1-c1ccc(N(c3ccc(-c4ccccc4)cc3)c3cc(N(c4ccccc4)c4cccc(-c5ccccc5)c4)cc(N(c4ccccc4)c4cccc(-c5ccccc5)c4)c3)cc1C2(C)C]

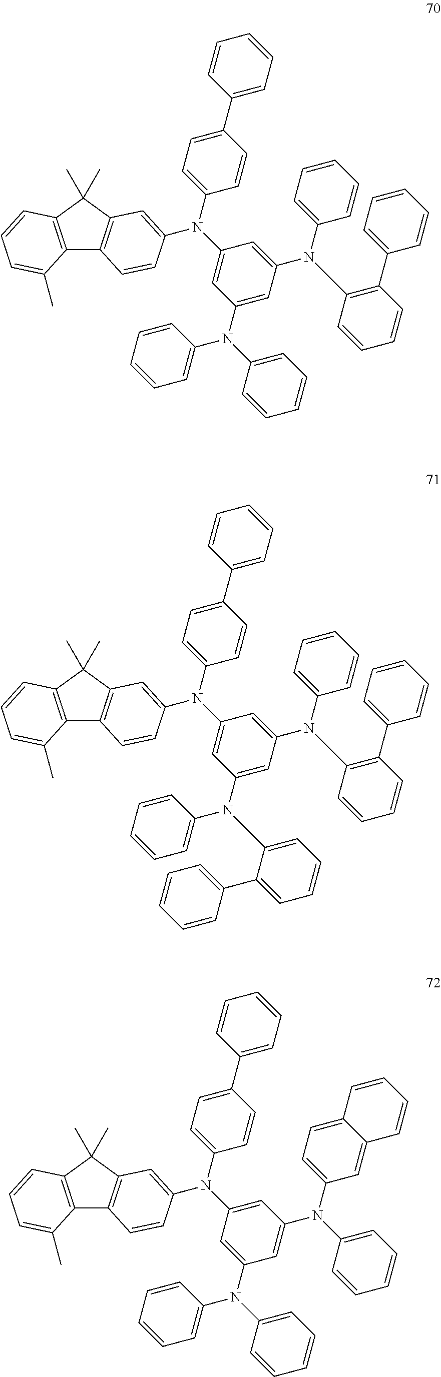 [SMILES: Cc1cccc2c1-c1ccc(N(c3ccc(-c4ccccc4)cc3)c3cc(N(c4ccccc4)c4ccccc4)cc(N(c4ccccc4)c4ccccc4-c4ccccc4)c3)cc1C2(C)C ; Cc1cccc2c1-c1ccc(N(c3ccc(-c4ccccc4)cc3)c3cc(N(c4ccccc4)c4ccccc4-c4ccccc4)cc(N(c4ccccc4)c4ccccc4-c4ccccc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4ccc5ccccc5c4)c3)cc1C2(C)C]

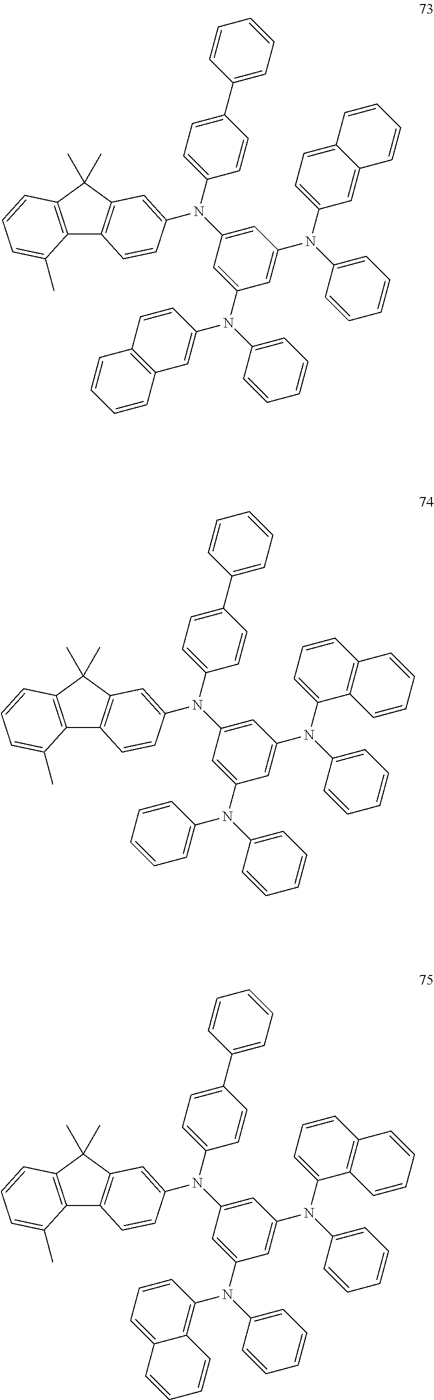 [SMILES: Cc1cccc2c1-c1ccc(N(c3ccc(-c4ccccc4)cc3)c3cc(N(c4ccccc4)c4ccc5ccccc5c4)cc(N(c4ccccc4)c4ccc5ccccc5c4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4cccc5ccccc45)c3)cc1C2(C)C ; Cc1cccc2c1-c1ccc(N(c3ccc(-c4ccccc4)cc3)c3cc(N(c4ccccc4)c4cccc5ccccc45)cc(N(c4ccccc4)c4cccc5ccccc45)c3)cc1C2(C)C]

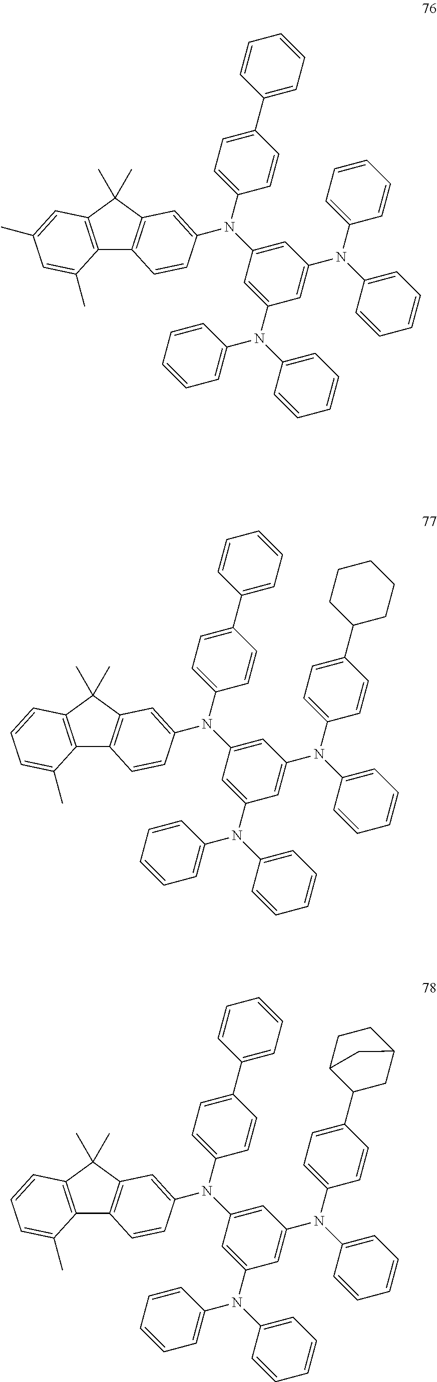 [SMILES: Cc1cc(C)c2c(c1)C(C)(C)c1cc(N(c3ccc(-c4ccccc4)cc3)c3cc(N(c4ccccc4)c4ccccc4)cc(N(c4ccccc4)c4ccccc4)c3)ccc1-2 ; Cc1cccc2c1-c1ccc(N(c3ccc(-c4ccccc4)cc3)c3cc(N(c4ccccc4)c4ccccc4)cc(N(c4ccccc4)c4ccc(C5CCCCC5)cc4)c3)cc1C2(C)C ; Cc1cccc2c1-c1ccc(N(c3ccc(-c4ccccc4)cc3)c3cc(N(c4ccccc4)c4ccccc4)cc(N(c4ccccc4)c4ccc(C5CC6CCC5C6)cc4)c3)cc1C2(C)C]

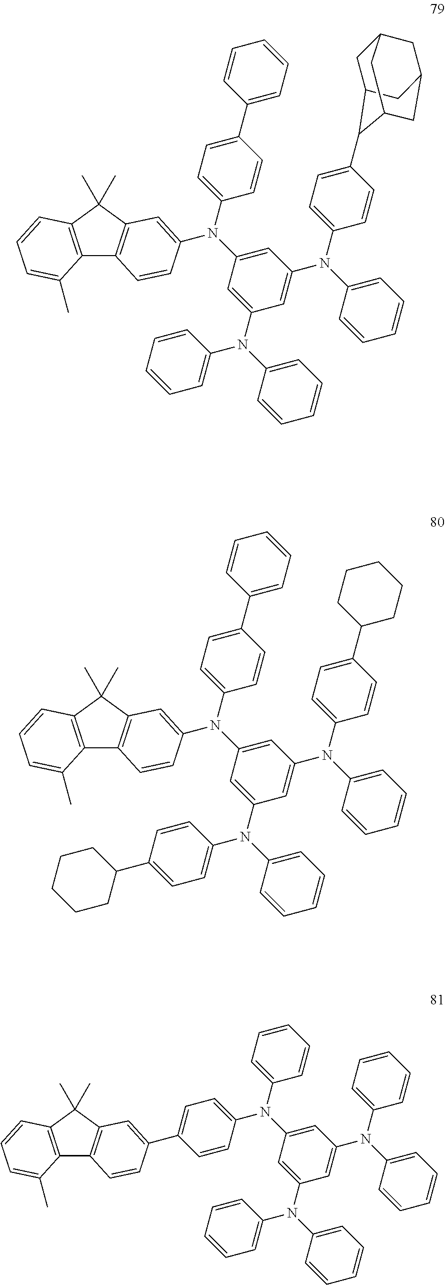 [SMILES: Cc1cccc2c1-c1ccc(N(c3ccc(-c4ccccc4)cc3)c3cc(N(c4ccccc4)c4ccccc4)cc(N(c4ccccc4)c4ccc(C5C6CC7CC(C6)CC5C7)cc4)c3)cc1C2(C)C ; Cc1cccc2c1-c1ccc(N(c3ccc(-c4ccccc4)cc3)c3cc(N(c4ccccc4)c4ccc(C5CCCCC5)cc4)cc(N(c4ccccc4)c4ccc(C5CCCCC5)cc4)c3)cc1C2(C)C ; Cc1cccc2c1-c1ccc(-c3ccc(N(c4ccccc4)c4cc(N(c5ccccc5)c5ccccc5)cc(N(c5ccccc5)c5ccccc5)c4)cc3)cc1C2(C)C]

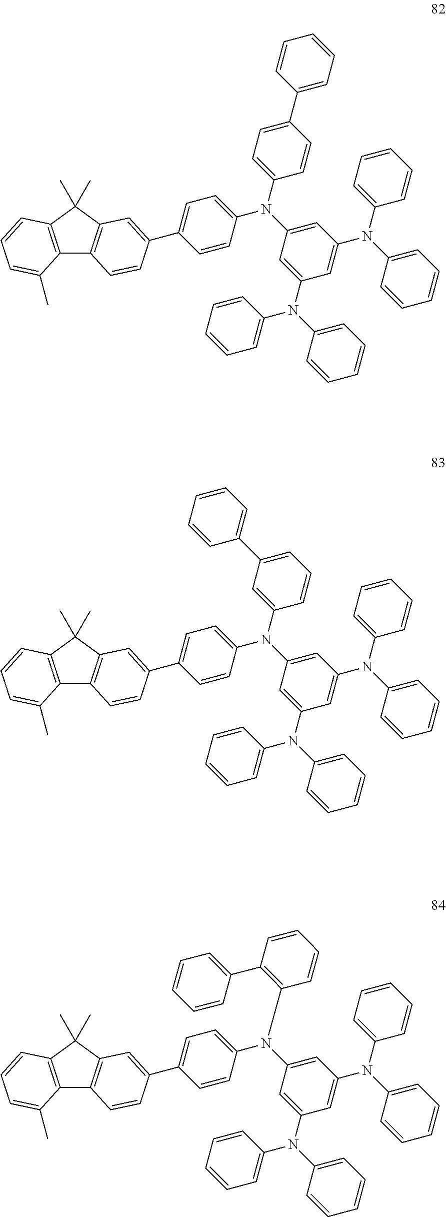 [SMILES: Cc1cccc2c1-c1ccc(-c3ccc(N(c4ccc(-c5ccccc5)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4cccc(-c5ccccc5)c4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4cc(N(c5ccccc5)c5ccccc5)cc(N(c5ccccc5)c5ccccc5)c4)c4ccccc4-c4ccccc4)cc3)cc1C2(C)C]

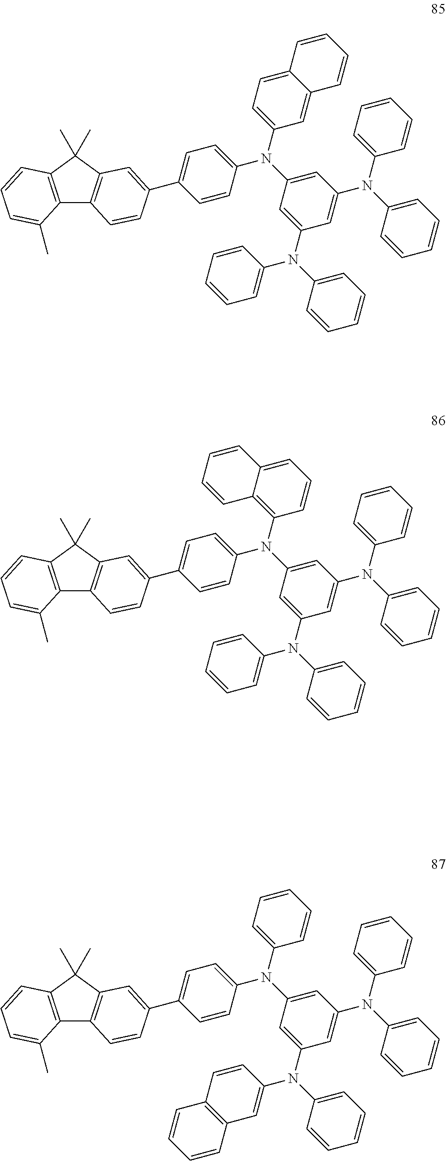 [SMILES: Cc1cccc2c1-c1ccc(-c3ccc(N(c4cc(N(c5ccccc5)c5ccccc5)cc(N(c5ccccc5)c5ccccc5)c4)c4ccc5ccccc5c4)cc3)cc1C2(C)C ; Cc1cccc2c1-c1ccc(-c3ccc(N(c4cc(N(c5ccccc5)c5ccccc5)cc(N(c5ccccc5)c5ccccc5)c4)c4cccc5ccccc45)cc3)cc1C2(C)C ; Cc1cccc2c1-c1ccc(-c3ccc(N(c4ccccc4)c4cc(N(c5ccccc5)c5ccccc5)cc(N(c5ccccc5)c5ccc6ccccc6c5)c4)cc3)cc1C2(C)C]

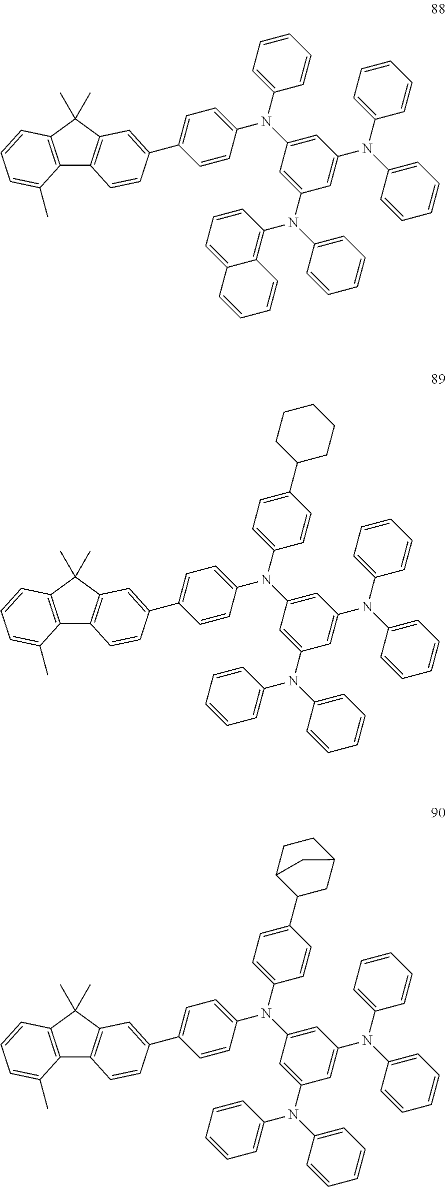 [SMILES: Cc1cccc2c1-c1ccc(-c3ccc(N(c4ccccc4)c4cc(N(c5ccccc5)c5ccccc5)cc(N(c5ccccc5)c5cccc6ccccc56)c4)cc3)cc1C2(C)C ; Cc1cccc2c1-c1ccc(-c3ccc(N(c4ccc(C5CCCCC5)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4ccc(C5CC6CCC5C6)cc4)c4cc(N(c5ccccc5)c5ccccc5)cc(N(c5ccccc5)c5ccccc5)c4)cc3)cc1C2(C)C]

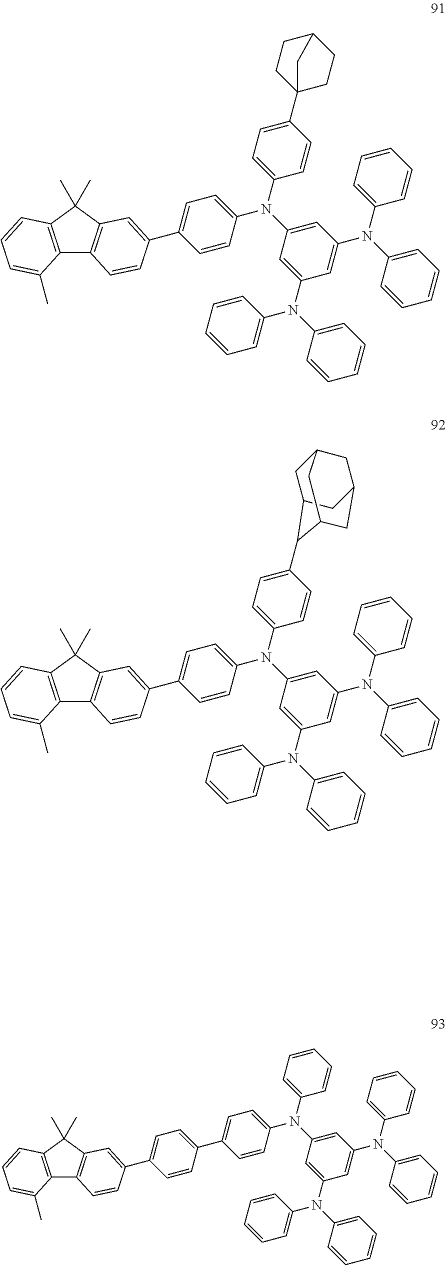 [SMILES: Cc1cccc2c1-c1ccc(-c3ccc(N(c4ccc(C56CCC(CC5)C6)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(N(c4ccc(C5C6CC7CC(C6)CC5C7)cc4)c4cc(N(c5ccccc5)c5ccccc5)cc(N(c5ccccc5)c5ccccc5)c4)cc3)cc1C2(C)C ; Cc1cccc2c1-c1ccc(-c3ccc(-c4ccc(N(c5ccccc5)c5cc(N(c6ccccc6)c6ccccc6)cc(N(c6ccccc6)c6ccccc6)c5)cc4)cc3)cc1C2(C)C]

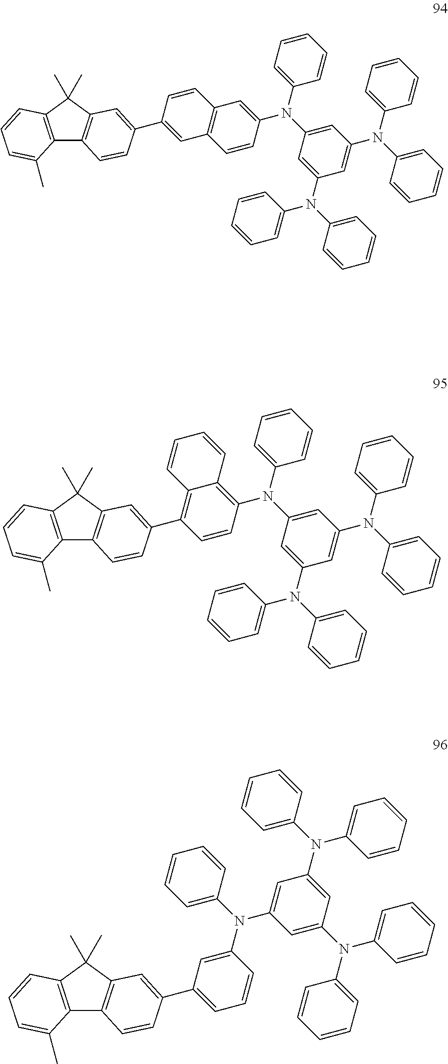 [SMILES: Cc1cccc2c1-c1ccc(-c3ccc4cc(N(c5ccccc5)c5cc(N(c6ccccc6)c6ccccc6)cc(N(c6ccccc6)c6ccccc6)c5)ccc4c3)cc1C2(C)C ; Cc1cccc2c1-c1ccc(-c3ccc(N(c4ccccc4)c4cc(N(c5ccccc5)c5ccccc5)cc(N(c5ccccc5)c5ccccc5)c4)c4ccccc34)cc1C2(C)C ; Cc1cccc2c1-c1ccc(-c3cccc(N(c4ccccc4)c4cc(N(c5ccccc5)c5ccccc5)cc(N(c5ccccc5)c5ccccc5)c4)c3)cc1C2(C)C]

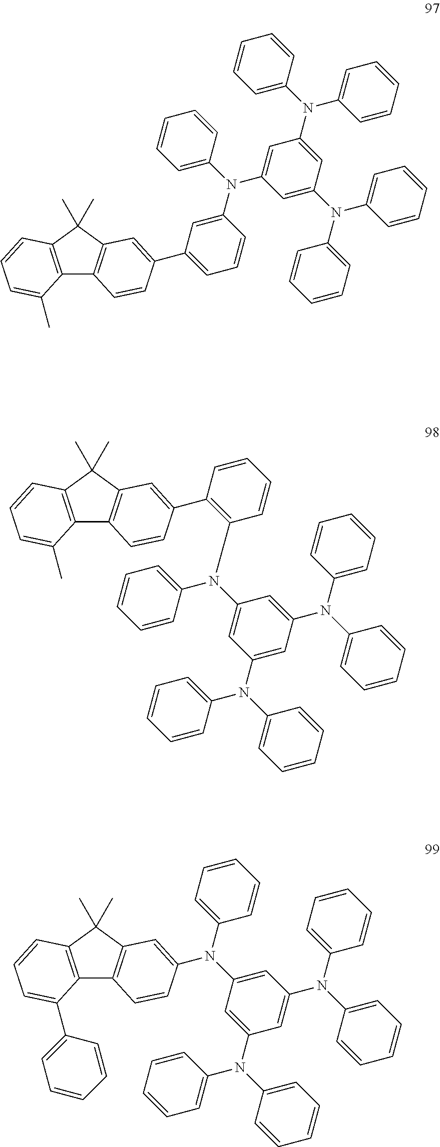 [SMILES: Cc1cccc2c1-c1ccc(-c3cccc(N(c4ccccc4)c4cc(N(c5ccccc5)c5ccccc5)cc(N(c5ccccc5)c5ccccc5)c4)c3)cc1C2(C)C ; Cc1cccc2c1-c1ccc(-c3ccccc3N(c3ccccc3)c3cc(N(c4ccccc4)c4ccccc4)cc(N(c4ccccc4)c4ccccc4)c3)cc1C2(C)C ; CC1(C)c2cc(N(c3ccccc3)c3cc(N(c4ccccc4)c4ccccc4)cc(N(c4ccccc4)c4ccccc4)c3)ccc2-c2c(-c3ccccc3)cccc21]

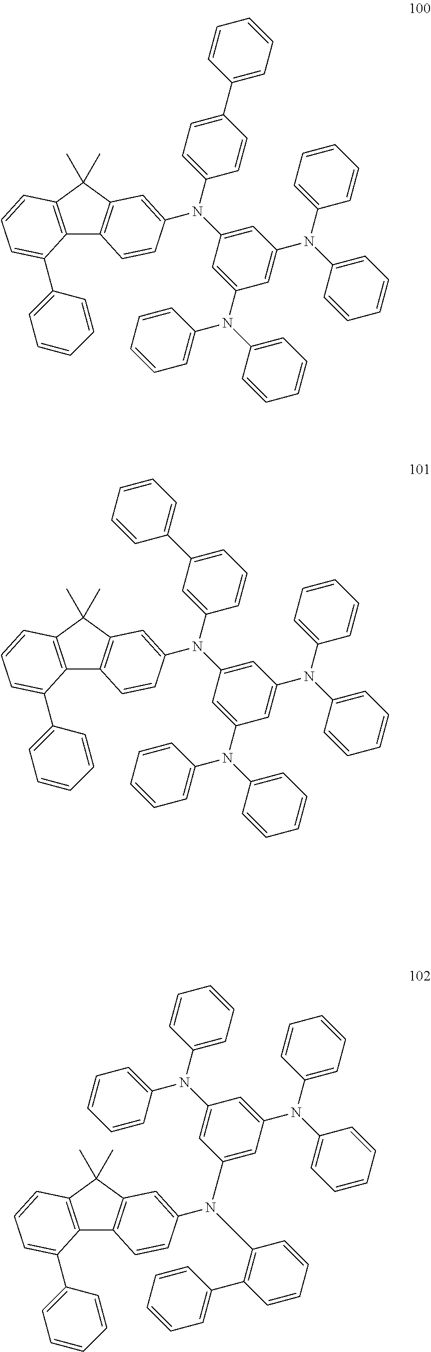 [SMILES: CC1(C)c2cc(N(c3ccc(-c4ccccc4)cc3)c3cc(N(c4ccccc4)c4ccccc4)cc(N(c4ccccc4)c4ccccc4)c3)ccc2-c2c(-c3ccccc3)cccc21 ; CC1(C)c2cc(N(c3cccc(-c4ccccc4)c3)c3cc(N(c4ccccc4)c4ccccc4)cc(N(c4ccccc4)c4ccccc4)c3)ccc2-c2c(-c3ccccc3)cccc21 ; CC1(C)c2cc(N(c3cc(N(c4ccccc4)c4ccccc4)cc(N(c4ccccc4)c4ccccc4)c3)c3ccccc3-c3ccccc3)ccc2-c2c(-c3ccccc3)cccc21]

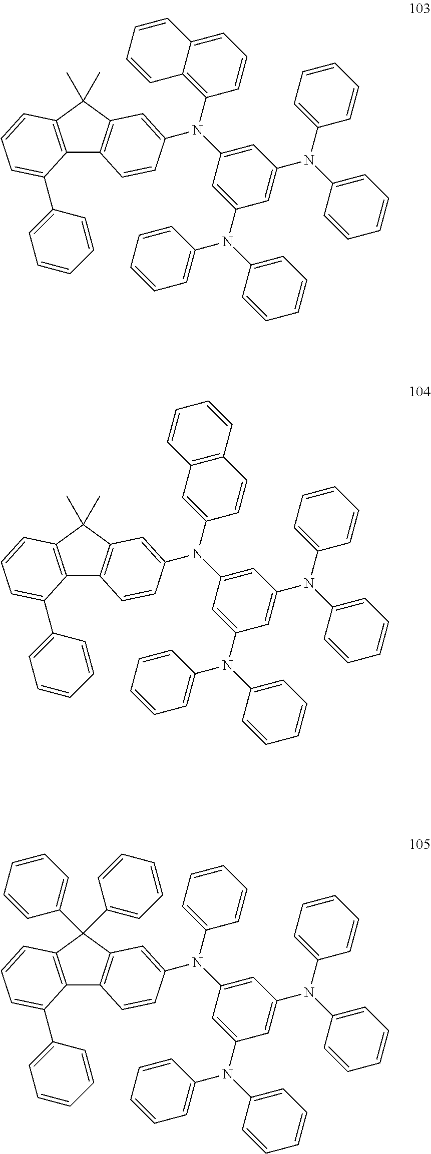 [SMILES: CC1(C)c2cc(N(c3cc(N(c4ccccc4)c4ccccc4)cc(N(c4ccccc4)c4ccccc4)c3)c3cccc4ccccc34)ccc2-c2c(-c3ccccc3)cccc21 ; CC1(C)c2cc(N(c3cc(N(c4ccccc4)c4ccccc4)cc(N(c4ccccc4)c4ccccc4)c3)c3ccc4ccccc4c3)ccc2-c2c(-c3ccccc3)cccc21 ; c1ccc(-c2cccc3c2-c2ccc(N(c4ccccc4)c4cc(N(c5ccccc5)c5ccccc5)cc(N(c5ccccc5)c5ccccc5)c4)cc2C3(c2ccccc2)c2ccccc2)cc1]

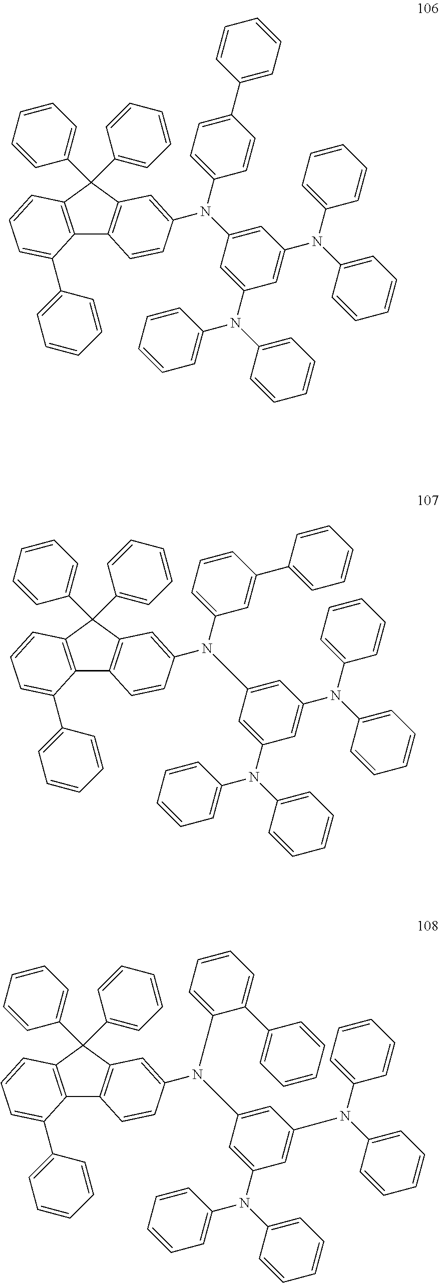 [SMILES: c1ccc(-c2ccc(N(c3cc(N(c4ccccc4)c4ccccc4)cc(N(c4ccccc4)c4ccccc4)c3)c3ccc4c(c3)C(c3ccccc3)(c3ccccc3)c3cccc(-c5ccccc5)c3-4)cc2)cc1 ; c1ccc(-c2cccc(N(c3cc(N(c4ccccc4)c4ccccc4)cc(N(c4ccccc4)c4ccccc4)c3)c3ccc4c(c3)C(c3ccccc3)(c3ccccc3)c3cccc(-c5ccccc5)c3-4)c2)cc1 ; c1ccc(-c2ccccc2N(c2cc(N(c3ccccc3)c3ccccc3)cc(N(c3ccccc3)c3ccccc3)c2)c2ccc3c(c2)C(c2ccccc2)(c2ccccc2)c2cccc(-c4ccccc4)c2-3)cc1]

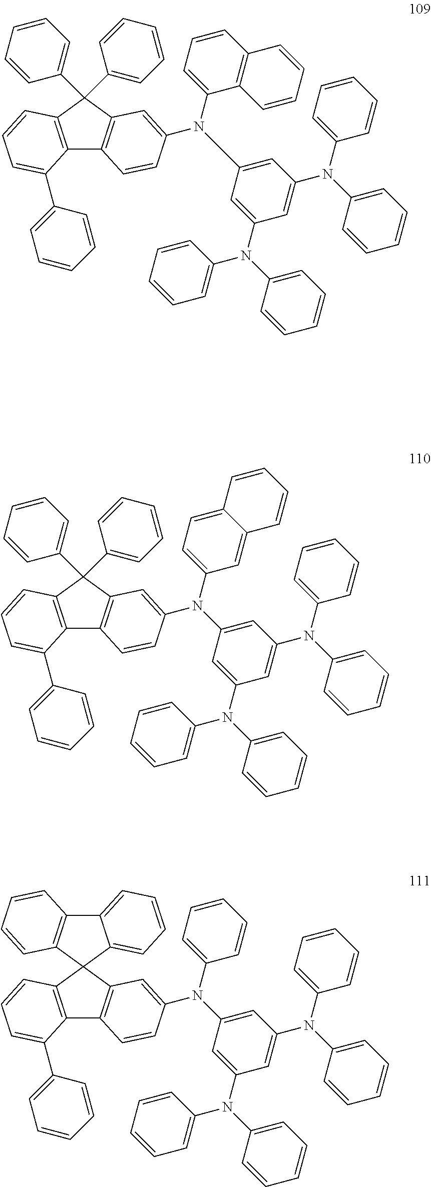 [SMILES: c1ccc(-c2cccc3c2-c2ccc(N(c4cc(N(c5ccccc5)c5ccccc5)cc(N(c5ccccc5)c5ccccc5)c4)c4cccc5ccccc45)cc2C3(c2ccccc2)c2ccccc2)cc1 ; c1ccc(-c2cccc3c2-c2ccc(N(c4cc(N(c5ccccc5)c5ccccc5)cc(N(c5ccccc5)c5ccccc5)c4)c4ccc5ccccc5c4)cc2C3(c2ccccc2)c2ccccc2)cc1 ; c1ccc(-c2cccc3c2-c2ccc(N(c4ccccc4)c4cc(N(c5ccccc5)c5ccccc5)cc(N(c5ccccc5)c5ccccc5)c4)cc2C32c3ccccc3-c3ccccc32)cc1]

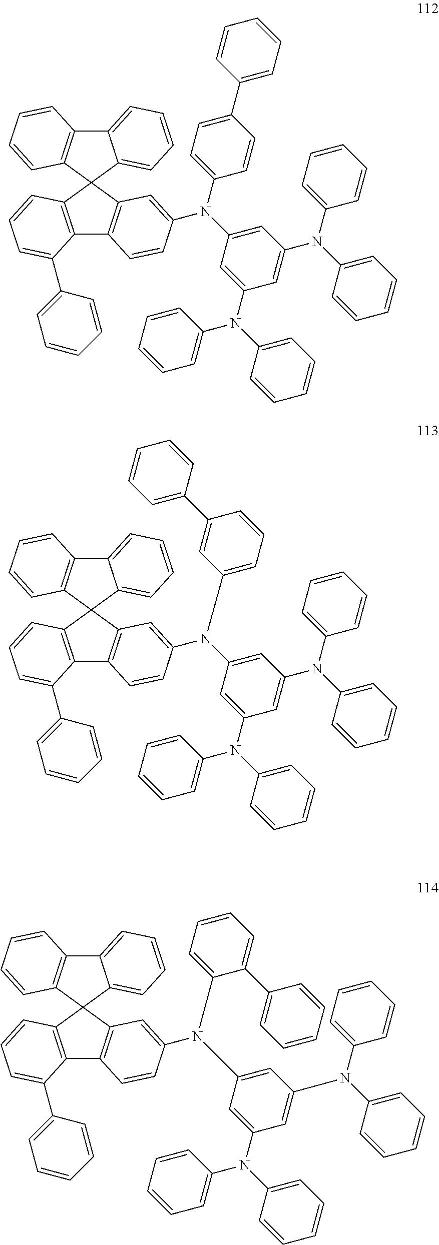 [SMILES: c1ccc(-c2ccc(N(c3cc(N(c4ccccc4)c4ccccc4)cc(N(c4ccccc4)c4ccccc4)c3)c3ccc4c(c3)C3(c5ccccc5-c5ccccc53)c3cccc(-c5ccccc5)c3-4)cc2)cc1 ; c1ccc(-c2cccc(N(c3cc(N(c4ccccc4)c4ccccc4)cc(N(c4ccccc4)c4ccccc4)c3)c3ccc4c(c3)C3(c5ccccc5-c5ccccc53)c3cccc(-c5ccccc5)c3-4)c2)cc1 ; c1ccc(-c2ccccc2N(c2cc(N(c3ccccc3)c3ccccc3)cc(N(c3ccccc3)c3ccccc3)c2)c2ccc3c(c2)C2(c4ccccc4-c4ccccc42)c2cccc(-c4ccccc4)c2-3)cc1]

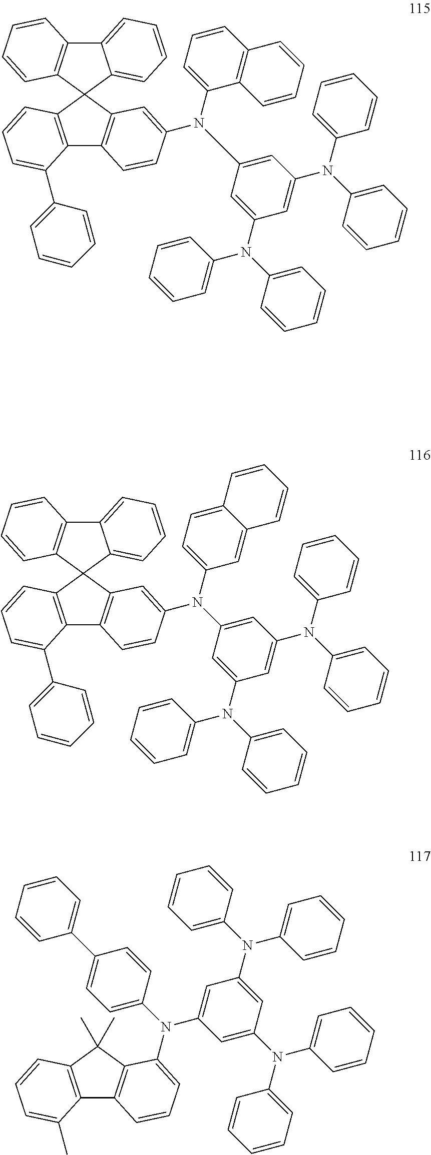 [SMILES: c1ccc(-c2cccc3c2-c2ccc(N(c4cc(N(c5ccccc5)c5ccccc5)cc(N(c5ccccc5)c5ccccc5)c4)c4cccc5ccccc45)cc2C32c3ccccc3-c3ccccc32)cc1 ; c1ccc(-c2cccc3c2-c2ccc(N(c4cc(N(c5ccccc5)c5ccccc5)cc(N(c5ccccc5)c5ccccc5)c4)c4ccc5ccccc5c4)cc2C32c3ccccc3-c3ccccc32)cc1 ; Cc1cccc2c1-c1cccc(N(c3ccc(-c4ccccc4)cc3)c3cc(N(c4ccccc4)c4ccccc4)cc(N(c4ccccc4)c4ccccc4)c3)c1C2(C)C]

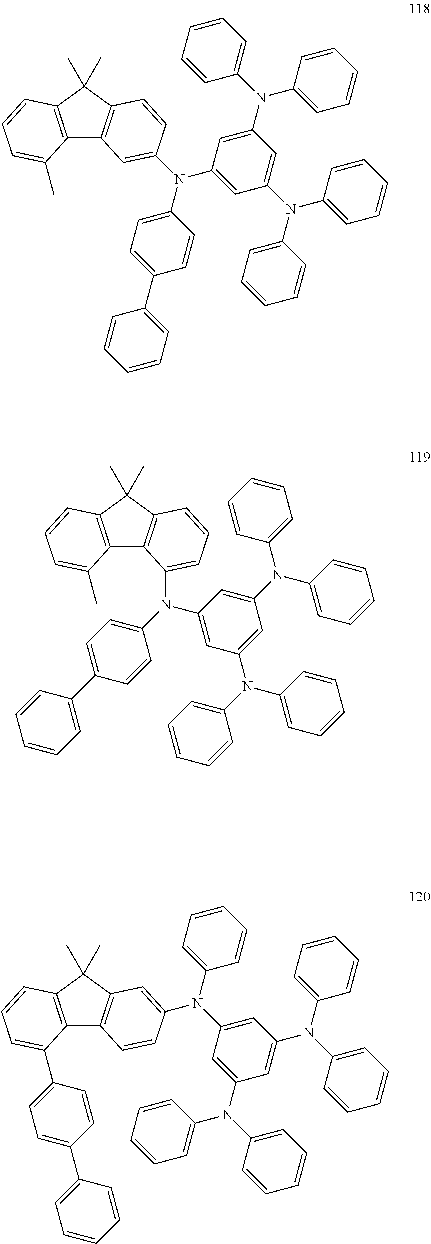 [SMILES: Cc1cccc2c1-c1cc(N(c3ccc(-c4ccccc4)cc3)c3cc(N(c4ccccc4)c4ccccc4)cc(N(c4ccccc4)c4ccccc4)c3)ccc1C2(C)C ; Cc1cccc2c1-c1c(N(c3ccc(-c4ccccc4)cc3)c3cc(N(c4ccccc4)c4ccccc4)cc(N(c4ccccc4)c4ccccc4)c3)cccc1C2(C)C ; CC1(C)c2cc(N(c3ccccc3)c3cc(N(c4ccccc4)c4ccccc4)cc(N(c4ccccc4)c4ccccc4)c3)ccc2-c2c(-c3ccc(-c4ccccc4)cc3)cccc21]

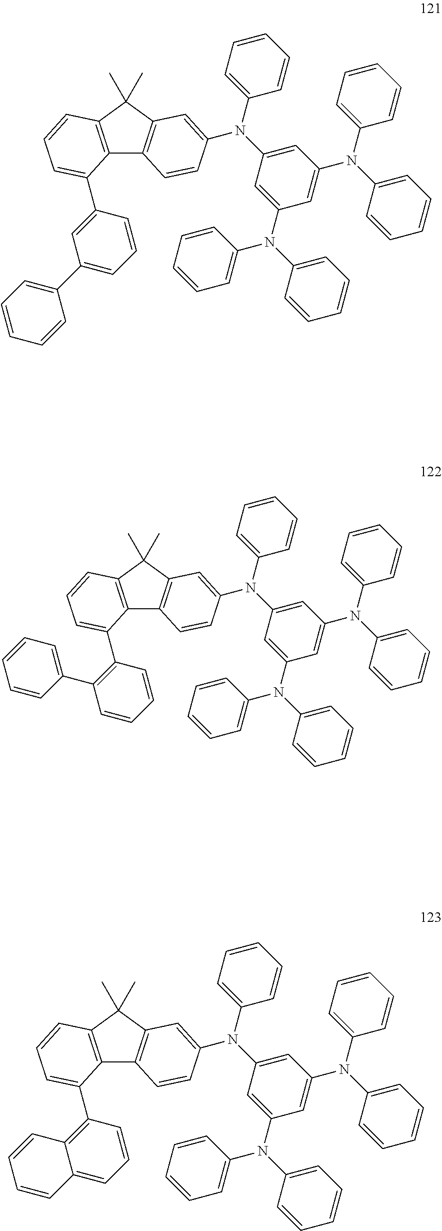 [SMILES: CC1(C)c2cc(N(c3ccccc3)c3cc(N(c4ccccc4)c4ccccc4)cc(N(c4ccccc4)c4ccccc4)c3)ccc2-c2c(-c3cccc(-c4ccccc4)c3)cccc21 ; CC1(C)c2cc(N(c3ccccc3)c3cc(N(c4ccccc4)c4ccccc4)cc(N(c4ccccc4)c4ccccc4)c3)ccc2-c2c(-c3ccccc3-c3ccccc3)cccc21 ; CC1(C)c2cc(N(c3ccccc3)c3cc(N(c4ccccc4)c4ccccc4)cc(N(c4ccccc4)c4ccccc4)c3)ccc2-c2c(-c3cccc4ccccc34)cccc21]

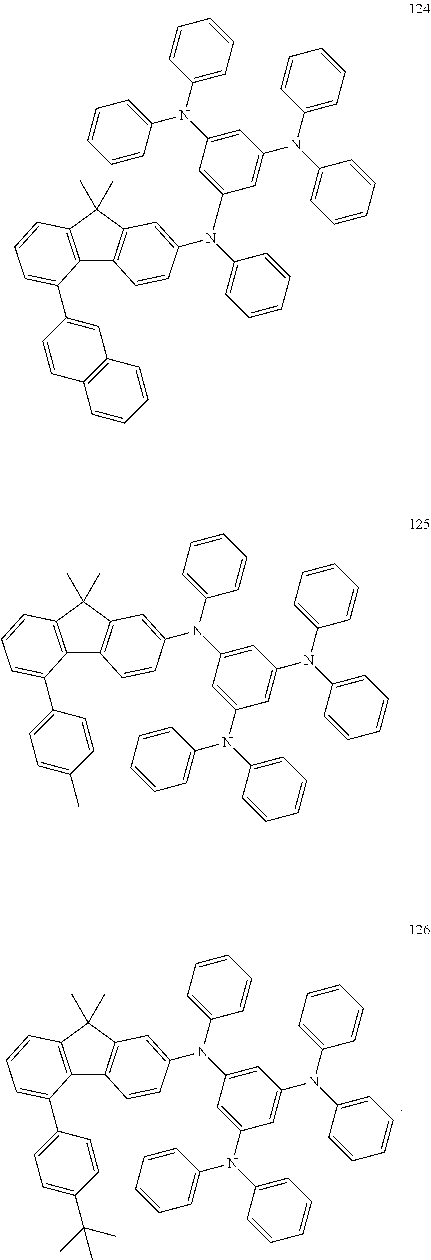 [SMILES: CC1(C)c2cc(N(c3ccccc3)c3cc(N(c4ccccc4)c4ccccc4)cc(N(c4ccccc4)c4ccccc4)c3)ccc2-c2c(-c3ccc4ccccc4c3)cccc21 ; Cc1ccc(-c2cccc3c2-c2ccc(N(c4ccccc4)c4cc(N(c5ccccc5)c5ccccc5)cc(N(c5ccccc5)c5ccccc5)c4)cc2C3(C)C)cc1 ; CC(C)(C)c1ccc(-c2cccc3c2-c2ccc(N(c4ccccc4)c4cc(N(c5ccccc5)c5ccccc5)cc(N(c5ccccc5)c5ccccc5)c4)cc2C3(C)C)cc1]

10. A light-emitting device comprising:
a first electrode;
a second electrode facing the first electrode; and
an interlayer between the first electrode and the second electrode and comprising an emission layer,
wherein the interlayer comprises the compound of claim 1.

11. The light-emitting device of claim 10, wherein the first electrode is an anode,
the second electrode is a cathode, and
the interlayer further comprises a hole transport region between the first electrode and the emission layer and comprising:
a hole transport layer; and
a hole injection layer, an emission auxiliary layer, an electron blocking layer, or any combination thereof.

12. The light-emitting device of claim 11, wherein the hole transport layer comprises the compound.

13. The light-emitting device of claim 10, wherein the first electrode is an anode,
the second electrode is a cathode, and
the interlayer further comprises an electron transport region between the second electrode and the emission layer and comprising a hole blocking layer, an electron transport layer, an electron injection layer, or any combination thereof.

14. The light-emitting device of claim 10, wherein the emission layer comprises two types of hosts.

15. The light-emitting device of claim 10, wherein the emission layer comprises a hole transporting host and an electron transporting host.

16. The light-emitting device of claim 10, wherein the emission layer comprises two types of dopants.

17. The light-emitting device of claim 10, wherein the emission layer comprises a phosphorescent dopant and a fluorescent dopant.

18. An electronic apparatus comprising the light-emitting device of claim 10.

19. The electronic apparatus of claim 18, further comprising a thin-film transistor,
wherein the thin-film transistor comprises a source electrode and a drain electrode, and
the first electrode of the light-emitting device is electrically connected to the source electrode or the drain electrode of the thin-film transistor.

20. An electronic equipment comprising the electronic apparatus of claim 18, wherein the electronic equipment is at least one selected from among a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented reality display, a vehicle, a video wall comprising multiple displays tiled together, a theater or stadium screen, a phototherapy device, a signboard, and combinations thereof.
